(19) United States
(12) Patent Application Publication
(10) Pub. No.: US 2021/0330744 A1
(43) Pub. Date: Oct. 28, 2021

(54) Methods and Compositions for Treating Neurodegeneration and Fibrosis

(71) Applicant: Temple University-Of The Commonwealth System of Higher Education, Philadelphia, PA (US)

(72) Inventors: John W. Elrod, Philadelphia, PA (US); Pooja Jadiya, Philadelphia, PA (US); Alyssa A. Lombardi, Philadelphia, PA (US); Timothy S. Luongo, Philadelphia, PA (US)

(21) Appl. No.: 16/484,953

(22) PCT Filed: Feb. 12, 2018
(86) PCT No.: PCT/US18/17766

§ 371 Date: Aug. 9, 2019

Related U.S. Application Data
(60) Provisional application No. 62/457,578, filed on Feb. 10, 2017.

(51) Int. Cl. A61K 38/17; A61P 25/28; A61K 31/7105; C07K 16/24; A61K 35/761

(52) U.S. Cl. CPC A61K 38/1738; A61P 25/28; A61K 48/00; C07K 16/24; A61K 35/761; A61K 31/7105

(57) ABSTRACT

This invention is generally related to novel compositions and methods for treating or preventing fibrosis, diseases or disorders associated with fibrosis, neurodegeneration, diseases or disorders associated with neurodegeneration and cardiovascular disease or disorders.

### CROSS-REFERENCE TO RELATED APPLICATIONS

The present application claims priority to U.S. Provisional Application No. 62/457,578, filed Feb. 10, 2017, which is hereby incorporated by reference in its entirety herein.

### BACKGROUND

Fibrosis is a disease or disorder eliciting abnormal formation, accumulation and precipitation of an extracellular matrix. Cardiac fibroblasts make up a significant portion of the adult heart and play a pivotal role in regulating the structural integrity of the heart by maintaining the extracellular matrix as well as coordinating cell-to-cell and cell-to-matrix interactions. In addition to this important physiological function, when the heart is injured fibroblasts transition from a quiescent structural role into contractile and synthetic myofibroblasts. This is crucial for the initial healing response, for example scar formation to prevent ventricular wall rupture after myocardial infarction, but excessive fibrosis is maladaptive, impairs cardiac function and contributes to heart failure progression. While cytosolic calcium (ᵢCa²⁺) elevation has been shown to be necessary for myofibroblast transdifferentiation, other Ca²⁺ domains have not been explored. Recent studies have reported that the Mcu gene encodes the channel-forming portion of the mitochondrial calcium uniporter complex (MCU) and is required for acute mitochondrial calcium (ₘCa²⁺) uptake. Mitochondria are theorized to buffer significant amounts of ᵢCa²⁺ in non-excitable cells and they also serve as a bioenergetic control point of cellular metabolism. In addition, metabolic switching is thought be a key signal driving cellular differentiation in numerous tissue types. Currently, there are no good drugs or treatments for fibrosis.

Alzheimer's disease (AD) is characterized by neurodegeneration, specifically the progressive loss of neuronal populations in the frontal cortex and hippocampus. Numerous studies have shown that neuronal cell death and metabolic dysregulation are fundamental cellular mechanisms driving the progression of AD and other dementia-related diseases. Previous studies have suggested numerous mechanisms whereby intracellular Ca²⁺ load is increased in AD and thereby likely significantly impacts ₘCa²⁺ signaling. Currently, there are no effective treatments for neurodegeneration or Alzheimer's.

Thus, there is a need in the art for compositions and methods for treating fibrosis and diseases or disorders associated with fibrosis and neurodegeneration and diseases or disorders associated with neurodegeneration. The present invention satisfies this need.

### SUMMARY OF THE INVENTION

In one aspect, the invention provides a method for treating or preventing neurodegeneration or a neurodegeneration-related disease or disorder. In one embodiment, the method comprises administering a composition comprising an activator of mitochondrial Na⁺/Ca²⁺ exchanger (mNCX) to a subject in need thereof. In one embodiment, the activator increases one or more of transcription, translation, and activity of mNCX. In one embodiment, the activator is selected from the group consisting of a chemical compound, a protein, a peptide, a peptidomemetic, an antibody, a ribozyme, a small molecule chemical compound, a nucleic acid, a vector, and an antisense nucleic acid molecule.

In one embodiment, the neurodegeneration-related disease or disorder is selected from the group consisting of Alzheimer's Disease, amyotrophic lateral sclerosis, Parkinson's, Alzheimer's, Huntington's, Batten disease, prion disease, motor neuron diseases, traumatic brain injury, blast injury, dementia, Tay-Sachs, Niemann-Pick, PDH deficiency, aggregation disorders, encephalopathies, ataxia disorders, and neurodegeneration associated with aging.

In one aspect, the invention provides a method fort treating or preventing fibrosis or a fibrosis-related disease or disorder. In one embodiment, the method comprises administering a composition comprising a modulator of a target to a subject in need thereof. In one embodiment, the target is selected from the group consisting of mitochondrial Na⁺/Ca²⁺ exchanger (mNCX), a PDH kinase, a PDH phosphatase, an alpha-ketoglutarate dependent demethylase, phosphofructokinase-2 (PFK-2), calcium sensitive alpha-ketoglutarate dehydrogenase, and the ratio of alpha-ketoglutarate to succinate. In one embodiment, the alpha-ketoglutarate dependent demethylase is selected from the group consisting of a Ten-eleven translocation (TET) enzyme and a JmjC-domain containing histone demethylase (JHDM).

In one embodiment, the modulator is an activator. In one embodiment, the modulator is an inhibitor. In one embodiment, inhibitor prevents one or more of transcription, translation, and activity of mNCX. In one embodiment, the modulator is selected from the group consisting of a chemical compound, a protein, a peptide, a peptidomemetic, an antibody, a ribozyme, a small molecule chemical compound, a nucleic acid, a vector, and an antisense nucleic acid molecule.

In one embodiment, the fibrosis-related disease or disorder is selected from the group consisting of cardiac fibrosis, interstitial lung diseases, liver cirrhosis, wound healing, systemic scleroderma, and Sjogren syndrome.

In one aspect, the invention provides a method fort treating or preventing neurodegeneration or a cardiovascular disease or disorder. In one embodiment, the method comprises administering a composition comprising a modulator of mitochondrial Na⁺/Ca²⁺ exchanger (mNCX) to a subject in need thereof. In one embodiment, the modulator decreases one or more of transcription, translation, and activity of mNCX. In one embodiment, the modulator increases one or more of transcription, translation, and activity of mNCX.

In one embodiment, the modulator is selected from the group consisting of a small interfering RNA (siRNA), a microRNA, an antisense nucleic acid, a ribozyme, an expression vector encoding a transdominant negative mutant, an antibody, a peptide, a nucleic acid, a protein, a peptide, a peptidomemetic, a chemical compound and a small molecule.

In one embodiment, the cardiovascular disease or disorder is selected from the group consisting of carotid artery disease, arteritis, myocarditis, cardiovascular inflammation, myocardial infarction, and ischemia.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1J depicts experimental results demonstrating that ₘCa²⁺ exchange gene expression and ₘCa²⁺ handling is significantly altered in Alzheimer's disease. FIG. 1A depicts qPCR analysis for changes in mRNA expression of mCa²⁺ exchanger gene in frontal cortex samples collected postmortem from non-familial human AD patients and those of age-matched controls. FIG. 1B depicts western blot analysis in non-familial AD patients and age-matched controls. FIG. 1C depicts qPCR quantification of gene expression in brain tissue isolated from the frontal cortex of 2-month old 3x-Tg AD mutant mice and age-matched outbred non-transgenic controls (NTg). FIG. 1D depicts qPCR quantification of gene expression in brain tissue isolated from the frontal cortex of 4-month old 3x-Tg AD mutant mice and age-matched outbred non-transgenic controls (NTg). FIG. 1E depicts qPCR quantification of gene expression in brain tissue isolated from the frontal cortex of 8 month old 3x-Tg AD mutant mice and age-matched outbred non-transgenic controls (NTg). FIG. 1F depicts qPCR quantification of gene expression in brain tissue isolated from the frontal cortex of aged (12 mo.) 3x-Tg AD mutant mice and outbred non-transgenic controls (NTg). FIG. 1G depicts qPCR analysis to study age dependent effects on Slc8b1/mNCX mRNA expression in brain tissue isolated from the frontal cortex of 3x-Tg AD mutant mice and age-matched outbred non-transgenic controls (NTg). FIG. 1H depicts western blot analysis in 3x-Tg AD mutant mice (12 mo.) and age-matched outbred nontransgenic controls (NTg). FIG. 11 depicts representative traces of ₘCa²⁺retention capacity assay (CRC) using the reporter Ca-Green-5n after mitochondria isolation from 3x-Tg AD mutant mice (12 mo.) and age-matched non-transgenic controls. FIG. 1J depicts the percent change in n,Ca retention capacity of 3x-Tg AD mutant mice (12 mo.) and age-matched non-transgenic controls.

FIG. 2, comprising FIG. 2A through FIG. 20 depicts experimental results demonstrating expression of mNCX rescued APPswe-induced defects in ₘCa²⁺ handling. FIG. 2A depicts western blot analysis in neuroblastoma control cell line (N2a) vs. cells stably expressing cDNA encoding the APP Swedish mutant (K670N, M671L, APPswe). FIG. 2B depicts western blot analysis of mNCX protein expression in N2a, APPswe and APPswe+Ad-mNCX from three independent experiments. FIG. 2C depicts quantification of ₘCa²⁺ rise time. FIG. 2D depicts fold change in ₘCa²⁺ uptake rate of APPswe and APPswe+Ad-mNCX vs. N2a cells. FIG. 2E depicts time to 50% ᵢCa²⁺ transient decay (T-50%). FIG. 2F depicts representative trace for ₘCa⁺ retention capacity in N2a, APPswe and APPswe cells infected with adenovirus encoding mitochondrial Na⁺/Ca²⁺ exchanger (mNCX). Cells were permeabilized with digitonin and treated with thapsigargin to inhibit SERCA and loaded cells with the ratiometric reporters FuraFF (Ca²⁺) and JC1 (mitochondrial membrane potential). The protonophore, FCCP, was used at the conclusion of the experiment to correct for total Ca²⁺ in the system. FIG. 2G depicts the percent ₘCa²⁺ efflux vs. N2a. con. FIG. 2H depicts representative fluorescence traces of ᵢCa²⁺ transients recoded in cells loaded with the ᵢCa²⁺ reporter Fluo4-AM. FIG. 2I depicts the quantification of ᵢCa²⁺ peak amplitude. FIG. 2J depicts the fold change in ₘCa²⁺ uptake rate of APPswe and APPswe+Ad-mNCX vs. N2a cells. FIG. 2K depicts the time to 50% ᵢCa²⁺ transient decay (T-50%). FIG. 2L depicts a representative trace for ₘCa²⁺ retention capacity in N2a, APPswe and APPswe cells infected with adenovirus encoding mitochondrial Na⁺/Ca²⁺ exchanger (mNCX). Cells were permeabilized with digitonin and treated with thapsigargin to inhibit SERCA and loaded cells with the ratiometric reporters FuraFF (Ca²⁺) and JC1 (mitochondrial membrane potential). The protonophore, FCCP, was used at the conclusion of the experiment to correct for total Ca²⁺ in the system. FIG. 2M depicts the percent change in ₘCa²⁺ retention capacity of APPswe (n=3) and APPswe+Ad-mNCX (n=3) vs. N2a (con) cells (n=4). FIG. 2N depicts representative traces for basal ₘCa²⁺ in N2a, APPswe and APPswe+AdmNCX. FIG. 2O depicts quantification of basal ₘCa²⁺ content.

FIG. 3, comprising FIG. 3A through FIG. 3E, depicts experimental results demonstrating enhancing ₘCa²⁺ efflux reduced oxidative stress in APPswe cells. FIG. 1A depicts quantification of cell rox green fluorescent intensity (the total cellular ROS production); fold change vs. N2a con. FIG. 3B depicts representative images of dihydroethidium (DHE) staining (518ex/605em) and differential interference contrast (DIC) merge. FIG. 3C depicts quantification of DHE fluorescent intensity; fold change vs. N2a con. FIG. 3D depicts representative images of mitosox staining (510ex/580em) and differential interference contrast (DIC) merge. FIG. 3E depicts quantification of mitosox fluorescent intensity; fold change vs. N2a con.

FIG. 4, comprising FIG. 4A through 4F, depicts experimental results demonstrating OxPhos defects in APPswe cells is rescued after mNCX expression. FIG. 4A depicts representative OCRs at baseline and following: oligomycin (oligo; CV inhibitor; to uncover ATP-linked respiration), FCCP (protonophore to induce max respiration), and rotenone+antimycin A (Rot/AA; complex I and III inhibitor; complete OxPhos inhibition). FIG. 4B depicts quantification of basal respiration (base OCR—non-mito respiration (post-Rot/AA). FIG. 4C depicts quantification of ATP-linked respiration (post-oligo OCR—base OCR). FIG. 4D depicts max respiratory capacity (post-FCCP OCR—post-Rot/AA). FIG. 4E depicts spare respiratory capacity (post-FCCP OCR—basal OCR). FIG. 4F depicts proton leak (post-Oligo OCR—post Rot/AA OCR).

FIG. 5, comprising FIG. 5A through FIG. 5F, depicts experimental results demonstrating that enhancing ₘCa²⁺ efflux decreased membrane rupture in APPswe cells. FIG. 5A depicts plasma membrane rupture of N2a, N2a-APPswe and N2a-APPswe infected with Ad-mNCX and treated with Ionomycin. FIG. 5B depicts cell viability of N2a, N2a-APPswe and N2a-APPswe infected with Ad-mNCX and treated with Ionomycin. FIG. 5C depicts plasma membrane rupture of N2a, N2a-APPswe and N2a-APPswe infected with Ad-mNCX and treated with glutamate. FIG. 5D depicts cell viability of N2a, N2a-APPswe and N2a-APPswe infected with Ad-mNCX and treated with glutamate. FIG. 5E depicts plasma membrane rupture of N2a, N2a-APPswe and N2a-APPswe infected with Ad-mNCX and treated with tert-butyl hydroperoxide. FIG. 5F depicts cell viability of N2a, N2a-APPswe and N2a-APPswe infected with Ad-mNCX and treated with tert-buyl hydroperoxide.

FIG. 6, comprising FIG. 6A through FIG. 6H, depicts experimental results demonstrating mNCX expression reduced the amyloidogenic Aβ pathway. FIG. 6A depicts western blots of full-length APP, ADAM-10 (α-secretase) BACE1 (β-secretase), PS1, Nicastrin, APH (γ-secretase), and tubulin (load con). FIG. 6B depicts quantification of APP protein expression corr. to tubulin. FIG. 6C depicts quantification of BACE1 protein expression corr. to tubulin. FIG. 6D depicts fluorometric quantification of β-secretase activity. FIG. 6E depicts representative images of intracellular protein aggregate accumulation in N2a, N2a-APPswe and APPswe+Ad-mNCX cells stained with proteostat aggresome detection reagent (red) and Hoechst 33342 nuclear stain (blue). FIG. 6F depicts quantitative analysis of protein aggregates per cell. FIG. 6G depicts ELISA quantification of extracellular Aβ1-40 levels. FIG. 6H depicts ELISA quantification of extracellular Aβ1-42 levels.

FIG. 7, comprising FIG. 7A through FIG. 7D, depicts densitometry analysis of western blots. FIG. 7A depicts Western blot analysis in 3x-Tg AD mutant mice (2 mo.) and age-matched outbred non-transgenic controls (NTg). FIG. 7B depicts densitometry analysis of all the western blots in 3x-Tg AD mutant mice (2 mo.) and age-matched outbred non-transgenic controls (NTg). FIG. 7C depicts densitometry analysis all the western blots in non-familial human AD patients and age-matched controls corr. to VDAC. FIG. 7D depicts densitometry analysis all the western blots in 3x-Tg AD mutant mice (12 mo.) and age-matched outbred non-transgenic controls (NTg).

FIG. 8, comprising FIG. 8A and FIG. 8B, depicts densitometry analysis of all the western blots and compete traces of ₘCa²⁺ retention capacity. FIG. 8A depicts representative trace for ₘCa²⁺ retention capacity in N2A, N2AAPP and APP cells infected with adenovirus encoding mitochondrial Na⁺/Ca²⁺ exchanger (mNCX). FIG. 8B depicts Densitometry analysis of all the western blots in N2a and N2a-APPswe cell lines corr. to VDAC.

FIG. 9 depicts densitometry analysis of all the western blots in N2a and N2a-APPswe and APPswe+Ad-mNCX cell lines corr. to tubulin.

FIG. 10 depicts full-length western blots in Experimental Example 1.

FIG. 11 depicts metabolome profiles of Mcu^{-/-} (Ad-Cre) and control (Ad-βgal) MEFs at baseline and post-TGFβ (12h).

FIG. 12 depicts molecular mechanisms of ₘCa²⁺ exchange and candidate genes.

FIG. 13 depicts experimental results demonstrating that ₘCa²⁺ exchange gene expression is significantly altered in human AD.

FIG. 14 depicts experimental results demonstrating neuronal cell line expressing human APPswe display altered ₘCa²⁺ exchanger expression, elevated ᵢCa²⁺ and ₘCa²⁺ transients and increased susceptibility to MPTP activation.

FIG. 15 depicts experimental results demonstrating expression of mNCX rescues APPswe-induced defects in ₘCa²⁺ handling.

FIG. 16 depicts experimental results demonstrating expression of mNCX reduces superoxide generation in a neuronal AD model.

FIG. 17 depicts experimental results demonstrating expression of mNCX rescues OxPhos defects in APPswe cells.

FIG. 18 depicts experimental results demonstrating enhancing ₘCa²⁺ efflux decreases the amyloidogenic Aβ pathway.

FIG. 19 depicts experimental results demonstrating enhancing ₘCa²⁺ efflux reduces cell death by a variety of stressors

FIG. 20 depicts experimental results demonstrating ₘCa²⁺ exchange gene expression and ₘCa²⁺handling is significantly altered in 3xTG-AD mice.

FIG. 21, comprising FIG. 21A through FIG. 21E, depicts generation of mNCX conditional mutant mouse.

FIG. 21A depicts schematic of KO-1^{st} gene targeting strategy. LoxP sites (red triangles) flank exons 5-7 and FRT sites (green halfcircles) flank a splice acceptor site (En2-SA), β-galactosidase (βgal) reporter, and neomycin resistance (Neo) cassette. KO-1st mutant mice were crossed with flippase expressing mice (ROSA26-FLPe) for removal of FRT flanked region resulting in an allele with conditional potential. Homozygous LoxP ‘foxed’ mice (Slc8b1fl/fl) were crossed with neuron-restricted (Camk2a-promoter) CreERT2-recombinase transgenic mice resulting in tamox-mediated deletion of Slc8b1. FIG. 21B depicts images of chimeric founder mice and estimated percent chimerism, black coat color correlates with mutant ES contribution to development. FIG. 21C depicts genotyping gel of Slc8b1 mutant mice. FIG. 21D depicts qPCR analysis of mNCX, Mcu and Micu1 mRNA expression in tissue isolated from cortex of brains. FIG. 7E depicts ₘCa²⁺uptake and efflux in isolated adult cardiomyocytes (ACMs) from an ongoing study. ACMs were permeabilized with digitonin (dg), treated thapsigargin (thaps) and a 20-μM Ca²⁺ pulse was delivered at 350 s. Recordings were analyzed for changes in ₘCa²⁺ uptake (FuraFF, left y-axis) and mitochondrial membrane potential (JC-1, right y-axis). ACMs were treated with the MCU inhibitor, Ru360, at 550s to evaluate the rate of efflux independent of uptake. At 650 s, the mNCX inhibitor, CGP-37157, was injected.

FIG. 22, comprising FIG. 22A and FIG. 22B, depicts results of experiments demonstrating mNCX-nTg mutant mouse model. FIG. 22A depicts schematic of tet-responsive transgenic construct and neuronal specific driver, Camk2a-tTA. FIG. 22B depicts qPCR analysis of mRNA expression corrected to the housekeeping gene Rps13 expressed as fold-change vs. tTA con.

FIG. 23 depicts results of experiments demonstrating genotyping of mNCX-nKO×3xTg-AD mice. Genotyping gel displaying PCR analysis of mutant and WT alleles for mNCX mutant, Camk2a-Cre, Psen1 knock-in, and APP and MAPT transgenes (co-injected, incorporated at same loci).

FIG. 24, comprising FIG. 24A through FIG. 24E, depicts results of experiments demonstrating generation of a Mcu conditional knockout mouse. FIG. 24A depicts Mcu targeting construct containing FRT and loxP sites for conditional potential. FRT recombination after crossing with Rosa26-FLPe mice generates a Mcu ‘foxed’ (Mcu^{fl/fl}) mouse with loxP sites flanking critical exons 5-6. Crossing a Mcu floxed mouse with a transgenic mouse expressing a tamoxifen-inducible, fibroblast-specific Cre recombinase under control of the collagen type I, alpha 2 promoter (Col1a2-Cre/ERT) generates fibroblast-restricted deletion of Mcu in adult mice. FIG. 24B depicts protocol for generation of Mcu^{-/-} mouse embryonic fibroblasts (MEFs). MEFs isolated from Mcu^{fl/fl} embryos at E13.5 and infected with adenovirus encoding Cre recombinase (Ad-Cre) or β-galactosidase (Ad-βgal) as a control for 24 h. FIG. 24C depicts 96 h post-infection with Ad-Cre or Ad-βgal, MCU protein expression was examined by western blot. FIG. 24D depicts MEFs loaded with the calcium sensitive dye Fluo-4 AM. The fluorescent signal was recorded and a single pulse of 1 mM ATP or 100 nM Angiotensin II (AngII) was delivered to liberate ᵢCa²⁺ stores. FIG. 24E depicts MEFs infected with adenovirus encoding the mitochondrial calcium sensor, Miro R GECO. The fluorescent signal was recorded and a single pulse of 1 mM ATP or 100 nM AngII was delivered to liberate ᵢCa²⁺ stores.

FIG. 25, comprising FIG. 25A through FIG. 25F depicts results of experiments demonstrating deletion of fibroblast Mcu potentiates LV dysfunction and fibrosis after MI. FIG. 25A depicts Outline of experimental procedure. Mcu floxed mice were crossed with a transgenic mouse expressing a conditional, fibroblast-specific Cre recombinase (Col1a2-Cre/ERT). 8-12 w old mice were treated with tamoxifen (40 mg/kg/day) for 10 d to induce fibroblast-restricted Cre expression and allowed to rest for 3 w prior to permanent ligation of the left coronary artery. Mice were analyzed by echocardiography 1 w prior to MI and every week thereafter. FIG. 25B depicts mice were analyzed by M-mode echocardiography and measurements of ejection fraction (EF), LV end systolic volume (LVESV), and LV end-diastolic volume (LVEDV) were acquired. Mice were sacrificed 4 w post-MI. FIG. 25C depicts ratio of heart weight to tibia length. FIG. 25D depicts quantification of wet—dry lung weight as a measurement of lung edema. FIG. 25E depicts left ventricular sections were stained with Masson's trichrome. Representative images, 4 weeks post-MI are presented. FIG. 25F depicts quantification of fibrosis.

FIG. 26, comprising FIG. 26A through FIG. 26H depicts results of experiments demonstrating ablation of ₘCa²⁺ uptake enhancing myofibroblast trans differentiation. FIG. 26A depicts immunofluorescence that was performed by co-staining with α-smooth muscle actin (α-SMA) antibody (red) and DAPI (blue). Z-stack images were captured and representative de-convoluted images are presented. FIG. 26B depicts mean fluorescence intensity was calculated. More than 200 cells in each group were used for statistical comparisons. FIG. 26C depicts collagen gel contraction assay (measure of myofibroblast contractile phenotype). Representative images are presented. FIG. 26D depicts quantification of gel contraction calculated as percent change from time 0 h. FIG. 26E depicts scratch assay (measure of wound healing). Representative images are presented. FIG. 26F depicts wound closure was quantified as percent change from time Oh. FIG. 26G depict cell proliferation measured by DNA Content using CyQUANT. FIG. 26H depicts fold change in expression of myofibroblast genes.

FIG. 27, comprising FIG. 27A through FIG. 27U depicts results of experiments demonstrating Mcu^{-/-} are more glycolytic and PDH activation in response to fibrotic agonists is altered. FIG. 27A depicts schematic of experimental timeline and figure legend. MEFs were treated with pro-fibrotic stimuli or vehicle for 12, 24, 48 or 72h and assayed for Glycolytic function and Oxidative Phosphorylation using a Seahorse XF96 to measure extracellular acidification rates (ECAR, glycolysis) or oxygen consumption rates (OCR, OxPhos). FIG. 27B through FIG. 27D depicts results from the glycolytic stress test following treatment with 10 ng/mL TGF-β+10 μM Angiotensin II. FIG. 27B depicts ECAR traces. FIG. 27C depicts glycolytic Rate. FIG. 27D depicts glycolytic Capacity. FIG. 27E through FIG. 27F depicts results from the glycolytic stress test following treatment with AngII. FIG. 27E depicts glycolytic Rate. FIG. 27F depicts glycolytic Capacity. FIG. 27G through FIG. 27J depicts Results from the mito stress test following treatment with 10 ng/mL TGF-β+10 μM AngII. FIG. 27G depicts OCR traces, FIG. 27H depicts Basal Respiration. FIG. 27I depicts ATP Production. FIG. 27J depicts Maximal Respiration. FIG. 27K through FIG. 27M depicts results from the mito stress test following treatment with AngII. FIG. 27K depicts Basal Respiration. FIG. 27L depicts ATP Production. FIG. 27M depicts Maximal Respiration. FIG. 27N depicts Percent change in Glycolytic Rate following treatment with TGF-β+ Angiotensin II. FIG. 27O depicts percent change in Glycolytic Rate following treatment with Angiotensin II. FIG. 27o depicts percent change in the ratio of Basal Respiration/Glycolysis following treatment with TGF-β+AngII. FIG. 27Q depicts percent change in the ratio of Basal Respiration/Glycolysis following treatment with Angiotensin II. FIG. 27R depicts MEFs immunoblotted for phosphorylated PDH E1α S293 (p-PDH), PDH-E1α, PDPc, IDH3, GAPDH and Tubulin. FIG. 14S depicts MEFs treated with AngII for 0, 24, 48, or 72 h and immunoblotted for p-PDH, PDH-E1α and OxPhos Components. FIG. 27T depicts MEFs treated with TGF-β for 0, 24, 48, or 72 h and immunoblotted for p-PDH, PDH components and OxPhos Components. FIG. 27U depicts MEFs treated with TGF-β+Angiotensin II for 0, 24, 48, or 72 h and immunoblotted for p-PDH, PDH components and OxPhos Components.

FIG. 28, comprising FIG. 28A through FIG. 28G depicts results of experiments demonstrating enhanced glycolysis drives myofibroblast transdifferentiation. FIG. 28A depicts Schematic of the major rate limiting and committed step in glycolysis: Phosphofructokinase 1 (PFK1) phosphorylates Fructose-6-Phosphate (F-6-P) to F-1,6-P2. Phosphofructokinase 2 (PFK2) or fructose bisphosphatase 2 (FBP2) catalyzes the synthesis and degradation, respectively, of Fructose-2,6-Bisphosphate (Fru-2,6-P2), an important regulator of PFK1. Here, we used adenovirus co-expressing mutant PFK2 and GFP to alter glycolysis. FIG. 28B depicts phosphatase-deficient PFK2 (Ad-Glyco-High) only exhibits PFK2 activity, which increases intracellular levels of Fru-2,6-P2 to activate PFK1 and glycolysis. FIG. 28C through FIG. 28D depicts MEFs were infected with Ad-Glyco-High and treated with AngII for 48 h. Immunofluorescence was performed for α-smooth muscle actin (α-SMA). Representative images are presented. FIG. 28D depicts percentage of cells expressing α-SMA alone or co-expressing Ad-Glyco-High (GFP) and α-SMA was calculated. FIG. 28E depicts kinase-deficient PFK2 (Ad-Glyco-Low) is unable to increase intracellular levels of Fru-2,6-P2, thus PFK1 is not activated and glycolysis is reduced. FIG. 28F through FIG. 28G depicts MEFs infected with Ad-Glyco-Low and treated with TGF-fβ+AngII for 48 h. Immunofluorescence was performed for α-SMA. Representative images are presented. FIG. 28G depicts percentage of cells expressing α-SMA alone or co-expressing Ad-Glyco-Low and a-SMA was calculated.

FIG. 29 comprising FIG. 29A through FIG. 29B depicts results of experiments demonstrating the pro-fibrotic stimulus TGF-β changes expression of MCU components. FIG. 29A depicts wild-type MEFs treated with 10 ng/mL TGF-β for 12, 24, 48, or 72 h and cell lysates immunoblotted for components of the mitochondrial calcium uniporter (MCU) complex, including the pore forming subunit MCU, regulatory subunits MCUb, MICU1 (Mitochondrial Ca²⁺ uptake 1), MICU2 (Mitochondrial Ca²⁺ uptake 2), and MCUR1 (Mitochondrial Ca²⁺ uniporter regulator 1), as well as OxPhos Complexes CV (ATP5A) and CIII (UQCRC2), VDAC (Voltage-dependent anion channel), α-Tubulin. FIG. 29B depicts fold change in protein expression vs Vehicle. Band signal intensity was normalized to CIII.

FIG. 30 depicts a summary and conclusion of the experimental results. Deletion of Mcu attenuates ₘCa²⁺ uptake and increases ᵢCa²⁺ amplitude upon stimulation with ATP, AngII, and ET1, suggesting that the mitochondria buffer ᵢCa²⁺ in fibroblasts. Deletion of Mcu in fibroblasts worsens left ventricular function and cardiac fibrosis following MI. Mcu ablation enhances myofibroblast transdifferentiation. Mcu−/− MEFs are more glycolytic and have increased inactivation of PDH, suggesting changes in metabolic flux. Increasing glycolysis augments myofibroblast transdifferentiation while decreasing glycolysis attenuates the enhanced transdifferentiation in Mcu−/− MEFs. TGF-β changes the expression of key MCU components, suggesting that inhibition of mitochondrial Ca²⁺ uptake may be an endogenous mechanism whereby pro-fibrotic stimuli elicit myofibroblast transdifferentiation.

FIG. 31, comprising FIG. 31A through FIG. 31P depicts experimental results demonstrating loss of ₘCa²⁺ uptake enhances the myofibroblast differentiation. FIG. 31A depicts Mcu conditional allele with LoxP sites flanking exons 5-6. Cre recombinase (Cre) drives deletion of Mcu in floxed cells. FIG. 31B depicts experimental timeline for deletion of Mcu in mouse embryonic fibroblasts (MEFs). MEFs were isolated from Mcu^{fl/fl} embryos at E13.5 and infected with adenovirus encoding Cre recombinase (Ad-Cre) or the experimental control beta-galactosidase (Ad-βgal) for 24 h. FIG. 31C depicts expression of mtCU components was examined by Western blot in Mcu^{-/-} (Ad-Cre) and control (Ad-βgal) MEFs. MICU1—mitochondrial Ca²⁺ uptake 1, MCUR1—mitochondrial Ca²⁺ uniporter regulator 1, MCUb—mitochondrial Ca²⁺ uniporter subunit b, EMRE—essential MCU regulator. Voltage-dependent anion channel (VDAC) and complex III (CIII, subunit UQCRC2 (Ubiquinol-cytochrome-c reductase complex core protein 2)) were used as mitochondrial loading controls and Tubulin served as a total lysate loading control. FIG. 31D depicts Mcu^{-/-} and control MEFs transduced with adenovirus encoding the mitochondrial calcium sensor, Miro R-GECO. 1 mM ATP was delivered to initiate purinergic receptor-mediated IP3R Ca²⁺release. FIG. 31E depicts amplitude (peak intensity—baseline). F) Mcu^{-/-} and control MEFs were loaded with the Ca²⁺-sensitive dye Fluo-4 AM and fluorescence was recorded during 1 mM ATP treatment. FIG. 31G depicts amplitude (peak intensity—baseline). FIG. 31H depicts immunofluorescence performed by co-staining with α-smooth muscle actin (α-SMA) antibody (red) and DAPI (blue) OF MEFs treated with control vehicle. FIG. 311 depicts immunofluorescence performed by co-staining with α-smooth muscle actin (α-SMA) antibody (red) and DAPI (blue) OF MEFs treated with TGFβ for 24 h. FIG. 31J depicts immunofluorescence performed by co-staining with α-smooth muscle actin (a-SMA) antibody (red) and DAPI (blue) OF MEFs treated with AngII for 24 h. FIG. 31K depicts the percentage of α-SMA positive cells. FIG. 31L depicts α-SMA expression (fluorescence intensity). FIG. 31M depicts representative images at 0 and 24 h of the collagen gel contration assay. FIG. 31N depicts gel contraction calculated as percent change from time 0 h. FIG. 31O depicts the fold change in expression of myofibroblast genes (vs. Ad-βgal control). Colla1—collagen type I alpha 1 chain; Colla2—collagen type I alpha 2 chain; Col3a1—collagen type III alpha 1 chain; α-SMA (Acta2)—α-smooth muscle actin; Postn—periostin; Lox—lysyl oxidase; Fn1—fibronectin 1; Pdgfra—platelet derived growth factor receptor alpha. FIG. 31P depicts cell proliferation measured by quantifying DNA content.

FIG. 32, comprising FIG. 32A through FIG. 32K, depicts experimental results demonstrating pro-fibrotic stimuli alter mtCU gating to reduce ₘCa²⁺ uptake. FIG. 32A depicts representative Ca²⁺ traces in untreated WT MEFs (black traces) and TGFβ-treated MEFs (blue traces). FIG. 32B depicts JC-1 derived ΔΨ in untreated WT MEFs (black) and TGFβ-treated MEFs (blue). FIG. 32C depicts dose response curve of ₘCa²⁺ uptake following [Ca²⁺] boluses. FIG. 32D depicts dose response curve of ₘCa²⁺ uptake following [Ca²⁺] boluses. FIG. 32E depicts Kinetic parameters derived from Hill equation fits of data. FIG. 32F depicts immunoblots of WT MEFs treated with TGFβ for 12, 24, 48, or 72 h and cell lysates were immunoblotted for components of the mtCU, including the pore forming subunit MCU and regulatory subunits MICUl (mitochondrial Ca²⁺ uptake 1), MCUR1 (mitochondrial Ca²⁺ uniporter regulator 1), MCUb, and EMRE (essential MCU regulator), as well as OxPhos Complexes CV (ATP5A) and CIII (subunit UQCRC2 (Ubiquinol-cytochrome-c reductase complex core protein 2)), VDAC (Voltage-dependent anion channel), and Tubulin. FIG. 32G depicts the fold change of MICU1 expression in WT MEFs were treated with TGFβ for 12, 24, 48, or 72 h. FIG. 32H depicts the fold change in the ratio of MICU1/MCU expression in WT MEFs treated with TGFβ for 12, 24, 48, or 72 h. FIG. 32I depicts immunoblots of WT MEFs treated with AngII for 12, 24, 48, or 72 h and cell lysates were immunoblotted for components of the mtCU. FIG. 32J depicts the fold change of MICU1 expression in WT MEFs treated with AngII for 12, 24, 48, or 72 h. FIG. 32K depicts the fold change in the ratio of MICU1/MCU expression in WT MEFs treated with AngII for 12, 24, 48, or 72 h.

FIG. 33, comprising FIG. 32A through FIG. 32B′, depicts experimental results demonstrating TGFβ/AngII signaling elicits rapid and dynamic changes in fibroblast metabolism. FIG. 33A depicts the percent change in glycolysis (y-axis) vs. percent change in basal respiration (x-axis) following stimulation with TGFβ for 0, 12, 24, or 48 h. FIG. 33B depicts the percent change in glycolysis (y-axis) vs. percent change in basal respiration (x-axis) following stimulation with AngII for 0, 12, 24, or 48 h. FIG. 33C depicts a schematic representation of changes in glycolysis (blue) and oxidative phosphorylation (red) during myofibroblast differentiation induced by TGFβ. FIG. 33D depicts a schematic representation of changes in glycolysis (blue) and oxidative phosphorylation (red) during myofibroblast differentiation induced by AngII. FIG. 33E depicts quantification of glycolysis 12 h post-TGFβ or -AngII. Percent change vs. Ad-βgal vehicle. FIG. 33F depicts a simplified outline of glycolysis depicting the metabolites: glucose-6-phosphate (G-6-P), fructose-6-phosphate (F-6-P), fructose-1,6-bisphosphate (F-1,6-BP), fructose-2,6-bisphosphate (F-2,6-BP), dihydroxyacetone phosphate (DHAP), glycerol-3-phosphate (G-3-P), glyceraldehyde-3-phosphate (GA3P), 1,3-bisphosphoglyceric acid (1,3-BPG), 3-phosphoglyceric acid (3-PG), and the enzymes: phosphofructokinase 2/fructose bisphosphatase 2 (PFK2/FBP2), phosphofructokinase 1 (PFK1). FIG. 33G depicts absolute concentration of glycolytic intermediate G-6-P in Mcd^{-/-} (Ad-Cre) and control (Ad-βgal) MEFs at baseline and 12 h post-TGFP. FIG. 33H depicts absolute concentration of glycolytic intermediate F-6-P in Mar^{-/-} (Ad-Cre) and control (Ad-βgal) MEFs at baseline and 12 h post-TGFβ. FIG. 33I depicts absolute concentration of glycolytic intermediate F-1,6-BP in Mcd^{-/-} (Ad-Cre) and control (Ad-βgal) MEFs at baseline and 12 h post-TGFP. FIG. 33J depicts absolute concentration of glycolytic intermediate GA3P in Mcd^{-/-} (Ad-Cre) and control (Ad-βgal) MEFs at baseline and 12 h post-TGFβ. FIG. 33K depicts absolute concentration of glycolytic intermediate 3-PG in Mcu^{-/-} (Ad-Cre) and control (Ad-βgal) MEFs at baseline and 12 h post-TGFβ. FIG. 33L depicts absolute concentration of glycolytic intermediate DHAP in Mcu^{-/-} (Ad-Cre) and control (Ad-βgal) MEFs at baseline and 12 h post-TGFβ. FIG. 33M depicts absolute concentration of glycolytic intermediate G-3-P in Mcu^{-/-} (Ad-Cre) and control (Ad-βgal) MEFs at baseline and 12 h post-TGFβ. FIG. 33N depicts adenoviruses co-expressing mutant PFK2/FBP2 and GFP—phosphatase-deficient PFK2/FBP2 (S32A, H258A; Ad-Glyco-High). FIG. 33O depicts adenoviruses co-expressing mutant kinase-deficient PFK2/FBP2 (S32D, T55V; Ad-Glyco-Low). FIG. 33P depicts Mcu^{-/-} and control MEFs were transduced with Ad-Glyco-High, Ad-Glyco-Low, or control Ad-GFP and 24 h later assayed for glycolysis using a Seahorse XF96 analyzer to measure extracellular acidification rates (ECAR, glycolysis). FIG. 33Q depicts immunofluorescence images for α-SMA of MEFs transduced with Ad-Glyco-High and 24 h later treated with a control vehicle. FIG. 33R depicts quantification of immunofluorescence images for α-SMA of MEFs transduced with Ad-Glyco-High and 24 h later treated with a control vehicle. FIG. 33S depicts immunofluorescence images for α-SMA of MEFs transduced with Ad-Glyco-High and 24 h later treated with TGFβ. FIG. 33T depicts quantification of immunofluorescence images for α-SMA of MEFs transduced with Ad-Glyco-High and 24 h later treated with TGFβ. FIG. 33U depicts immunofluorescence images for α-SMA of MEFs transduced with Ad-Glyco-High and 24 h later treated with AngII. FIG. 33V depicts quantification of immunofluorescence images for α-SMA of MEFs transduced with Ad-Glyco-High and 24 h later treated with AngII. FIG. 33W depicts immunofluorescence images for α-SMA of MEFs transduced with Ad-Glyco-Low and 24 h later treated with a control vehicle. FIG. 33X depicts quantification of immunofluorescence images for α-SMA of MEFs transduced with Ad-Glyco-Low and 24 h later treated with a control vehicle. FIG. 33Y depicts immunofluorescence images for a-SMA of MEFs transduced with Ad-Glyco-Low and 24 h later treated with a TGFβ. FIG. 33Z depicts quantification of immunofluorescence images for α-SMA of MEFs transduced with Ad-Glyco-Low and 24 h later treated with TGFβ. FIG. 33A′ depicts immunofluorescence images for α-SMA of MEFs transduced with Ad-Glyco-Low and 24 h later treated with a AngII. FIG. 33B′ depicts quantification of immunofluorescence images for α-SMA of MEFs transduced with Ad-Glyco-Low and 24 h later treated with AngII.

FIG. 34, comprising FIG. 34A through FIG. 34N depicts experimental results demonstrating loss of ₘCa²⁺ uptake reduces pyruvate entry into the TCA cycle. FIG. 34A depicts TCA cycle with emphasis on key ₘCa²⁺-control points—pyruvate dehydrogenase (PDH) and α-ketoglutarate dehyodrogenase (αKGDH). FIG. 34B depicts Mcd^{-/-} (Ad-Cre) and control (Ad-βgal) MEFs were immunoblotted for p-PDH E1α (phosphorylated pyruvate dehydrogenase, inactivate), total PDH E1α, PDPc (pyruvate dehydrogenase phosphatase catalytic subunit 1), IDH3A (mitochondrial isocitrate dehydrogenase subunit alpha), GAPDH (glyceraldehyde 3-phosphate dehydrogenase) and Tubulin. FIG. 34C depicts the ratio of p-PDH E1α/PDH E1α. FIG. 34D depicts Mar^{-/-} and control MEFs were treated with TGFβ or AngII for 0, 24, 48, or 72 h and immunoblotted for p-PDH E1α, PDH E1α and OxPhos Complex V. FIG. 34E depicts absolute concentration of metabolic intermediate pyruvate in Mcu^{-/-} (Ad-Cre) and control (Ad-βgal) MEFs at baseline and 12 h post-TGFP. FIG. 34F depicts absolute concentration of metabolic intermediate acetyl-CoA in Mcu^{-/-} (Ad-Cre) and control (Ad-βgal) MEFs at baseline and 12 h post-TGF62 . FIG. 34G depicts absolute concentration of metabolic intermediate citrate in Mcu^{-/-} (Ad-Cre) and control (Ad-βgal) MEFs at baseline and 12 h post-TGFβ. FIG. 34H depicts absolute concentration of metabolic intermediate dKG in Mcu^{-/-} (Ad-Cre) and control (Ad-βgal) MEFs at baseline and 12 h post-TGFP. FIG. 341 depicts absolute concentration of metabolic intermediate succinate in Mcu^{-/-} (Ad-Cre) and control (Ad-βgal) MEFs at baseline and 12 h post-TGFβ. FIG. 34J depicts absolute concentration of metabolic intermediate fumarate in Mcu^{-/-} (Ad-Cre) and control (Ad-βgal) MEFs at baseline and 12 h post-TGFβ. FIG. 34K depicts absolute concentration of metabolic intermediate maltate in Mcu^{-/-} (Ad-Cre) and control (Ad-βgal) MEFs at baseline and 12 h post-TGFβ. FIG. 34L depicts absolute concentration of metabolic intermediate glutamate in Mcu^{-/-} (Ad-Cre) and control (Ad-βgal) MEFs at baseline and 12 h post-TGFβ. FIG. 34M depicts absolute concentration of metabolic intermediate glutamine in Mcu^{-/-} (Ad-Cre) and control (Ad-βgal) MEFs at baseline and 12 h post-TGFβ. FIG. 34N depicts absolute concentration of metabolic intermediate dkG/Gln in Mcu^{-/-} (Ad-Cre) and control (Ad-βgal) MEFs at baseline and 12 h post-TGFβ.

FIG. 35, comprising FIG. 35A-FIG. 35K depicts experimental results demonstrating Loss of ₘCa²⁺ uptake drives myofibroblast differentiation through epigenetic reprogramming. FIG. 35A depicts a simplified schematic of the reaction mechanism of α-ketoglutarate (αKG)-dependent dioxygenases: ten-eleven translocation (TET) enzymes and Jumonji-C (JmjC)-domain-containing demethylases (JmjC-KDMs). FIG. 35B depicts levels of 5-methylcytosine (5-mC) were measured in Mcu^{-/-} (Ad-Cre) and control (Ad-βgal) MEFs by ELISA. Fold change vs. Ad-βgal veh. FIG. 35C depicts MEFs were treated with TGFβ for 0, 12 or 24 h and cell lysates were immunoblotted for specific methylated histone 3 lysine (H3K) residues. Total H3 and Tubulin were used as loading controls. FIG. 35D depicts quantification of H3K27me2 protein expression. Band density was normalized to total H3. FIG. 35E depicts H3K27me2 chromatin immunoprecipitation followed by qPCR (ChIP-qPCR) of Periostin in Mcu^{-/-} (Ad-Cre) and control (Ad-βgal) MEFs at baseline (veh) and following 12 h TGFβ. Schematic shows loci of qPCR primers in relationship to myofibroblast transcription factor binding sites—NFAT (nuclear factor of activated T-cells), SRF (serum response factor). FIG. 35F depicts expression of Periostin mRNA in Mcu^{-/-} (Ad-Cre) and control (Ad-βgal) MEFs at baseline (veh) and post-TGFβ. FIG. 35G depicts H3K27me2 ChIP-qPCR of platelet-derived growth factor receptor alpha (Pdgfra) in Mcu^{-/-} (Ad-Cre) and control (Ad-βgal) MEFs at baseline (veh) and following 12 h TGFβ. Schematic shows loci of qPCR primers in relationship to myofibroblast transcription factor binding sites—MEF2 (myocyte enhancer factor 2), SMAD3 (SMAD family member 3). FIG. 35H depicts qPCR of Pdgfra mRNA in Mcu^{-/-} (Ad-Cre) and control (Ad-βgal) MEFs at baseline (veh) and post-TGFP. FIG. 35I depicts immunofluorescence for α-SMA of wildtype MEFS treated +/− cell-permeable, di-methyl-αKG and with a control vehicle for 48 h. FIG. 35J depicts immunofluorescence for α-SMA of wildtype MEFS treated +/− cell-permeable, di-methyl-αKG and with TGFβ for 48 h. FIG. 35K depicts the quantification of immunofluorescence. FIG. 36, comprising FIG. 35A—FIG. 36O, depicts experimental results demonstrating adult deletion of fibroblast Mcu exacerbates cardiac dysfunction, fibrosis, and myofibroblast formation post-MI and chronic angiotensin II administration. FIG. 36A depicts Mac^{fl/fl} mice were crossed with a transgenic mouse expressing a tamoxifen (tamox)-inducible, fibroblast-specific Cre recombinase (Col1a2-CreERT). Tamox administration (40 mg/kg/day) for 10 d induces fibroblast-restricted Cre expression. FIG. 36B depicts adult cardiac fibroblasts were isolated from Mcu^{fl/fl}×Col1a2-CreERT and control Col1a2-CreERT mice post-tamox treatment and immunoblotted for MCU expression. CIII (Complex III, subunit UQCRC2) was used as a loading control. FIG. 36C depicts experimental timeline: 8-12 wk old mice were treated with tamox and allowed to rest before permanent ligation of the left coronary artery. FIG. 36D depicts the M-mode echo measurements of left ventricular end diastolic diameter (LVEDD) 1 week prior to MI and every week thereafter. FIG. 36E depicts the M-mode echo measurements of left ventricular left ventricular end systolic diameter (LVESD) 1 week prior to MI and every week thereafter. FIG. 36D depicts the M-mode echo measurements of percent fractional shortening (FS) 1 week prior to MI and every week thereafter. FIG. 36G depicts the ratio of heart weight to tibia length 4 wks post-MI. Sham: n=5 Col1a2-Cre, n=7 Mcu^{fl/fl}×Col1a2-Cre; post MI: n=10 Col1a2-Cre, n=20 Mcu^{fl/fl}×Col1a2-Cre. FIG. 36H depicts quantification of wet-dry lung weight as a measurement of lung edema 4 wks post-MI. n=10 Col1a2-Cre, n=20 Mcu^{fl/fl}×Col1a2-Cre. FIG. 36I depicts representative images of LV sections stained with Masson's trichrome. FIG. 36J depicts percent fibrotic area per infarct border and remote zones of LV sections stained with Masson's trichrome. FIG. 36K depicts the percent change in myofibroblast number (α-SMA+/CD31-) in the remote zone 4 wks post-MI. n=4 Col1a2-Cre, n=8 Mcu^{fl/fl}×Col1a2-Cre; multiple non-consecutive heart sections in the remote zone were quantified per mouse. FIG. 36L depicts Experimental timeline: mini-osmotic pumps were subcutaneously implanted in mice to deliver AngII for 4 wks. FIG. 36M depicts representative images of LV sections stained with Masson's trichrome. FIG. 36N depicts percent fibrotic area per infarct border and remote zones of LV sections stained with Masson's trichrome. FIG. 36O depicts the percent change in myofibroblast number (α-SMA+/CD31-) 4 wks post-AngII infusion.

FIG. 37 depicts a schematic demonstrating the changes in mtCU gating is essential for myofibroblast differentiation. Signaling model for myofibroblast differentiation whereby fibrotic stimuli acutely upregulate MICU1 to inhibit ₘCa²⁺ uptake. Enhanced mtCU gating leads to a cascade of changes driving myofibroblast differentiation. Decreased ₘCa²⁺ uptake downregulates the activity of ₘCa²⁺-dependent dehydrogenases (PDH, αKGDH). This causes an increase in glycolysis, which supports energetic demands of the differentiation process. In addition, there are changes to TCA cycle intermediates, including increased αKG, which increases JmjC-KDM-dependent histone demethylation to activate the myofibroblast gene program.

FIG. 38, comprising FIG. 38A and FIG. 38B, depicts experimental results demonstrating loss of ₘCa²⁺ uptake enhances cytosolic signaling. FIG. 38A depicts fluorescence microscopy images of Mcu^{-/-} (Ad-Cre) and control (Ad-βgal) MEFs transduced with adenovirus-encoding NFATc1-GFP and 24 h later treated with TGFβ or AngII for 24 h. FIG. 38B depicts the percentage of cells with nuclear NFATc1.

FIG. 39, comprising FIG. 39A through FIG. 39I, depicts experimental results demonstrating calibration of Fura-2 Ca²⁺ reporter and quantification of expression of mtCU components post-TGFβ or AngII. FIG. 39A depicts that fura-2 was calibrated by the generation of a standard curve of Ca²⁺ (0.01-100 μm) in experimental intracellular buffer to quantify actual Ca²⁺ content. Fura-2 fluorescence ratio was converted to [Ca²⁺] by the following equation: [Ca²⁺]=K_{d}*(R−Rₘᵢₙ)/(Rₘₐₓ−R)*Sf2/Sb2. (Rₘᵢₙ=ratio in 0-Ca²⁺; Rₘₐₓ=ratio at saturation; Sf2=380/510 reading in 0-Ca²⁺; Sb2=380/510 reading with Ca²⁺ saturation. FIG. 39B depicts the fold change in expression of MCU. FIG. 39C depicts the fold change in expression of MCUb. FIG. 39D depicts the fold change in expression of MCUR1. FIG. 39E depicts the fold change in expression of EMRE. FIG. 39F depicts the fold change in expression of MCU. FIG. 39G depicts the fold change in expression of MCUb. FIG. 39H depicts the fold change in expression of MCUR1. FIG. 391 depicts the fold change in expression of EMRE.

FIG. 40, comprising FIG. 40A through FIG. 40G, depicts experimental results demonstrating seahorse analysis of glycolysis and oxidative phosphorylation. FIG. 40A depicts a schematic of experimental timeline. MEFs were treated with fibrotic stimuli for 12, 24, 48, or 72 h and assayed for Glycolysis and Oxidative Phosphorylation using a Seahorse XF96 analyzer to measure extracellular acidification rates (ECAR, glycolysis) or oxygen consumption rates (OCR, OxPhos). FIG. 40B depicts a schematic of experimental timeline. FIG. 40C depicts a schematic of experimental timeline. FIG. 40D depicts quantification of glycolysis, glycolytic capacity, and glycolytic reserve in Mcu^{-/-} (Ad-Cre) and control (Ad-βgal) MEFs post-TGFβ. FIG. 40E depicts quantification of basal respiration, ATP-linked respiration, maximal respiration, reserve capacity, and proton leak in Mcu^{-/-} and control MEFs post-TGFβ. FIG. 40F depicts quantification of glycolysis, glycolytic capacity, and glycolytic reserve in Mcu^{-/-} and control MEFs post-AngII. FIG. 40G depicts quantification of basal respiration, ATP-linked respiration, maximal respiration, reserve capacity, and proton leak in Mcu^{-/-} and control MEFs post-AngII.

FIG. 41, comprising FIG. 41A through FIG. 41D, depicts experimental results demonstrating quantification of metabolites involved in the pentose phosphate pathway. FIG. 41A depicts a schematic of the pentose phosphate pathway: glucose-6-phosphate (G-6-P), 6-phosphogluconate (6-PG), ribulose-5-phosphate (Ru-5-P), ribose-5-phosphate (R-5-P), glyceraldehyde-3-phosphate (GA3P), fructose-6-phosphate (F-6-P). FIG. 41B depicts absolute concentration of pentose phosphate pathway metabolite 6-phosphogluconate. FIG. 41C depicts absolute concentration of pentose phosphate pathway metabolite ribulose-5-P phosphate. FIG. 41D depicts absolute concentration of pentose phosphate pathway metabolite ribose-5-phosphate.

FIG. 42 depicts a heat map of metabolites. Heat map representation of metabolome profiles of Mcu^{-/-} (Ad-Cre) and control (Ad-βgal) MEFs at baseline and post-TGFβ (12 h). Unit variance scaling is applied to rows. Rows are clustered using Manhattan distance and average linkage.

FIG. 43, comprising FIG. 43A through FIG. 43D, depicts echocardiographic parameters and representative immunohistochemistry images of myofibroblast identification. FIG. 43A depicts M-mode echo measurements LVEDV 1 wk prior to MI and every week thereafter of Mcu^{fl/fl}×Col1a2-CreERT and control Colla2-CreERT mice treated with tamoxifen (40 mg/kg/day) for 10 d and allowed to rest 10 d before permanent ligation of the left coronary artery. FIG. 43B depicts M-mode echo measurements LVESV 1 wk prior to MI and every week thereafter of Mcu^{fl/fl}×Col1a2-CreERT and control Col1a2-CreERT mice treated with tamoxifen (40 mg/kg/day) for 10 d and allowed to rest 10 d before permanent ligation of the left coronary artery. FIG. 43A depicts M-mode echo measurements ejection fraction (EF) 1 wk prior to MI and every week thereafter of Mcu^{fl/fl}×Col1a2-CreERT and control Col1a2-CreERT mice treated with tamoxifen (40 mg/kg/day) for 10 d and allowed to rest 10d before permanent ligation of the left coronary artery. FIG. 43D depicts representative immunohistochemistry images showing identification of myofibroblasts (α-SMA+/CD31−) vs. smooth muscle cells (α-SMA+/CD31+).

FIG. 44, comprising FIG. 44A through FIG. 44N, depicts results of experiments demonstrating Mitochondrial Na+/Ca2+ exchanger (NCLX) expression and ₘCa²⁺ handling is significantly altered in Alzheimer's disease. FIG. 44A depicts western blots for expression of proteins associated with ₘCa²⁺ exchange in non-familial AD patients and age-matched controls. n=7 for both groups. MCU, mitochondrial calcium uniporter; MCUb, mitochondrial calcium uniporter β subunit; MICU1, mitochondrial calcium uptake 1; MICU2, mitochondrial calcium uptake 2; EMRE, essential MCU regulator; NCLX, Na⁺/Ca²⁺ exchanger. Voltage dependent anion channel (VDAC) and oxidative phosphorylation component CV-Sα, complex V α subunit; were used as mitochondrial loading controls. FIG. 44B depicts NCLX mRNA expression in brain tissue isolated from the frontal cortex of 3xTg-AD mutant mice at 2, 4, 8 and 12m and age-matched outbred nontransgenic controls (NTg). FIG. 44C depicts western blots for expression of ₘCa²⁺ exchanger in 3xTg-AD mutant mice (12m) and age-matched outbred nontransgenic controls (NTg). FIG. 44D depicts NCLX mRNA expression in con (N2a)+Ad-NCLX, APPswe and APPswe+Ad-NCLX vs. con (N2a). FIG. 44E depicts western blots for NCLX expression in Con (N2a), Con+Ad-NCLX, APPswe and APPswe+Ad-NCLX from three independent experiments. FIG. 37F depicts Representative fluorescence traces of ₘCa²⁺ transients recoded in cells expressing the genetic ₘCa²⁺ sensor, mitoR-GECO after stimulation with KCl. FIG. 37G depicts quantification of ₘCa²⁺ transient peak amplitude. FIG. 37H depicts percent ₘCa²⁺ efflux vs. con, was calculated FIG. 37I depicts representative fluorescence traces of _{c}Ca²⁺ transients recoded in cells loaded with the _{c}Ca²⁺ reporter Fluo4-AM. FIG. 37J depicts quantification of _{c}Ca²⁺ peak amplitude. FIG. 44K depicts representative trace for ₘCa²⁺ retention capacity in con, con+Ad-NCLX, APPswe and APPswe cells infected with adenovirus encoding mitochondrial Na⁺/Ca²⁺ exchanger (NCLX). Cells were permeabilized with digitonin and treated with thapsigargin to inhibit SERCA and loaded cells with the ratiometric reporters FuraFF (Ca²⁺) and JC1 (mitochondrial membrane potential). The protonophore, FCCP, was used at the conclusion of the experiment to correct for total Ca²⁺ in the system. FIG. 44I depicts the percent change in ₘCa²⁺ retention capacity of con+Ad-NCLX (n=4), APPswe (n=3) and APPswe+Ad-NCLX (n=3) vs. con (N2a) cells (n=4) (Ca²⁺ load prior to membrane collapse), was calculated from traces shown in (k). FIG. 44M depicts representative traces for basal ₘCa²⁺ in con, con+Ad-NCLX, APPswe and APPswe+Ad-NCLX. Cells were loaded with Fura2 and treated with digitonin and thapsigargin. Upon reaching a steady state recording, the protonophore, FCCP, was used to collapse AN and initiate the release of all matrix free Ca². FIG. 44N depicts quantification of basal ₘCa²⁺ content.

FIG. 45, comprising FIG. 45A through FIG. 45S, depicts results of experiments demonstrating neuronal deletion of NCLX causes memory impairment associated with increased amyloidosis and tau-pathology in AD. FIG. 45A depicts schematic of gene targeting strategy. LoxP sites (red triangles) flank exons 5-7 and FRT sites (green half-circles) flank a splice acceptor site (En2-SA), β-galactosidase (βgal) reporter, and neomycin resistance (Neo) cassette. KO-1^{st} mutant mice were crossed with flippase expressing mice (ROSA26-FLPe) for removal of FRT flanked region resulting in an allele with conditional potential. Homozygous LoxP ‘foxed’ mice (NCLX^{fl/fl}) were crossed with neuron-restricted (Camk2a-promoter) Cre recombinase transgenic mice resulting in deletion of NCLX in brain cortex. Resultant neuronalspecific loss-of-function models (NCLX KO- NCLX^{fl/fl}×Camk2a-Cre) were crossed with 3xTg- AD mutant mouse, to generate 3xTg-AD×NCLX-KO (3xTg-AD×NCLX^{fl/fl}×Camk2a-Cre) mutant mice. FIG. 45B depicts qPCR analysis of NCLX mRNA expression corrected to the housekeeping gene Rps13 expressed as fold-change vs. Camk2a-Cre con.in tissue isolated from the brain cortex of 2m old mice. FIG. 45C depicts western blots for NCLX expression in tissue isolated from the hippocampus of 2m old 3xTg-AD×NCLX^{fl/fl}×Camk2a-Cre mutant mice compared to age-matched control. FIG. 45D Through FIG. 45E depicts working memory that was assessed in the Y-maze spontaneous alternation test in mice at the age of 6, 9 and 12 m in Camk2a-Cre, 3xTg-AD×Camk2a-Cre and 3xTg-AD×NCLX^{fl/fl}×Camk2a-Cre mice FIG. 45D depicts percentage spontaneous alternations. FIG. 45E depicts the number of total arm entries. FIG. 45F through FIG. 45H depicts hippocampus and amygdala associated memory was assessed in the fear conditioning test in mice at the age of 6, 9 and 12 m in Camk2a-Cre, 3xTg-AD×Camk2a-Cre and 3xTg-AD×NCLX^{fl/fl}×Camk2a-Cre mice FIG. 45F depicts freezing responses in the training phase. FIG. 45G depicts contextual recall freezing responses FIG. 45H depicts cued recall freezing responses. FIG. 451 through FIG. 45J depicts soluble (RIPA) and insoluble (formic acid extractable) A131-40 and A131-42 levels in brain cortex of 3xTg-AD×Camk2a-Cre and 3xTg-AD×NCLX″×Camk2a-Cre mice were measured by sandwich ELISA. FIG. 45K depicts representative sections of brains from 3xTg-AD×Camk2a-Cre and 3xTg-AD×NCLXW^{fl}×Camk2a-Cre mice immunostained with 4G8 antibody (Scale bar: 50 μm). FIG. 45L depicts quantification of the integrated optical density area occupied by Aβ immunoreactivity in brain of 3xTg-AD×Camk2a-Cre and 3xTg-AD×NCLX^{fl/fl}×Camk2a-Cre (n =4; *p,0.05). (Scale bar: 100 μm). FIG. 45M depicts Western blots of full-length APP, ADAM-10 (α-secretase) BACE1 (β-secretase), PS1, Nicastrin, APH (γ-secretase), and tubulin (load con). FIG. 45N depicts representative western blots of soluble and insoluble total tau (HT7), phosphorylated tau at residues Ser202/Thr205 (AT8), T231/S235 (AT180), T181 (AT270), and 5396 (PHF13) in soluble brain cortex homogenate from Camk2a-Cre, 3xTg-AD×Camk2a-Cre and 3xTg-AD×NCLXfl/fl×Camk2a-Cre mice (n =3 for all groups. *p,0.05). FIG. 450 depicts representative immunohistochemical staining for HT7 and AT8 in hippocampus of 3xTg-AD×Camk2a-Cre and 3xTg-AD×NCLX^{fl/fl}×Camk2a-Cre mice, (Scale bar: 50 μm). FIG. 45P through FIG. 45Q depicts quantification of the integrated optical density by the HT7 and AT8 immunoreactivity (n=4 for all groups. *p,0.05). FIG. 45R depicts representative immunohistochemical staining for 4-HNE in hippocampus of 3xTg-AD×Camk2a Cre and 3xTg-AD×NCLX^{fl/fl}×Camk2a-Cre mice, (Scale bar: 50 μm). FIG. 45S depicts quantification of the integrated optical density by the 4-HNE immunoreactivity (n=3 for 3xTg-AD×Camk2a-Cre; n=4 for 3xTg-AD×NCLX^{fl/fl}×Camk2a-Cre group. *p,0.05).

FIG. 46, comprising FIG. 46A through FIG. 46S, depicts results of experiments demonstrating Neuronal overexpression of NCLX restores memory and reduces AD pathology. FIG. 46A depicts schematic of tet-responsive transgenic construct and neuronal-specific driver, Camk2a-tTA. Resultant neuronal-specific gain-of-function models (NCLX nTg-TRE-NCLX×Camk2a-tTA) were crossed with 3xTg-AD mutant mouse to generate 3xTg-AD×TRE-NCLX×Camk2a-tTA mice. FIG. 46B depicts qPCR analysis of NCLX mRNA expression corrected to the housekeeping gene Rps13 expressed as fold-change vs. tTA con in tissue isolated from brain cortex of 2m old mice. FIG. 46C depicts western blots for NCLX expression in tissue isolated from the hippocampus of 2m old 3xTg-AD×TRE-NCLX×Camk2a-tTA mutant mice compared to age-matched control. FIG. 46D through FIG. 46E depicts working memory was assessed in the Y-maze spontaneous alternation test in mice at the age of 6, 9 and 12 m in Camk2a-tTA, 3xTg-AD×Camk2a-tTA and 3xTg-AD×TRE-NCLX×Camk2a-tTA mice. FIG. 46D depicts percentage spontaneous alternations. FIG. 46E depicts number of total arm entries. FIG. 46F through FIG. 46H depict hippocampus and amygdala associated memory was assessed in the fear conditioning test in mice at the age of 6, 9 and 12 m in Camk2a-tTA, 3xTg-AD×Camk2a-tTA and 3xTg-AD×TRENCLX×Camk2a-tTA mice FIG. 46F depicts freezing responses in the training phase FIG. 46H depicts contextual recall freezing responses FIG. 46H depicts Cued recall freezing responses. FIG. 461 through FIG. 46J depicts soluble (RIPA) and insoluble (formic acid extractable) Aβ1-40 and Aβ1-42 levels in brain cortex of 3xTg-AD×Camk2a-tTA and 3xTg-AD×TRE-NCLX x Camk2a-tTA mice were measured by sandwich ELISA. (n=7 for all groups. *p<0.05). FIG. 46K depicts representative sections of brains from 3xTg-AD×Camk2a-Cre and 3xTg-AD×TRE-NCLX×Camk2a-tTA mice immunostained with 4G8 antibody (Scale bar: 50 μm). FIG. 46L depicts quantification of the integrated optical density area occupied by Aβ immunoreactivity in brain of 3xTg-AD×Camk2a-Cre and 3xTg-AD×TRE-NCLX×Camk2a-tTA (n=4; *p<0.05). (Scale bar: 100 μm). FIG. 46M depicts western blots of full-length APP, ADAM-10 (α-secretase) BACE1 ((3-secretase), PS1, Nicastrin, APH (γ-secretase), and tubulin (load con). FIG. 46N depicts representative western blots of soluble and insoluble total tau (HT7), phosphorylated tau at residues Ser202/Thr205 (AT8), T231/5235 (AT180), T181 (AT270), and 5396 (PHF13) in soluble brain cortex homogenate from Camk2atTA, 3xTg-AD×Camk2a-tTA and 3xTg-AD×TRE-NCLX×Camk2a-tTA mice (n=3 for all groups. *p<0.05). FIG. 46O depicts representative immunohistochemical staining for HT7 and AT8 in hippocampus of 3xTg-AD×Camk2a-tTA and 3xTg-AD×TRE-NCLX×Camk2a-tTA, (Scale bar: 50 μm). FIG. 46P through FIG. 46Q depicts quantification of the integrated optical density by the HT7 and AT8 immunoreactivity (n=4 for all groups. *p<0.05). FIG. 46R depicts representative immunohistochemical staining for 4-HNE in hippocampus of 3xTg-AD×Camk2a-tTA and 3xTg-AD×TRE-NCLX×Camk2a-tTA mice, (Scale bar: 50 μm).

FIG. 46S depicts quantification of the integrated optical density by the 4-HNE immunoreactivity (n=4 for all group. *p,0.05).

FIG. 47, comprising FIG. 47A through FIG. 47R, depicts results of experiments demonstrating enhancing ₘCa²⁺ efflux reduces OxPhos defects, oxidative stress, amyloidogenic Aβ pathway and membrane rupture in APPswe cells. FIG. 47A depicts the timeline for experimental protocol of cell differentiation assay and infection of maturated con and APPswe cells with adenovirus encoding NCLX (Ad-NCLX). FIG. 47B depicts representative OCRs at baseline and following: oligomycin (oligo; CV inhibitor; to uncover ATP-linked respiration), FCCP (protonophore to induce max respiration), and rotenone+antimycin A (Rot/AA; complex I and III inhibitor; complete OxPhos inhibition). FIG. 47C depicts quantification of basal respiration (base OCR—nonmito respiration (post-Rot/AA). FIG. 47D depicts quantification of ATP-linked respiration (post-oligo OCR base OCR). FIG. 47E depicts max respiratory capacity (post-FCCP OCR—post-Rot/AA). FIG. 47F depicts spare respiratory capacity (post-FCCP OCR—basal OCR). FIG. 47G depicts proton leak (post-Oligo OCR—post Rot/AA OCR). FIG. 47H depicts quantification of cell rox green fluorescent intensity (the total cellular ROS production); fold change vs. N2a con. FIG. 47I depicts quantification of DHE fluorescent intensity; fold change vs. N2a con. FIG. 47J depicts quantification of mitosox fluorescent intensity; fold change vs. N2a con. FIG. 47K depicts western blots of full-length APP, ADAM-10 (α-secretase) BACE1 (β-secretase), PS1, Nicastrin, APH (γ-secretase), and tubulin (load con). FIG. 47L depicts fluorometric quantification of β-secretase activity. FIG. 47M depicts ELISA quantification of extracellular Aβ1-40 and Aβ1-42 levels. FIG. 47N depicts representative images of intracellular protein aggregate accumulation in con, APPswe and APPswe+Ad-NCLX cells stained with proteostat aggresome detection reagent (red) and Hoechst 33342 nuclear stain (blue). Scale bars, 20 μm. FIG. 47O depicts quantitative analysis of protein aggregates per cell. FIG. 47P through FIG. 47R depicts con, APPswe and APPswe infected with Ad-NCLX for 48 h were assessed for plasma membrane rupture (hallmark of cell death) using the cell membrane impermeable dye, Sytox Green after treatment. FIG. 47P depicts treatment with Ionomycin (Ca²⁺ overload, 1-5 μM), FIG. 47Q depicts treatment with tert-Butyl hydroperioxide (TBH, oxidizing agent, 10-30 μM), FIG. 47R depicts treatment with glutamate (NDMARagonist, neuroexcitotoxicity agent, 10-50 μM).

FIG. 48, comprising FIG. 48A through FIG. 48Y, depicts results of experiments demonstrating ₘCa²⁺ exchanger expression and ₘCa²⁺ handling in Alzheimer's disease. FIG. 48A depicts mRNA expression of ₘCa²⁺ exchanger in brain tissue isolated from the frontal cortex of 2-month-old 3xTg-AD mutant mice and age-matched outbred non-transgenic controls (NTg). FIG. 48B depicts mRNA expression of ₘCa²⁺ exchanger in brain tissue isolated from the frontal cortex of 4-month-old 3xTg-AD mutant mice and age-matched outbred nontransgenic controls (NTg). FIG. 48C depicts mRNA expression of ₘCa²⁺ exchanger in brain tissue isolated from the frontal cortex of 8-month-old 3xTg-ADmutant mice and age-matched outbred non-transgenic controls (NTg). FIG. 48D depicts mRNA expression of ₘCa²⁺ exchanger in brain tissue isolated from the frontal cortex of aged (12 mo.) 3xTg-AD mutant mice and outbred non-transgenic controls (NTg). n=3 for both groups; **p<0.01, one-way ANOVA, Sidak's multiple comparisons test. FIG. 48E depicts western blots for expression of proteins associated with ₘCa²⁺ exchange in 3xTg-AD mutant mice (2 mo.) and age-matched outbred non-transgenic controls (NTg). FIG. 48f depicts western blots for expression of proteins associated with ₘCa²⁺ exchange in neuroblastoma control cell line (N2a) vs. cells stably expressing cDNA encoding the APP Swedish mutant (K670N, M671L, APPswe). FIG. 48G depicts quantification of ₘCa²⁺ rise time. FIG. 48H depicts fold change in cCa²⁺ uptake rate of con+Ad-NCLX, APPswe and APPswe+Ad-NCLX vs. con (N2a) cells. FIG. 48I depicts time to 50% _{c}Ca²⁺ transient decay (T-50%). FIG. 48J through FIG. 48M depicts representative traces for ₘCa²⁺ retention capacity in con, con+Ad-NCLX, APPswe and APPswe cells infected with adenovirus encoding mitochondrial Na+/Ca2+ exchanger (NCLX). Cells were loaded with the ratiometric Ca²⁺ reporter, Fura-FF (1 uM), and ΔΨ indicator (JC-1). Cells were permeabilized with digitonin (40 μg/ml) to block all Ca²⁺ flux and treated with thapsigargin (2 μM) to inhibit SERCA and block ER Ca²⁺ uptake for simultaneous ratiometric monitoring during repetitive additions of 10 μM Ca²⁺ (blue arrow). FCCP was used as a control to collapse ΔΨ at the conclusion of each experiment. FIG. 48N through FIG. 48Y depicts densitometry analysis of all the western blots.

FIG. 49, comprising FIG. 49A through FIG. 49U, depicts results of experiments demonstrating studying the neuronal specific NCLX deletion effect on the amyloidogenic Aβ and tau pathway. FIG. 49A depicts western blots for expression of proteins associated with ₘCa²⁺ exchange in tissue isolated from the hippocampus of 2m old Camk2a-Cre, 3xTg-AD×Camk2a-Cre and 3xTg-AD×NCLXfl/fl×Camk2a-Cre mutant mice FIG. 49B through FIG. 49C depicts working memory was assessed in the Y-maze spontaneous alternation test in mice at the age of 6m in Camk2a-Cre and NCLX^{fl/fl}×Camk2a-Cre mice FIG. 49B depicts percentage spontaneous alternations. FIG. 49c depicts number of total arm entries. FIG. 49D through FIG. 49F depicts hippocampus and amygdala associated memory was assessed in the fear conditioning test in mice at the age of 6m in Camk2a-Cre and NCLXfl/fl×Camk2a-Cre mice FIG. 49D depicts freezing responses in the training phase. FIG. 49E depicts contextual recall freezing responses FIG. 49F depicts cued recall freezing responses. FIG. 49G depicts soluble (RIPA) and insoluble (formic acid extractable) Aβ1-42/Aβ1-40 ratio in brain cortex of 3xTg-AD×Camk2a-Cre and 3xTg-AD×NCLXfl/fl×Camk2a-Cre mice were measured by sandwich ELISA. FIG. 49H through FIG. 49U depicts densitometry analysis of all the western blots.

FIG. 50, comprising FIG. 50A through FIG. 50U, depicts results of experiments demonstrating studying the neuronal specific NCLX deletion effect on the amyloidogenic and tau pathway. FIG. 50A depicts western blots for expression of proteins associated with mCa²⁺ exchange in tissue isolated from the hippocampus of 2m old 3xTg-AD×TRE-NCLX×Camk2a-tTA mutant mice. FIG. 50B through FIG. 50C depicts working memory was assessed in the Y-maze spontaneous alternation test in mice at the age of 6m in Camk2a-tTA, 3xTg-AD×Camk2a-tTA and 3xTg-AD×TRE-NCLX x Camk2a-tTA mice. FIG. 50B depicts percentage spontaneous alternations. FIG. 50C depicts number of total arm entries. FIG. 50D through FIG. 5OF depicts hippocampus and amygdala associated memory was assessed in the fear conditioning test in mice at the age of 6m in Camk2a-tTA, 3xTg-AD×Camk2a-tTA mice. FIG. 50D depicts freezing responses in the training phase FIG. 50E depicts contextual recall freezing responses. FIG. 5OF depicts cued recall freezing responses. FIG. 50G depicts Soluble (RIPA) and insoluble (formic acid extractable) Aβ1-42/Aβ1-40 ratio in brain cortex of 3xTg-AD×Camk2a-Cre and 3xTg-AD×NCLX^{fl/fl}×Camk2a-Cre mice were measured by sandwich ELISA. FIG. 50H through FIG. 50U depicts densitometry analysis of all the western blots.

FIG. 51, comprising FIG. 51A through FIG. 51I, depicts experimental results demonstrating enhancing ₘCa²⁺ efflux effect on cell viability and amyloidogenic Aβ pathway in APPswe cells. FIG. 51A depicts cell viability of N2a, APPswe and APPswe infected with Ad-NCLX for 48h and treated with Ionomycin (Ca²⁺ overload, 1-5 μM). FIG. 51B depicts cell viability of N2a, APPswe and APPswe infected with Ad-NCLX for 48 h and treated with tert-Butyl hydroperioxide (TBH, oxidizing agent, 10-30 μM). FIG. 51C depicts cell viability of N2a, APPswe and APPswe infected with Ad-NCLX for 48 h and treated with glutamate (NDMAR-agonist, neuroexcitotoxicity agent, 10-50 μM). FIG. 51D through 51I depicts densitometry analysis of western blots.

FIG. 52 depicts the full-length western blots shown in Example 5.

FIG. 53 depicts densitometry analysis of all the western blots shown in FIGS. 48-51.

FIG. 54 depicts experimental results demonstrating tamoxifen-induced ablation of mNCX resulted in sudden death with most mice dying the first week after cre-mediated deletion.

FIG. 55 depicts experimental results demonstrating mNCX overexpression mouse model displayed preserved LV function, structure and a reduction in HF indices in myocardial infarction (LCA ligation) and pressure-overload induced HF (transverse aortic constriction).

### DETAILED DESCRIPTION

The present invention provides compositions and methods for treating or preventing neurodegeneration. In certain embodiments, the invention relates to treating Alzheimer's Disease (AD) amyotrophic lateral sclerosis, Parkinson's, Alzheimer's, Huntington's, Batten disease, prion disease, motor neuron diseases, traumatic brain injury, blast injury, dementia, Tay-Sachs, Niemann-Pick, PDH deficiency, aggregation disorders, encephalopathies, ataxia disorders, or neurodegeneration associated with aging.

In one aspect, the invention relates to the discovery that mitochondrial Ca²⁺ (mCA²⁺) overload is a primary contributor to AD pathology by promoting metabolic dysfunction and neuronal cell death and that enhancing ₘCa²⁺ efflux via adenoviral expression of the mitochondrial Na⁺/Ca²⁺ exchanger (mNCX) represents a new therapeutic target to inhibit or reverse AD progression. In one embodiment, the method comprises treating or preventing neurodegeneration by modulating mitochondrial calcium uniporter complex (MCU) expression, activity, or both. In one embodiment, modulating the mitochondrial calcium uniporter complex includes modulating a component of the MCU. Components of the MCU include, but are not limited to mNCX, MCU, MCUb, EMRE, MICU1, and MICU2. In one embodiment, the method comprises treating or preventing neurodegeneration by modulating mNCX expression, activity, or both.

The present invention also provides compositions and methods for inhibiting myofibroblast transdifferentiation and for treating or preventing fibrosis or a cardiovascular disease or disorder. In certain embodiments, the invention relates to treating diseases and disorders associated with fibrosis.

In one aspect, the invention relates to the discovery that mitochondrial calcium uptake is associated myofibroblast transdifferentiation and cardiac fibrosis post injury. Modulating mitochondrial calcium efflux via the mitochondrial calcium/sodium exchanger (mNCX) is a novel therapeutic angle to treat pathological fibrosis. Modulating MCU is a novel therapeutic angel to treat pathological fibrosis. In one embodiment, the method comprises treating or preventing myofibroblast transdifferentiation by modulating mNCX expression, activity, or both. In one embodiment, the method comprises treating or preventing fibrosis by modulating mNCX expression, activity, or both. In one embodiment, the method comprises treating or preventing a disease or disorder associated with fibrosis by modulating mNCX expression, activity, or both. In one embodiment, the method comprises treating or preventing myofibroblast.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In some embodiments, the patient, subject or individual is a mammal such as a non-primate (e.g., cows, pigs, horses, cats, dogs, rats, etc.) and a primate (e.g., monkey and human), most preferably a human. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and FIT fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, a “peptidomimetic” is a compound containing non-peptidic structural elements that is capable of mimicking the biological action of a parent peptide. A peptidomimetic may or may not comprise peptide bonds.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

As used herein, the term “transdominant negative mutant gene” refers to a gene encoding a polypeptide or protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides compositions and methods for treating or preventing neurodegeneration. In certain embodiments, the invention relates to treating Alzheimer's Disease (AD) amyotrophic lateral sclerosis, Parkinson's, Alzheimer's, Huntington's, Batten disease, prion disease, motor neuron diseases, traumatic brain injury, blast injury, dementia, Tay-Sachs, Niemann-Pick, PDH deficiency, aggregation disorders, encephalopathies, ataxia disorders, or neurodegeneration associated with aging.

In one aspect, the invention relates to the discovery that mitochondrial Ca²⁺ (ₘCA²⁺) overload is a primary contributor to AD pathology by promoting metabolic dysfunction and neuronal cell death and that enhancing ₘCa²⁺ efflux via adenoviral expression of the mitochondrial Na⁺/Ca²⁺ exchanger (mNCX) represents a new therapeutic target to inhibit or reverse AD progression. In one embodiment, the method comprises treating or preventing neurodegeneration by modulating mitochondrial calcium uniporter complex (MCU) expression, activity, or both. In one embodiment, modulating the mitochondrial calcium uniporter complex includes modulating a component of the MCU. Components of the MCU include, but are not limited to mNCX, MCU, MCUb, EMRE, MICU1, and MICU2. In one embodiment, the method comprises treating or preventing neurodegeneration by modulating mNCX expression, activity, or both.

The present invention also provides compositions and methods for inhibiting myofibroblast transdifferentiation and for treating or preventing fibrosis or a cardiovascular disease or disorder. In certain embodiments, the invention relates to treating diseases and disorders associated with fibrosis.

In one aspect, the invention relates to the discovery that mitochondrial calcium uptake is associated myofibroblast transdifferentiation and cardiac fibrosis post injury. Modulating mitochondrial calcium efflux via the mitochondrial calcium/sodium exchanger (mNCX) is a novel therapeutic angle to treat pathological fibrosis. Modulating MCU is a novel therapeutic angel to treat pathological fibrosis. In one embodiment, the method comprises treating or preventing myofibroblast transdifferentiation by modulating mNCX expression, activity, or both. In one embodiment, the method comprises treating or preventing fibrosis by modulating mNCX expression, activity, or both. In one embodiment, the method comprises treating or preventing a disease or disorder associated with fibrosis by modulating mNCX expression, activity, or both. In one embodiment, the method comprises treating or preventing myofibroblast. transdifferentiation by modulating MCU expression, activity, or both. In one embodiment, the method comprises treating or preventing fibrosis by modulating MCU expression, activity, or both. In one embodiment, the method comprises treating or preventing a disease or disorder associated with fibrosis by modulating MCU expression, activity, or both.

In one aspect, the invention relates to the discovery that increased phosphorylated PDH increases myofibroblast transdifferentiation. Modulating the activity of PDH through calcium, PDH kinase or PDH phosphatase is a novel therapeutic angle to attenuate pathological fibrosis. In one embodiment, the method comprises treating or preventing myofibroblast. transdifferentiation by modulating PDH kinase or PDH phosphatase expression, activity, or both. In one embodiment, the method comprises treating or preventing fibrosis by modulating PDH kinase or PDH phosphatase expression, activity, or both. In one embodiment, the method comprises treating or preventing a disease or disorder associated with fibrosis by modulating PDH kinase or PDH phosphatase expression, activity, or both.

In one aspect, the invention relates to the discovery that many metabolic changes are associated with increased myofibroblast transdifferentiation and fibrosis. Alpha-ketoglutarate increases while succinate decreases myofibroblast transdifferentiation. In one aspect, the metabolic changes may be related to changes in alpha-ketoglutarate dependent demethylases (Ten-eleven translocation (TET) enzymes and the JmjC-domain containing histone demethylases (JHDMs)). Modulating alpha-ketoglutarate dependent demethylases is a novel therapeutic angle to attenuate pathological fibrosis. In one embodiment, the method comprises treating or preventing myofibroblast transdifferentiation by modulating an alpha-ketoglutarate dependent demethylase expression, activity, or both. In one embodiment, the method comprises treating or preventing fibrosis by modulating an alpha-ketoglutarate dependent demethylase expression, activity, or both. In one embodiment, the method comprises treating or preventing a disease or disorder associated with fibrosis by modulating an alpha-ketoglutarate dependent demethylase expression, activity, or both.

In one aspect, the invention relates to the discovery that increased glycolysis by activating the kinase activity of phosphofructokinase-2 (PFK-2) increases myofibroblast transdifferentiation while activating the phosphatase activity of PFK-2 decreases myofibroblast transdifferentiation. Modulating the activity of PFK-2 is a novel therapeutic angle to attenuate pathological fibrosis. In one embodiment, the method comprises treating or preventing myofibroblast transdifferentiation by modulating PFK-2 expression, activity, or both. In one embodiment, the method comprises treating or preventing fibrosis by modulating PFK-2 expression, activity, or both. In one embodiment, the method comprises treating or preventing a disease or disorder associated with fibrosis by modulating PFK-2 expression, activity, or both.

In one aspect, the invention relates to the discovery that alpha-ketoglutarate increases while succinate decreases myofibroblast transdifferentiation. Modulating metabolic changes that underlie myofibroblast transdifferentiation is a novel therapeutic angle to attenuate pathological fibrosis. In one embodiment, modulating the alpha-ketoglutarate to succinate ratio or the calcium sensitive alpha-ketoglutarate dehydrogenase is a novel therapeutic angle to attenuate pathological fibrosis. In one embodiment, the method comprises treating or preventing myofibroblast transdifferentiation by modulating alpha-ketoglutarate to succinate ratio. In one embodiment, the method comprises treating or preventing fibrosis by modulating alpha-ketoglutarate to succinate ratio. In one embodiment, the method comprises treating or preventing a disease or disorder associated with fibrosis by modulating alpha-ketoglutarate to succinate ratio. In one embodiment, the method comprises treating or preventing myofibroblast transdifferentiation by modulating calcium sensitive alpha-ketoglutarate dehydrogenase expression, activity, or both. In one embodiment, the method comprises treating or preventing fibrosis by modulating calcium sensitive alpha-ketoglutarate dehydrogenase expression, activity, or both. In one embodiment, the method comprises treating or preventing a disease or disorder associated with fibrosis by modulating calcium sensitive alpha-ketoglutarate dehydrogenase expression, activity, or both.

In one embodiment, fibrosis is a disease or disorder eliciting abnormal formation, accumulation and precipitation of an extracellular matrix, caused by fibroblasts, and refers to abnormal accumulation of a collagen matrix due to injury or inflammation that changes the structures and functions of various types of tissue. Regardless of where fibrosis arises, most etiology of fibrosis includes excessive accumulation of a collagen matrix substituting normal tissue. Exemplary fibrotic diseases include, but are not limited to, cardiac fibrosis, interstitial lung diseases, liver cirrhosis, wound healing, systemic scleroderma, and Sjogren syndrome. In one embodiment, cardiac fibrosis results from a cardiac injury. For example, in one embodiment cardiac fibrosis results from a injury including, but not limited to, myocardial infarction, aortic stenosis, restrictive cardiomyopathy, systemic and pulmonary hypertension, or carcinoid heart disease. In one embodiment, interstitial lung diseases include, but are not limited to idiopathic pulmonary fibrosis, interstitial pulmonary fibrosis, Coal workers' pneumosoniosis, asbestosis, ARDS. In one embodiment, wound healing diseases and disorders include, but are not limited to, hypertrophic scars, keloid scars.

### Compositions

In one embodiment, the invention provides a modulator (e.g., an inhibitor or activator) of mitochondrial Na⁺/Ca²⁺ exchanger (mNCX), a PDH kinase, a PDH phosphatase, an alpha-ketoglutarate dependent demethylase, phosphofructokinase-2 (PFK-2), calcium sensitive alpha-ketoglutarate dehydrogenase, and the ratio of alpha-ketoglutarate to succinate or ₘCa²⁺ efflux. In one embodiment, the present invention includes compositions for modulating the level or activity of mNCX in a subject, a cell, a tissue, or an organ in need thereof. In one embodiment, the compositions of the invention modulate the amount of polypeptide of mNCX, the amount of mRNA of mNCX, the amount of activity of mNCX, or a combination thereof. In one embodiment, the compositions of the invention modulate ₘCa²⁺ efflux.

The compositions of the invention include compositions for treating or preventing cardiovascular diseases, neurodegenerative diseases, fibrosis, and fibrosis-related diseases. In one embodiment, an activator of mNCX of the invention is useful for treating a neurodegenerative disease. In one embodiment, an inhibitor of mNCX of the invention is useful for treating fibrosis, fibrosis-related diseases and cardiovascular diseases.

### Activators

In various embodiments, the present invention includes compositions and methods of treating a neurodegenerative disease or disorder in a subject. In one embodiment, the composition for treating a neurodegenerative disease or disorder comprises an activator of mNCX. In one embodiment, the activator of the invention increases the amount of mNCX polypeptide, the amount of mNCX mRNA, the amount of mNCX activity, or a combination thereof.

In various embodiments, the present invention includes compositions and methods of treating a cardiovascular disease or disorder in a subject. In one embodiment, the composition for treating a cardiovascular disease or disorder comprises an activator of mNCX. In one embodiment, the activator of the invention increases the amount of mNCX polypeptide, the amount of mNCX mRNA, the amount of mNCX activity, or a combination thereof.

It will be understood by one skilled in the art, based upon the disclosure provided herein, that an increase in the level of mNCX encompasses the increase in mNCX expression, including transcription, translation, or both. The skilled artisan will also appreciate, once armed with the teachings of the present invention, that an increase in the level of mNCX includes an increase in mNCX activity (e.g., ₘCa²⁺ efflux). Thus, increasing the level or activity of mNCX includes, but is not limited to, increasing the amount of mNCX polypeptide, increasing transcription, translation, or both, of a nucleic acid encoding mNCX; and it also includes increasing any activity of a mNCX polypeptide as well.

Thus, the present invention relates to the prevention and treatment of a neurodegenerative disease or disorder by administration of a mNCX polypeptide, a recombinant mNCX polypeptide, an active mNCX polypeptide fragment, or an activator of mNCX expression or activity.

It is understood by one skilled in the art, that an increase in the level of mNCX encompasses the increase of mNCX protein expression. Additionally, the skilled artisan would appreciate, that an increase in the level of mNCX includes an increase in mNCX activity. Thus, increasing the level or activity of mNCX includes, but is not limited to, increasing transcription, translation, or both, of a nucleic acid encoding mNCX; and it also includes increasing any activity of mNCX as well.

Activation of mNCX can be assessed using a wide variety of methods, including those disclosed herein, as well as methods well-known in the art or to be developed in the future. That is, the routineer would appreciate, based upon the disclosure provided herein, that increasing the level or activity of mNCX can be readily assessed using methods that assess the level of a nucleic acid encoding mNCX (e.g., mRNA) and/or the level of mNCX polypeptide in a biological sample obtained from a subject.

A mNCX activator can include, but should not be construed as being limited to, a chemical compound, a protein, a peptidomemetic, an antibody, a nucleic acid molecule. One of skill in the art would readily appreciate, based on the disclosure provided herein, that a mNCX activator encompasses a chemical compound that increases the level, enzymatic activity, or the like of mNCX. In some embodiments, the enzymatic activity is ₘCa²⁺ efflux. Additionally, a mNCX activator encompasses a chemically modified compound, and derivatives, as is well known to one of skill in the chemical arts.

It will be understood by one skilled in the art, based upon the disclosure provided herein, that an increase in the level of mNCX encompasses the increase in mNCX expression, including transcription, translation, or both. The skilled artisan will also appreciate, once armed with the teachings of the present invention, that an increase in the level of mNCX includes an increase in mNCX activity (e.g., enzymatic activity, receptor binding activity, etc.). Thus, increasing the level or activity of mNCX includes, but is not limited to, increasing the amount of mNCX polypeptide, increasing transcription, translation, or both, of a nucleic acid encoding mNCX; and it also includes increasing any activity of a mNCX polypeptide as well. The mNCX activator compositions and methods of the invention can selectively activate mNCX. Thus, the present invention relates to neuroprotection by administration of a mNCX polypeptide, a recombinant mNCX polypeptide, an active mNCX polypeptide fragment, or an activator of mNCX expression or activity.

Further, one of skill in the art would, when equipped with this disclosure and the methods exemplified herein, appreciate that a mNCX activator includes such activators as discovered in the future, as can be identified by well-known criteria in the art of pharmacology, such as the physiological results of activation of mNCX as described in detail herein and/or as known in the art. Therefore, the present invention is not limited in any way to any particular mNCX activator as exemplified or disclosed herein; rather, the invention encompasses those activators that would be understood by the routineer to be useful as are known in the art and as are discovered in the future.

Further methods of identifying and producing a mNCX activator are well known to those of ordinary skill in the art, including, but not limited, obtaining an activator from a naturally occurring source. Alternatively, a mNCX activator can be synthesized chemically. Further, the routineer would appreciate, based upon the teachings provided herein, that a mNCX activator can be obtained from a recombinant organism. Compositions and methods for chemically synthesizing mNCX activators and for obtaining them from natural sources are well known in the art and are described in the art.

One of skill in the art will appreciate that an activator can be administered as a small molecule chemical, a protein, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of mNCX.

One of skill in the art will realize that diminishing the amount or activity of a molecule that itself diminishes the amount or activity of mNCX can serve to increase the amount or activity of mNCX. Any inhibitor of a regulator of mNCX is encompassed in the invention. As a non-limiting example, antisense is described as a form of inhibiting a regulator of mNCX in order to increase the amount or activity of mNCX. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity mNCX, thereby increasing the amount or activity of mNCX. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of mNCX can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that a mNCX polypeptide, a recombinant mNCX polypeptide, or an active mNCX polypeptide fragment can be administered singly or in any combination thereof. Further, a mNCX polypeptide, a recombinant mNCX polypeptide, or an active mNCX polypeptide fragment can be administered singly or in any combination thereof in a temporal sense, in that they may be administered simultaneously, before, and/or after each other. One of ordinary skill in the art will appreciate, based on the disclosure provided herein, that a mNCX polypeptide, a recombinant mNCX polypeptide, or an active mNCX polypeptide fragment can be used to prevent or treat a neurodegenerative disease or disorder, and that an activator can be used alone or in any combination with another mNCX polypeptide, recombinant mNCX polypeptide, active mNCX polypeptide fragment, or mNCX activator to effect a therapeutic result.

One of skill in the art, when armed with the disclosure herein, would appreciate that the treating a neurodegenerative disease or disorder encompasses administering to a subject a mNCX mNCX polypeptide, a recombinant mNCX polypeptide, an active mNCX polypeptide fragment, or mNCX activator as a preventative measure against a neurodegenerative disease or disorder. As more fully discussed elsewhere herein, methods of increasing the level or activity of a mNCX encompass a wide plethora of techniques for increasing not only mNCX activity, but also for increasing expression of a nucleic acid encoding mNCX. Additionally, as disclosed elsewhere herein, one skilled in the art would understand, once armed with the teaching provided herein, that the present invention encompasses a method of preventing a wide variety of diseases where increased expression and/or activity of mNCX mediates, treats or prevents the disease. Further, the invention encompasses treatment or prevention of such diseases discovered in the future.

The invention encompasses administration of a mNCX polypeptide, a recombinant mNCX polypeptide, an active mNCX polypeptide fragment, or a mNCX activator to practice the methods of the invention; the skilled artisan would understand, based on the disclosure provided herein, how to formulate and administer the appropriate mNCX polypeptide, recombinant mNCX polypeptide, active mNCX polypeptide fragment, or mNCX activator to a subject. However, the present invention is not limited to any particular method of administration or treatment regimen. This is especially true where it would be appreciated by one skilled in the art, equipped with the disclosure provided herein, including the reduction to practice using an art-recognized model of a neurodegenerative disease, that methods of administering a mNCX polypeptide, a recombinant mNCX polypeptide, an active mNCX polypeptide fragment, or mNCX activator can be determined by one of skill in the pharmacological arts.

As used herein, the term “pharmaceutically-acceptable carrier” means a chemical composition with which an appropriate mNCX polypeptide, recombinant mNCX polypeptide, active mNCX polypeptide fragment, or mNCX activator, may be combined and which, following the combination, can be used to administer the appropriate mNCX polypeptide, recombinant mNCX polypeptide, active mNCX polypeptide fragment, or mNCX activator to a subject.

### Inhibitors

In various embodiments, the present invention includes compositions and methods of treating fibrosis, fibrosis-related diseases or disorders and cardiovascular diseases or disorders in a subject. In various embodiments, the composition for treating fibrosis, fibrosis-related diseases or disorders and cardiovascular diseases or disorders comprises an inhibitor of mNCX. In one embodiment, the inhibitor of the invention decreases the amount of mNCX polypeptide, the amount of mNCX mRNA, the amount of mNCX activity, or a combination thereof.

It will be understood by one skilled in the art, based upon the disclosure provided herein, that a decrease in the level of mNCX encompasses the decrease in the expression, including transcription, translation, or both. The skilled artisan will also appreciate, once armed with the teachings of the present invention, that a decrease in the level of mNCX includes a decrease in the activity of mNCX. Thus, decrease in the level or activity of mNCX includes, but is not limited to, decreasing the amount of polypeptide of mNCX, and decreasing transcription, translation, or both, of a nucleic acid encoding mNCX; and it also includes decreasing any activity of mNCX as well.

In one embodiment, the invention provides a generic concept for inhibiting mNCX as an anti-fibrotic therapy. In one embodiment, the composition of the invention comprises an inhibitor of mNCX. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of mNCX in a cell is by reducing or inhibiting expression of the nucleic acid encoding mNCX. Thus, the protein level of mNCX in a cell can also be decreased using a molecule or compound that inhibits or reduces gene expression such as, for example, siRNA, an antisense molecule or a ribozyme. However, the invention should not be limited to these examples.

Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to treat an autoimmune disease or disorder.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Nucleic Acid Inhibitors

In other related aspects, the invention includes an isolated nucleic acid. In some instances, the inhibitor is an siRNA, miRNA, or antisense molecule, which inhibits mNCX. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the invention, mNCX, can be inhibited by way of inactivating and/or sequestering mNCX. As such, inhibiting the activity of mNCX can be accomplished by using a transdominant negative mutant.

In one embodiment, siRNA is used to decrease the level of mNCX. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of mNCX at the protein level using RNAi technology.

In another aspect, the invention includes a vector comprising an siRNA or antisense polynucleotide. Preferably, the siRNA or antisense polynucleotide is capable of inhibiting the expression of a target polypeptide, wherein the target polypeptide is mNCX. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCRTM, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used to inhibit mNCX protein expression. The antisense expressing vector is used to transfect a mammalian cell or the mammal itself, thereby causing reduced endogenous expression of mNCX.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit mNCX protein expression. Ribozymes useful for inhibiting the expression of a target molecule may be designed by incorporating target sequences into the basic ribozyme structure, which are complementary, for example, to the mRNA sequence encoding mNCX. Ribozymes targeting mNCX, may be synthesized using commercially available reagents (Applied Biosystems, Inc., Foster City, Calif.) or they may be genetically expressed from DNA encoding them.

In one embodiment, the inhibitor of mNCX may comprise one or more components of a CRISPR-Cas system, where a guide RNA (gRNA) targeted to a gene encoding mNCX, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

Polypeptide Inhibitors

In other related aspects, the invention includes an isolated peptide inhibitor that inhibits mNCX. For example, in one embodiment, the peptide inhibitor of the invention inhibits mNCX directly by binding to mNCX thereby preventing the normal functional activity of mNCX. In another embodiment, the peptide inhibitor of the invention inhibits mNCX by competing with endogenous mNCX. In yet another embodiment, the peptide inhibitor of the invention inhibits the activity of mNCX by acting as a transdominant negative mutant.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Antibody Inhibitors

The invention also contemplates an inhibitor of mNCX comprising an antibody, or antibody fragment, specific for mNCX. That is, the antibody can inhibit mNCX to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Combinations

In one embodiment, the composition of the present invention comprises a combination of mNCX inhibitors described herein. In certain embodiments, a composition comprising a combination of inhibitors described herein has an additive effect, wherein the overall effect of the combination is approximately equal to the sum of the effects of each individual inhibitor. In other embodiments, a composition comprising a combination of inhibitors described herein has a synergistic effect, wherein the overall effect of the combination is greater than the sum of the effects of each individual inhibitor.

In some embodiments, the composition of the present invention comprises a combination of a mNCX inhibitor and second therapeutic agent. In one embodiment the second therapeutic agent includes cardiovascular therapies and fibrosis therapies. For example, in one embodiment the second therapeutic agents include, but are not limited to, Angiotensin-converting-enzyme (ACE) inhibitors (e.g. captopril, enalapril), Angiotensin II receptor blockers (e.g. losartan, valsartan), beta blockers (e.g. atenolol, carvedilol, metoprolol), aldosterone antagonists (e.g. spironolactone), calcium channel blockers (e.g. amlodipine, diltiazem, verapamil), idiopathic pulmonary fibrosis drugs (e.g. nintedanib, pirfenidone, Tralokinumab (anti-IL-13)), diffuse systemic sclerosis (e.g. Fresolimumab (anti-TGFb)), or topical treatments such as corticosteroids or calcineurin inhibitors.

A composition comprising a combination of inhibitors comprises individual inhibitors in any suitable ratio. For example, in one embodiment, the composition comprises a 1:1 ratio of two individual inhibitors. However, the combination is not limited to any particular ratio. Rather any ratio that is shown to be effective is encompassed.

### Therapeutic Methods

In one embodiment, the present invention provides methods for treatment, inhibition, prevention, or reduction of a neurodegenerative disease using an activator of mNCX of the invention. In one embodiment, the method comprises administering to the subject in need an effective amount of a composition that reduces or inhibits the expression or activity of mNCX.

In one embodiment neurodegenerative disease or disorder includes, but is not limited to, Parkinson's, Alzheimer's, Huntington's, Batten disease, prion disease, motor neuron diseases, traumatic brain injury, blast injury, dementia, Tay-Sachs, Niemann-Pick, PDH deficiency, encephalopathies, ataxia disorders, neurodgeneration associated with aging, autoimmune encephalomyelitis, degenerative nerve diseases, encephalitis (e.g. Rasmussen's encephalitis), Amyotrophic lateral sclerosis (ALS), Myasthenia gravis, Epilepsy, Autism, Pick's and Creutzfeldt Jakob's diseases, Charcot-Marie-Tooth Disease, Multiple sclerosis, Behcet's disease, Alexander disease, Krabbe disease, Guillain-Barre Syndrome, Spinal muscular atrophy, Gaucher's disease, Dentato-rubro-pallido-luysian atrophy (DRPLA), Hallervorden-Spatz Disease, Infantile Neuroaxonal Dystrophy, Kennedy's Disease, Kinsbourne syndrome, Lambert-Eaton Myasthenic Syndrome, Meningitis, Muscular Dystrophy, Multiple System Atrophy, Sydenham chorea (SD), Sandhoff Disease, Tourette syndrome, Transverse Myelitis, Alpers' disease, Gerstmann-Straussler-Scheinker disease (GSS), Leigh's disease, Cerebro-oculo-facio-skeletal syndrome (COFS), Progressive multifocal leukoencephalopathy (PML), Andermann syndrome, Corticobasal degeneration, frontotemporal dementia with parkinsonism liked to chromosome 17 (FTDP-17), primary age-related tauopathy (PART), chronic traumatic encephalopathy (CTE), progressive supranuclear palsy, Lytico-Bodig disease, ganglioglioma and gangliocytoma, meningioangiomatosis, postencephalitic Parkinsonism, subacute sclerosing panencephalitis, tauopathies, amyloid beta diseases and aggregation disorders.

In one embodiment, the present invention provides methods for treatment, inhibition, prevention, or reduction of a cardiovascular disease using a modulator of mNCX of the invention. In one embodiment, the method comprises administering to the subject in need an effective amount of a composition that modulates the expression or activity of mNCX. In one embodiment, the method comprises administering to the subject in need an effective amount of a composition that reduces or inhibits the expression or activity of mNCX. In one embodiment, the method comprises administering to the subject in need an effective amount of a composition that increases or activates the expression or activity of mNCX.

The following are non-limiting examples of cardiovascular diseases that can be treated by the disclosed methods and compositions: heart failure arterial cardiovascular thromboembolic disorders, venous cardiovascular thromboembolic disorders, and thromboembolic disorders in the chambers of the heart; ahtherosclerosis; restensosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acute myocardial infarction (AMI); myocardial infarction; non-Q wave myocardial infarction; non-STE myocardial infarction; coronary artery disease; cardiac ischemia; ischemia; ischemic sudden death; transient ischemic attack; stroke; atherosclerosis; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhythmias including supraventricular arrhythmias, atrial arrhythmias, atrial flutter, and atrial fibrillation.

In another embodiment, the present invention provides methods for treatment, inhibition, prevention, or reduction of fibrosis, a fibrosis-related disease or disorder or a cardiovascular disease or disorder using an inhibitor of mNCX of the invention. In one embodiment, the method comprises administering to the subject in need an effective amount of a composition that increases or activates the expression or activity of mNCX.

PDH is active in the dephosphorylated state and inactive in the phosphorylated state. Ca²⁺ activates PDH phosphatase leading to dephosphorylation of PDH and subsequently increases acetyl-CoA availability for the TCA cycle. In support of this theory, MCU-mediated uptake is required for PDH activation in the context of ‘fight or flight’ signaling. Ca²⁺ also increases the activity of a-ketoglutarate dehydrogenase (KGD) and isocitrate dehydrogenase (IDH) through yet unknown mechanisms. ₘCa²⁺ also modulates energy production by altering F 1-F0 ATPase function independent of changes in electron motive force (ΔΨ). In summation, ₘCa²⁺ can modify ATP.

Accordingly, in one embodiment, the activator of mNCX also modulates a PDH kinase, a PDH phosphatase, an alpha-ketoglutarate dependent demethylase, phosphofructokinase-2 (PFK-2), calcium sensitive alpha-ketoglutarate dehydrogenase, or the ratio of alpha-ketoglutarate to succinate. In one embodiment, wherein the alpha-ketoglutarate dependent demethylase is selected from the group consisting of a Ten-eleven translocation (TET) enzyme and a JmjC-domain containing histone demethylase (JHDM).

In one embodiment, the invention provides a method of treating or preventing fibrosis comprising administering a modulator of a PDH kinase, a PDH phosphatase, an alpha-ketoglutarate dependent demethylase, phosphofructokinase-2 (PFK-2), calcium sensitive alpha-ketoglutarate dehydrogenase, or the ratio of alpha-ketoglutarate to succinate. In one embodiment, wherein the alpha-ketoglutarate dependent demethylase is selected from the group consisting of a Ten-eleven translocation (TET) enzyme and a JmjC-domain containing histone demethylase (JHDM).

One aspect of the invention provides a method of treating or preventing fibrosis, a fibrosis-related disease or disorder or a cardiovascular disease or disorder using an inhibitor of the invention. In one embodiment, fibrotic diseases include, but are not limited to, cardiac fibrosis, interstitial lung diseases, liver cirrhosis, wound healing, systemic scleroderma, and Sjogren syndrome. In one embodiment, cardiac fibrosis results from a cardiac injury. For example, in one embodiment cardiac fibrosis results from a injury including, but not limited to, myocardial infarction, aortic stenosis, restrictive cardiomyopathy, systemic and pulmonary hypertension, or carcinoid heart disease. In one embodiment, interstitial lung diseases include, but are not limited to idiopathic pulmonary fibrosis, interstitial pulmonary fibrosis, Coal workers' pneumosoniosis, asbestosis, ARDS. In one embodiment, wound healing diseases and disorders include, but are not limited to, hypertrophic scars, keloid scars.

In one embodiment, fibrosis includes the formation or development of excess fibrous connective tissue in an organ or tissue as a reparative or reactive process, as opposed to a formation of fibrous tissue as a normal constituent of an organ or tissue. Skin and lungs are susceptible to fibrosis. Exemplary fibrotic conditions are scleroderma idiopathic pulmonary fibrosis, morphea, fibrosis as a result of Graft-Versus-Host Disease (GVHD), keloid and hypertrophic scar, and subepithelial fibrosis, endomyocardial fibrosis, uterine fibrosis, myelofibrosis, retroperitoneal fibrosis, nephrogenic systemic fibrosis, scarring after surgery, asthma, cirrhosis/liver fibrosis, aberrant wound healing, glomerulonephritis, and multifocal fibrosclerosis.

In some instances, fibrotic diseases are characterized by the activation of fibroblasts, increased production of collagen and fibronectin, and transdifferentiation into contractile myofibroblasts. This process usually occurs over many months and years, and can lead to organ dysfunction or death. Examples of fibrotic diseases include diabetic nephropathy, liver cirrhosis, idiopathic pulmonary fibrosis, rheumatoid arthritis, atherosclerosis, cardiac fibrosis and scleroderma (systemic sclerosis; SSc). Fibrotic disease represents one of the largest groups of disorders for which there is no effective therapy and thus represents a major unmet medical need. Often the only redress for patients with fibrosis is organ transplantation; since the supply of organs is insufficient to meet the demand, patients often die while waiting to receive suitable organs. Lung fibrosis alone can be a major cause of death in scleroderma lung disease, idiopathic pulmonary fibrosis, radiation- and chemotherapy-induced lung fibrosis and in conditions caused by occupational inhalation of dust particles.

The invention may be practiced in any subject diagnosed with, or at risk of developing, fibrosis. Fibrosis is associated with many diseases and disorders. Preferably, the fibrosis is idiopathic pulmonary fibrosis. The subject may be diagnosed with, or at risk for developing interstitial lung disease including idiopathic pulmonary fibrosis, scleroderma, radiation-induced pulmonary fibrosis, bleomycin lung, sarcoidosis, silicosis, familial pulmonary fibrosis, an autoimmune disease or any disorder wherein one or more fibroproliferative matrix molecule deposition, enhanced pathological collagen accumulation, apoptosis and alveolar septal rupture with honeycombing occurs. The subject may be identified as having fibrosis or being at risk for developing fibrosis because of exposure to asbestos, ground stone and metal dust, or because of the administration of a medication, such as bleomycin, busulfon, pheytoin, and nitro furantoin, which are risk factors for developing fibrosis. Preferably, the subject is a mammal and more preferably, a human. It is also contemplated that the compositions and methods of the invention may be used in the treatment of organ fibrosis secondary to allogenic organ transplant, e.g., graft transplant fibrosis. Non-limiting examples include renal transplant fibrosis, heart transplant fibrosis, liver transplant fibrosis, etc.

In certain embodiments, the methods of the present invention are used to treat multiple fibrotic diseases with underlying causes including myocardial infarct, cirrhosis, hepatitis, etc.

The invention may be practiced in any subject diagnosed with, or at risk of developing, scleroderma. Scleroderma is a chronic autoimmune disease characterized by fibrosis (or hardening), vascular alterations, and autoantibodies. There are two major forms: limited systemic scleroderma and diffuse systemic scleroderma. The cutaneous symptoms of limited systemic scleroderma affect the hands, arms and face. Patients with this form of scleroderma frequently have one or more of the following complications: calcinosis, Raynaud's phenomenon, esophageal dysfunction, sclerodactyl), and telangiectasias.

Diffuse systemic scleroderma is rapidly progressing and affects a large area of the skin and one or more internal organs, frequently the kidneys, esophagus, heart and/or lungs.

Scleroderma affects the small blood vessels known as arterioles, in all organs. First, the endothelial cells of the arteriole die off apoptotically, along with smooth muscle cells. These cells are replaced by collagen and other fibrous material. Inflammatory cells, particularly CD4+ helper T cells, infiltrate the arteriole, and cause further damage.

The skin manifestations of scleroderma can be painful, can impair use of the affected area (e.g., use of the hands, fingers, toes, feet, etc.) and can be disfiguring. Skin ulceration may occur, and such ulcers may be prone to infection or even gangrene. The ulcerated skin may be difficult or slow to heal. Difficulty in healing skin ulcerations may be particularly exacerbated in patients with impaired circulation, such as those with Raynaud's phenomenon. In certain embodiments, the methods of the present disclosure are used to treat scleroderma, for example skin symptoms of scleroderma. In certain embodiments, treating scleroderma comprises treating skin ulceration, such as digital ulcers. Administration of the peptides of the invention can be used to reduce the fibrotic and/or inflammatory symptoms of scleroderma in affected tissue and/or organs.

In addition to skin symptoms/manifestations, scleroderma may also affect the heart, kidney, lungs, joints, and digestive tract. In certain embodiments, treating scleroderma includes treating symptoms of the disease in any one or more of these tissues, such as by reducing fibrotic and/or inflammatory symptoms.

Lung problems are amongst the most serious complications of scleroderma and are responsible for much of the mortality associated with the disease. The two predominant lung conditions associated with scleroderma are pulmonary fibrosis and pulmonary hypertension. A patient with lung involvement may have either or both conditions. Lung fibrosis associated with scleroderma is one example of pulmonary fibrosis that can be treated using the peptides of the invention.

Scleroderma involving the lung causes scarring (pulmonary fibrosis). Such pulmonary fibrosis occurs in about 70% of scleroderma patients, although its progression is typically slow and symptoms vary widely across patients in terms of severity. For patients that do have symptoms associated with pulmonary fibrosis, the symptoms include a dry cough, shortness of breath, and reduced ability to exercise. About 16% of patients with some level of pulmonary fibrosis develop severe pulmonary fibrosis. Patients with severe pulmonary fibrosis experience significant decline in lung function and alveolitis.

In certain embodiments, the methods of the present invention include the use of the peptides of the invention to treat scleroderma, for example lung fibrosis associated with scleroderma. Administration of the peptides of the invention can be used to reduce the fibrotic symptoms of scleroderma in lung. For example, the methods can be used to improve lung function and/or to reduce the risk of death due to scleroderma. For example, the peptides of the invention can be used to treat scleroderma associated interstitial lung disease.

Kidney involvement is also common in scleroderma patients. Renal fibrosis associated with scleroderma is an example of renal fibrosis that can be treated by administration of an inhibitor of the invention.

In certain embodiments, the methods of the present invention are used to treat scleroderma, for example kidney fibrosis associated with scleroderma. Administration of a inhibitor of the invention can be used to reduce the fibrotic symptoms of scleroderma in kidney. For example, the methods can be used to improve kidney function, to reduce protein in the urine, to reduce hypertension, and/or to reduce the risk of renal crisis that may lead to fatal renal failure.

In certain embodiments, methods of treating scleroderma include administering a inhibitor of the invention as part of a therapeutic regimen along with one or more other drugs, biologics, or therapeutic interventions appropriate for scleroderma. In certain embodiments, the additional drug, biologic, or therapeutic intervention is appropriate for particular symptoms associated with scleroderma. By way of example, an inhibitor of the invention may be administered as part of a therapeutic regimen along with one or more immunosuppressive agents, such as methotrexate, cyclophosphamide, azathioprine, and mycophenolate mofetil. By way of further example, an inhibitor of the invention may be administered as part of a therapeutic regimen along with one or more agents designed to increase blood flow, such as blood flow to ulcerated digits (e.g., nifedipine, amlodipine, diltiazem, felodipine, or nicardipine). By way of further example, an inhibitor of the invention may be administered as part of a therapeutic regimen along with one or more agents intended to decrease fibrosis of the skin, such as d-penicillamine, colchicine, PUVA, Relaxin, and cyclosporine. By way of further example, a inhibitor of the invention may be administered as part of a therapeutic regimen along with steroids or broncho-dilators.

It will be appreciated by one of skill in the art, when armed with the present disclosure including the methods detailed herein, that the invention is not limited to treatment of autoimmune disease that is already established. Particularly, the disease or disorder need not have manifested to the point of detriment to the subject; indeed, the disease or disorder need not be detected in a subject before treatment is administered. That is, significant signs or symptoms of autoimmune disease do not have to occur before the present invention may provide benefit. Therefore, the present invention includes a method for preventing autoimmune disease, in that a composition, as discussed previously elsewhere herein, can be administered to a subject prior to the onset of autoimmune disease, thereby preventing autoimmune disease.

One of skill in the art, when armed with the disclosure herein, would appreciate that the prevention of an autoimmune disease or disorder, encompasses administering to a subject a composition as a preventative measure against the development of, or progression of autoimmune disease. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

The invention encompasses administration of a modulator of mNCX, or a combination thereof. To practice the methods of the invention; the skilled artisan would understand, based on the disclosure provided herein, how to formulate and administer the appropriate modulator composition to a subject. The present invention is not limited to any particular method of administration or treatment regimen.

One of skill in the art will appreciate that the inhibitors of the invention can be administered singly or in any combination. Further, the inhibitors of the invention can be administered singly or in any combination in a temporal sense, in that they may be administered concurrently, or before, and/or after each other. One of ordinary skill in the art will appreciate, based on the disclosure provided herein, that the inhibitor compositions of the invention can be used to prevent or to treat an autoimmune disease or disorder, and that an inhibitor composition can be used alone or in any combination with another modulator to effect a therapeutic result. In various embodiments, any of the inhibitor compositions of the invention described herein can be administered alone or in combination with other modulators of other molecules associated with autoimmune diseases.

In one embodiment, the invention includes a method comprising administering a combination of inhibitors described herein. In certain embodiments, the method has an additive effect, wherein the overall effect of the administering a combination of inhibitors is approximately equal to the sum of the effects of administering each individual inhibitor. In other embodiments, the method has a synergistic effect, wherein the overall effect of administering a combination of inhibitors is greater than the sum of the effects of administering each individual inhibitor.

The method comprises administering a combination of inhibitors in any suitable ratio. For example, in one embodiment, the method comprises administering two individual inhibitors at a 1:1 ratio. However, the method is not limited to any particular ratio. Rather any ratio that is shown to be effective is encompassed.

### Pharmaceutical Compositions and Formulations

The invention also encompasses the use of pharmaceutical compositions of the invention or salts thereof to practice the methods of the invention. Such a pharmaceutical composition may consist of at least one modulator composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, parenteral, sublingual, transdermal, transmucosal (e.g., sublingual, lingual, (trans)buccal, and (intra)nasal,), intravesical, intraduodenal, intragastrical, rectal, intra-peritoneal, subcutaneous, intramuscular, intradermal, intra-arterial, intravenous, or administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1. Genetic Rescue of Mitochondrial Calcium Efflux in Alzheimer's Disease Preserves Mitochondrial Function and Protects Against Neuronal Cell Death

It is described herein that 3xTg-AD mice and human AD brain samples have significant alterations in the expression of key ₘCa²⁺ exchange genes, most notably a reduction in the expression of the mitochondrial Na⁺/Ca²⁺ exchanger (mNCX, SLC8B1), the major efflux pathway in excitable cells. It was discovered that ₘCa²⁺ efflux and ₘCa²⁺ retention capacity was severely impaired in N2a/APPswe cells. Rescue of ₘCa²⁺ extrusion, via adenoviral expression of mNCX, enhanced the clearance of pathogenic ₘCa²⁺, recovered (AT), enhanced OxPhos, reduced extracellular Aβ1-40 levels and protected from ionomycin-, glutamate- and ROS-induced cell death. These data suggest that impaired ₘCa²⁺ exchange is a central contributor to neuronal cell death in AD and that mNCX represents a new therapeutic target to inhibit or reverse AD progression.

The materials and methods employed in these experiments are now described.

Mice

Triple-transgenic AD mice (3xTg-AD; APPswe, PS1-M146V, tau-P301L), and wild type mice of the same genetic backgrounds were maintained in animal facility under pathogen-free conditions on a 12-hour light/12-hour dark cycle with continuous access to food and water (Giannopoulos, P. F. et al., 2015, Biol Psychiatry 78: 693-701; Li et al., 2014, Ann Neurol 75:851-863; Di Meco et al., 2014, Neurobiology of aging 35:1813-1820). 3xTg Mice are homozygous for the Psen1 mutation (M146V knock-in), and contain transgenes inserted into the same loci expressing the APPswe mutation (APP KM670/671NL) and tau mutation (MAPT P301L).

Human AD Tissue Samples

Frontal cortex samples were collected post-mortem from non-familial AD patients and age matched controls with no history of dementia. All tissue samples were rapidly frozen in liquid nitrogen and stored at -80° C. until isolation of RNA and/or protein (n=3 for non- familial AD and n=3 for familial AD).

Cell cultures and Differentiation

Mouse neuroblastoma N2a cell line (N2a/Wt) and N2A cells stably expressing human APP carrying the K670 N, M671 L Swedish mutation (APPswe) were grown in Dulbecco's modified Eagle's medium supplemented with 10% fetal bovine serum, 1% penicillin/streptomycin and in the absence (N2a/Wt) or presence of 400 μg/mL G418 (APPswe) at 37° C. in the presence of 5% CO₂ (Chu et al., 2012, Annals of Neurology 72:442-54). In differentiation studies, cells were grown in 50% Dulbecco's modified Eagle's medium (DMEM), 50% OPTIMEM, 1% penicillin/streptomycin for 72 hours. Only cells with passage number <20 were used. For all imaging studies, cells were plated on glass coverslips pre-coated with poly-D-lysine. For overexpression of mNCX, maturated N2a-APPswe cells were infected with adenovirus encoding mNCX (Ad-mNCX) for 48 hrs.

qPCR mRNA Analysis

RNA was extracted using the Qiagen RNeasy Kit (Luongo et al., 2015, Cell reports 12:23-34). Briefly, 1 μg of total RNA was used to synthesize cDNA in a 20 μL reaction using the High-Capacity cDNA Reverse Transcription Kit (Applied Biosystems). qPCR analysis was conducted following manufacturer instructions. RPS-13 was always used as an internal control gene to normalize for the amount of RNA. Each sample was run in triplicate, and analysis of relative gene expression was done by using the 2^{-ΔΔCt} method.

Western Blot Analysis

All protein samples from brain or cell lysates (n=3/gp) were lysed by homogenization in RIPA buffer and used for western blot analyses (Luongo et al., 2015, Cell reports 12:23-34). Samples were run by electrophoresis on polyacrylamide Tris-glycine SDS gels. The following antibodies were used in the study: mNCX (1:500, NCKX6 Santa Cruz, sc-161921); MCU, (1:1,000, Sigma-Aldrich, HPA016480); MCUb (1:1,000, Abgent, AP12355b); MICU1 (1:500, Custom generation by Yenzyme); MICU2 (1:1,000, Abcam, ab101465); VDAC (1:2,500,Abcam, ab15895); ETC respiratory chain complexes (1:5,000, OxPhos Cocktail, Abcam, MS604) anti-APP N-terminal raised against amino acids 66-81 for total APP (22C11, 1:1500, Chemicon International, Temecula, Calif.), BACE1 (1:500,IBL America, USA), ADAM10 (1:500 dilution, Chemicon International), PSEN1 (1:500 dilution, Sigma-Aldrich, St Louis, Mo.), nicastrin (1:200 dilution, Cell Signaling Technology, Danvers, Mass.), APH-1 (1:200 dilution, Millipore, Billerica, Mass.), beta- Tubulin (1: 1000, Abcam,ab6046) and Licor IR secondary antibodies (1:12,000). All blots were imaged on a Licor Odyssey system (anti-mouse, 926-32210; anti-rabbit, 926-68073; anti-goat, 926-32214).

Live-Cell Imaging of Ca²⁺Transients

Maturated neuronal cells were infected with Ad-mitoR-GECO-1 to measure ₘCa²⁺ dynamics or loaded with the cytosolic Ca²⁺ indicator, 5-μM Fluo4-AM to study cytosolic Ca²⁺ dynamics. Cells were imaged continuously in Tyrode's buffer (150-mM NaCl, 5.4-mM KCl, 5-mM HEPES, 10-mM glucose, 2-mM CaCl2, 2-mM sodium pyruvate at pH 7.4) on a Zeiss 510 confocal microscope. Cell were treated with the depolarizing agent, 100 mM KCl, to activate voltage gated calcium channels during continuous live-cell imaging (Luongo et al., 2015, Cell reports 12:23-34).

Mitochondria Isolation

Brain cortex and hippocampus were excised from mice and mitochondria were isolated (Luongo et al., 2015, Cell reports 12:23-34). In brief, tissue was homogenized in ice-cold mitochondrial isolation buffer. The homogenate was centrifuged for 10 minutes at 700×g, and the supernatant was centrifuged again at 7,200×g for 10 minutes. The mitochondrial pellets were washed twice and were suspended in buffer containing 125 mM KCl, 20 mM Hepes, 2 mM MgCl2, 2 mM potassium phosphate, and 40 μM EGTA, pH 7.2, and supplemented with 5 mM Malate, 10 mM Glutamate, and 10 mM succinate.

Evaluation of ₘCa²⁺ Retention Capacity and Content

To evaluate ₘCa²⁺ retention capacity and content, N2a, N2a-APPswe and N2a-APPswe infected with Ad-mNCX for 48 hours were transferred to an intracellular-like medium containing (120-mM KCl, 10-mM NaCl, 1-mM KH2PO4, 20-mM HEPES-Tris), 3-μM thapsigargin to inhibit SERCA so that the movement of Ca²⁺ was only influenced by mitochondrial uptake, 80-μg/ml digitonin, protease inhibitors, supplemented with 10-μM succinate and pH to 7.2. All solutions were cleared with Chelex 100 to remove trace Ca²⁺. For ₘCa²⁺ retention capacity: 2×106 digitonin permeabilized neuronal cells were loaded with the ratiometric reporters FuraFF at concentration of 1-μM (Ca²⁺). At 20 s JC-1 was added to monitor (Δψm) mitochondrial membrane potential. Fluorescent signals were monitored in a spectrofluorometer at 340- and 380-nm ex/510-nm em. After acquiring baseline recordings, at 400 seconds, a repetitive series of Ca²⁺ boluses (10 μM) were added at the indicated time points. At completion of the experiment the protonophore, 10-μM FCCP, was added to uncouple the Avm and release matrix free-Ca²⁺. All experiments (3 replicates) were conducted at 37° C. For ₘCa²⁺ content maturated N2a cells from all 3 groups were loaded with Fura2 and treated with digitonin and thapsigargin (Luongo et al., 2015, Cell reports 12:23-34). Upon reaching a steady state recording, the protonophore, FCCP, was used to collapse AT and initiate the release of all matrix free Ca²⁺. To monitor ₘCa²⁺ retention capacity in 3xTg AD and NTG mice, mitochondria isolated from brain cortex and hippocampus were loaded with calcium green-5N (Molecular Probes), and continuously monitored for changes in fluorescence using a spectrofluorometer during 10-Mm bath Ca²⁺ additions every 50 seconds (Elrod, J. W., et al., 2010, Journal of clinical investigation 120:3680-3687).

Evaluation of Reactive Oxygen Species Production

To measure the total cellular ROS, fluorogenic probes CellROX Green was employed which is a cell-permeable non-fluorescent or very weakly fluorescent in a reduced state and exhibit strong fluorogenic signal upon oxidation. In this assay, cells were loaded with CellROX green Reagent at a final concentration of 5 μM for 30 minutes at 37° C. and measured the fluorescence at 485/ex and 520/em using a Tecan Infinite M1000 Pro plate reader. Cells from three groups (n=29 for N2a; n=30 N2a-APPswe; n=31 for N2a-APPswe+Ad-mNCX) were stained with 20-μm dihydroethidium for 30 minutes at 37° C. and imaged on Carl Zeiss 510 confocal microscope at 490/20ex and 632/60em. To measure mitochondrial superoxide production cells were loaded with 10-μM MitoSOX Red for 45 minutes at 37° C. and imaged at 490/20ex and 585/40em (n=52 for N2a, n=59 N2a-APPswe, and n=59 N2a-APPswe+Ad-mNCX). Images were quantified using ImageJ (Luongo et al., 2015, Cell reports 12:23-34).

Oxygen Consumption Rate

N2a, N2a-APPswe and N2a-APPswe infected with Ad-mNCX for 48 hours were subjected to oxygen consumption rate (OCR) measurement at 37° C. in an XF96 extracellular flux analyzer. Cells (3×104) were plated in XF media pH 7.4 supplemented with 25-mM glucose and 1-mM sodium pyruvate and sequentially exposed to oligomycin, FCCP, and rotenone plus antimycin A (Luongo et al., 2015, Cell reports 12:23-34).

Membrane Rupture and Cell Viability Assay

Membrane rupture was evaluated using SYTOX Green a membrane impermeable fluorescent stain, which upon membrane rupture enters the cell, intercalates DNA and increases fluorescence >500-fold and also examined general cell viability using Cell Titer Blue. This Cell Titer Blue assay uses the indicator dye resazurin to measure the metabolic capacity of cells. Viable cells retain the ability to reduce resazurin into resorufin, which is highly fluorescent. Nonviable cells rapidly lose metabolic capacity, do not reduce the indicator dye, and thus do not generate a fluorescent signal. N2a, N2a-APPswe and N2a-APPswe infected with Ad-mNCX for 48 hours were treated with Iono, (1-5 μM) for 24 hours and oxidizing agent tert-Butyl hydroperioxide (TBH) (10-30 μM) for 14 hours and glutamate (neuroexcitotoxicity agent) (10-50 μM) for 24 hours. On the day of the experiment, cells were loaded with 1-μM Sytox green for 15 minutes at 37° C. and measured the fluorescence at 504/ex and 523/em using a Tecan Infinite M1000 Pro plate reader. To measure number of viable cells, CellTiter-Blue Reagent (10 μl/well in 96 well plate) is added directly to each well, incubated at 37° C. for 2 hrs and the fluorescent signal at (560(20)Ex /590(10)Em).was measured using plate reader.

Sandwich ELISA Assay

For quantitative analysis of Aβ in conditioned medium, a sandwich enzyme linked immunosorbent assay (ELISA) was performed (Chu, J., et al., 2012, Annals of neurology 72:442-454). In brief, equal numbers of cells were plated in six well plates. For in vitro analysis of Aβ 1-40 and Aβ 1-42 levels, conditioned media from human APP-overexpressing N2a cells and cells infected AdmNCX were collected and analyzed at a 1:100 dilution. Aβ 1-40 and Aβ 1-42 in samples were captured with the monoclonal antibody BAN50, which specifically detects the N-terminal of human Aβ(1-16). Captured human Aβ is recognized by another antibody, BA27 F(ab′)2-HRP, a mAb specifically detects the C-terminal of Aβ40, or BC05 F(ab′)2-HRP, a mAb specific for the C-terminal of Aβ 42, respectively. HRP activity was assayed by color development using TMB. The absorbance was then measured at 450 nm. Values were reported as percentage of Aβ1-40 and Aβ1-42 secreted relative to control-APPswe.

Fluorometric Detection of β Secretase Activity

β-secretase activity was determined using fluorescent transfer peptides consisting of APP amino acid sequences containing the cleavage sites of BACE secretase. The method is based on the secretase-dependent cleavage of a secretase-specific peptide conjugated to the fluorescent reporter molecules EDANS and DABCYL, which results in the release of a fluorescent signal that was detected using a fluorescent microplate reader with excitation wavelength of 355 nm and emission at 510 nm. The level of secretase enzymatic

activity is proportional to the fluorometric reaction, and the data are expressed as fold increase in fluorescence over that of background controls. BACE1 activity was assayed by a

fluorescence-based in vitro assay kit (Yang, H. et al., 2010, Biological Psychiatry 68:922-929).

Detection of Protein Aggregates

For determination of misfolded protein aggregates, cells were fixed with 4% paraformaldehyde at RT for 15 min and, permeabilized in PB ST (0.15% TritonX-100 in PBS) at RT for 15 min. Cells were then stained with proteostat aggresome detection dye at RT for 30 min and Hoechst 33342 nuclear stain, using the method described in the manual. Proteostat, a molecular rotor dye that becomes fluorescent when binding to the β-sheet structure of misfolded proteins. All components of proteostat aggresome detection kit were prepared according to the manufacturer's instructions. Aggregated protein accumulation was detected using a Carl Zeiss 710 confocal microscope. (standard red laser set for the aggresome signal and DAPI laser set for the nuclear signal imaging). Further quantitative analyses, number of protein aggregates deposits per cell (n=41 for N2a, n=62 N2a-APPswe and n=69 N2a-APPswe+Ad-mNCX), were counted.

The results of the experiments are now described.

ₘCa²⁺ Exchanger Expression is Significantly Altered in AD

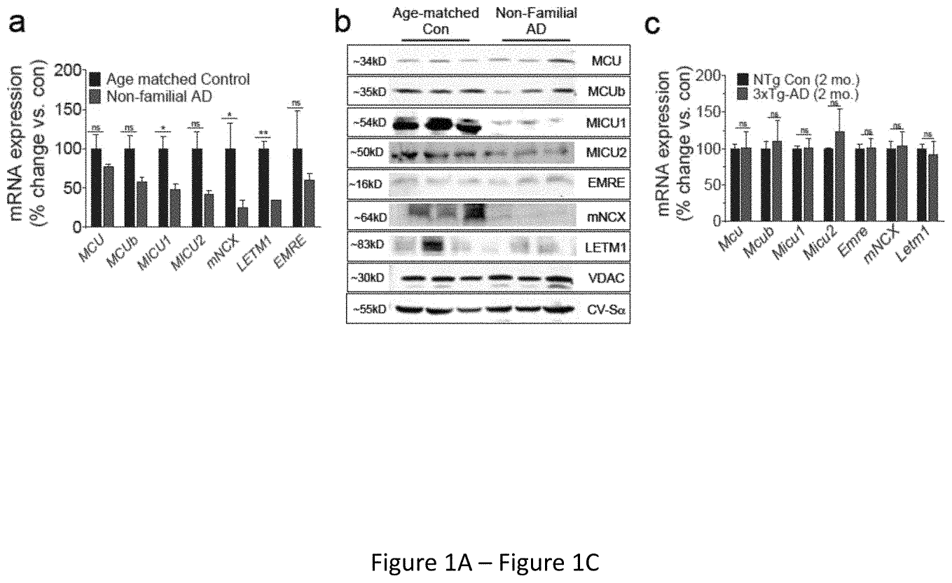

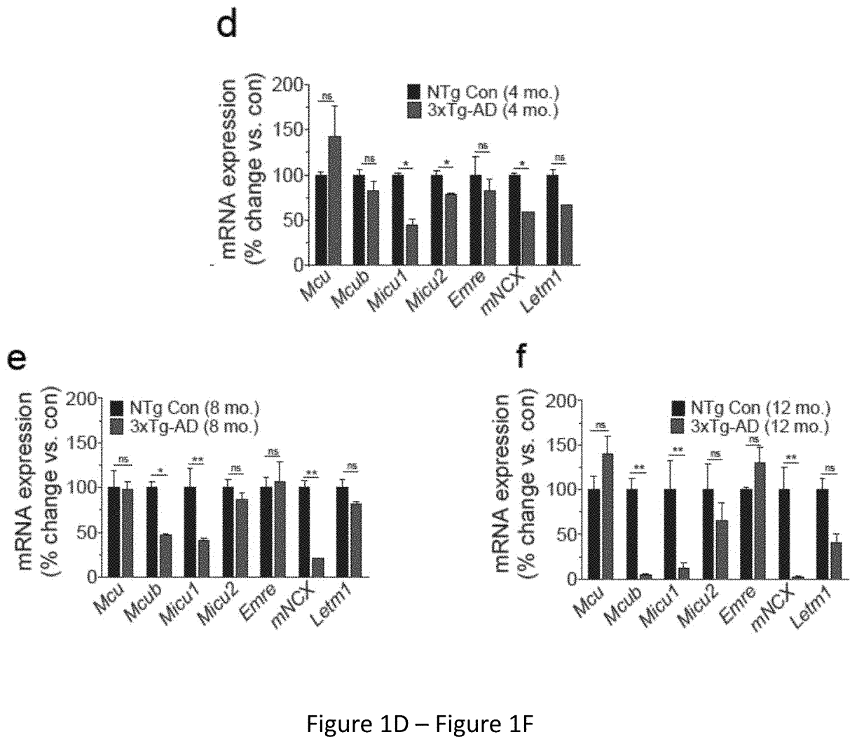

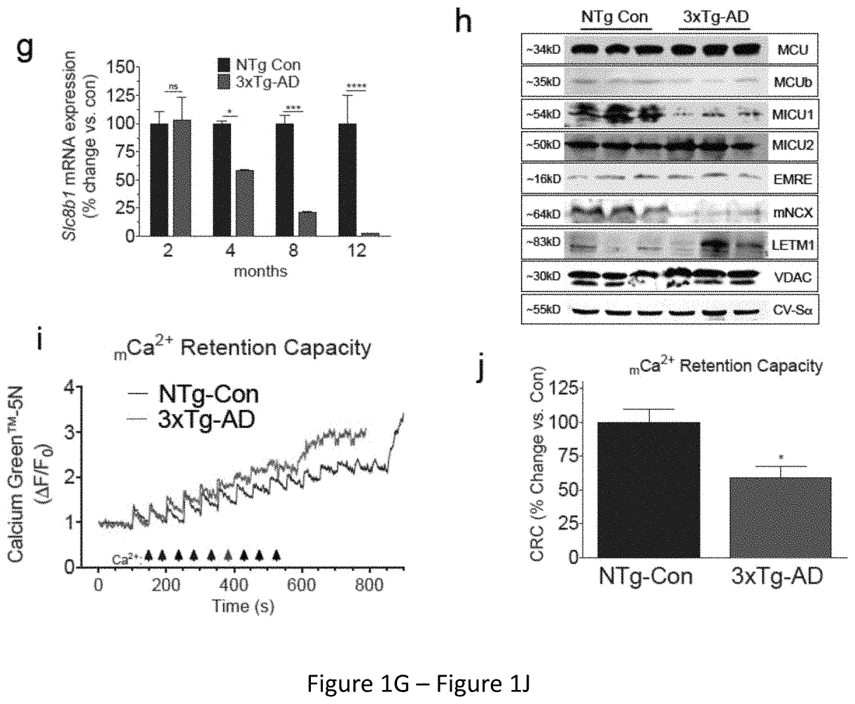

To decipher the role of ₘCa²⁺ signaling in AD human AD brain samples, the triple mutant AD mouse model (3x-Tg) and a neuroblastoma cell line stably expressing the human Swedish mutant amyloid precursor protein (N2a/APPswe) were examined for alterations in expression of genes associated with mitochondrial calcium exchange (FIG. 1). Frontal cortex samples were collected post-mortem from non-familial AD patients and age-matched controls with no history of dementia. RNA was isolated and SYBR-green qPCR was performed with all data corrected to the housekeeping gene, RPS13. A significant reduction in the MCU negative regulator, MICU1, and a substantial reduction in the ₘCa²⁺ efflux exchangers, mNCX (SLC8B1) and LEM1 was discovered. In addition, a trend towards a reduction in MCUb (CCDC109B) was noted (FIG. 1A). Next, protein was isolated and probed for changes in expression using standard western blot techniques. AD displayed a profound loss in expression of mNCX and MICU1, and a reduction in MCUb, confirming the mRNA results. VDAC and Complex V-Sα were used as mitochondrial loading controls (FIG. 1B).

To examine if alterations in ₘCa²⁺ transporter expression observed in AD patients is recapitulated in a murine model of AD, mutant mice harboring three mutations associated with familial AD (3xTg-AD, Psen1 mutation (M146V knock-in), APPswe mutation

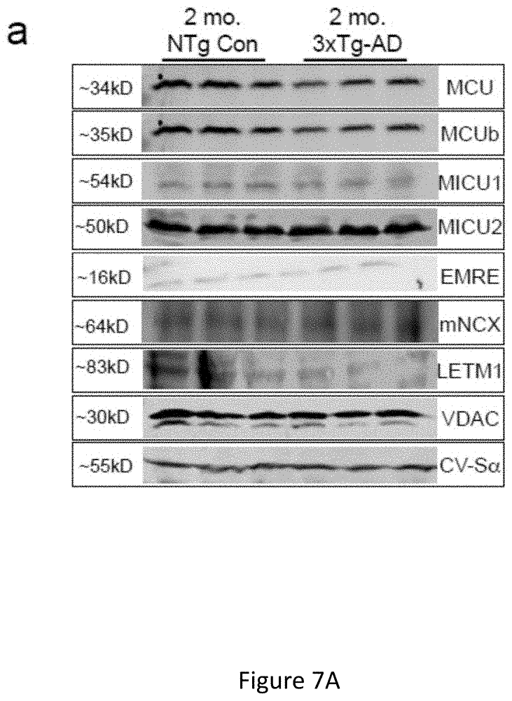

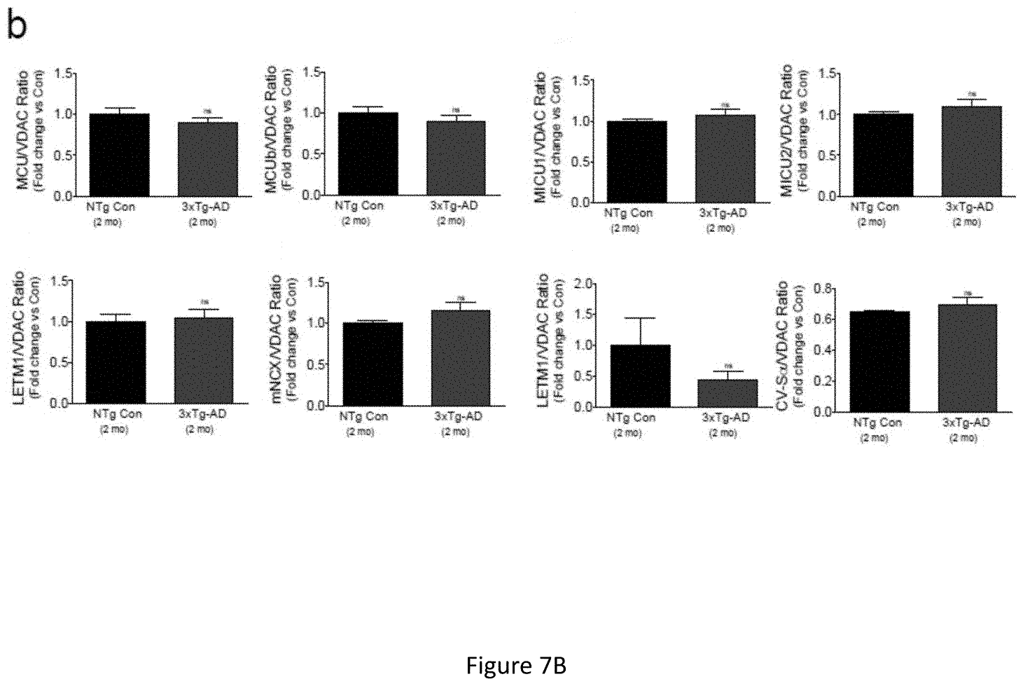

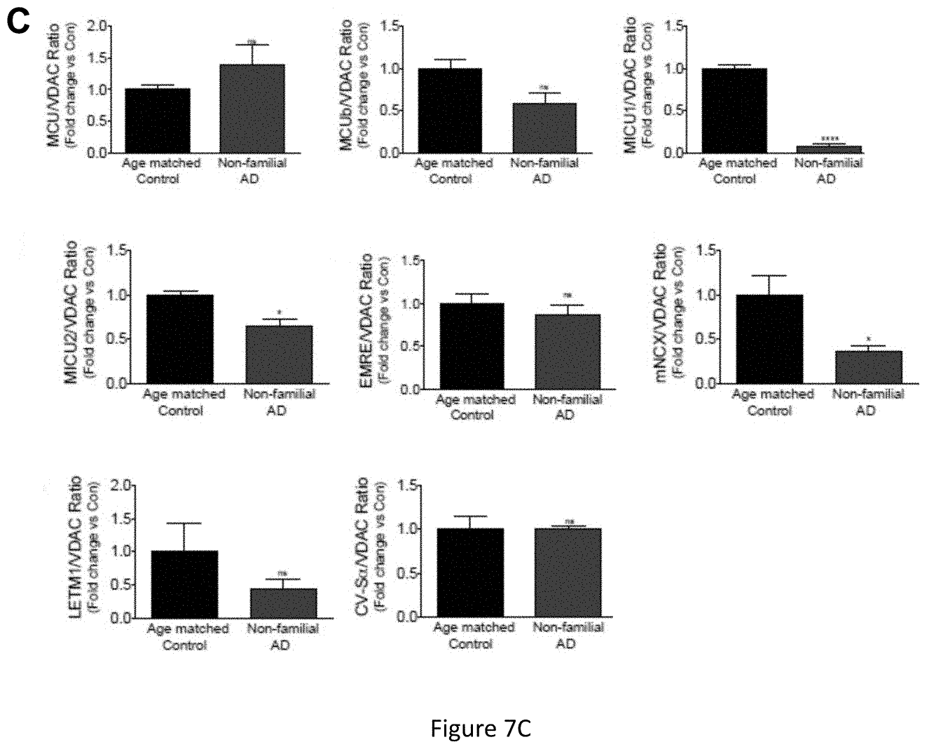

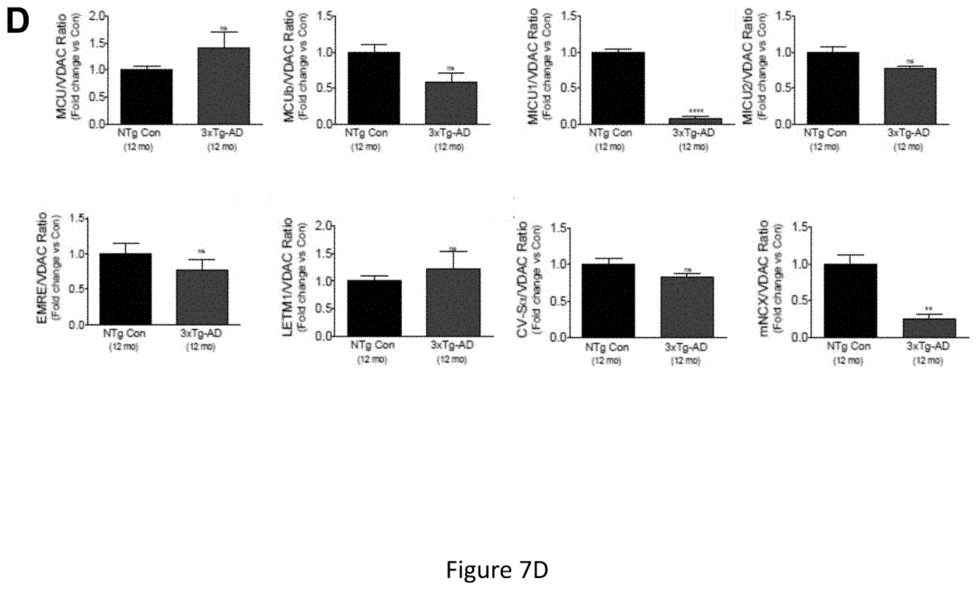

(APP KM670/671NL) and tau mutation (MAPT P301L)) was acquired. These mice develop age-progressive pathology similar to that observed in AD patients including: impaired synaptic transmission, Aβ deposition, and plaque and tangle histopathology. Brain tissue was isolated from the frontal cortex of 2, 4, 8 and 12 month old 3x-Tg AD mutant mice and outbred age-matched nontransgenic controls (NTg) and RNA was isolated for qPCR quantification of gene expression. 3xTg-AD mice displayed an age dependent reduction in Mcub and Micu1 RNA levels, which given the hypothesized role of these proteins as negative regulators of the uniporter channel would promote ₘCa²⁺ overload (FIGS. 1C-1F). Strikingly, mNCX expression in 3xTg-AD mice was decreased in an age-dependent manner (FIG. 1G) and completely absent in 12 month-old 3xTg-AD mice as compared to age-matched controls mirroring the results obtained from human AD brain samples (FIG. 1F). No alteration in gene expression or protein levels in frontal cortex tissue isolated from 2-month-old 3x-Tg mice, an age prior to any detectable neuropathology or altered cognition was found (Billings, L. M. et al., 2005, Neuron 45:675-688) (FIG. 1C and FIG. 7). This result suggests the changes in gene expression are age-dependent and not merely the result of developmental expression changes associated with this mutant model. To confirm that the changes in mRNA expression were manifested at the protein level, tissue samples from 12-month-old mice were examined using standard Western blot techniques. Almost complete loss of mNCX immunoreactivity was confirmed, as was a significant reduction in MICU1, and a slight reduction in MCUb; Complex V-subunit alpha served as a mitochondrial loading control (FIG. 1H).

To discern if ₘCa²⁺-overload is a feature of the 3xTg-AD model, mitochondria from the frontal cortex and hippocampus from 12-month-old 3x-Tg mice, and performed a Ca²⁺

retention capacity assay (CRC) using the reporter Ca-Green-5n was isolated. Isolated mitochondria were continuously monitored for changes in fluorescence using a spectrofluorometer during 10-μM bath Ca²⁺ additions every 50 seconds (Elrod, J. W., et al., 2010, Journal of clinical investigation 120:3680-3687). A ˜50% reduction in CRC in mitochondria isolated from 3xTg-AD mice vs. NTg con was quantified. This result suggests that MPTP activation occurs in this AD model at about half the Ca²⁺ load as WT controls (FIGS. 1I and 1J).

ₘCa²⁺ Overload and Increased Susceptibility to MPTP Activation in APPswe Cells is Rescued by mNCX expression

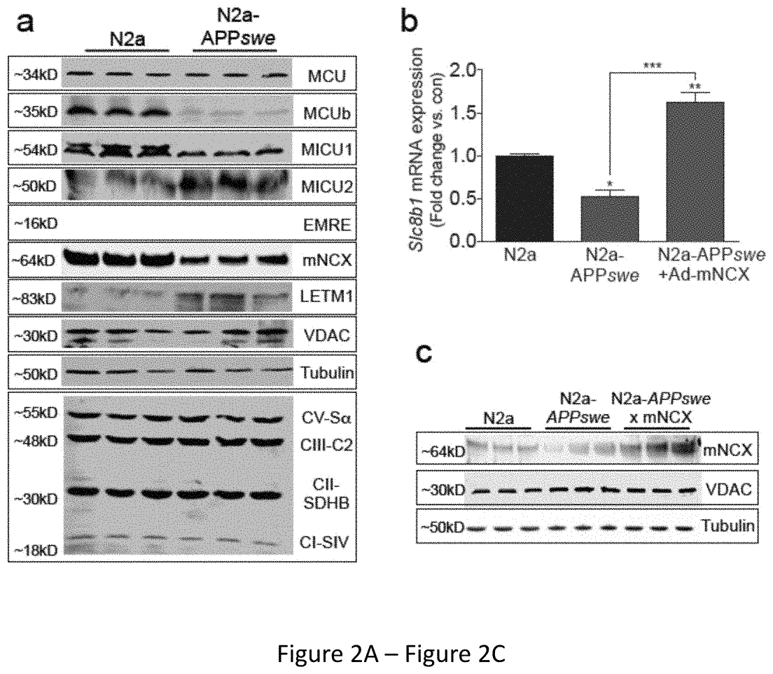

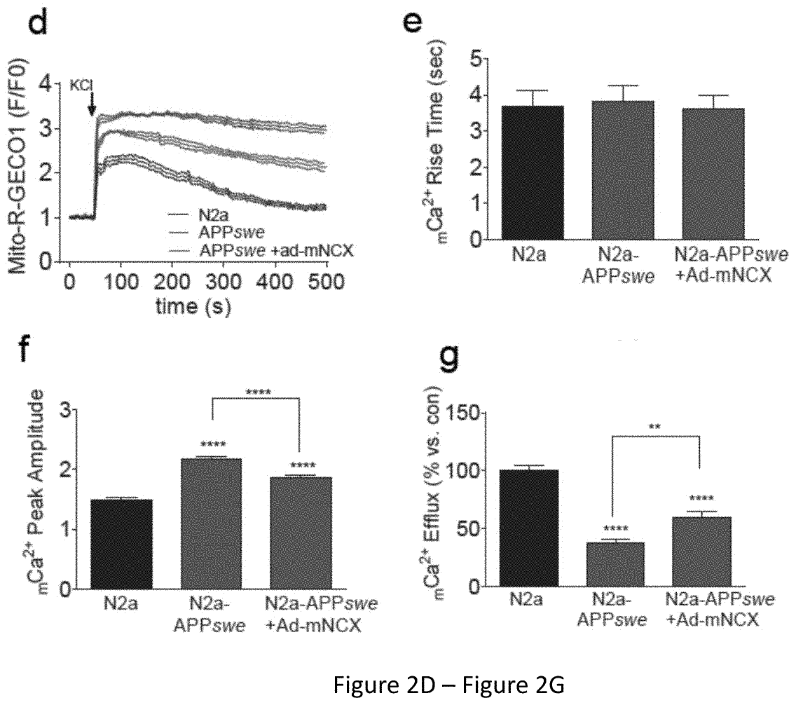

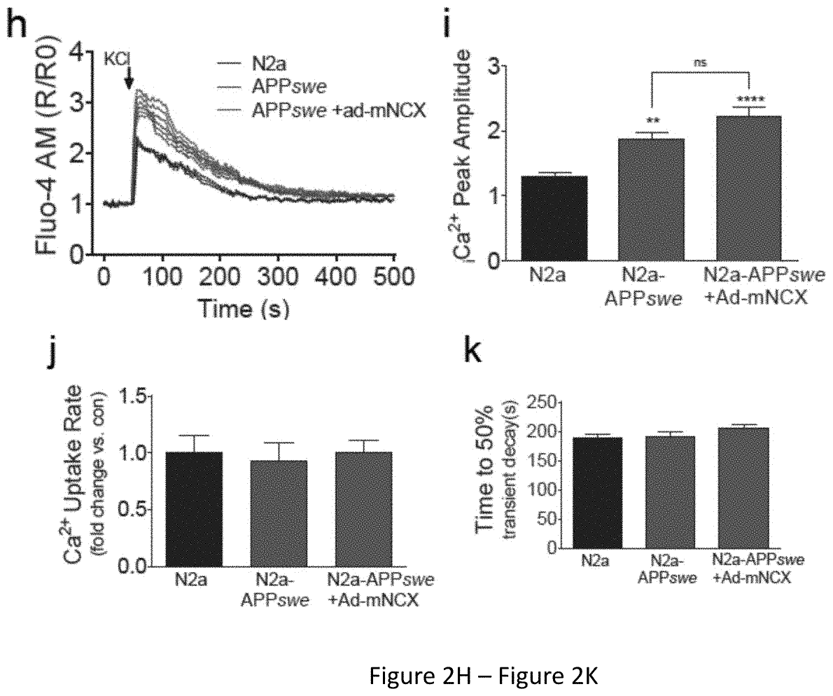

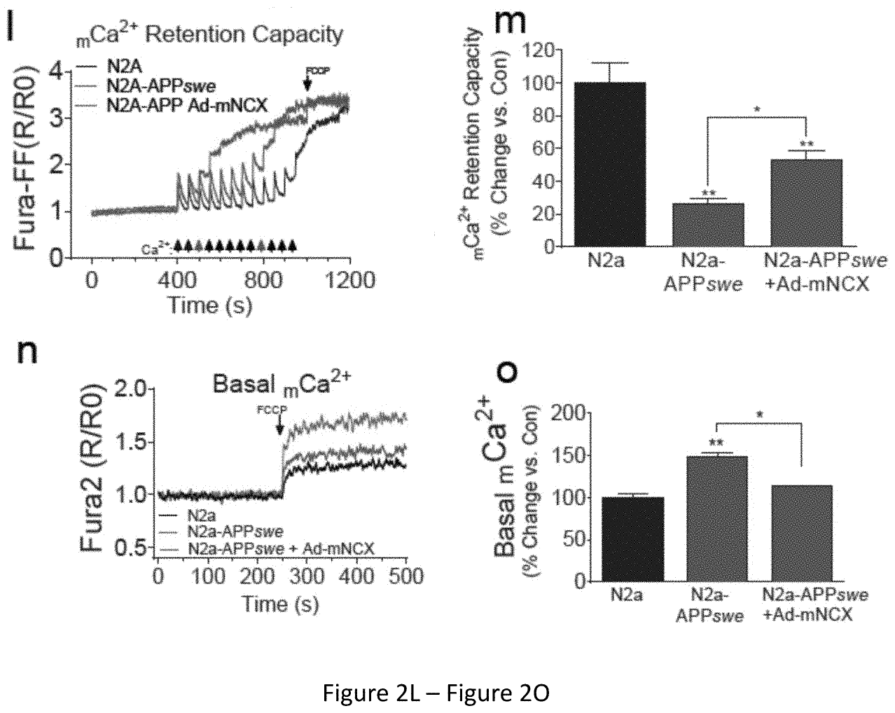

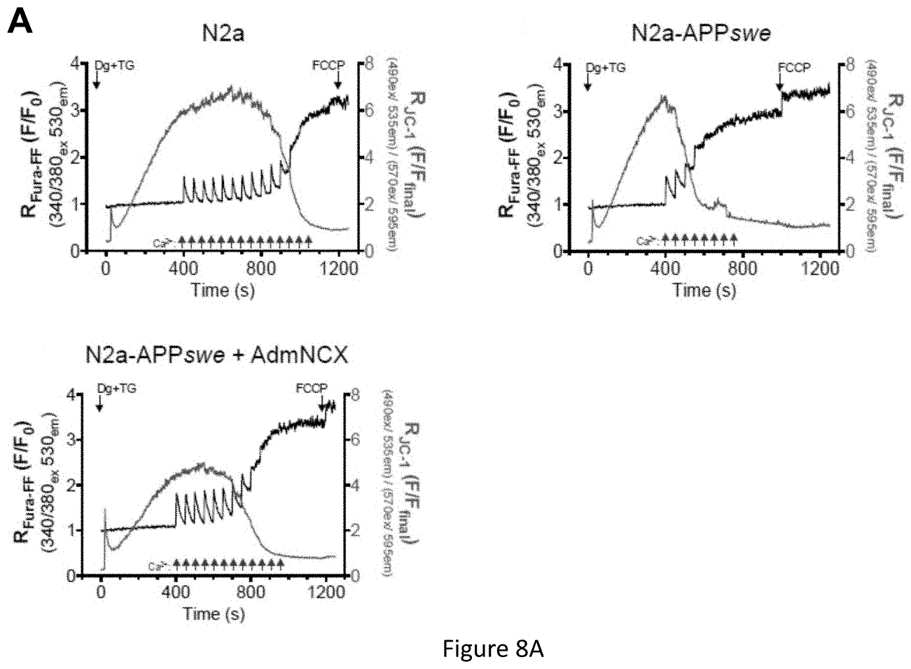

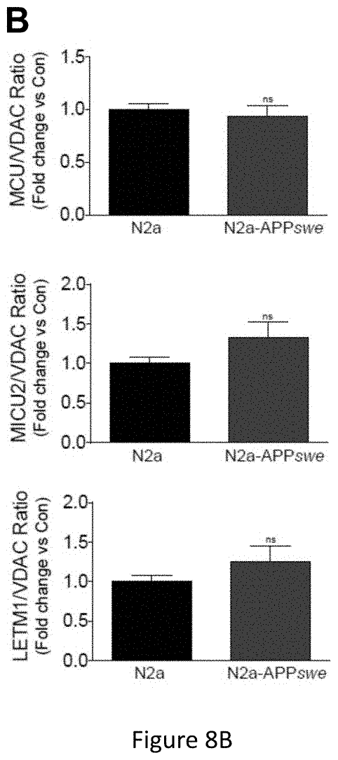

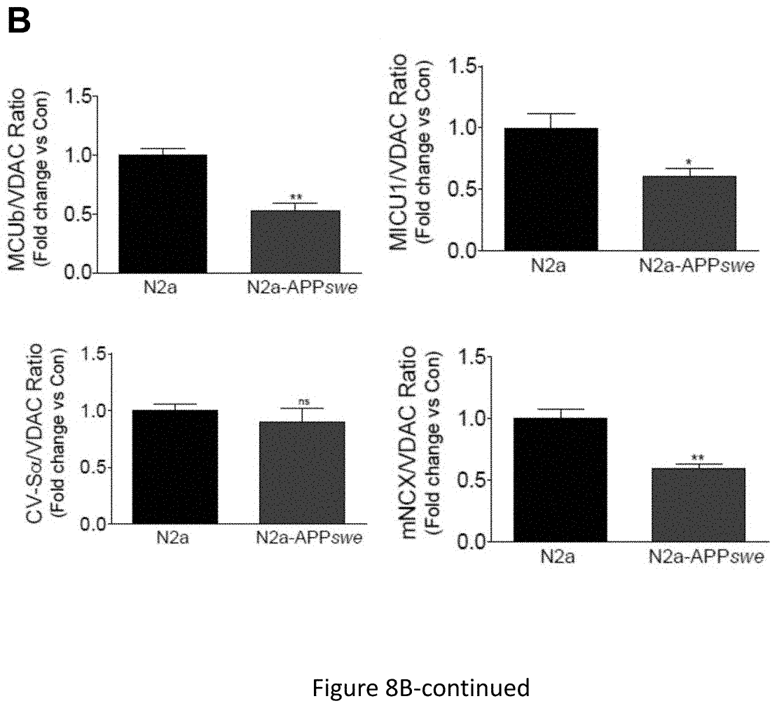

Next, to move towards a system more amendable to real-time mechanistic studies a neuroblastoma cell line (N2a) stably expressing an APP mutant protein (K670N, M671L, APPswe) (Thinakaran G. et al., 1996, The journal of biological chemistry 271:9390-9397) was examined. APPswe cells displayed a significant reduction in protein expression of mNCX (major efflux mediator), MCUb (possible negative regulator of uptake) and MICU1 (inhibitor of uptake at low iCa²⁺) protein expression, mirroring the results obtained from human AD samples. Surprisingly, these alterations in expression are consistent with molecular changes that would drive ₘCa²⁺ overload, in contrast to the compensatory alterations it was previously reported in heart failure samples (Luongo, S. T., et al., 2017, Nature). Tubulin and OxPhos complex expression served as total and mitochondrial loading controls respectively. Importantly, no change in OxPhos component expression was observed, suggesting no change in overall mitochondrial content (FIG. 2A, and FIG. 8D-8K). In total, these data suggest that alterations in the expression of the ₘCa²⁺ exchange machinery may be a significant contributor to mCa²⁺-overload in AD.

Next it was examined if restoring ₘCa²⁺ efflux capacity is sufficient to rescue impairments in ₘCa²⁺ handling and reduce ₘCa²⁺ overload in maturated N2a-APPswe cells using adenovirus encoding Mncx (Ad-mNCX). The mRNA expression of mNCX was significantly decreased by ˜50% in N2a- APPswe as compared to N2a control cells and this was significantly restored to ˜60% in APPswe cells after 48 hours post-infection with adenovirus encoding mNCX (Ad-mNCX) in Qper studies. All data corrected to the housekeeping gene, RPS13 (FIG. 2B). 48 hours after Ad-Mncx infection, western blot assessment showed a complete rescue of mNCX expression in APPswe cells. VDAC and tubulin served as loading controls (FIG. 2C).

Next to evaluate the iCa²⁺ and ₘCa²⁺ transients, N2a, N2a-APPswe and N2a-APPswe+AdmNCX cells were infected with adenovirus encoding the mitochondrial-targeted ₘCa²⁺ reporter, R-GECO1 (Ad-mitoR-GECO) (FIG. 2D, solid line=mean, dashed line=SEM), and loaded with the iCa²⁺ reporter, Fluo4-AM (FIG. 2H, solid line=mean, dashed line=SEM) and imaged continuously during stimulation with KCl to depolarize the plasma membrane and activate voltage-gated Ca²⁺ entry. No significant changes in ₘCa²⁺ rise time was found in all three groups (FIG. 2E). However, N2a-APPswe cells displayed a significant increase (˜45%) in ₘCa²⁺ transient peak amplitude as compared to N2a control cells, and this was significantly reduced (˜20%) by Ad-mNCX (FIG. 2F). Quantification of the ₘCa²⁺ efflux rate revealed >60% decrease in APPswe cells as compared to N2a cells and Infection with Ad-mNCX increased the efflux rate in APPswe cells by ˜50% vs. APPswe cells (FIG. 2G). Quantification of iCa²⁺ peak amplitude revealed a significant increase (˜40%) in N2a cells expressing APPswe vs. N2a. While expression of mNCX did not alter the APP-mediated increase in iCa^{2′} flux, it restored the mitochondrial transient towards that of control N2a cells (FIG. 2I). In these studies, cells from all three groups didn't show any significant differences on MCU-mediated ₘCa²⁺ uptake rate (FIG. 2J).

To evaluate if impaired ₘCa²⁺ efflux may contribute to ₘCa²⁺-overload, a ₘCa²⁺

retention capacity assay using the ratiometric reporters FuraFF (Ca²⁺) and JC1 (mitochondrial

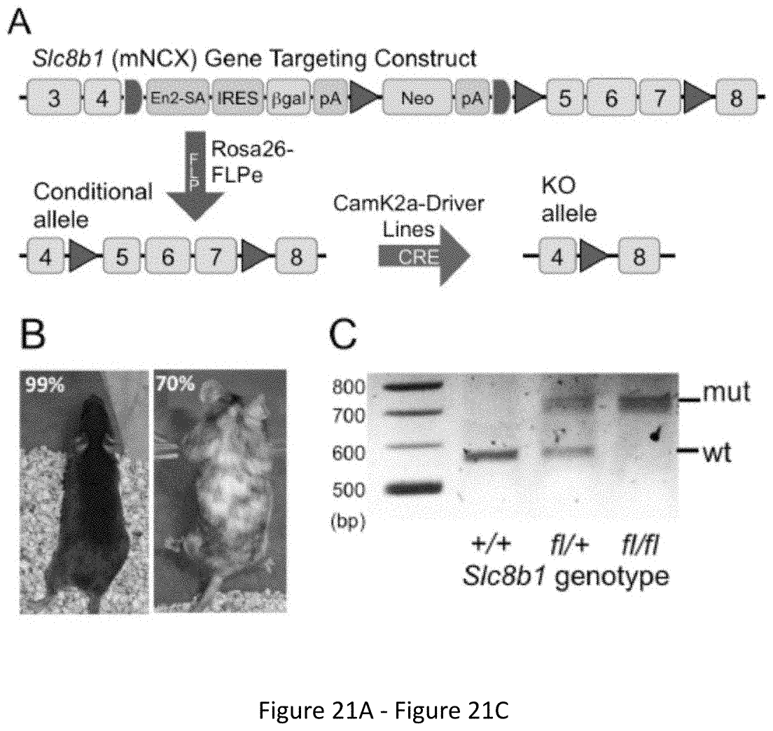

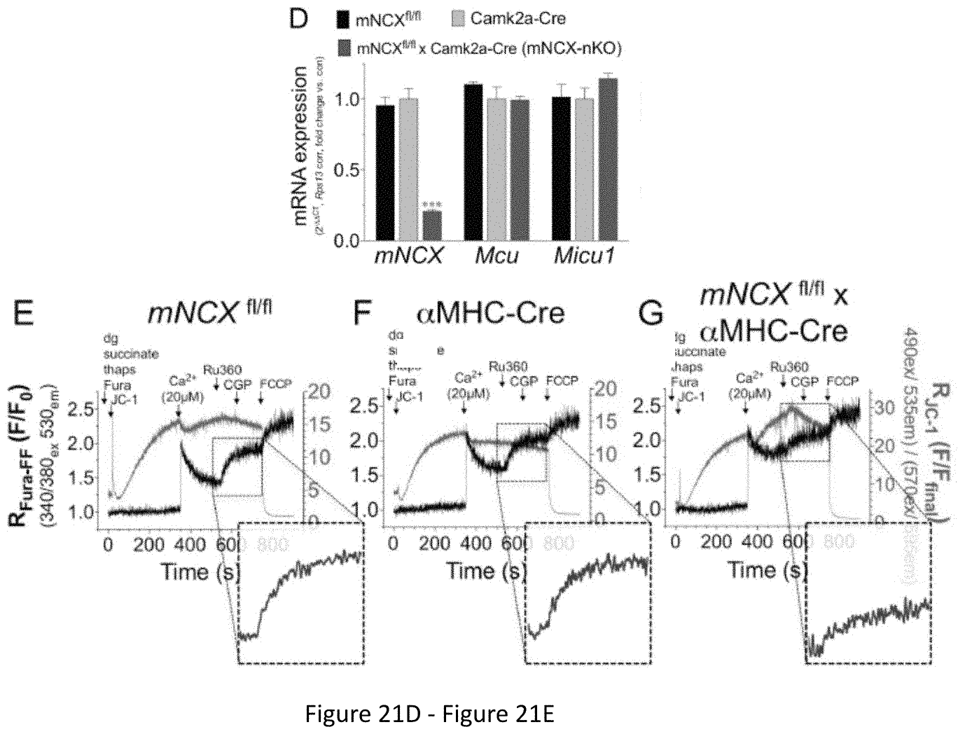

membrane potential) was employed. Recordings are only shown for FuraFF for clarity. Cells were permeabilized with digitonin and treated with thapsigargin to inhibit SERCA so that the movement of Ca²⁺ was only influenced by mitochondrial uptake. The protonophore, FCCP, was used at the conclusion of the experiment to correct for total Ca²⁺ in the system. N2a cells expressing the APPswe mutation underwent permeability transition after the 3rd 10-μM pulse of Ca²⁺ (red arrow, in representative recordings). This was in striking contrast to the control, which sustained 3x the concentration of bath Ca²⁺ before collapse of ΔΨ and loss of ₘCa²⁺. Rescue of Mncx expression greatly increased the mitochondrial calcium retention capacity (˜9 pulses versus ˜3 pulses in N2a APPswe cells (FIGS. 21 & 2J). To discover if enhancing mNCX-mediated efflux was sufficient to reduce ₘCa²⁺ overload and restore matrix Ca²⁺ levels, maturated N2a cells from all 3 groups were loaded with Fura2 and treated with digitonin and thapsigargin (Luongo et al., 2015, Cell reports 12:23-34). Upon reaching a steady state recording of Fura2, the protonophore, FCCP, was used to collapse ΔΨ and release all matrix free-Ca²⁺. Quantification of basal ₘCa²⁺ content found that mNCX expression completely corrected APPswe-mediated Ca²⁺ overload (FIGS. 2K & 2L).

Expression of mNCX Reduces Superoxide (O₂°—) Generation in a Neuronal AD Model

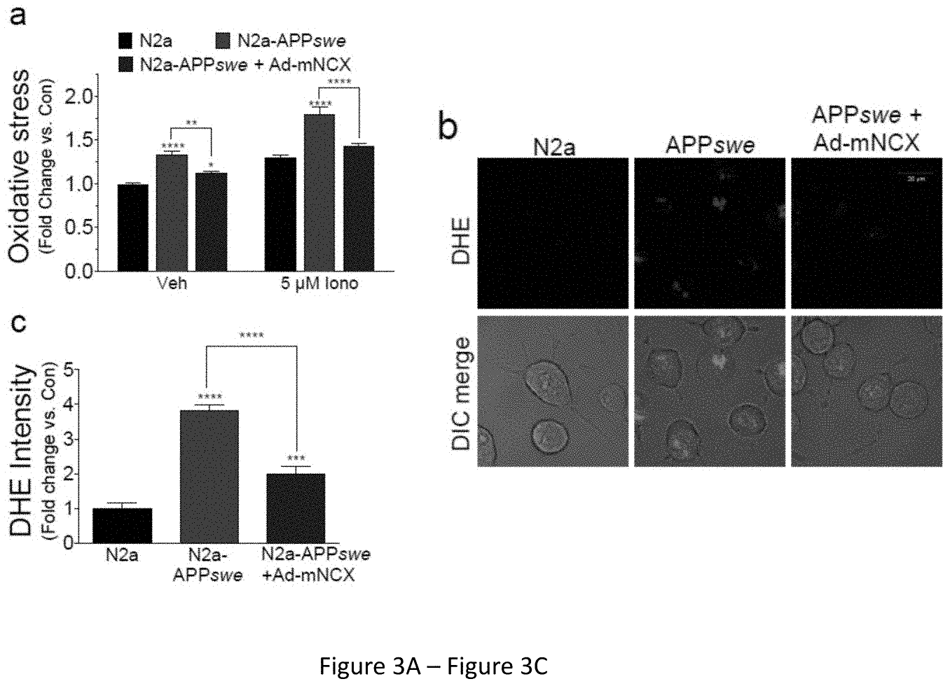

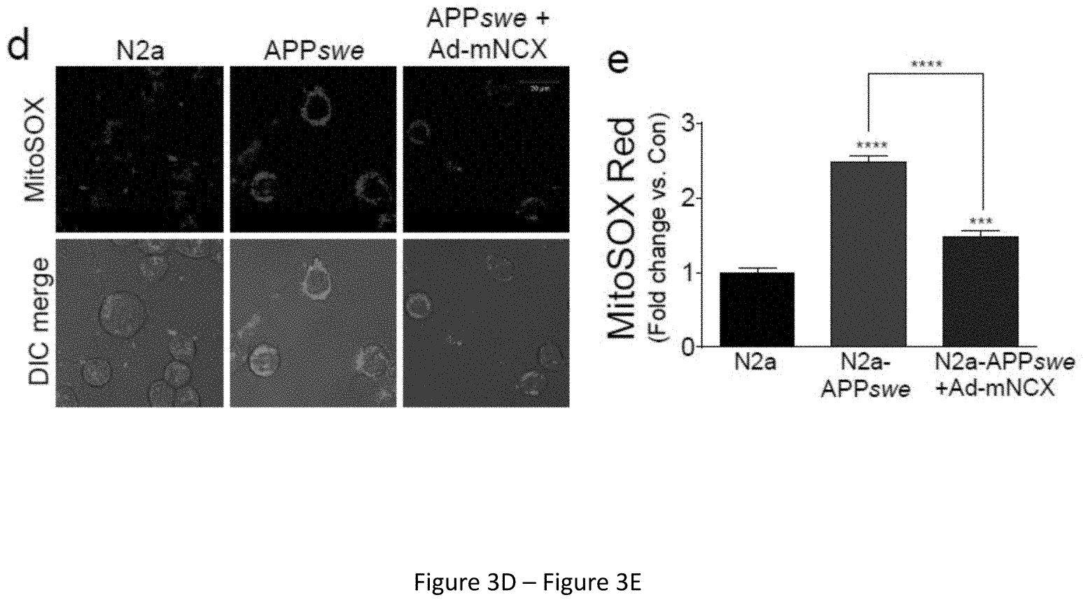

ₘCa²⁺-overload is known to elicit increased ROS generation and suppression of ROS scavenging pathways via numerous molecular mechanisms (Muller et al., 2011, Antioxid Redox Signal 14:1225-1235; Andreyev, A. Y. et al., 2005, Biochemistry 70:200-214; Andreyev, A. Y. et al., 2015, Biochemistry 80:517-531). Here maturated cells (N2a, N2a-APPswe, and APPswe+Ad-mNCX) were examined for changes in redox status utilizing 3 different ROS sensors. 30m following treatment with vehicle (Veh) or the Ca²⁺ ionophore, ionomycin (Iono), cells were loaded with the total cellular ROS indicator, CellROX Green. N2a cells expressing APPswe displayed an increase in total ROS that was significantly reduced in APPswe cells expressing mNCX (48 hours post-adeno) (FIG. 3A). Next, the O₂^{°—} specific probe dihydroethidium (DHE) was employed. DHE when oxidized to 2-hydroxyethidium intercalates DNA and increases fluorescent intensity (>500-fold). FIG. 3B depicts representative images of DHE staining (518ex/605em) and differential interference contrast (DIC) merge. N2a-APPswe had a ˜4-fold increase in O₂^{°—} production that was reduced by ˜50% with mNCX expression (AdmNCX) (FIGS. 3B-3C). To further define the subcellular site of ROS generation the mito-targeted O₂^{°—} indicator, MitoSOX Red was employed. FIG. 3D depicts representative images of MitoSOX staining (510ex/580em) and differential interference contrast (DIC) merge. Quantification of MitoSOX fluorescent intensity showed ˜3-fold increase in O₂^{°—}production in N2a-APPswe vs. N2a con that was reduced by ˜50% with mNCX expression (Ad-mNCX). These results support the notion that expression of mNCX, in the context of AD-like stress, reduces mitochondrial O₂^{°—} production.

Expression of mNCX Rescues OxPhos Defects in APPswe Cells

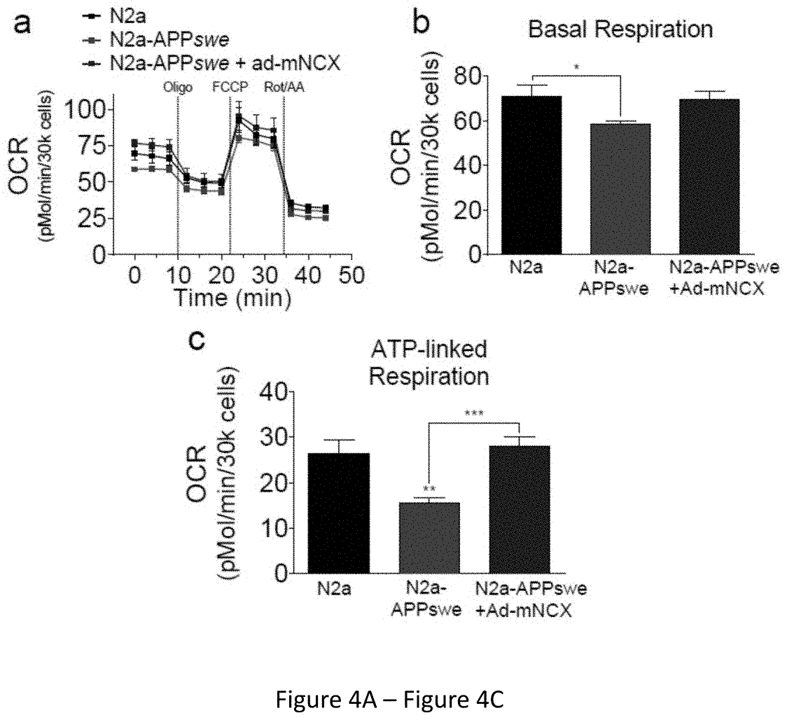

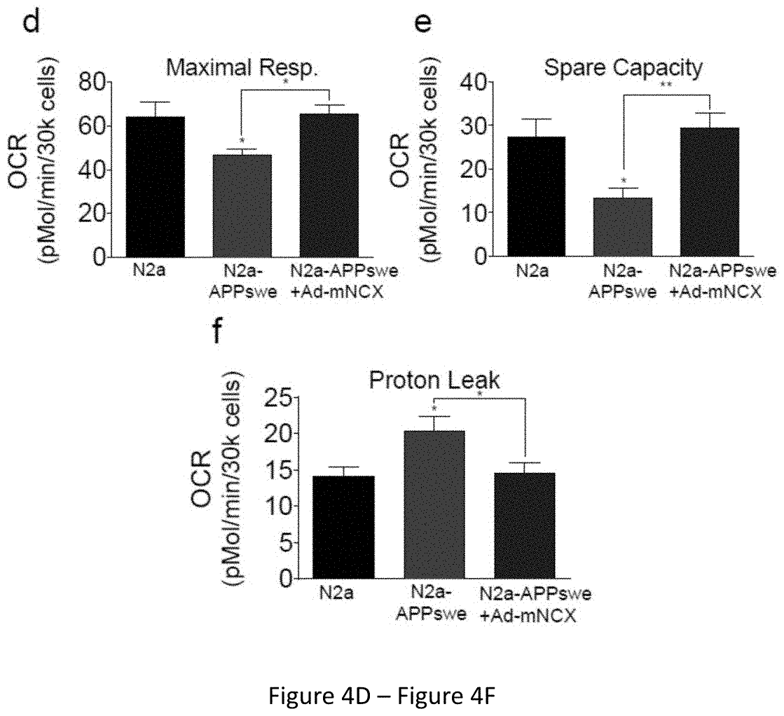

It's well known that excessive matrix Ca²⁺ augments mito O₂^{°—} generation, as shown in FIG. 3, and thereby can negatively impact OxPhos. AD is characterized by neuronal metabolic dysfunction, with studies suggesting that mitochondrial defects in energy production may underlie neurodegeneration and cognitive decline (Jha, S. K., 2016, Biochimica et biophysica acta 1863.5:1132-1146). Maturated N2a-APPswe cells were examined for changes in OxPhos using a Seahorse XF96 extracellular flux analyzer to monitor oxygen consumption rates (OCR). FIG. 4A shows representative OCRs at baseline and following: oligomycin (oligo; CV inhibitor; to uncover ATP-linked respiration), FCCP (protonophore to induce max respiration), and rotenone+antimycin A (Rot/AA; complex I and III inhibitor; complete OxPhos inhibition). APPswe mutant cells displayed a significant decrease in all respiratory parameters examined. Specifically, ˜1.5 fold lower basal respiration, 2-fold lower ATP-linked respiration, 1.5 fold lower max respiratory capacity and 1.5 fold lower spare respiratory capacity in N2a-APPswe vs. N2a controls. Amazingly, rescue of ₘCa²⁺ efflux with Ad mNCX infection for 48 hours corrected all OCR measurements back to N2a control levels (FIG. 4). These results show that ₘCa²⁺ overload is a significant contributor to AD-mediated impairments in OxPhos and that mNCX is sufficient to restore bioenergetics.

Enhancement of ₘCa²⁺ Efflux Reduces Cell Death Induced by a Variety of Stressors.

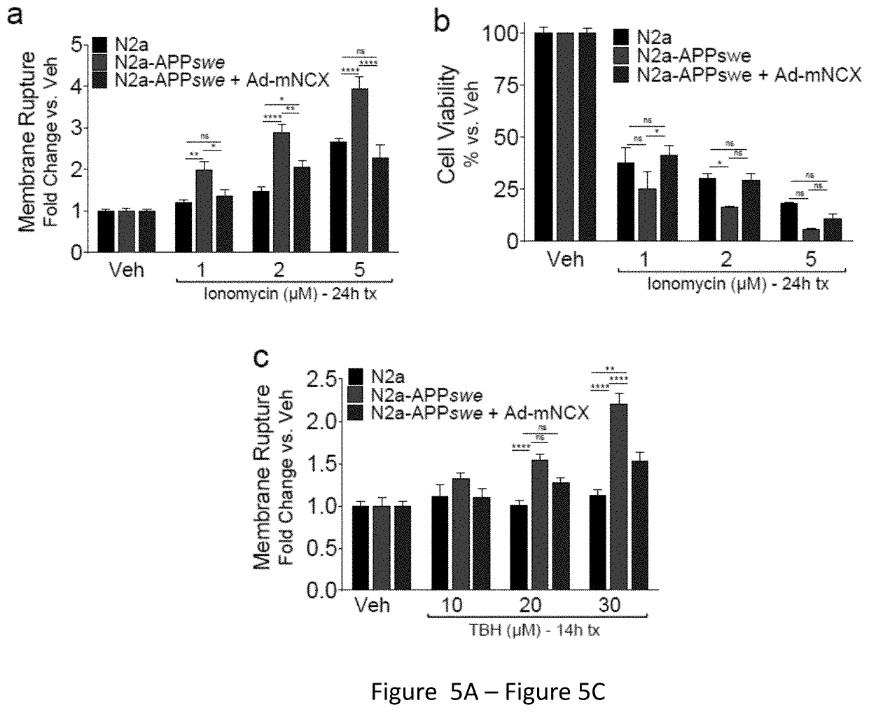

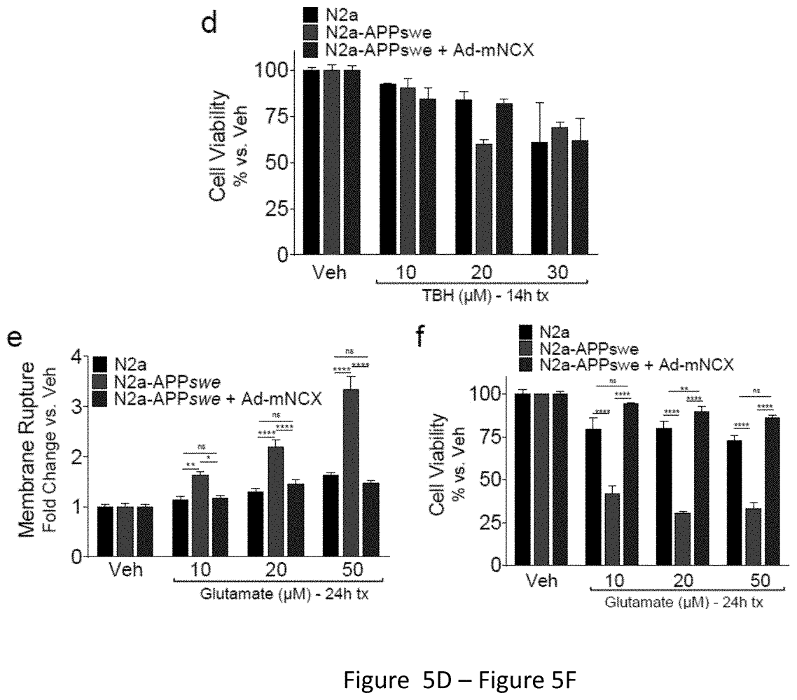

ₘCa²⁺-overload has been shown to augment neuronal cell death both through primary (MPTP and ROS) and secondary signaling mechanisms (metabolic derangement, etc.). Given that mNCX expression reduced O₂^{°—} production and MPTP activation and enhanced OxPhos capacity tests were performed to study if these protective mechanisms coalesced to reduce neuronal demise. N2a, N2a-APPswe and N2a-APPswe infected with Ad-mNCX for 48 hours were treated with Iono, (1-5 μM) for 24 hours and examined for plasma membrane rupture (hallmark of cell death) using the cell membrane impermeable dye, Sytox Green. Iono significantly increased membrane rupture in APPswe expressing cells over the N2a control at all doses and this was attentuated with mNCX expression (FIG. 5A). General cell viability was also examined using Cell Titer Blue (resazurin,) and it was found that rescue of mNCX expression in N2a-APPswe profoundly increased cell viability at all doses as compared to N2a-APPswe cells (FIG. 5B). Similarly, all groups were treated with the oxidizing agent and free-radical generator, tert-Butyl hydroperioxide (TBH), which is preferred over H₂O₂ due to its increased stability in solution. Treatment with 20 and 30 μM TBH for 14 hours significantly increased membrane rupture in APPswe expressing cells over the N2a control, which was reduced with increased mNCX expression (FIG. 5C). Cell Titer Blue was utilized to monitor cell viability and it was found that mNCX expression partially increased cell viability in N2a-APPswe in response to oxidative stress (FIG. 5D). Likewise, treatment with glutamate (NDMAR-agonist, neuroexcitotoxicity agent) significantly increased cell death in APPswe expressing cells across all doses and this was completely ablated by mNCX expression. Similarly, cell viability in N2a-APPswe with increased mNCXexpression was significantly enhanced at all doses of glutamate as compared to N2a-APPswe cells. These results strongly support that rescue of mNCX expression in the context of AD may be a powerful therapeutic to impede cell loss and AD progression (FIGS. 5E & 5F).

Enhancing ₘCa²⁺ Efflux Decreases the Amyloidogenic Aβ Pathway

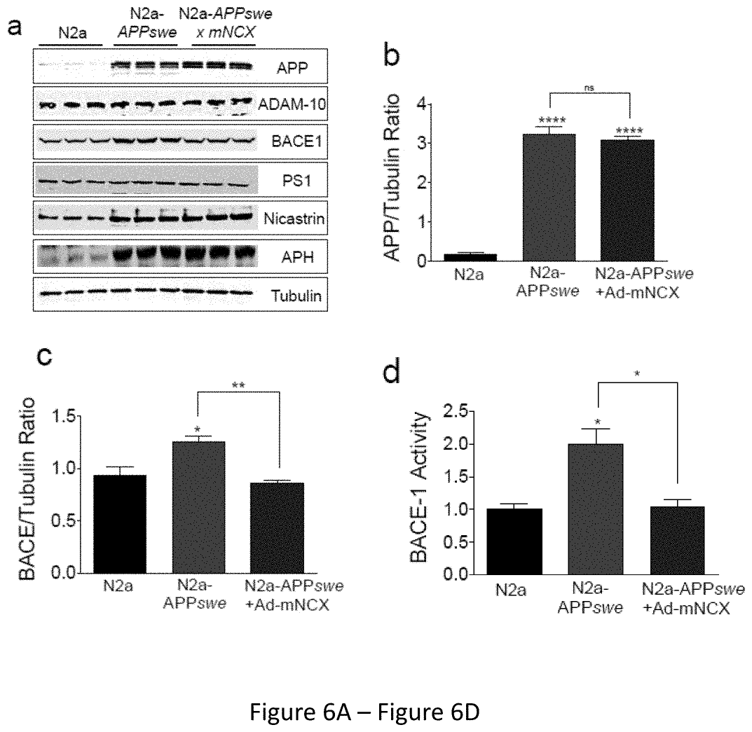

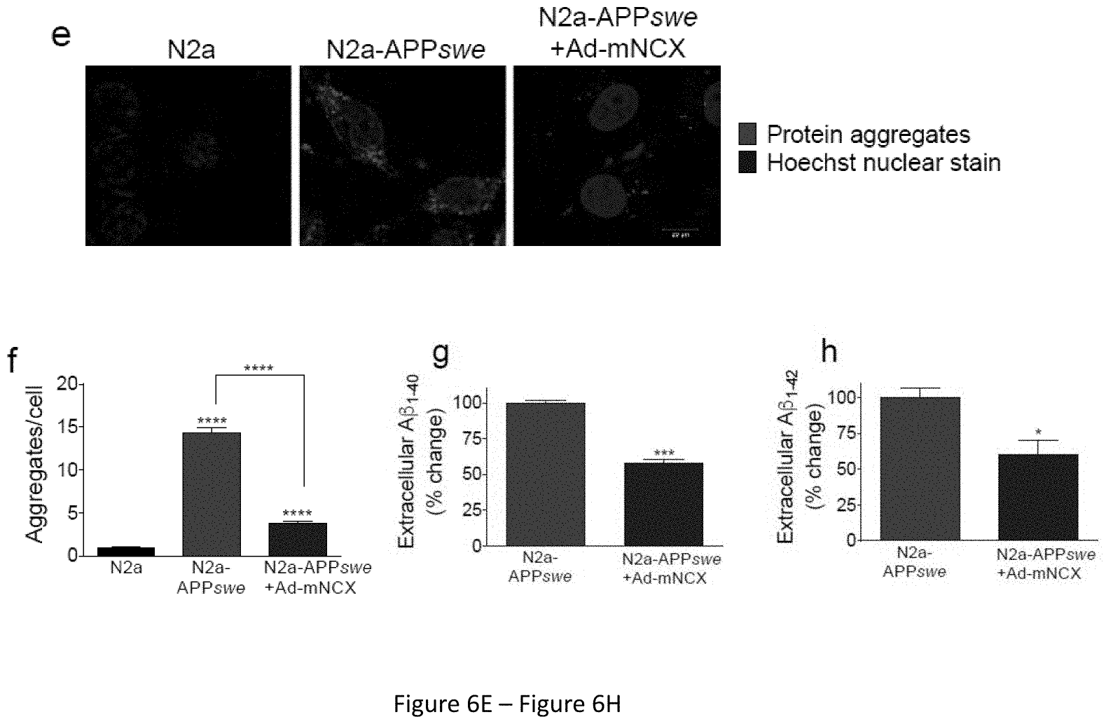

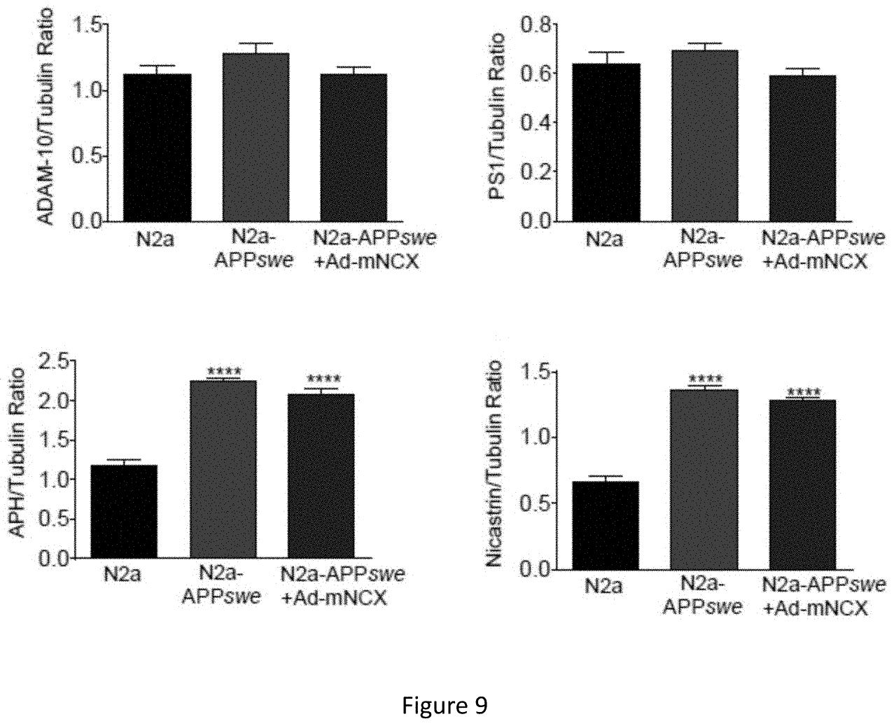

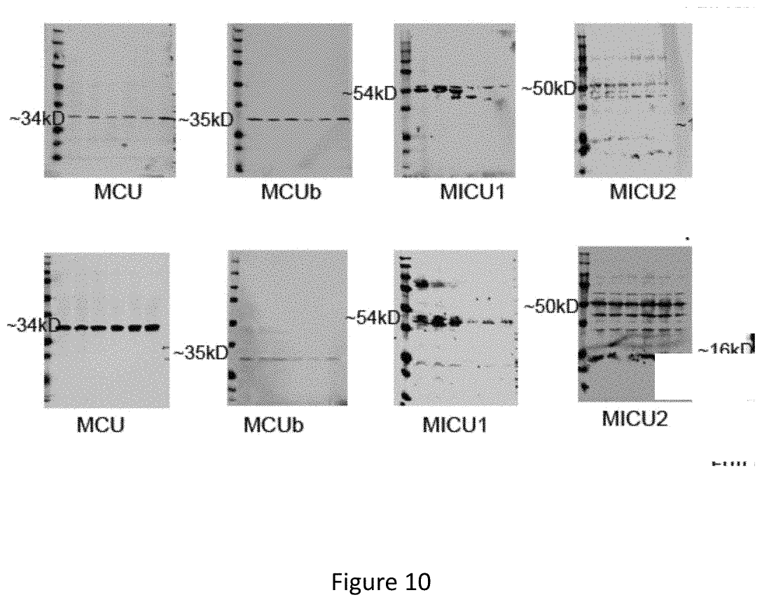

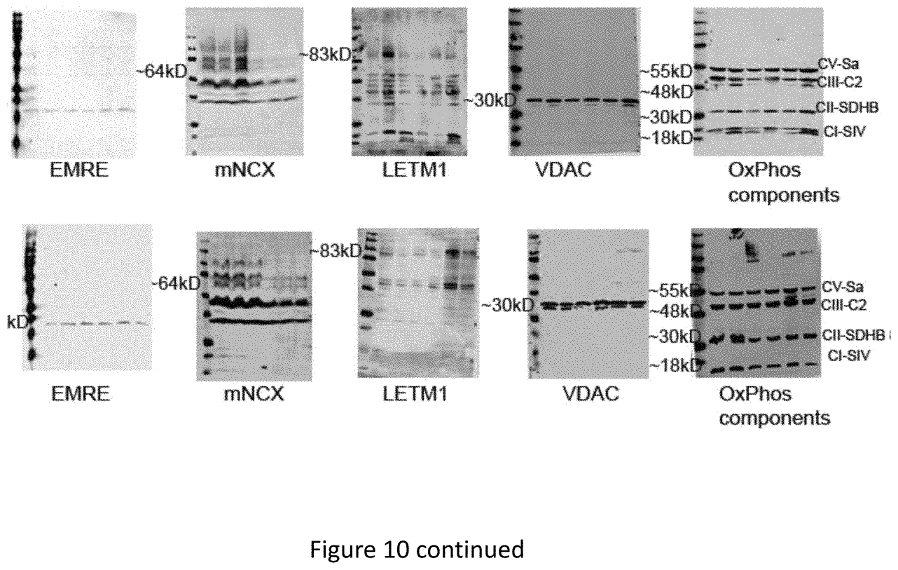

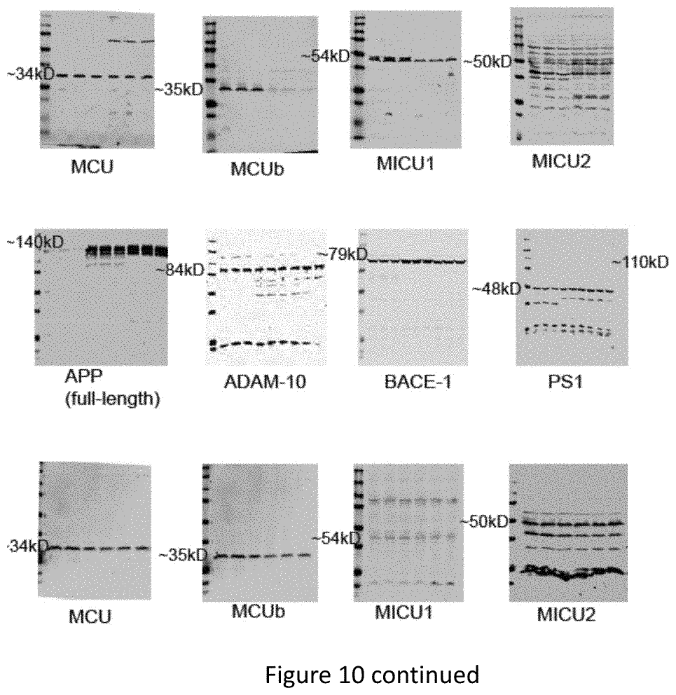

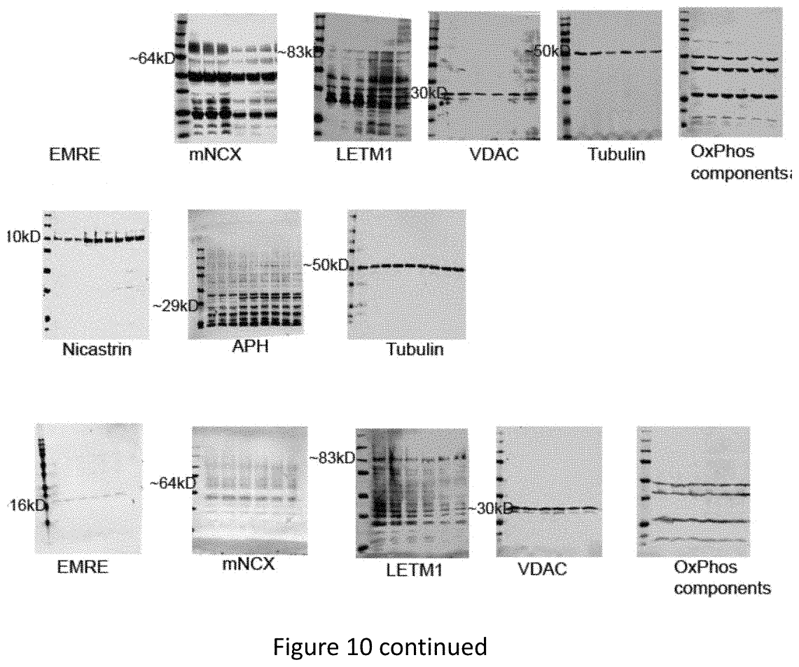

An intense research effort has been placed on identifying the link between Ca²⁺ dysregulation and the Aβ amyloidogenic pathway. Studies have suggested that Aβ increases iCa²⁺ levels by numerous mechanisms and vice versa, increased iCa²⁺ augments Aβ production and tau hyperphosphorylation (Berridge, M. J., 2010, Pflugers Archiv: European journal of physiology 459:441-449; Abeti, R. et al., 2015, Pharmacological research 99:377-381; Shilling, D., et al., 2014, J Neurosci 34:6910-6923; Mak, D. O. et al., 2015, PLoS Comput Biol 11: e1004529), two hallmarks of AD. Thus, in this study how altering ₘCa²⁺ levels impact Aβ production, toxicity and clearance was investigated. First, APP processing was investigated by Western blot. It was discovered that enhancing ₘCa²⁺ efflux (mNCX expression for 48 hours) reduced β-secretase (BACE1) expression in N2a-APPswe cells (FIGS. 6a & 6c). No change was found in full-length APP expression given the AD cell model features stable overexpression of mutant APP (FIGS. 6a & 6b). In addition, no significant change in the levels of, ADAM10, or the protein components of the γ-secretase complex was found (FIG. 6a & FIG. 9). In addition, further a fluorescence enzymatic assay using a synthetic peptide was performed, which has previously been shown to be highly specific. BACE1 activity was significantly increased in N2a-APPswe cells by ˜2-fold vs N2a con. A significant ˜50% decrease in BACE1 activity in N2a-APPswe infected with Ad-mNCX vs. N2a-APPswe was observed (FIG. 6d). These results suggest a direct involvement of the BACE-1 protease in the observed biological effect.

To further evaluate the effect of mNCX expression on Aβ generation, an ELISA for quantification of extracellular Aβ₁₋₄₀ and Aβ₁₋₄₂ levels was performed. Compared with N2a-APPswe controls a significant decrease in Aβ₁₋₄₀ ( 40% of decrease) and Aβ₁₋₄₂ formation (˜40% of decrease) in N2a-APPswe infected with Ad-mNCX was observed (FIGS. 6g & 6h). Moreover, it is the Aβ aggregate formation that plays a central role in the pathogenesis of AD.

To determine whether the mNCX have any effect on Aβ oligomerization, a fluorescence-based assay using Proteostat dye, was used to detect aggregated protein. This dye is essentially non-fluorescent unless it binds to a β-sheet structure of misfolded proteins in which case it fluoresces as a punctate pattern of cytoplasmic staining. N2a-APPswe cells showed increased accumulation of cytoplasmic inclusion bodies/aggregates vs N2a con. Rescue of mNCX expression in N2a-APPswe significantly decreased the protein aggregation ˜70% as compared to N2a-APPswe cells. (FIGS. 6e & 6f). These results are intriguing and suggest that elevated ₘCa²⁺ signaling may contribute to the amyloid cascade. In total this data demonstrates that mNCX modulates Aβ formation by regulating BACE1 activity and protein levels.

Role of ₘCa²⁺ Efflux in Alzheimer's Disease

The data presented herein demonstrates for the first-time role of ₘCa²⁺ efflux in Alzheimer's disease and its associated mitochondrial dysfunction. In this study, several dramatic alterations in the expression of ₘCa²⁺ exchangers were found, most significantly a reduction in the expression of the mitochondrial Na⁺/Ca²⁺ exchanger (mNCX), in a murine transgenic 3xTg-AD model and brain samples from AD patients and severe ₘCa²⁺ signaling abnormalities in an AD mutant cell line. A profound reduction in the expression of MICU1 (inhibitor of uptake at low iCa²⁺), and a slight reduction in MCUb (possible negative regulator of uptake) was also observed. MICU1, acts as a gatekeeper by negatively regulating uptake at low iCa²⁺ levels (Mallilankaraman, K. et al., 2012, Cell 151:630-644; Patron, M. et al., 2014, Molecular cell 53:726-737).

Moreover, the first biological evidence is provided herein that enhancing the clearance of pathogenic ₘCa²⁺ via rescuing mNCX expression preserved mitochondria function, biogenetics and reduced oxidative stress. These preservative functions ultimately decreased BACE1 expression and activity and in turn regulates APP processing to generate Aβ in APPswe cell lines. Several reports show increased levels and activity of BACE1 protein in the brain of sporadic and familial AD patients, compared to normal age controls (Citron, M. et al., 1992, Nature 360:672-674; Yang, L. B. et al., 2003, Nature medicine 9:3-4). The AD associated Swedish mutant APP is also associated with increased β-secretase activity (Luo, Y. et al., 2001, Nature neuroscience 4:231-232) as observed in APP swe cells. One of the therapeutic approach for AD, is to reduce Aβ production by either inhibiting β-secretase or γ-secretase activity. In the presented studies herein, no change in full-length APP expression and α and γ-secretase expression was found, which makes mNCX an important therapeutic target because previous studies suggested that inhibition of γ-secretase has multiple off-target effects and showed severe developmental abnormalities (Vassar, R. et al., 1999, Science 286:735-741) (De Strooper, B. et al., 1999, Nature 398:518-522). On the other side, mice deficient in BACE1, develop normally without any detectable physiological defects with a significant reduction in Aβ formation (Cai, H. et al., 2001, Nature neuroscience 4: 233-234) (Luo, Y. et al., 2001, Nature neuroscience 4: 231-232). In conclusion, mNCX significantly contributes to neuronal ₘCa²⁺ efflux and thus rescuing mNCX expression provide significant rationale towards the future development of therapeutics aimed at increasing ₘCa²⁺ efflux in neurodegenerative AD diseases.

### Example 2: ₘCa²⁺ Dysregulation in Neurodegeneration

The data presented herein demonstrates several dramatic alterations in the expression of mCa²⁺ exchangers in a murine transgenic AD model and brain samples from AD patients and severe ₘCa²⁺ signaling abnormalities in an AD mutant cell line. To elucidate if alterations in mCa²⁺ exchange are causative in the development of AD neuronal-specific, gain- and loss-of-function mutant mouse models were generated targeting the mitochondrial Na⁺/Ca²⁺ exchanger (mNCX, Slc8b1 gene). mNCX is reported to be the primary mechanism for ₘCa²⁺ efflux in excitable cells, and thereby is an excellent target to modulate ₘCa²⁺ load in neurons. It is hypothesized herein that ₘCa²⁺ overload is a primary contributor to AD pathology by promoting metabolic dysfunction and neuronal cell death, and that enhancing ₘCa²⁺ efflux impedes neurodegeneration and AD pathogenesis. The studies described herein examination of the role of ₘCa²⁺ in neurodegeneration and associated mitochondrial dysfunction.

Mechanisms of Neuronal ₘCa²⁺ Exchange.

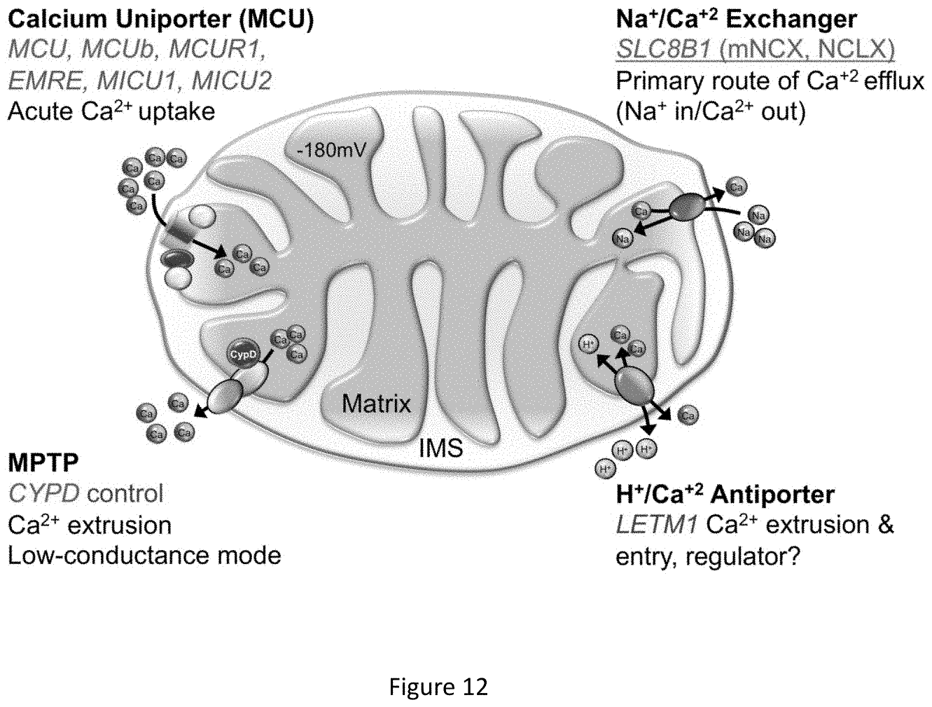

The neuron is unique in that it is an electrically excitable cell wherein an action potential is chemically coupled to neurotransmission; cellular signaling that is intricately linked with the flux of ᵢCa²⁺. Thus, a complex system has evolved to regulate Ca²⁺ exchange to maintain homeostatic conditions. Numerous genetic components have been identified and shown to mediate the passage of Ca²⁺ across the plasma membrane and endoplasmic reticulum (ER), and while great strides have been made in understanding the temporal and spatial relationship of Ca²⁺ in regards to neurotransmitter release and receptor-mediated signaling, our understanding of other subcellular Ca²⁺ domains remains elementary. Elevations in intracellular calcium (ᵢCa²⁺) are theorized to be rapidly integrated into mitochondria due to the high electromotive force generated by the electron transport chain (Δψ=˜−180 mv). The everchanging ᵢCa²⁺ environment and high driving force for ₘCa²⁺ requires that neuronal mitochondria possess a tightly controlled exchange system. While many classical biophysical studies have characterized the properties of ₘCa²⁺ flux, there have been virtually no causative studies defining the role of ₘCa²⁺ in neuronal physiology due to the unknown genetic identities of the exchange components. Just recently, the ₘCa²⁺ field has been transformed by the discovery of many genes that encode ₘCa²⁺ transporters and channels (FIG. 12). Generation of gain- and loss-of-function mutant mice have been generated with a goal of defining the function of ₘCa²⁺ in physiology and disease. It's important to note that the IMM must be impermeable to solutes and ions to maintain the proton gradient and drive oxidative phosphorylation (OxPhos).

Molecular Mechanism of ₘCa²⁺ Uptake

Ca²⁺ enters the mitochondrial matrix via the mitochondria calcium uniporter complex (MCUc). The uniporter is an inward rectifying, high-capacity, Ca²⁺-specific channel whose uptake is mediated by Δψ. The biophysical properties of MCUc-mediated ₘCa²⁺ influx have been extensively studied in many cell types, aided by pharmacologic inhibition with ruthenium red derivatives (a general non-specific inhibitor). Recently genetic components of the MCUc have been identified including: MCU, MICU1, MICU2, MCUR1, EMRE and MCUb, now allowing for the first-time causative study into the role of ₘCa²⁺ uptake in physiology and disease. The majority of work to date has focused on the MCU gene, which encodes the channel-forming portion of the MCU complex and is required for Ca²⁺ permeation. (To clarify the nomenclature, the MCU gene is the core channel forming subunit of the MCU channel/supercomplex (MCUc)). Mcu has been conditionally deleted in adult mice (cardiomyocyte-restricted deletion) to demonstrate that ₘCa²⁺—uptake is required for increased cardiac contractility in response to adrenergic signaling and that genetic inhibition of Mcu is cardioprotective in the setting of acute ischemiareperfusion injury by limiting mitochondrial permeability transition pore (MPTP) activation. While other MCUc regulatory components have been identified their function in the regulation of channel activity remain to be fully elucidated. It has been proposed that the EF-hand containing, MICU1, acts as a gatekeeper by negatively regulating uptake at low ᵢCa²⁺ levels. The molecular mechanism for this inhibition of MCU remains unknown. Likewise, MCUb may act to negatively regulate the channel by replacing Mcu subunits and thereby lowering overall flux capacity; although only supported by a single publication. Both MCUR1, and EMRE appear to be required for channel formation, perhaps acting as scaffolds for uniporter assembly or as necessary regulatory subunits. While these studies present solid evidence in mostly non-excitable cells and these genes appear likely to be components of the long sought-after uniporter complex, many properties of the MCUc make it a challenging experimental/therapeutic target in the context of neurons and in vivo models of disease. First, genetic ablation of MCUc components has the potential to reduce homeostatic ₘCa²⁺ as this is the primary means of Ca²⁺ influx, and could thereby negatively impact basal metabolism and basic cellular function. Secondly, limiting the function of MCUc has the potential to alter ᵢCa²⁺ signaling, since MCU-mediated uptake is thought to buffer ᵢCa²⁺ transients in neurons. Finally, it has been proposed yet unidentified MCU-independent ₘCa²⁺-uptake pathways. Therefore, it is hypothesized herein the mitochondrial Na⁺/Ca²⁺ exchanger (mNCX) represents the best target for in vivo investigation into the role of ₘCa²⁺ in neurodegeneration. However, the genes comprising the MCUc remain experimentally germane to the current proposal as they represent targets to modulate ₘCa²⁺ influx, should this be needed for mechanistic purposes. Towards this end, a neuronal a conditional mutant mouse model targeting Mcu has been developed and confirmed herein to aid mechanistic study.

ₘCa2+ Signaling in Metabolic Regulation

The metabolic demand of synaptic transmission and neuronal signaling makes it essential that an efficient and tightly controlled system be in place to regulate ATP production. The importance of the astrocyte-neuron lactate shuttle has recently been questioned due to studies suggesting that neuronal glycolysis and oxidative phosphorylation (OxPhos) are more significant contributors to energetics. Utilizing live hippocampal slices to examine energetic responsiveness in stimulated neural networks it was reported that, the major mechanisms mediating brain information processing are all initially powered by oxidative phosphorylation. Indeed, simultaneous measurements of ₘCa²⁺ and NADH flux are strongly correlated with increased oxidative phosphorylation and ATP production. Thus, Ca²⁺ is proposed to be the key link between neurotransmission and OxPhos and has been shown to modulate mitochondrial metabolism by activation of Ca²⁺-dependent dehydrogenases and modulation of ETC complexes. mCa²⁺ activates three matrix dehydrogenases that are rate limiting in the tricarboxylic acid (TCA) cycle. Pyruvate dehydrogenase (PDH) is the main enzyme that converts pyruvate to acetyl-CoA for entry into the TCA cycle, and as such also links glycolysis with OxPhos. PDH is active in the dephosphorylated state and inactive in the phosphorylated state. Ca²⁺ activates PDH phosphatase leading to dephosphorylation of PDH and subsequently increases acetyl-CoA availability for the TCA cycle. In support of this theory, MCU-mediated uptake is required for PDH activation in the context of ‘fight or flight’ signaling. Ca²⁺ also increases the activity of α-ketoglutarate dehydrogenase (KGD) and isocitrate dehydrogenase (IDH) through yet unknown mechanisms. ₘCa²⁺ also modulates energy production by altering F1-F0 ATPase function independent of changes in electron motive force (Δψ). In summation, ₘCa²⁺ can modify ATP production, and thus it represents an important mechanism to modulate cellular respiration and cell death wherein ATP availability is critical in the initiation of programmed killing.

ᵢCa²⁺ Dysregulation in AD Neurodegeneration.

ᵢCa²⁺ signaling plays an essential role in synaptic transmission (SNARE mediated vesicle fusion and neurotransmitter exocytosis) and intra- and paracellular communication. Control of ᵢCa²⁺ levels is so critical that ˜80% of neuronal ATP is consumed to modulate ᵢCa²⁺ flux at the plasma membrane and ER. Therefore, it is not surprising that alterations in Ca²⁺ handling have been reported to be a central feature of neurodegeneration and age-related diseases. Numerous reports of Ca²⁺ dysregulation coalesced into the formation of the ‘calcium hypothesis’ of aging and AD. The calcium hypothesis theorizes that alterations in Ca²⁺ handling are a central mechanism linking amyloid metabolism to neuronal cell death and cognitive decline. Indeed, numerous molecular mechanisms have been shown to contribute to amyloid-mediated impairments in Ca²⁺ regulation at multiple cellular levels including: altered SERCA activity and increased RyR leak at the ER and the dysregulation of voltage-operated channels, calcium homeostasis modulator 1 (CALHM1), nicotinic acetylcholine receptors, N-methyl-D-aspartate receptors (NMDAR), amino-3-hydroxy-5-methyl-4-isoxazolepropionic acid receptors (AMPAR), and store-operated calcium channels (SOCC) at the plasma membrane. Inversely, there is growing evidence that Ca²⁺ dysregulation can influence and perhaps even precede amyloidogenic disease. This has prompted some investigators to propose that impairments in Ca²⁺ regulation may actually drive AD development. Regardless, there is clear evidence of Ca²⁺ dysregulation with numerous studies suggesting neurons are subjected to elevated ᵢCa²⁺ levels in AD, which would drive increased ₘCa²⁺ uptake.

Evidence of Impaired ₘCa²⁺ Exchange in Neurodegeneration

Numerous studies have implicated ₘCa²⁺ overload in the activation of cell death and neurodegeneration. ₘCa²⁺ is known to cause OMM permeability provoking the release of apoptogens. ₘCa²⁺ is also a central priming event in the opening of the mitochondrial permeability transition pore (MPTP) causing the collapse of membrane potential and loss of ATP production, resulting in necrotic cell death. In support of this critical function, inhibition of MPTP activation using both pharmacological (cyclosporine-A and derivatives) and genetic means (CypD KO) has been shown to reduce neuronal dysfunction and degeneration in both cell culture and mutant mouse AD models. Loss-of-function mutations in MICU1 (a negative regulator of MCU at low-ᵢCa²⁺; so loss of-function promotes increased ₘCa²⁺ uptake) led to severe brain and muscle disorders. While numerous groups have provided a clear link between MPTP opening and progressive AD pathology, to date there remain no reports of ₘCa²⁺ exchange dysfunction in AD, nor a single in vivo genetic exploration of ₘCa²⁺ exchange in brain physiology or disease.

Discovery of ₘCa²⁺ Dysregulation in AD

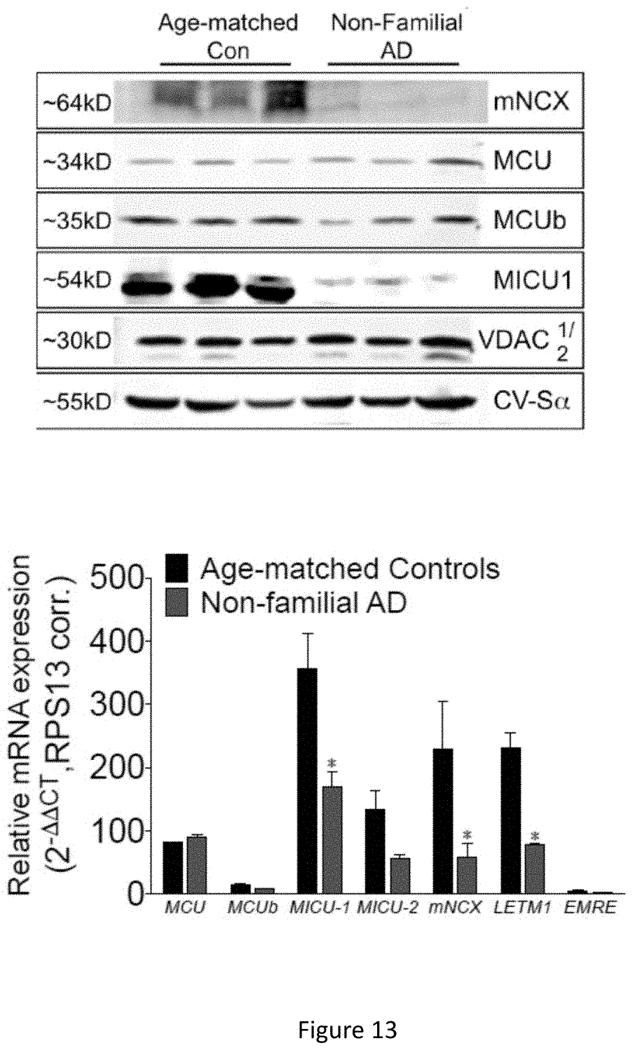

ₘCa²⁺ exchange gene expression is significantly altered in human AD (FIG. 13). Frontal cortex samples were collected postmortem from non-familial AD patients and age-matched controls with no history of dementia. RNA was isolated and SYBR-green qPCR was performed with all data corrected to the housekeeping gene, RPS13. A significant reduction in the MCU negative regulator, MICU1, and a huge reduction in the efflux exchangers, mNCX and LETM1 was observed. In addition, a trend towards a reduction in MCUb was noted. Next, protein was isolated and probed for changes in expression using standard western blot techniques. AD displayed a profound reduction, almost complete loss, in the expression of mNCX and MICU1, and a slight reduction in MCUb, confirming the qPCR results. VDAC and Complex V-Sa were used as mitochondrial loading controls. These data suggest that alterations in the expression of the ₘCa²⁺ efflux exchange machinery may be a significant contributor to ₘCa²⁺-overload in AD. More human samples are used to confirm these molecular changes across multiple AD etiologies.

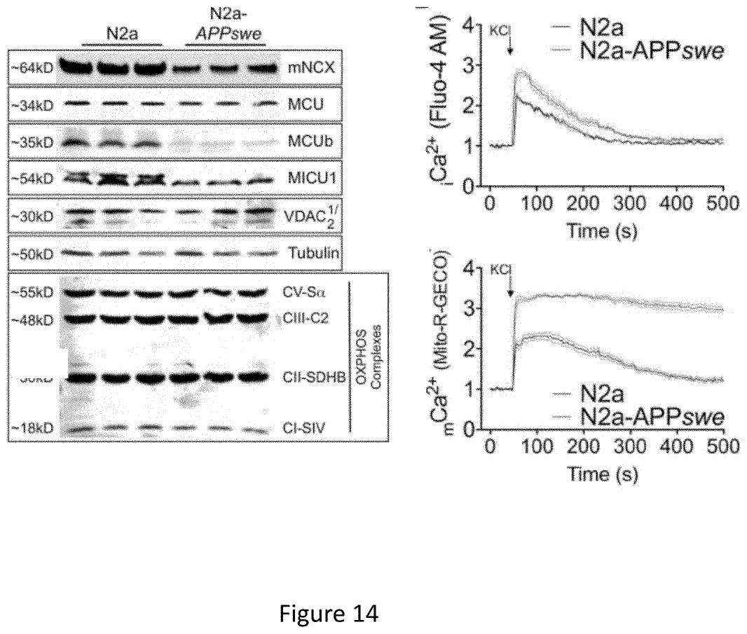

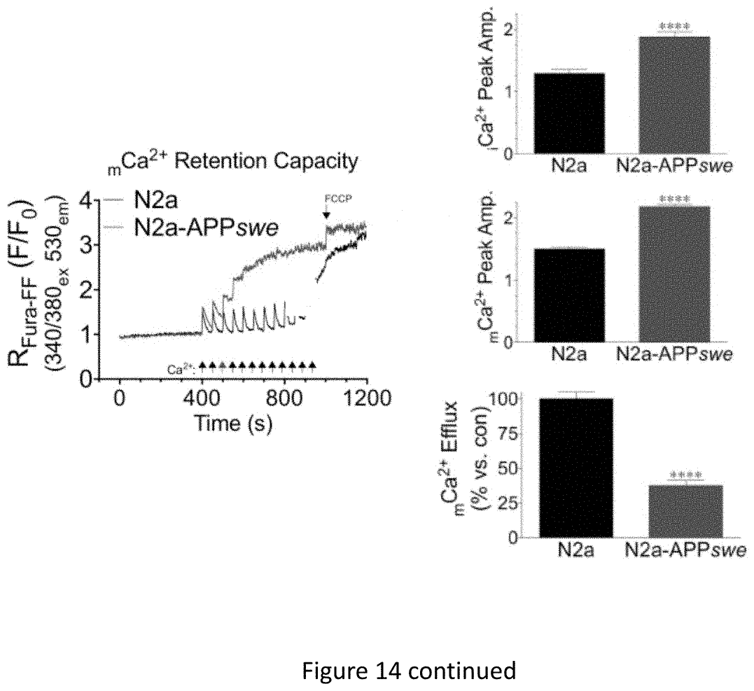

A neuronal cell line expressing human APPswe displays altered ₘCa²⁺ exchanger expression, elevated ᵢCa²⁺ and ₘCa²⁺ transients and increased susceptibility to MPTP activation (FIG. 14). A neuroblastoma cell line (N2a) stably expressing cDNA encoding the APP Swedish mutant (K670N, M671L, APPswe 53) was subjected to a maturation protocol (50% DMEM/50% OPTI-MEM with no serum for 72 h) prior to all experiments presented in the current application. N2a cells expressing APPswe displayed a significant reduction in mNCX (major efflux mediator), MCUb (possible negative regulator of uptake) and MICU1 (inhibitor of uptake at low ᵢCa²⁺) protein expression, mirroring the results obtained from human AD frontal cortex samples. These alterations in expression are consistent with molecular changes that would drive ₘCa2+ overload. Tubulin and OxPhos complex expression served as total and mitochondrial loading controls. Importantly, no change in total OxPhos component expression was observed, suggesting no change in overall mitochondrial content (FIG. 14B & 14D). N2a and N2a-APPswe cells were loaded with the ᵢCa²⁺ reporter, Fluo4-AM and infected with adenovirus encoding the mitochondrial-targeted ₘCa²⁺ reporter, RGECO1 (Ad-mitoR-GECO), and treated with the depolarizing agent, KCl, to activate voltage-gated calcium channels during continuous live-cell imaging (solid line=mean, dashed line=SEM). Quantification of peak amplitude revealed a significant increase (˜40%) in N2a cells expressing APPswe (FIG. 14C). Quantification of ₘCa²⁺ transient peak amplitude found a significant increase in N2a-APPswe, as compared to controls (FIG. 14E). Quantification of the ₘCa²⁺ efflux rate revealed a >60% decrease in APPswe cells (FIG. 14F). To evaluate if impaired ₘCa²⁺ efflux may contribute to ₘCa²⁺ overload, a ₘCa²⁺ retention capacity assay was used. Cells were loaded with the ratiometric reporters FuraFF (Ca²⁺) and JC1 (mitochondrial membrane potential). Recordings are only shown for FuraFF for clarity. (FIG. 14G). Next, cells were permeabilized with digitonin and treated with thapsigargin to inhibit SERCA so that the movement of Ca²⁺ was only influenced by mitochondrial uptake. The protonophore, FCCP, was used at the conclusion of the experiment to correct for total Ca²⁺ in the system. N2a cells expressing the APPswe mutation underwent complete permeability transition after the 3rd 10-μM pulse of Ca²⁺ (red arrow, in representative recordings). This was in striking contrast to control, which sustained 3× the concentration of bath Ca²⁺ before collapse of Δψ and loss of ₘCa²⁺.

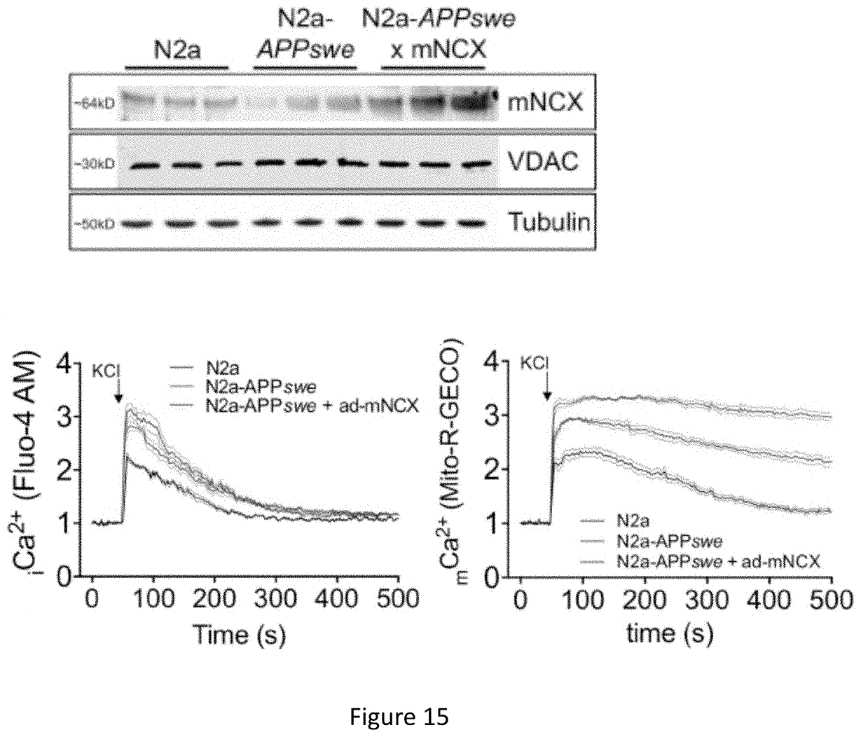

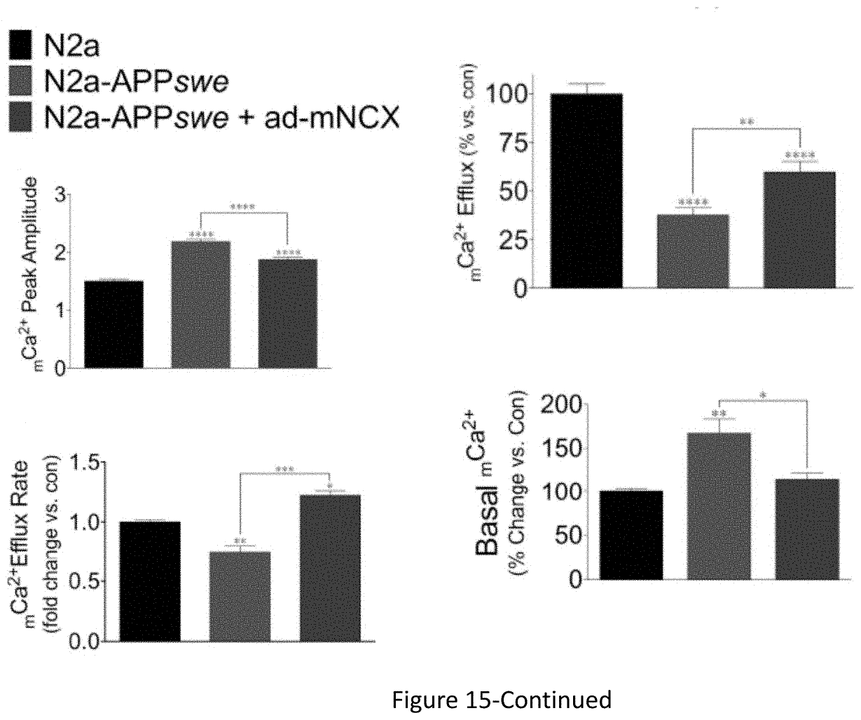

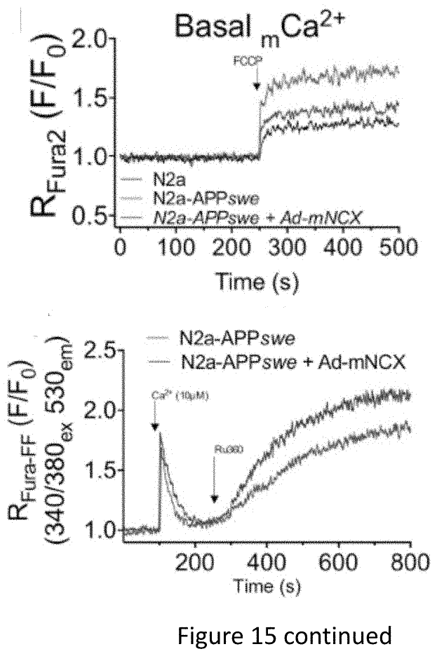

Expression of mNCX rescues APPswe-induced defects in ₘCa²⁺ handling (FIG. 15). To examine if restoring ₘCa²⁺ efflux capacity is sufficient to rescue impairments in ₘCa2+ handling and reduce ₘCa²⁺ overload maturated N2a-APPswe cells were infected with adenovirus encoding mNCX (Ad-mNCX). 48h after infection, western blot analysis revealed a complete rescue of mNCX expression in APPswe cells. VDAC and tubulin served as loading controls (FIG. 15A). Cells from all three groups were loaded with the ᵢCa²⁺ reporter Fluo4-AM and infected with Ad-mitoR-GECO and imaged continuously during stimulation with KCl (FIG. 15B-15E). Traces shown represent the mean±SEM. While expression of mNCX did not alter the APP-mediated increase in iCa2+ flux, it restored the mitochondrial transient towards that of control N2a cells (FIG. 15B-15C. mNCX expression significantly reduced the ₘCa²⁺ peak amplitude and increased the efflux rate by ˜50% vs. APPswe cells (FIG. 15D-15E). To corroborate this finding a biophysical assay was used to carefully quantify changes in ₘCa²⁺ uptake and efflux rates (FIG. 15F). Cells were loaded with FuraFF, permeabilized with digitonin and treated with thapsigargin. While no change was found in the uptake rate (downward slope following 10 μM-Ca2+ addition), there was a clear increase in the ₘCa²⁺ efflux rate following the addition of the MCU-inhibitor, Ru360 (blue trace). APPswe cells expressing mNCX displayed ₘCa²⁺ efflux rates equal to, or faster than, N2a control cells (FIG. 15G). To discover if enhancing mNCX-mediated efflux was sufficient to reduce ₘCa²⁺ overload and restore matrix Ca2+ levels, maturated N2a cells from all 3 groups were pretreated with the MCU-inhibitor, Ru360, and the mNCX inhibitor, CGP37157, to ‘lockin’ ₘCa²⁺ and then loaded with Fura2 and treated with digitonin and thapsigargin as previously reported. Upon reaching a steady state recording, the protonophore, FCCP, was used to collapse Δψ, and initiate the release of all matrix free Ca²⁺ (FIG. 15H). Quantification of basal ₘCa²⁺ content found that mNCX expression completely corrected APPswe-mediated Ca2+ overload (FIG. 15I).

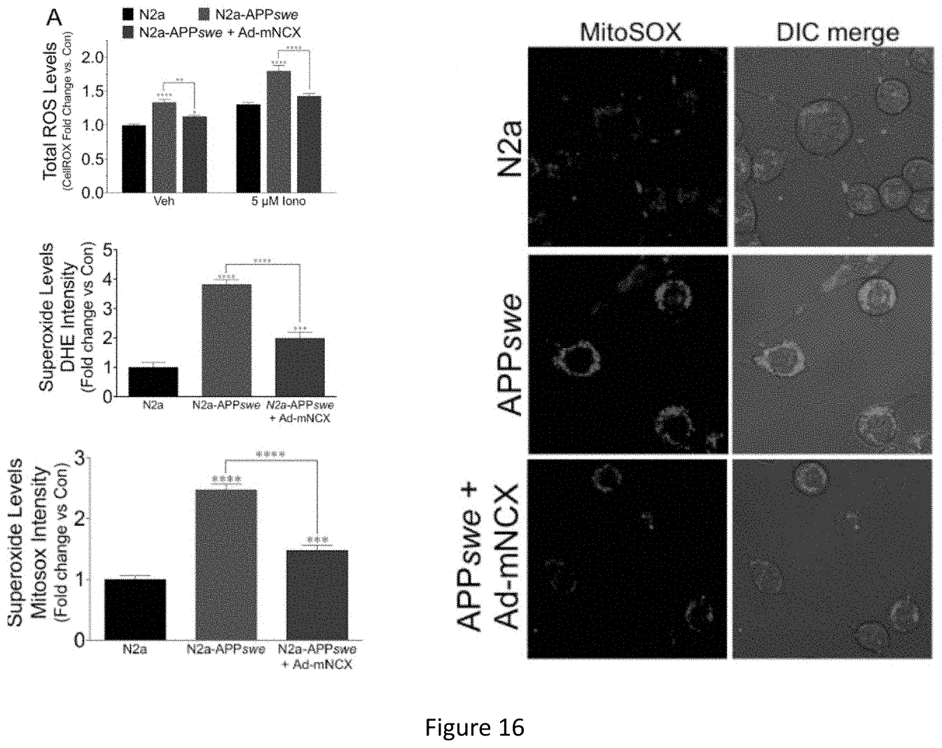

Expression of mNCX reduces superoxide (O2^{°—}) generation in a neuronal AD model (FIG. 16). ₘCa²⁺ overload is known to elicit increased ROS generation and inhibition of ROS scavenging pathways via numerous molecular mechanisms. Maturated cells (N2a, N2a-APPswe, and APPswe+Ad-mNCX) were examined for changes in redox status utilizing 3 different ROS sensors during live-cell imaging. The total cellular ROS indicator, CellROX Green, was loaded and cells were imaged 30m following treatment with vehicle (Veh) or the Ca²⁺ ionophore, ionomycin (Iono). N2a cells expressing APPswe displayed an increase in total ROS that was significantly reduced in cells expressing mNCX (48 h post-adeno). Next, employed the O2°—specific probe dihydroethidium (DHE) was used. DHE when oxidized to 2-hydroxyethidium intercalates DNA and increases fluorescent intensity (>500-fold). N2a-APPswe had a ˜4-fold increase in O2°—production that was reduced by ˜50% with mNCX expression (Ad-mNCX). To further define the subcellular site of ROS generation the mito-targeted O2°—indicator, MitoSOX Red was used. Representative images of MitoSOX staining (510ex/580em) and differential interference contrast (DIC) merge are shown in FIG. 16C. Quantification of MitoSOX fluorescent intensity; fold change vs. N2a con are shown. These results support the notion that expression of mNCX, in the context of AD-like stress, reduces mitochondrial O2°—production.

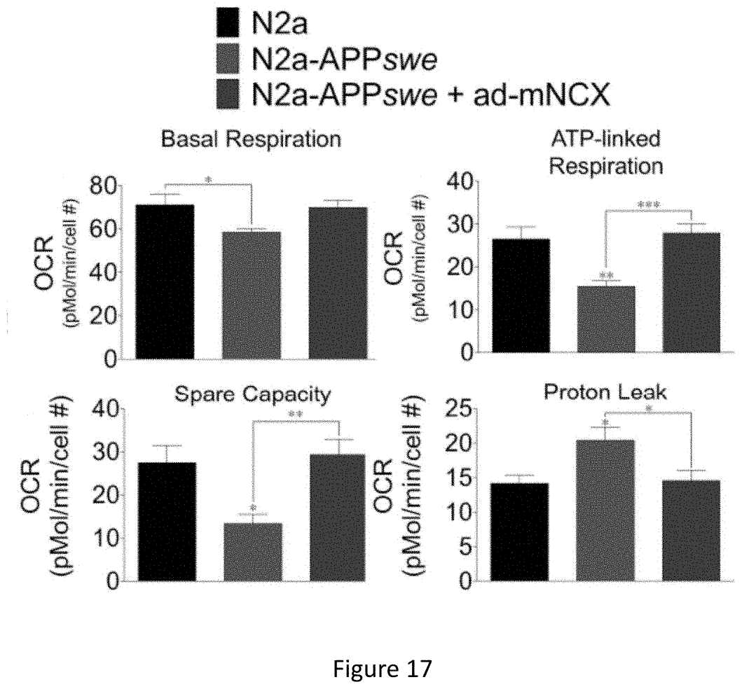

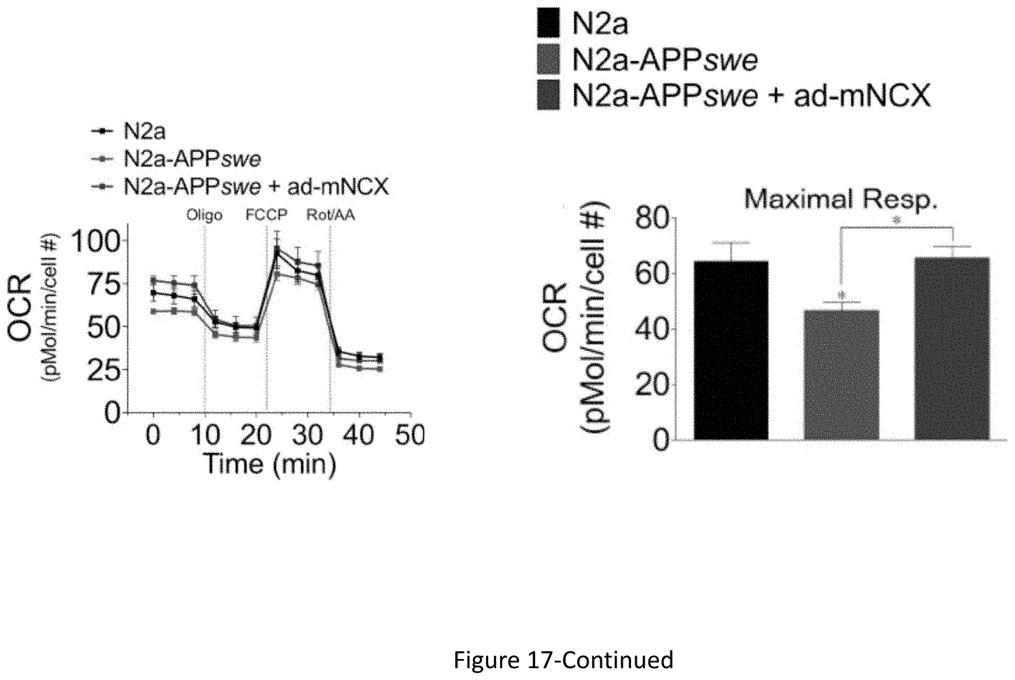

Expression of mNCX rescues OxPhos defects in APPswe cells (FIG. 17). It's well known that excessive matrix Ca²⁺ augments mito O2°—generation, as shown in FIG. 16, and thereby can negatively impact OxPhos. AD is characterized by neuronal hypometabolism, with studies suggesting that mitochondrial defects in energy production may underlie neurodegeneration and cognitive decline in AD. Maturated N2a-APPswe cells were examined for changes in OxPhos and mito function using a Seahorse XF96 extracellular flux analyzer to monitor oxygen consumption rates (OCR). Representative OCRs at baseline and following: oligomycin (oligo; CV inhibitor; to uncover ATPlinked respiration), FCCP (protonophore to induce max respiration), and rotenone+antimycin A (Rot/AA; complex I and III inhibitor; complete OxPhos inhibition). Quantification of basal respiration (base OCR non-mito respiration (post-Rot/AA) are shown. Quantification of ATP-linked respiration (post-oligo OCR—base OCR) are shown. Max respiratory capacity (post-FCCP OCR post-Rot/AA) are shown. Spare respiratory capacity (post-FCCP OCR—basal OCR) are shown. Proton leak (post-Oligo OCR—post Rot/AA OCR). Expression of APPswe mutant protein significantly decreased all mito respiratory parameters examined. Amazingly, rescue of ₘCa²⁺ efflux with AdmNCX infection for 48 h corrected all OCR measurements back to N2a control levels. These results support that ₘCa²⁺ overload is a significant contributor to AD-mediated impairments in OxPhos and that mNCX is sufficient to restore bioenergetics.

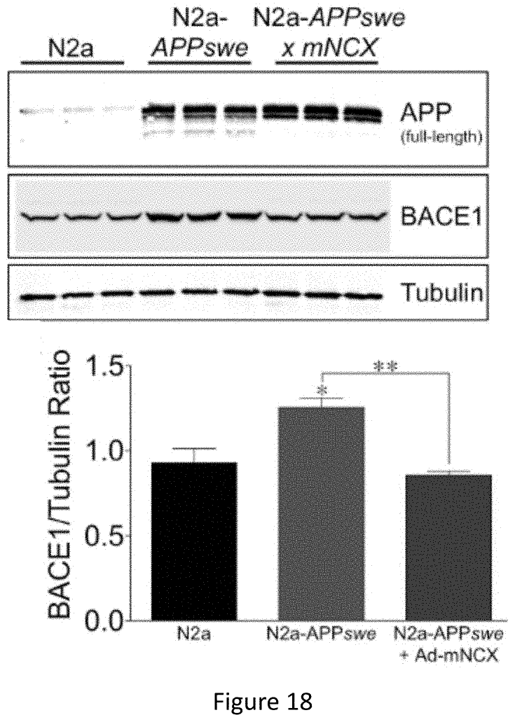

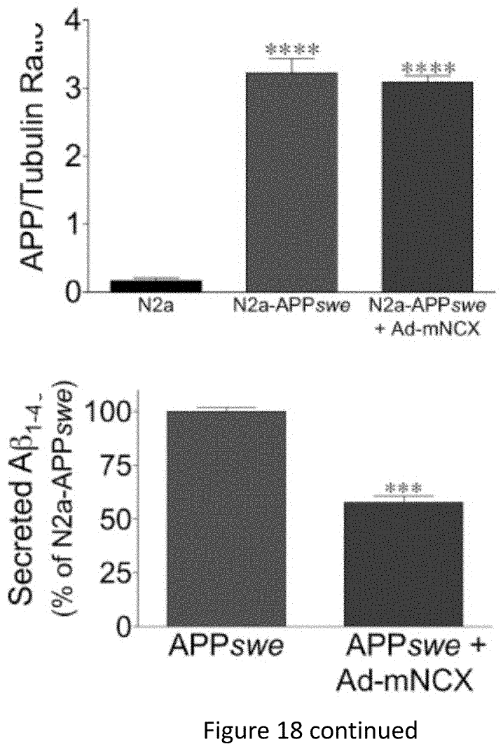

Enhancing ₘCa2+ efflux decreases the amyloidogenic Aβ pathway (FIG. 18). An intense research effort has been placed on identifying the link between Ca²⁺ dysregulation and the Aβ amyloidogenic pathway. Studies have suggested that Aβ increases ᵢCa²⁺ levels by numerous mechanisms and vice versa, increased ᵢCa²⁺ augments Aβ production and tau hyperphosphorylation. Enhancing ₘCa²⁺ efflux (mNCX expression for 48 hours) reduced β-secretase (BACE1) expression and extracellular Aβ1-40 levels in N2a-APPswe cells. No change in full-length APP expression was observed given the AD cell model features stable overexpression of mutant APP. These results are intriguing and suggest that elevated ₘCa²⁺ signaling may contribute to the amyloid cascade.

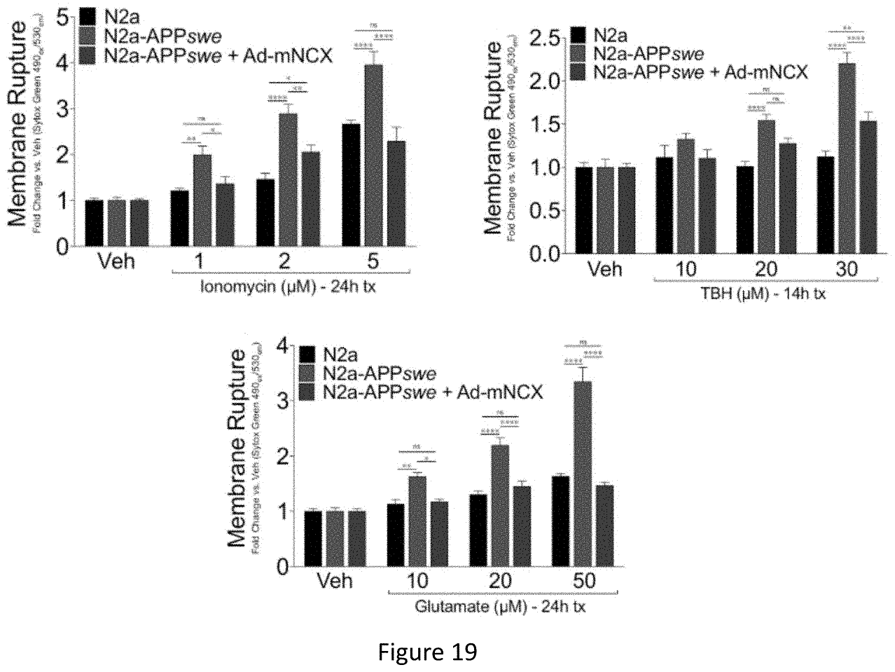

Enhancement of ₘCa2+ efflux reduces cell death induced by a variety of stressors (FIG. 19). ₘCa²⁺ overload has been shown to augment neuronal cell death both through primary (MPTP and ROS) and secondary signaling mechanisms (metabolic derangement, etc.). Given that mNCX expression reduced O2°—production, MPTP activation, and also enhanced OxPhos capacity it was tested if these protective mechanisms coalesced to reduce neuronal demise. N2a, N2a-APPswe and N2a-APPswe infected with Ad-mNCX for 48 h were treated with Iono, (1-5 μM) for 24 h and examined for plasma membrane rupture (hallmark of cell death) using the cell membrane impermeable dye, Sytox Green. Iono significantly increased membrane rupture in APPswe expressing cells over the N2a control at all doses and this was significantly reduced with mNCX expression. Similarly all groups were treated with the oxidizing agent and free-radical generator, tert-Butyl hydroperioxide (TBH), which is preferred over H2O2 due to its increased stability in solution. Treatment with 20 and 30 μM TBH for 14 h significantly increased membrane rupture in APPswe expressing cells over the N2a control, which was reduced with increased mNCX expression. Likewise, treatment with glutamate (NDMAR-agonist, neuroexcitotoxicity agent) significantly increased cell death in APPswe expressing cells across all doses and this was completely ablated with mNCX expression. These results strongly support that rescue of mNCX expression in the context of AD may be a powerful therapeutic to impede cell loss and AD progression.

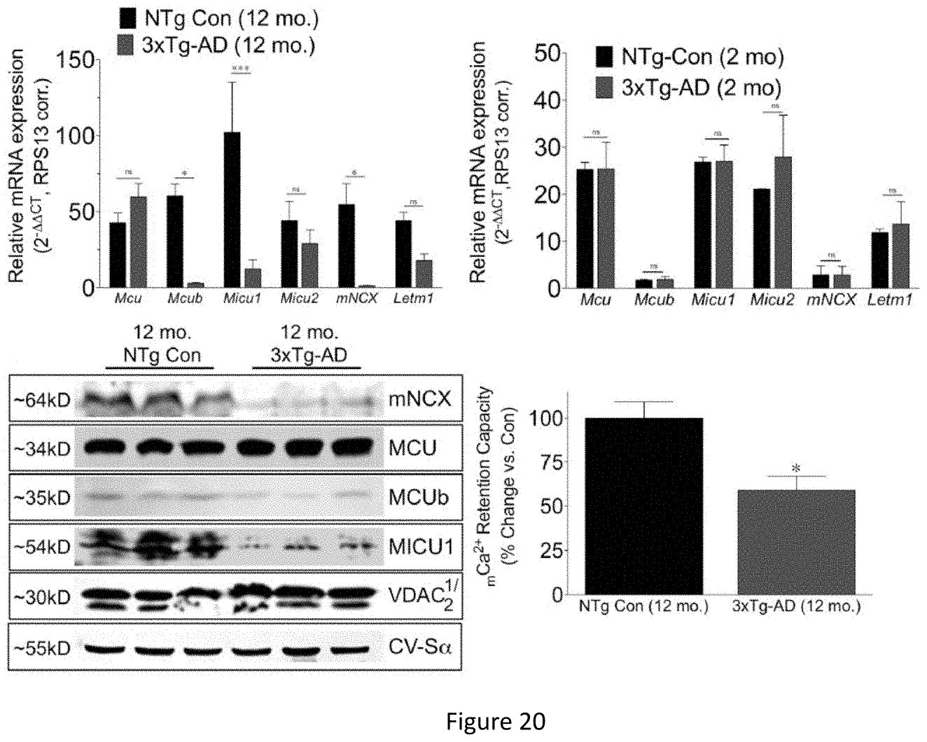

ₘCa²⁺ exchange gene expression and ₘCa2+ handling is significantly altered in 3xTg-AD mice (FIG. 20). To confirm that the alterations in ₘCa²⁺ transporter expression observed in AD patients is capitulated in a murine model of AD, mutant mice harboring 3 mutations associated with familial AD (3xTg-AD) were used. These mice develop age-progressive pathology similar to that observed in AD patients including: impaired synaptic transmission, Aβ deposition, and plaque and tangle histopathology. Mice are homozygous for the Psen1 mutation (M146V knock-in), and contain transgenes inserted into the same loci expressing the APPswe mutation (APP KM670/671NL) and tau mutation (MAPT P301L). Brain tissue was isolated from the frontal cortex of aged (12 mo.) 3x-Tg AD mutant mice and outbred non-transgenic controls (NTg) and RNA was isolated for qPCR quantification of gene expression. 3xTg-AD mice displayed a huge reduction in Mcub and Micu1 RNA levels, which given the hypothesized role of these proteins as negative regulators of MCUc would promote ₘCa²⁺ overload. Strikingly, mNCX expression was completely absent mirroring the results obtained from human AD brain samples (FIG. 13). Importantly, no alteration was observed in gene expression in frontal cortex tissue isolated from 2-month-old 3x-Tg mice, an age prior to any detectable neuropathology or altered cognition. This provides evidence that the alterations in ₘCa²⁺ exchanger expression are not merely the result of developmental expression changes associated with the mutant model. In future studies we will examine this model at 4 months of age, just prior to detectable changes in long-term retention and long-term potentiation (LTP). To confirm that the changes in mRNA expression were manifest at the protein level tissue samples from 12-mo.-old mice were examined using standard Western blot techniques. Loss of mNCX immunoreactivity was confirmed, as was a significant reduction in MICU1, and a slight reduction in MCUb (Complex V-subunit alpha served as a mitochondrial loading control, CV-Sα). To discern if ₘCa2+-overload is a feature of the 3xTg-AD model, mitochondria was isolated from the frontal cortex and hippocampus and a Ca²⁺ retention capacity assay (CRC) was performed using the reporter Ca-Green-5n. Isolated mitochondria were continuously monitored for changes in fluorescence using a spectrofluorometer during 10-μM bath Ca²⁺ additions every 50 s. A ˜50% reduction in CRC in mitochondria isolated from 3x-Tg mice vs. NTg con was observed. This result suggests that MPTP activation occurs in this AD model with ˜half the Ca2+ stress as WT controls.

Generation of Slc8b1 (mNCX) Conditional Knockout Mice.

A Slc8b1 conditional knockout mouse was generated by acquiring targeted ES cells generated by recombinant insertion of a knockout-1st mutant construct containing loxP sites flanking exons 5-7 of the Slc8b1 gene (ch12: 113298759-113359493) 63. ES cell lines (clone EPD0460_4_A08, EUCOMM) were confirmed by PCR and injected into C57BL/6N blastocysts with subsequent transplantation into pseudo- pregnant females. Germline mutant mice were crossed with ROSA26-FLPe knock-in mice for removal of the FRT-flanked splice acceptor site, βgal reporter, and neomycin resistance cassette. Resultant Slc8b1fl/+ mice were interbred to generate homozygous mutant mice with conditional knockout potential (S1c8b1fl/fl). Next, Slc8b1fl/fl mice were crossed with neuronal specific-Cre transgenic mouse models, Camk2a-Cre (Jax #5359) or tamoxifen-inducible Camk2a-Cre-ERT2 mice (Jax #12362) to generate neuronal restricted-deletion of Slc8b1 (FIG. 21). Camk2a-Cre is predominantly expressed in the forebrain, with strong expression in the frontal cortex and CA1 pyramidal cell layer in the hippocampus. The ERT2-inducible model was used to delete Slc8b1 in the adult brain and avoid any developmental issues associated with mNCX deletion. For temporal deletion of Slc8b1, 2-month-old mice are injected i.p. with tamoxifen (tamox, 20 mg/kg/day) for 5 consecutive days; importantly all groups including controls receive tamox. These neuronal, conditional knockouts are referred to as mNCX-nKO (mNCX neuron-restricted knockout). Functionality of the neuronal mNCX mutant at the RNA level was confirmed and functionality of the ‘foxed’ mice were confirmed in experiments where they were crossed with αNIFIC-Cre mice for cardiomyocyterestricted deletion and complete loss of ₘCa²⁺ efflux was noted (FIG. 21E-21G). Importantly, mNCX-nKO mice displayed no change in expression of MCUc components, Mcu or Micu1 (FIG. 21D).

Generation of a Neuronal-Specific mNCX Overexpression Mouse Model

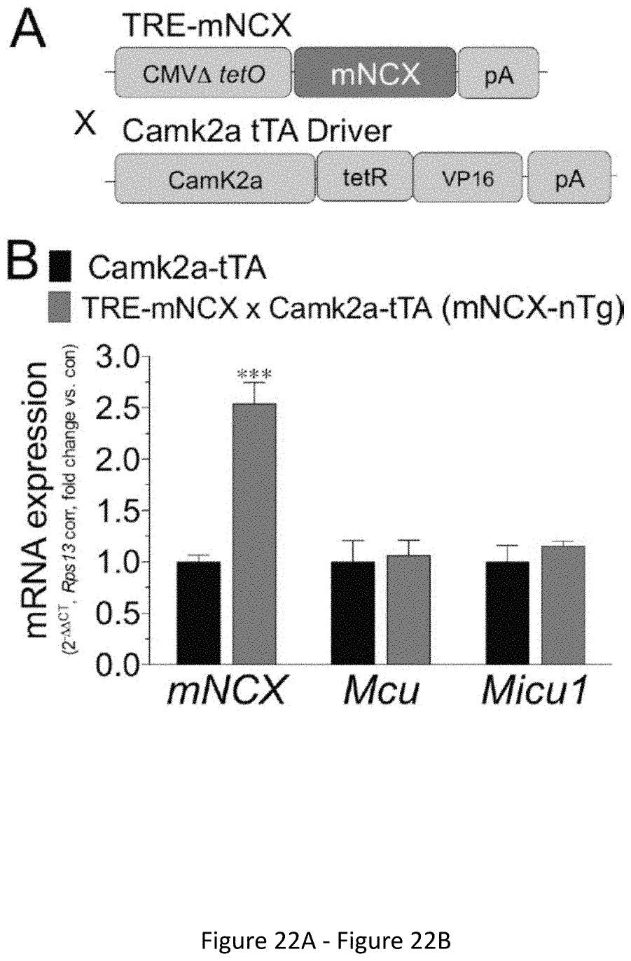

The human SLC8B1 sequence (NM_024959, mNCX) (5′ EcoRI, 3′ XmaO) was cloned into a plasmid containing the Ptight Tet-responsive promoter and SV40 poly A. Upon sequence confirmation the purified fragment was injected into the pronucleus of fertilized ovum and transplanted into pseudopregnant females (C57BL6N). After germline confirmation of founders, TRE-mNCX mice were crossed with the Camk2a-tTA transgenic model (FIG. 22A, inducible, neuronal restricted expression under the control of the CamK2a promoter, doxycycline (dox)-off) (Mayford, M. et al., 1996, Cold spring harb symp quant boil 61:219-224). This allows conditional overexpression upon the withdrawal of dox containing food. These animals are referred to as mNCX-nTg (mNCX neuron-specific transgenic and display ˜2.5-fold increase in mRNA expression (FIG. 22B). All mutant mice are fed dox until 2 mo. of age to inhibit embryonic and developmental mNCX expression. Both of these conditional models alleviate any concerns of lethality and given that both models have been confirmed to be functional there are no technical limitations with these studies.

TABLE 1
New Mutant Mouse Models

| Mutant Mouse | | | |
|---|---|---|---|
| Model | Abbreviation | Application | Control Group |
| Slc8b1^{fl/fl} × | mNCX-nKO | Conditional, neuronal deletion of mNCX in the | Camk2a-CreERT2 + |
| Camk2a-CreERT2 | | forebrain and hippocampus of adult mice. | tamoxifen |
| Slc8b1^{fl/fl} × | mNCX-KO | Neuronal deletion of mNCX in forebrain and | Camk2a-Cre |
| Camk2a-Cre | | hippocampus of adult mice. Cre is constitutively active. | |
| TRE-mNCX × | mNCX-nTg | Conditional, neuronal-specific overexpression of | Camk2a-tTA + |
| Camk2a-tTA | | mNCX in the forebrain and hippocampus of adult mice | doxycycline |
| MCU^{fl/fl} × | MCU-nKO | Conditional, neuronal deletion of MCU in the forebrain | Camk2a-CreERT2 + |
| Camk2a-CreERT2 | | and hippocampus of adult mice. | tamoxifen |

Deletion and Overexpression of mNCX in the 3xTg-AD Mutant Mouse.

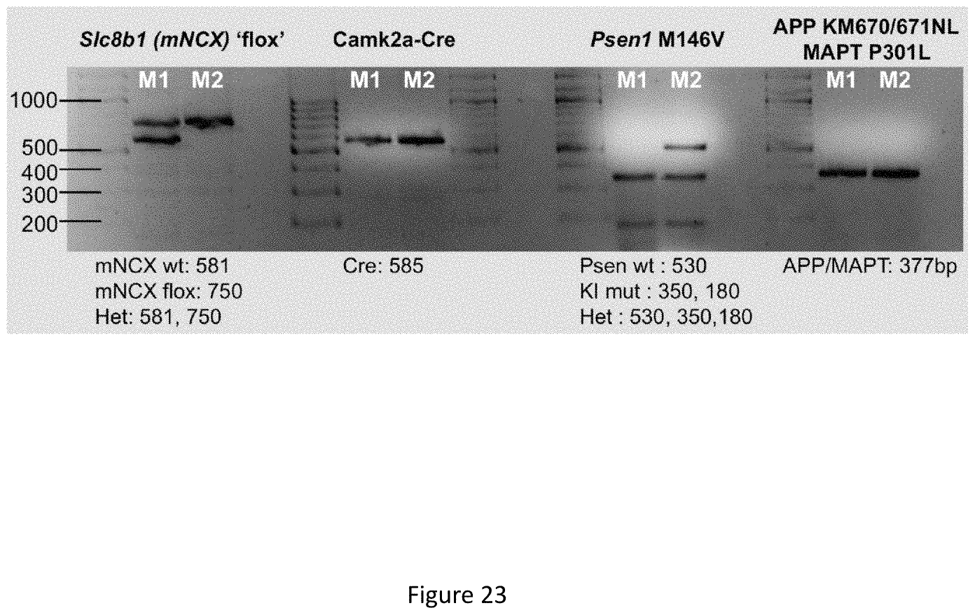

To definitively test if ₘCa²⁺ efflux plays a role in AD development and progression both conditional, neuronal-specific gain- and loss-of-function models (mNCX-nKO and mNCXnTg) to the 3xTg-AD mutant mouse were crossed. These crosses have taken over a year of intensive breeding to acquire the proper genotype (7 mutant alleles in the case of the nKO), but recently breeding pairs for all experimental and control groups were acquired. Proof of these animals can be seen in FIG. 23 where genotyping of 2 pups from the mNCX-nKO x 3xTg-AD cross is presented (FIG. 23). Table 2 outlines all the experimental and control groups that are utilized.

TABLE 2
Genotypes utilized for AD Mutant Mouse Studies

| Experimental Group | Abbreviation | Control Groups |
|---|---|---|
| mNCXnKO × 3xTg-AD | AD-mNCX-nKO | 1. Camk2a-CreERT2 × |
| (+tamoxifen @ 2 mo.) | | 3xTg-AD |
| | | 2. mNCX-nKO |
| | | 3. Camk2a-CreERT2 |
| | | (All groups will also |
| | | receive tamox inj.) |
| mNCX-nTg × 3xTg-AD | AD-mNCX-nTg | 1. Camk2a-tTA × |
| (dox withdraw at 2 mo.) | | 3xTg-AD |
| | | 2. mNCX-nTg |
| | | 3. Camk2a-tTA |
| | | (All groups will receive |
| | | dox until age 2 m) |

Examination of the Molecular Function of mNCX in Neuronal ₘCa2+ Regulation and Impact on Mitochondrial Function, Metabolism and Cell Death Signaling.

It is postulated that neuronal function is integrated with energy production via ₘCa²⁺ exchange. ᵢCa²⁺ cycling is fundamental to synaptic transmission and facilitates feed-forward signaling to the mitochondria to ensure that ATP production meets functional demand. The mitochondrial matrix contains multiple Ca²⁺ control points to modulate oxidative phosphorylation including Ca²⁺-dependent dehydrogenases and direct action on components of the electron transport chain (ETC). In addition, it is widely recognized that ₘCa²⁺ can directly influence cell death signaling by activating mitochondrial permeability transition, Ca²⁺-dependent proteases (calpains), and secondarily through its effects on ATP availability. The tight coupling of these two contrasting processes makes it a necessity to experimentally evaluate both metabolism and cell death in the context of AD. Both in vitro and in vivo gain/loss-of-function approaches are utilized to molecularly dissect the involvement of mNCX in these physiological cellular processes.

The greatest contributor to ₘCa²⁺ efflux in neurons is the Na⁺/Ca²⁺ exchanger making it the ideal target to truly discern a causative role for ₘCa²⁺ exchange in AD pathophysiology. There exists convincing data that the ₘCa²⁺ microdomain contributes significantly to neuronal metabolic regulation and the activation of cell death pathways. Both of these processes are thought to contribute to AD progression, providing strong rational to define ₘCa²⁺ exchange mechanisms. Here the biophysical properties of the exchanger are characterized and identified to see whether mNCX modulation impacts neuronal metabolism and cell death.

To examine mNCX function in instances of cellular stress, cortical/hippocampal neurons from the brains of E15 mutant pups are isolated (Cheung, K. H. et al., 2008, Neuron 58:871-883; Cheung, K. H. et al., 2010, Science signaling 3, ra22). For loss-of-function experiments, neurons from mNCXfl/fl pups are isolated and after culturing for 7 days to allow for maturation, cells with adenovirus (adeno) encoding Cre-recombinase (Ad-Cre) are infected for efficient deletion of mNCX or β-galactosidase (Ad-βgal, control infection). 96 hours following adeno infection, neurons are utilized in the various experiments. This period of time is needed for protein turnover as it is founded that the half-life of mNCX in culture is ˜40 hours. For gain-of-function experiments, neurons from TRE-mNCX pups in an identical fashion is isolated, but here neurons are infected with adeno encoding the tetracycline controlled transactivator (Ad-tTA) for overexpression of mNCX or β-gal as an adeno control. After 48 hours to allow for expression, neurons are utilized in the various experiments. This type of genetic system for in vitro functional studies is preferred as the primary neurons isolated are the same for both the control and experimental groups and thereby this avoids any issues with consistency and or heterogeneity of the population that can occur as a result of independent isolations.

Examine the Biophysical Properties of mNCX and its Contribution to Neuronal Ca²⁺ Dynamics.

Using the primary neuronal systems outlined above (mNCX+Ad-Cre and TRE-mNCX+tTA and controls), neurons are infected with adeno encoding the ₘCa²⁺ reporter mito-R-GECO1 and 24 h later load the same cells with the iCa²⁺ reporter Fluo4-AM for simultaneous imaging of iCa²⁺ and ₘCa²⁺ transients on high-speed imaging system. Neurons are treated with various iCa²⁺ activators during imaging including: field stimulation (40v, 0.2 ms), KCl (100-mM, general activation of voltage-gate channels), glutamate (10-μM, NMDAR agonist), bzATP (50 μM, puringenic agonist for IP3R Ca²⁺ release). Transients (iCa²⁺ and ₘCa²⁺) are analyzed using Chart 6.0 to quantify: peak amplitude, time-to-peak, decay time and tau (time-rate decay) (Luongo, T. S. et al., 2015, cell reports 12:23-34).

To further characterize mNCX, a high-fidelity spectrofluorometer is used to simultaneously record changes in Δψ and ₘCa²⁺ flux in mNCX deleted and overexpressed neurons by loading them with the ratiometric reporter dyes FuraFF and JC1. Briefly, FuraFF and JC1 loaded neurons are permeabilized with digitonin, and ER Ca²⁺ flux inhibited with thapsigargin (SERCA inhibitor), so that FuraFF ratiometric changes only reflect ₘCa²⁺ exchange. Then the bath Ca²⁺ levels are systematically altered and quantify Ca²⁺ uptake and, after Ru360 addition (MCU inhibitor) quantify Ca²⁺ efflux rates.

Analysis of Matrix Free-Ca²⁺ Content.

To examine if mNCX deletion or overexpression alters baseline ₘCa²⁺ levels a protocol using Fura2, rather than FuraFF, is used so that the kD of the reporter is more appropriate for matrix Ca²⁺ levels. In this experiment neurons are pretreated with the MCU inhibitor, Ru360, and mNCX inhibitor, CGP37157, to block ₘCa²⁺ movement during plasma membrane permeabilization and SERCA inhibition. Then after a stable baseline recording of Fura2 and JC1 is reached, FCCP is added to release all free-Ca²⁺ from the matrix (collapse of Δψ). Data is curve fitted to determine actual ₘCa²⁺ content.

In data generated in the N2a cell line it was found that the expression of mNCX reduced APPswe-mediated deficits in OxPhos (FIG. 17). Here first, brain tissue isolated from the frontal cortex of mNCX-nKO and mNCX-nTg mice and their respective controls for alterations in ₘCa²⁺-dependent metabolic processes is analyzed. Specifically, following the isolation of mitochondria the activity of the mitochondrial dehydrogenases (KGD and PDH) is probed and expression and phosphorylation status of (PDH, KGD, IDH) is examined (Luongo, T. S. et al., 2015, cell reports 12:23-34; Elrod, J. W. et al., 2010, J Clin Invest. 120:3680-3687). In addition, the redox status of the nicotinamide adenine dinucleotide (NAD) pool is examined using a fluorometric NAD/NADH assay. The primary neuronal culture system is utilized to measure NAD+ autofluorescence in real-time during application of KCl and glutamate,. To directly assess OxPhos, Seahorse Bioscience XF96 flux analyzer is utilized to analyze oxygen consumption rates (OCR) in mitochondria isolated from the frontal cortex of mNCX-nKO and mNCX-nTg mice and their respective controls. ATP production and ATP content in lysates isolated from the brains of the mNCX mutant mice is examined.

The primary mutant neuronal culture models are utilized to examine the totality of ₘCa²⁺ signaling in the regulation of cell death. Data generated in the N2a-APPswe cell line suggests that expression of mNCX may be a potent protective mechanism against cell death induced by a variety of stressors (FIG. 19). These provocative results suggest that enhancing ₘCa²⁺ efflux may be a powerful cytoprotective mechanism in AD, possibly by decreasing MPTP activation, preserving mitochondrial integrity and function, increasing OxPhos, and maintaining the ATP pool.

Analysis of Cell Death and APP Metabolism.

A variety of pharmacologic cell death inducers are examined in the primary mutant neurons including: TBH 10-30 μM (ROS), thapsigargin 10-30 μM (ER Ca²⁺ mobilization), ionomycin 1-10 μM (global Ca²⁺ overload), glutamate 10-50 μM (NMDAR excitotoxicity) and adenovirus delivery of familial AD mutant genes (APPswe and PSEN1 E280A)+ROS and Ca²⁺ stressors. 16-24 h after treatment, a number of end-points are analyzed to characterize the mechanism of cellular demise including: membrane rupture (Sytox green), general viability (resazurin blue), metabolic capacity (ATP levels, luciferase assay), and MPTP opening (calcein/cobalt assay). In experiments where mutant AD genes are delivered in combination with Ca²⁺ and ROS stress AP signaling is examined using the methods described in FIG. 18.

It is important to evaluate mitochondria isolated from the frontal cortex/hippocampus of mutant mice (overexpression and targeted deletion) to accurately assess MPTP regulation and corroborate the in vitro findings. These experiments include: the quantification of Ca²⁺ retention capacity using FuraFF, monitoring swelling in response to Ca²⁺ challenge (change in absorbance), examination of membrane potential changes (TMRE during death inducing stimuli), and structural assessment (electron microscopy). Calpain (Ca²⁺-activated proteases) activation is reported to be increased and widespread in the AD brain (Saito, K. et al., 1993, Proceedings of the national academy of sciences of the united states of America) and inhibition of calpains improved cognitive function in an APP/PSEN1 mutant mouse model (Trinchese, F. et al., 2008, J Clin Invest 118:2796-2807). μ-calpain and calpain 10 localization to mitochondria where they contribute to programmed cell death (Kar, P. et al., 2010, Archives of biochemistry and biophysics 495:1-7). Calpain activity is determined spectrophotometrically using the calpain-specific substrate Ac-LLY-AFC. In this assay, energized mitochondria are incubated with various concentrations of Ca²⁺ in the presence of substrate and activity (fluorescence) is measured using a plate reader.

It is hypothesized herein that mNCX significantly contributes to neuronal ₘCa²⁺ efflux and thus genetic loss results in ₘCa²⁺ overload, increased MPTP activation, metabolic derangement, and susceptibility to cell death. Conversely, is hypothesized herein that enhanced mNCX function (overexpression) will augment ₘCa²⁺ efflux in the face of stress stimuli promoting the maintenance of cellular function and survival.

Determine if ₘCa²⁺ Overload is a Key Contributor to Development and Progression of AD

Aβ deposition and aggregate-mediated cellular toxicity have been repeatedly linked to neuronal Ca²⁺ dysregulation in AD. Further, familial AD mutations have been reported to increase ,Ca²⁺ load and elicit mitochondrial dysfunction via numerous molecular mechanisms. To define if ₘCa²⁺ exchange abnormalities contribute to the progression of AD, neuronal mNCX are deleted in the adult brains of 3xTg-AD mice and evaluate neurodegeneration cognitive function, and neuropathology. These studies determine if loss of neuronal ₘCa²⁺ efflux exacerbates neuronal decline in a relevant animal model of AD.

Slc8b1fl/fl×Camk2a-CreERT2 (mNCX-nKO) mice have been crossed with 3xTg-AD mutant mice. This model is homozygous for the Psen1 mutation (M146V knock-in), and contains transgenes at the same loci expressing the APPswe mutation (APP KM670/671NL) and tau mutation (MAPT P301L). Breeding over the past 14 mo. has resulted in the establishment of breeding pairs for experimental study (see FIG. 23, Table 2). mNCX-nKO-AD mice and appropriate controls. Tamoxifen is administered at 2 mo. of age (20 mg/kg i.p. for 5 d) to all groups of mice. Deletion of mNCX at this age is to avoid developmental and compensatory gene modifications that may be caused by neonatal deletion. Mice undergo extensive phenotyping at both 6 and 12 mo. of age to examine: cognitive function, synaptic integrity, neurohistopathology, mitochondrial structure/function, redox state, metabolic alterations and neuronal demise. Extensive phenotyping with multiple time points is critical to establish if the noted pathology is progressive in nature. While some of the pathologic end-points are not observed at 6 mo. in the 3× model, it is still evaluated, because loss of mNCX, speeds disease progression.

Mice at 6 and 12 mo. of age are assessed for behavioral impairments in the following tests: novel object recognition, Y-maze, fear conditioning, and Morris water maze (Chu, J. et al., 2013, Translational psychiatry 3: e333; Giannopoulos, P.F. et al., 2014, Molecular psychiatry 19: 511-518; Chu, J. et al., 2012, Ann Neurol 72:442-454). At sacrifice, brains are harvested and immediately divided in two halves: one for biochemistry (cortex and hippocampus), the other half for immunohistochemistry looking at changes in: Aβ deposition and metabolism, tau phosphorylation and metabolism, synaptic function and integrity.

After the behavioral tests, a subgroup of mice are rapidly decapitated at 6 and 12 mo. of age, to harvest hippocampal slices for electrophysiological characterization of synaptic function to analyze input/output curves, paired-pulse facilitation (PFF), field excitatory post synaptic potentials (fEPSPs) and long-term potentiation (LTP).

A combination of techniques is employed to evaluate Aβ generation and metabolism in mice at 6 and 12 mo. of age including: immunohistochemistry, biochemistry, and quantitative ELISA assays. For immunodetection of Aβ deposits a pan anti-Aβ monoclonal antibody, 4G8, the classical dye Thioflavin S, and congo red are used. Aβ1-40 and Aβ 1-42 levels in both the RIPA and formic acid soluble fractions are quantified using a specific and sensitive ELISA kit. Brain homogenates is examined by Western blot for total APP (including full length and truncated APP isoforms: sAPPβ, sAPPα, and C-terminal fragments), ADAM-10, BACE-1, and the four components of the γ-secretase complex (PSEN1, nicastrin, APH-1, PSEN2). β-tubulin is used as a loading control (anti-TUB2.1). In addition, mRNA levels (qPCR) and activity levels of these proteases are also assayed.

Brain homogenates are assayed for total (soluble and insoluble) and phosphorylated tau by standard Western blot techniques at 12 mo. of age. Briefly, mouse monoclonal anti-tau (HT7) and mouse monoclonal antibodies against different phosphorylated tau epitopes AT8 (Ser202/Thr205); AT180 (Thr231/Ser235); PHF-13 (S396); PHF-1 (Ser396/Ser404); AT270 (Thr181) are used. Levels are expressed as the ratio of phospho/total tau.

Aliquots of brain homogenates from 6 and 12 mo. old mice are also assayed by Western blot for biochemical markers of synaptic integrity: synaptophysin, PSD-95, and MAP2. Mitochondria are isolated from the cortex/hippocampus of 6 and 12 mo. old mice and examined for: matrix Ca²⁺ content, MPTP opening by measuring mitochondrial swelling, ₘCa²⁺ retention capacity, and EM imaging to examine mitochondrial structure. Hippocampal slices are freshly prepared from 6 and 12 mo. old mice and stained with DHE to monitor O2^{°—} generation. Further, ROS-mediated changes in redox state is examined by quantifying biomarkers of lipid and protein oxidation including: protein carbonyl levels (histology and ELISA) and 4-HNE levels (histology and ELISA). GSH:GSSG ratios (glutathione oxidation is a strong indicator of redox status) are quantified in brain lysates using an ELISA.

Lysates isolated from the brains of mice at 6 and 12 mo. of age are examined for alterations in ₘCa²⁺-dependent metabolic processes. Specifically, following the isolation of mitochondria the activity of the mitochondrial dehydrogenases (KGD and PDH) is probed and expression and phosphorylation status of (PDH, KGD, IDH) is examined. In addition, the redox status of the nicotinamide adenine dinucleotide (NAD+) pool is examined using a fluorometric NAD/NADH assay. To directly assess OxPhos the Seahorse Bioscience XF96 flux analyzer is utilized to analyze OCR in mitochondria isolated from the frontal cortex of mNCX-nKO-AD mice and their respective controls in a similar fashion to what is presented in FIG. 17. ATP production and ATP content in brain lysates isolated from the mNCX mutant mice is examined using a luciferase-based assay.

While significant neuronal loss is not normally associated with pathology in the 3xTg-AD model, genetic deletion of mNCX accelerates pathology and neuronal demise. Therefore, markers of neuronal cell death in mice at age 6 and 12 mo are evaluated. The histological hairpin 1 and 2 probe ligation technique is implemented to specifically identify apoptosis (hairpin 1, 3′ overhangs) vs. necrosis (hairpin 2, blunt ends) in brain sections with co-immunostaining with an antibody against the neuronal-specific marker MAP2. Histological sections are also stained for GFAP (reactive gliosis), and H&E to assess inflammation.

It is hypothesized herein that loss of mNCX in 3xTg-AD mice will promote ₘCa²⁺ overload, MPTP activation, metabolic derangement, and synaptic dysfunction.

Establish if Enhancing ₘCa²⁺ Efflux Protects Against Neurodegeneration in AD.

Ca²⁺ enters the mitochondria matrix via the mitochondria uniporter channel complex (MCUc) to activate key metabolic control points in OxPhos ATP generation. MCU-mediated ₘCa²⁺-uptake is largely stress-responsive in the heart and necessary for adrenergic responsiveness. While little is known regarding neuronal ₘCa²⁺ flux, the findings presented herein suggest that mNCX-mediated efflux is more central to physiological ₘCa²⁺ regulation and thereby represents an intriguing therapeutic target. To ascertain if enhancing ₘCa²⁺ efflux capacity can limit neuronal dysfunction and AD progression, neuronal-specific, ₘCa²⁺ conditional transgenic mice were crossed with the 3xTg-AD model. Here, it is determined whether increasing mNCX expression and efflux capacity limits mitochondrial dysfunction, cognitive decline, and neuropathology. These studies identify mNCX as a new therapeutic target in AD.

TRE-mNCX×Camk2a-tTA (mNCX-nTg) mice have been crossed with the previously detailed 3xTg-AD mutant mouse model (FIG. 22). Breeding over the past 14 months has resulted in the establishment of breeding pairs for experimental study (see FIG. 21 and Table 2). Dox is administered to breeding pairs and weaned pups up to 2 mo. of age to all groups of mice. Withdrawal of dox at 2 mo. allows neuronal specific expression of mNCX (this mutant line produces ˜2-3-fold overexpression). Conditional expression in adult mice avoid a development and compensatory gene modifications that may be caused in a germline or postnatal expression system. Mice undergo extensive phenotyping at both 6 and 12 months of age to examine: cognitive function, synaptic integrity, neuronal histopathology, mitochondrial structure/function, redox state, and metabolic alterations. mNCX-nTg and respective controls undergo phenotyping identical to what is presented above.

For all in vivo studies mixed sex cohorts with equal numbers are used. In addition, male vs. female data is statistically evaluated to determine the relevancy of sex for all experiments. For all experiments, the appropriate controls are included and all experiments are performed in a blinded-fashion when possible.

### Example 3: Genetic Ablation of Fibroblast Mitochondrial Calcium Uptake Increases Myofibroblast Trans-Differentiation and Exacerbates Fibrosis in Myocardial Infarction

Cardiac fibroblasts make up a significant portion of the adult heart and play a pivotal role in regulating the structural integrity of the heart by maintaining the extracellular matrix as well as coordinating cell-to-cell and cell-to-matrix interactions. In addition to this important physiological function, when the heart is injured fibroblasts transition from a quiescent structural role into contractile and synthetic myofibroblasts. This is crucial for the initial healing response, for example scar formation to prevent ventricular wall rupture after myocardial infarction, but excessive fibrosis is maladaptive, impairs cardiac function and contributes to heart failure progression. While cytosolic calcium (ᵢCa²⁺) elevation has been shown to be necessary for myofibroblast transdifferentiation, other Ca²⁺ domains have not been explored. Recent studies have reported that the Mcu gene encodes the channel forming portion of the mitochondrial calcium uniporter complex (MCU) and is required for acute mitochondrial calcium (ₘCa²⁺) uptake. Mitochondria are theorized to buffer significant amounts of ᵢCa²⁺ in non-excitable cells and they also serve as a bioenergetic control point of cellular metabolism. In addition, metabolic switching is thought be a key signal driving cellular differentiation in numerous tissue types. It is described herein the molecular role of ₘCa²⁺ in cardiac myofibroblast trans-differentiation and fibrosis using an in vivo model of myocardial infarction.

Generation of a Mcu Conditional Knockout Mouse

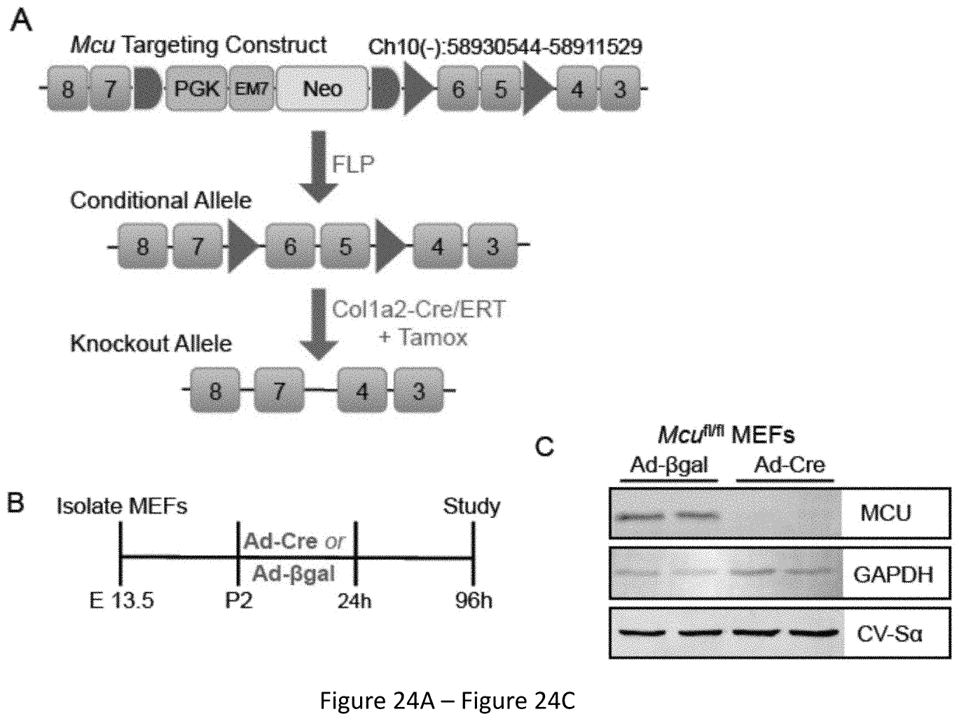

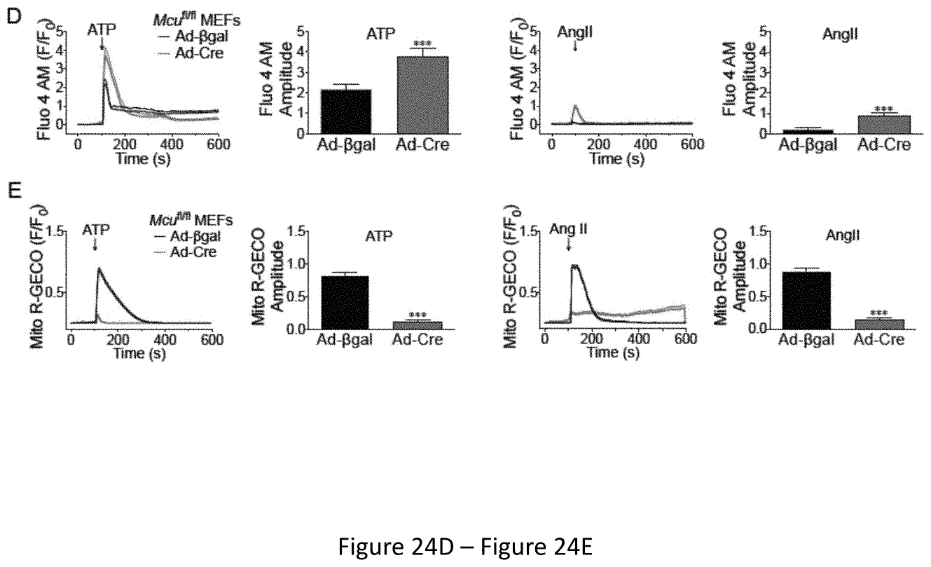

A conditional Mcu knockout mouse was generated using a Mcu targeting construct containing FRT and loxP sites for conditional potential (FIG. 24A-24B). Mouse embryonic fibroblasts (MEFswere isolated from Mcufl/fl embryos at E13.5 and analyzed (FIG. 24C-24E).

Deletion of Fibroblast Mcu Potentiates LV Dysfunction and Fibrosis After MI

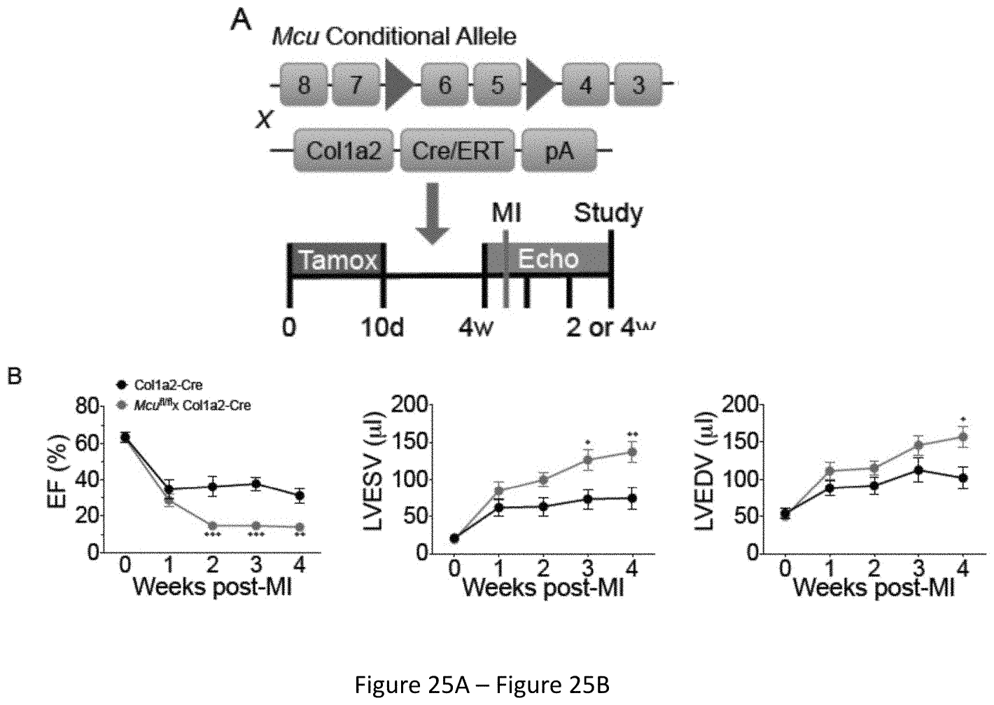

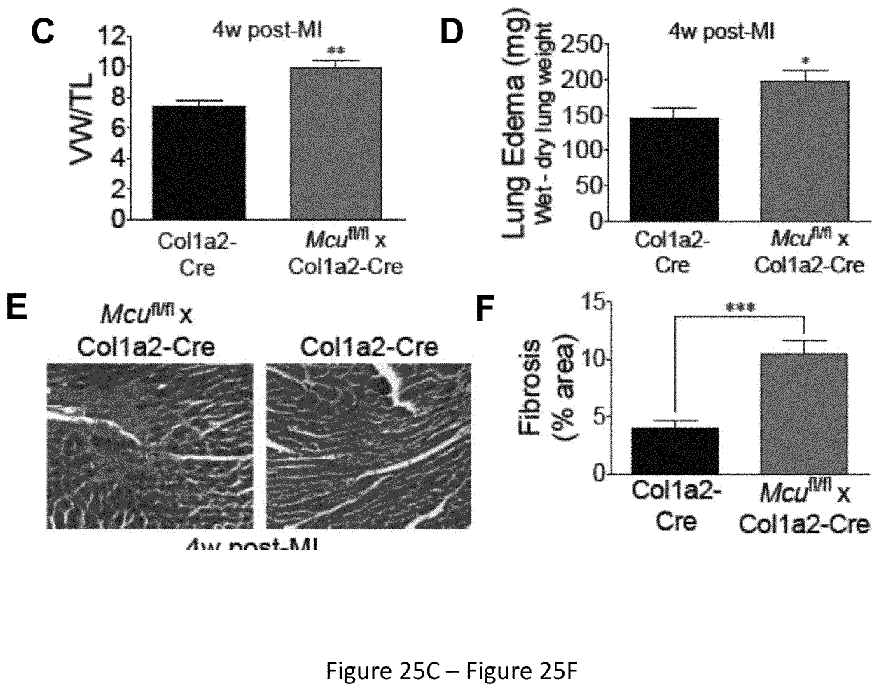

Mcu floxed mice were crossed with a transgenic mouse expressing a conditional, fibroblast-specific Cre recombinase (Col1a2-Cre/ERT). 8-12 w old mice were treated with tamoxifen (40 mg/kg/day) for 10 d to induce fibroblast-restricted Cre expression and allowed to rest for 3 w prior to permanent ligation of the left coronary artery. Mice were analyzed by echocardiography 1 w prior to MI and every week thereafter (FIG. 25).

Ablation of ₘCa²⁺ Uptake Enhances Myofibroblast Trans-Differentiation

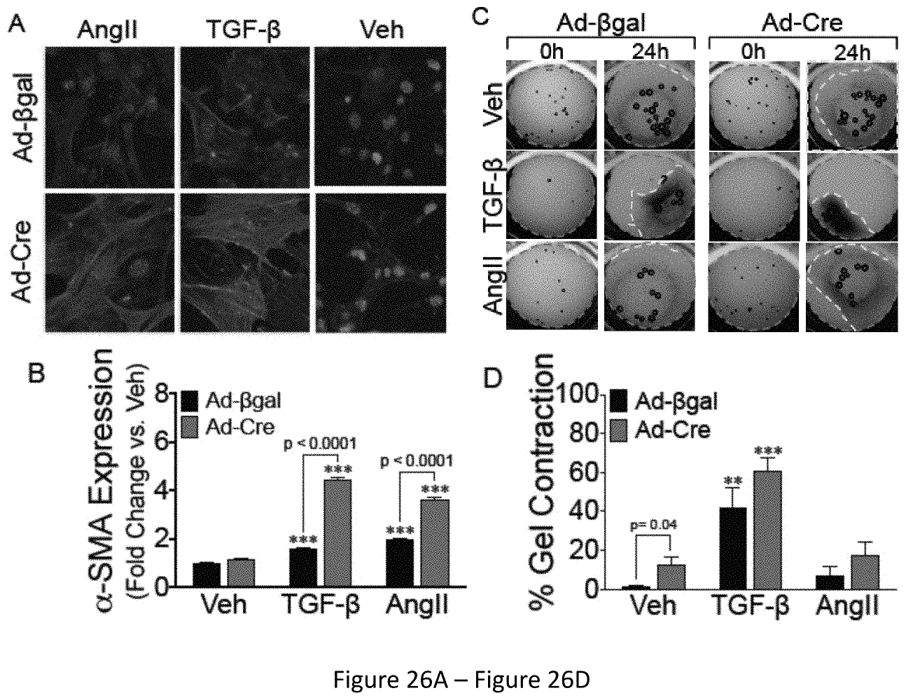

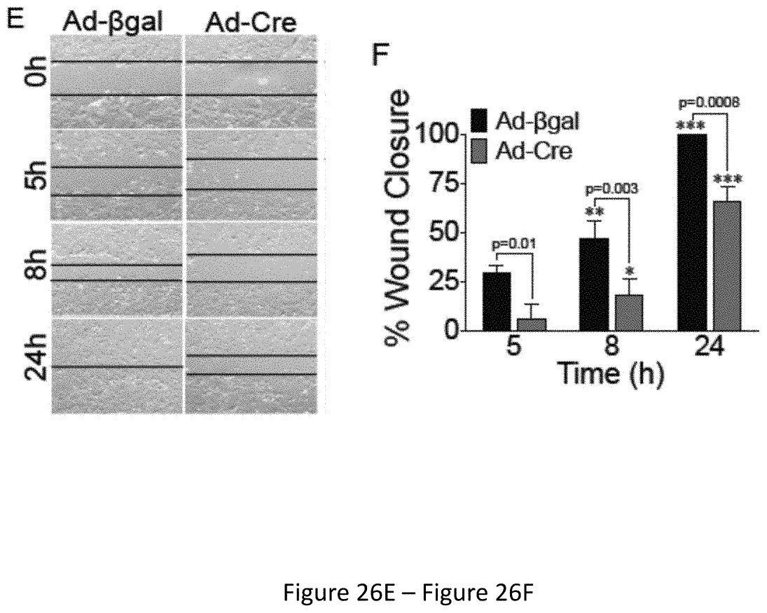

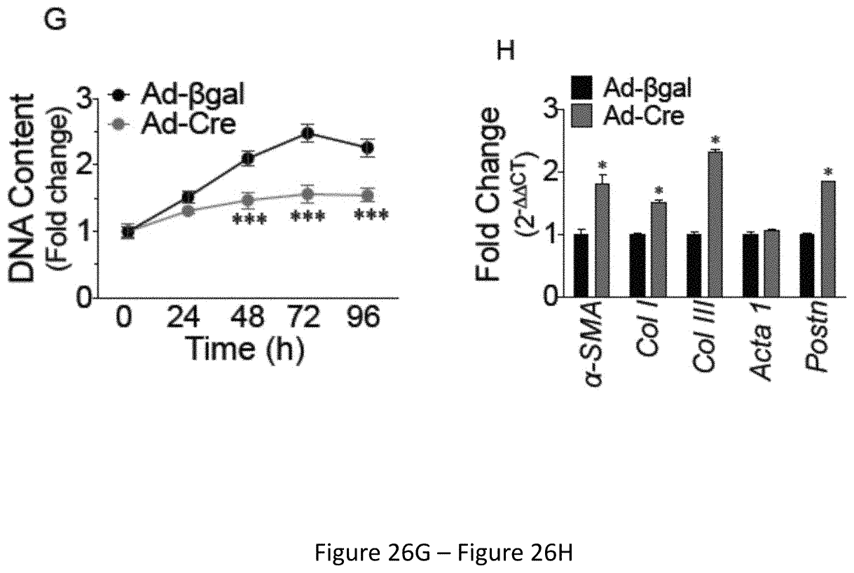

Mcufl/fl MEFs were infected with Ad-Cre or or Ad-βgal for 24 h and then 96 h later, treated with 10 μM Angiotensin II or 10 ng/mL TGF-β for 48 h and then analyzed (FIG. 26).

Mcu^{-/-} MEFs are More Glycolic and PDH Activation in Response to Fibrotic Agonists is Altered

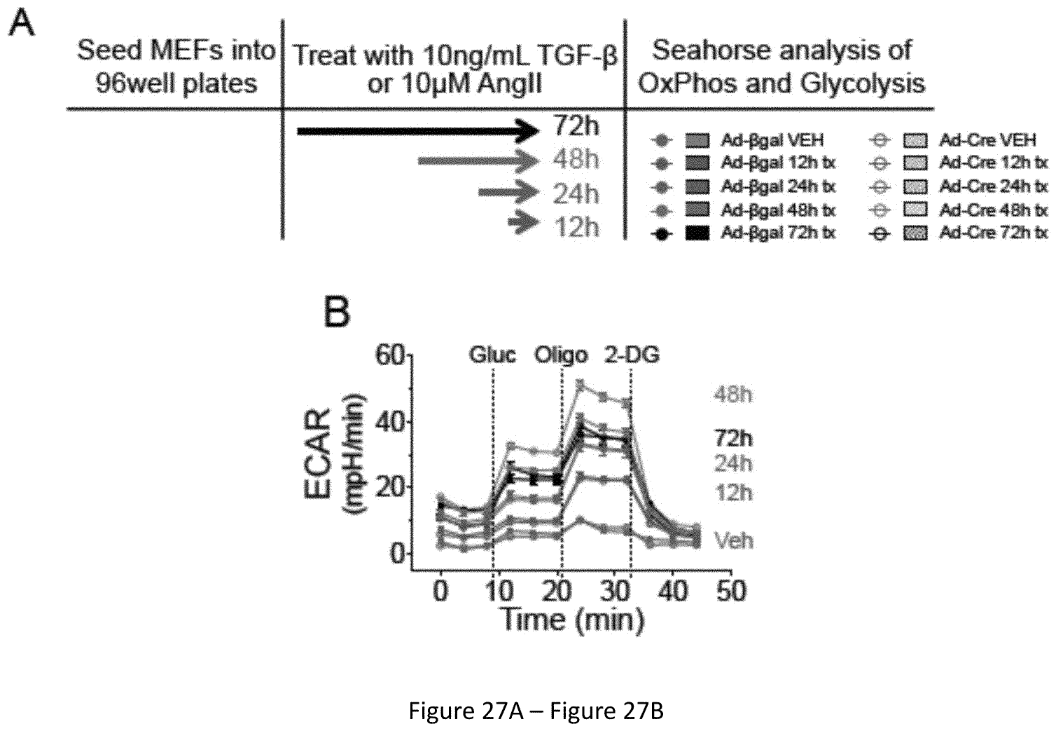

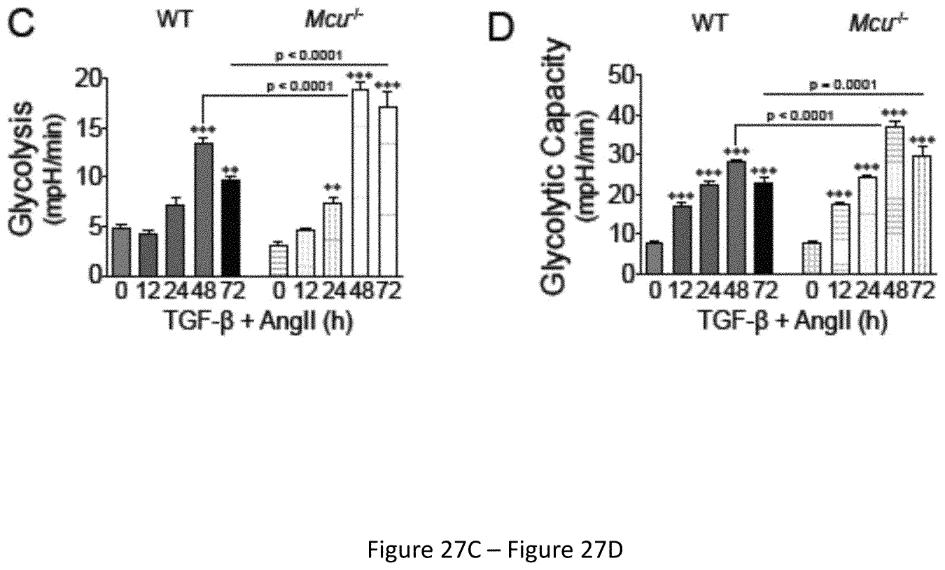

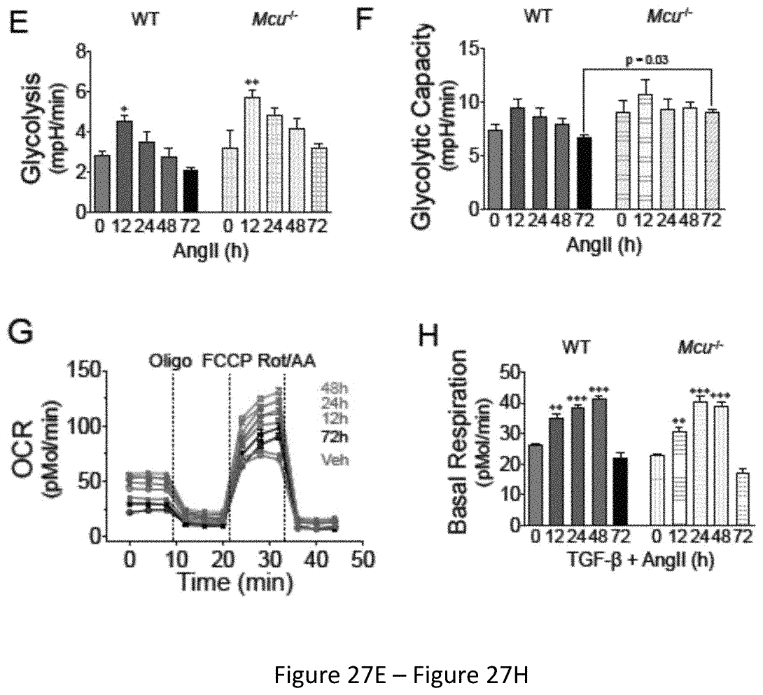

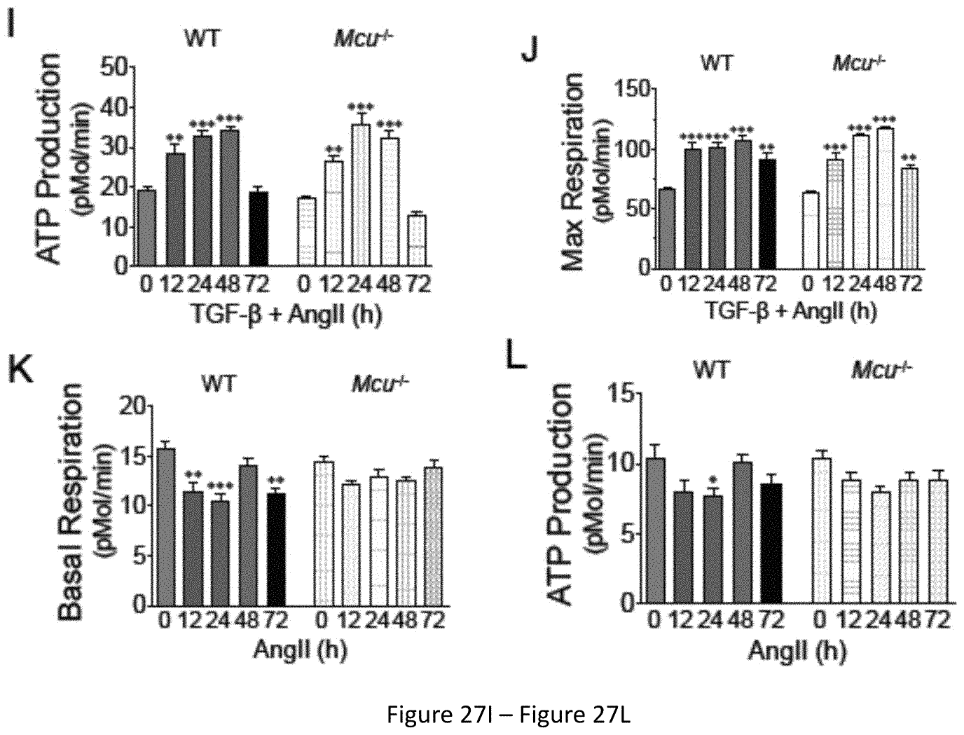

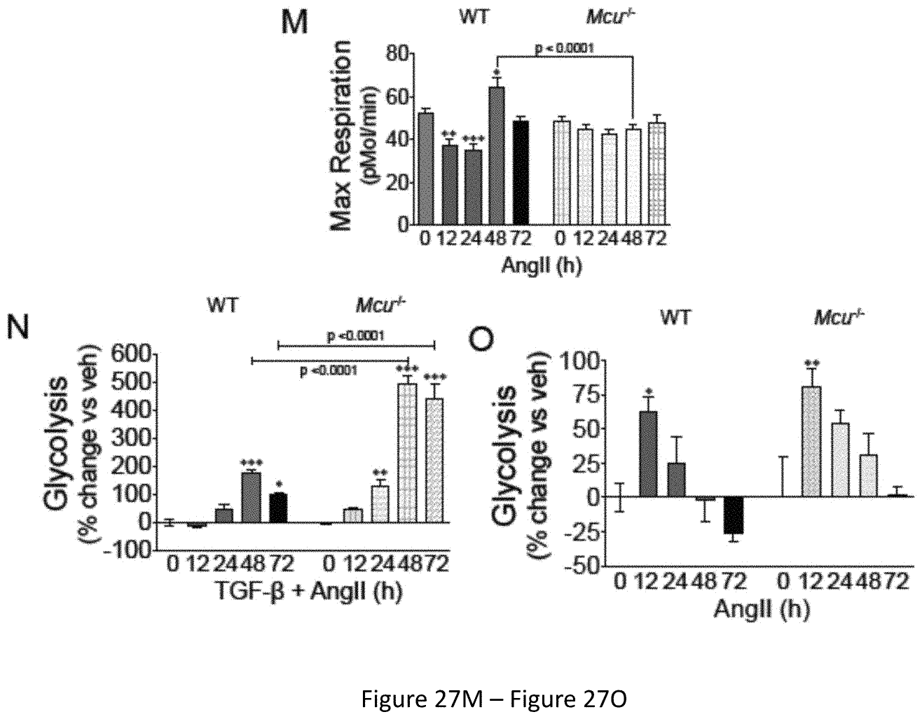

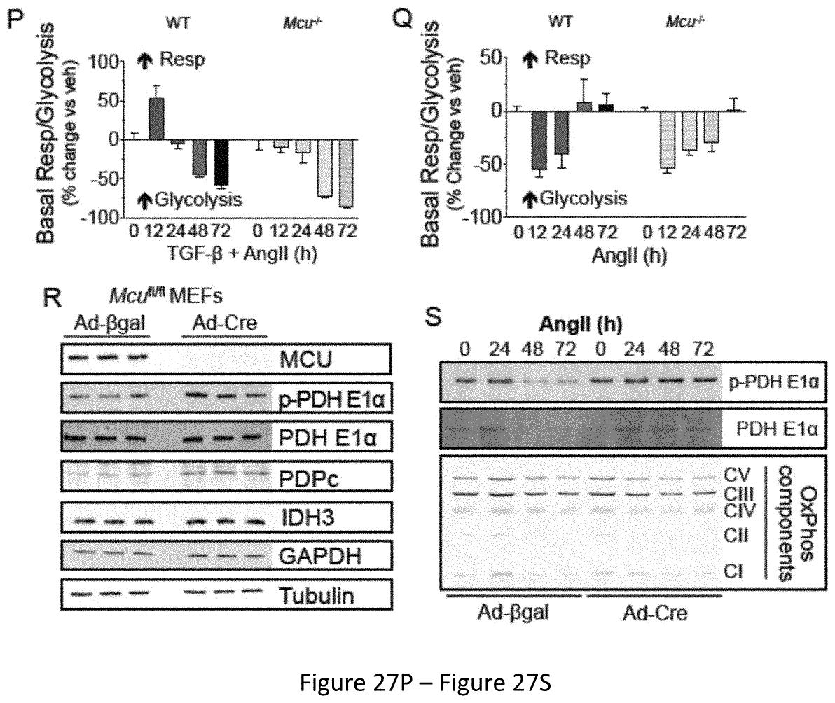

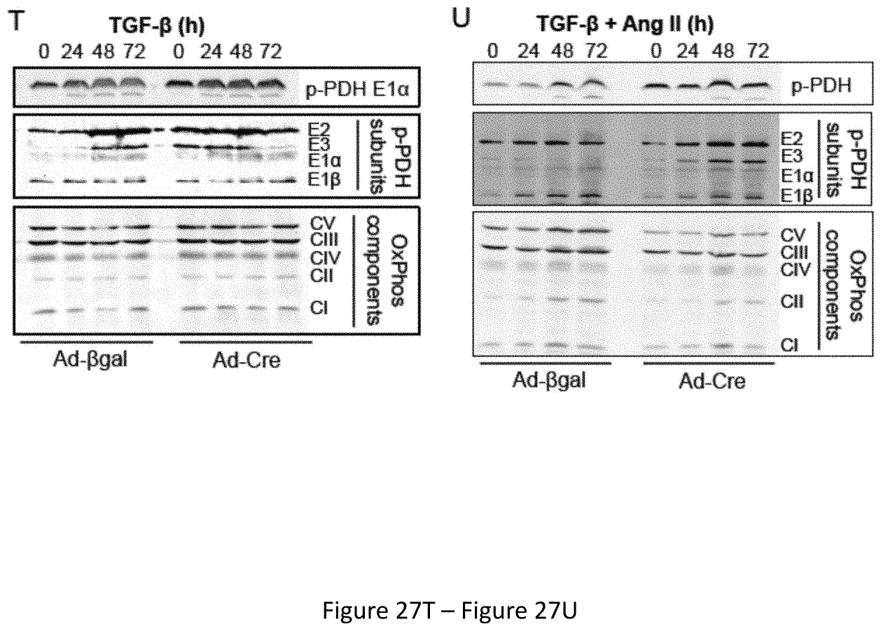

MEFs were treated with pro-fibrotic stimuli or vehicle for 12, 24, 48 or 72 h and assayed for Glycolytic function and Oxidative Phosphorylation using a Seahorse XF96 to measure extracellular acidification rates (ECAR, glycolysis) or oxygen consumption rates (OCR, OxPhos) (FIG. 27).

Enhanced Glycolysis Drives Myofibroblast Trans-Differentiation

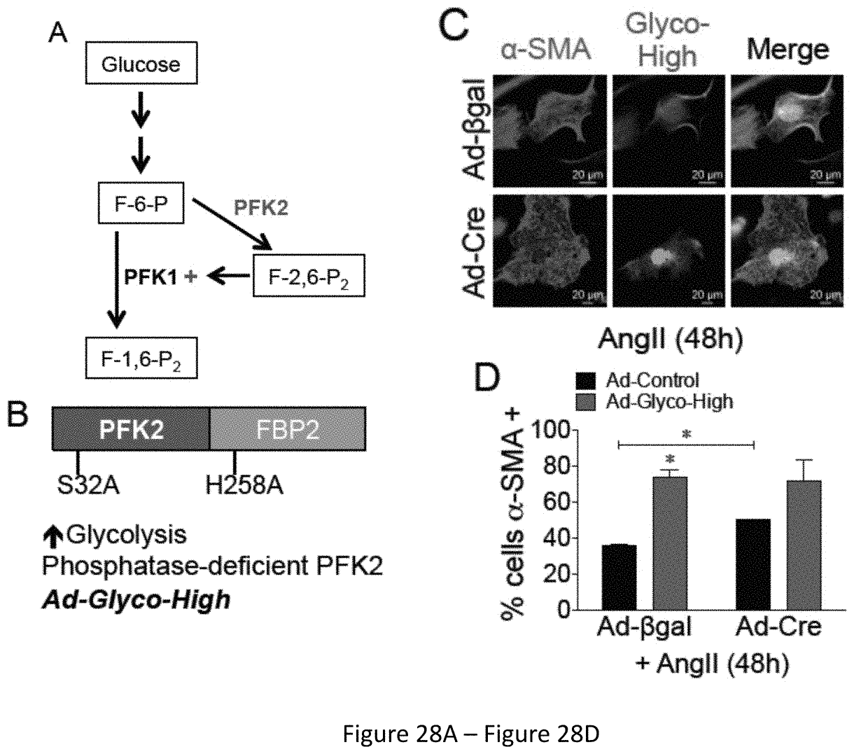

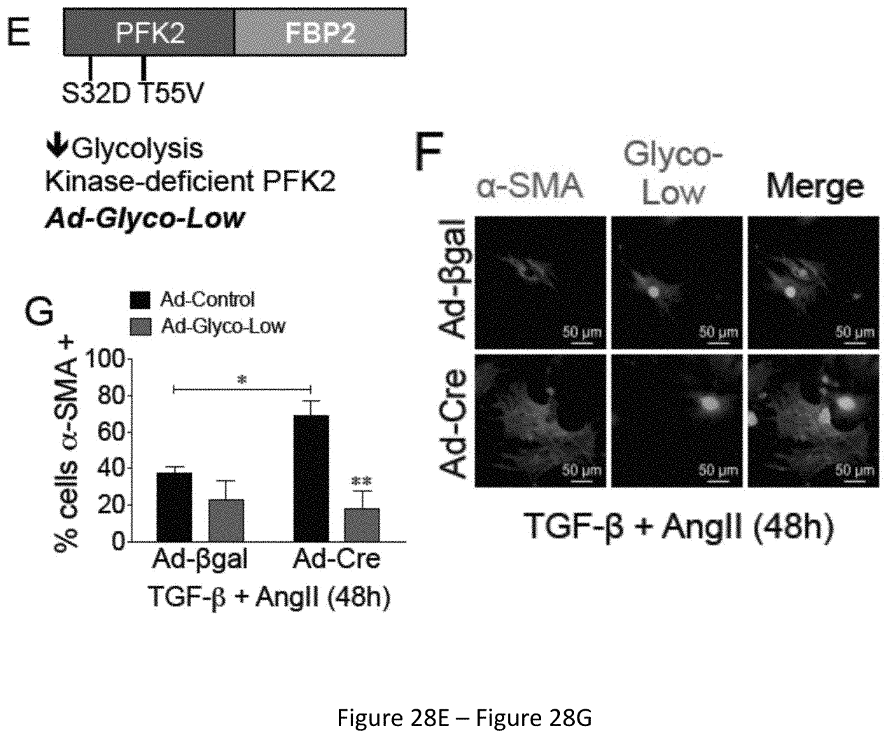

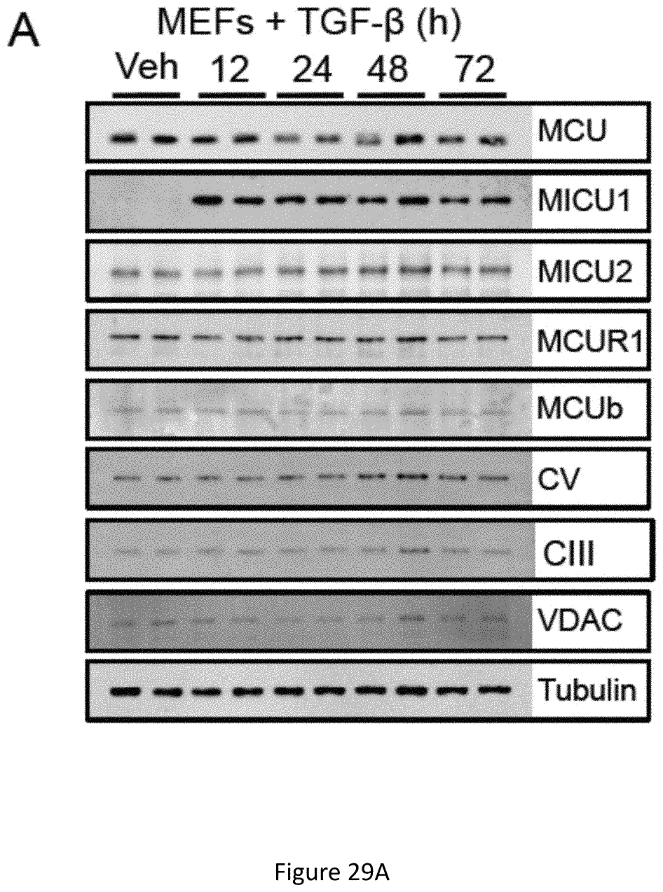

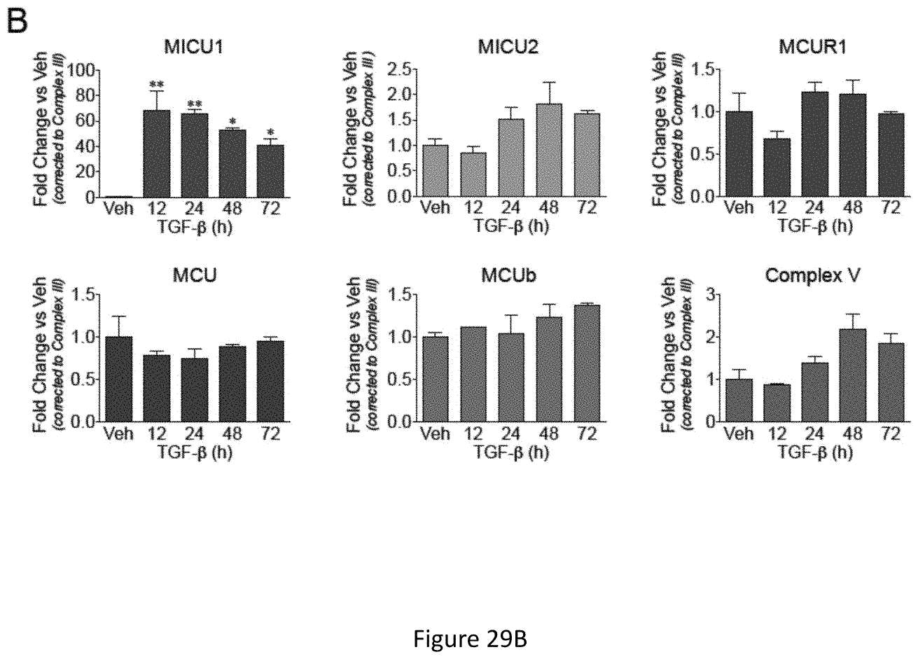

MEFs were infected with Ad-Glyco-High and treated with AngII for 48 h or with Ad-Glyco-Low and treated with TGF-β+AngII for 48 h and then analyzed. (FIG. 28)

The Pro-Fibrotic Stimulus TGF-β Changes Expression of MCU Components

Wild-type MEFs were treated with 10 ng/mL TGF-f3 for 12, 24, 48, or 72 h and cell lysates were immunoblotted for components of the mitochondrial calcium uniporter (MCU) complex (FIG. 28).

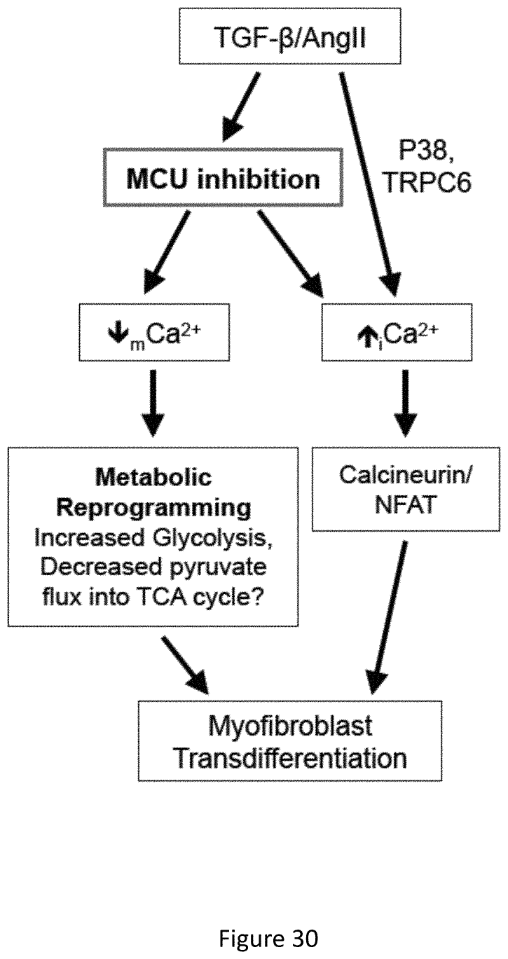

Deletion of Mcu attenuates ₘCa²⁺ uptake and increases ᵢCa²⁺ amplitude upon stimulation with ATP, AngII, and ET1, suggesting that the mitochondria buffer ᵢCa²⁺ in fibroblasts. Deletion of Mcu in fibroblasts worsens left ventricular function and cardiac fibrosis following MI. Mcu ablation enhances myofibroblast transdifferentiation. Mcu^{-/-} MEFs are more glycolytic and have increased inactivation of PDH, suggesting changes in metabolic flux. Increasing glycolysis augments myofibroblast transdifferentiation while decreasing glycolysis attenuates the enhanced transdifferentiation in Mcu^{-/-} MEFs. TGF-β changes the expression of key MCU components, suggesting that inhibition of mitochondrial Ca²⁺ uptake may be an endogenous mechanism whereby pro-fibrotic stimulus elicit myofibroblast transdifferentiation (FIG. 30).

### Example 4: Mitochondrial Calcium Exchange Links Metabolism with the Epigenome to Control Cellular Differentiation

The data presented herein uncovers an important role for ₘCa²⁺ uptake beyond metabolic regulation and cell death and demonstrate that ₘCa²⁺ signaling regulates epigenetics to influence cellular differentiation. It is demonstrated herein that an alteration in mtCU gating is critical to myofibroblast differentiation by directly modulating the levels of 3 metabolites to regulate histone demethylation. This study reveals that ₘCa²⁺ exchange is a central regulatory mechanism linking canonical signaling pathways with adaptive changes in mitochondrial metabolism and epigenetics that are necessary to drive cellular differentiation.

The materials and methods employed in these experiments are now described.

Generation of Fibroblast-Specific Mcu Conditional Knockout Mice

Generation of Mcu^{fl/fl} was previously reported (Luongo et al.). Mcufl/fl mice were crossed with fibroblast-specific Cre transgenic mice, Col1a2-CreERT, to generate tamoxifen-inducible, fibroblast specific Mcu knockouts. For temporal deletion of Mcu, mice 8-12 weeks of age were injected intraperitoneal with tamoxifen (40 mg/kg/day) for ten consecutive days. All mouse genotypes, including controls, received tamoxifen.

Mouse Embryonic Fibroblast Isolation

Mouse embryonic fibroblasts (MEFs) were isolated from Mcu^{fl/fl} or C57/BL6 (WT) mice. Embryos were isolated from pregnant females at E13.5. The embryos were decapitated and all the red organs removed. Tissue was minced and digested in 0.25% trypsin supplemented with DNase for 15 minutes at 37° C. in the presence of 5% CO2. Digested tissue was gently agitated by pipetting to dissociate cells. Cells from each embryo were suspended in Dulbecco's Modified Eagle's Medium (DMEM) supplemented with 10% fetal bovine serum, 1% penicillin/streptomycin, and 1% Non-Essential Amino Acids, plated on a 10 cm dish and incubated at 37° C. in the presence of 5% CO₂. For imaging studies, cells were plated on glass coverslips pre-coated with gelatin.

Adenoviral Transfer

For experiments that required adenoviral gene transfer, MEFs were incubated in adenovirus for 24 hours at which time the media was changed. To knockout Mat, MEFs were transduced with adenovirus encoding Cre-recombinase (Ad-Cre) or βgalactosidase (Ad-(βgal) for 24 h and experiments were performed 5 days post-infection in order to ensure sufficient time for protein turnover. For experiments using adenovirus encoding Glyco-High, Glyco-Low, or mito-R-GECO1, cells were incubated for an additional 24 hours prior to the experiment. The following adenoviruses have previously been described: NFAT-cl-GFP, Glyco-High, Glyco-Low, mito-R-GECO1 (De Windt et al., 2000; Kurland et al., 1992; Salabei et al., 2016; Zhao et al., 2011). Glyco-High and Glyco-Low adenoviruses were made and purified by Vector Labs using cDNA for a rat liver PFKFB1 isoform of phosphofructokinase 2 (PFK2)/ fructose-2,6-bisphosphatase (FBP2). The Glyco-High adenovirus has 2 single-amino acid point mutations (S32A and H258A) which result in the enzyme having only PFK2 activity, while the Glyco-Low adenovirus has 2 single amino acid point mutations (S32D and T55V) which result in the enzyme having only FBP2 activity (Kurland et al., 1992; Salabei et al., 2016).

Myofibroblast Differentiation

Myofibroblast differentiation was induced using 10 ng ml⁻¹ recombinant mouse Transforming growth factor-β (TGFβ) or 10 μM Angiotensin II (AngII,). In all experiments, FBS was reduced to 1% 24 hours prior to and during treatment with TGFβ or AngII.

Western Blot Analysis

All protein samples were lysed by homogenization in RIPA buffer supplemented with phosphatase inhibitors and protease inhibitors. Samples were sonicated briefly and centrifuged at 5,000 g for 10 minutes. The supernatant was collected and used for further analysis. Protein amount was quantified using the Bradford Protein Assay and equal amounts of protein (10-50 μg) were run by electrophoresis on polyacrylamide Tris-glycine SDS gels. Gels were transferred to PVDF and membranes were blocked for 1 hour in Blocking Buffer followed by incubation with primary antibody overnight at 4° C. Membranes were washed in TBS-T 3 times for 5 minutes each and then incubated with secondary antibody for 1 hour at room temperature. After incubation with fluorescent secondary antibodies, membranes were washed in TB S-T 3 times for 5 minutes each and then imaged on a Licor Odyssey system. The following antibodies were used in the study: MCU (1:1,000), MCUb (1:250,), MICU1 (1:500,), MCUR1 (1:500), EMRE (1:250, Santa Cruz, sc-86337), VDAC (1:1,000), PDHE1α phospho 5293 (1:1,000), PDHE1α (1:1,000,), IDH3A (1:500), α-tubulin (1:1,000), ETC respiratory chain complexes (1:2,500), H3K4me3 (1:2,000), H3K9me3 (1:2,000), H3K27me3 (1:2,000), H3K4me2 (1:2,000), H3K9me2 (1:2,000), H3K27me2 (1:2,000), H3 (1:2000); and secondary antibodies: anti-mouse (1:12,000), anti-rabbit, (1:12,000), and anti-goat (1:12,000).

Live Cell Imaging of Ca²⁺ Transients

Mcu^{fl/fl} MEFs were infected with Ad-Cre or Ad-βgal for 72 hours and then transduced with adenovirus encoding a mitochondrial-targeted Ca²⁺ reporter (Miro-R-GECO). 48 hours post-infection with Miro-RGECO, prior to live-cell imaging, MEFs were loaded with the calcium sensitive dye Fluo-4 AM (1 μM) to measure cytosolic calcium transients. Cells were placed in a 37° C. heated chamber in physiological Tyrode's buffer (150 mM NaCl, 5.4 mM KC, 5 mM HEPES, 10 mM glucose, 2 mM CaCl2, 2 mM sodium pyruvate, pH 7.4) and imaged on a Carl Zeiss Axio Observer Z1 microscope. Ca²⁺ transients were continuously recorded and analyzed on Zen software. After 2-3 minutes of baseline recording, a single pulse of 1 mM ATP was delivered to liberate intracellular Ca²⁺ (ᵢCa²⁺) stores. For Ca²⁺ fluorescence measurements, the F0 was measured as the average fluorescence of the cell prior to stimulation. The maximal fluorescence (F) was measured for peak amplitude. Background fluorescence was subtracted from each experiment before measuring the peak intensity as F/F₀.

Immunofluorescence

MEFs were seeded on coated 35-mm dishes. MEFs were fixed for 15 minutes in 4% paraformaldehyde, then permeabilized for 15 minutes with 0.15% Triton-X-100, and blocked in PBS containing 10% goat serum for 1 hour at room temperature. MEFs were incubated in primary antibody α-SMA (1:1,000) overnight at 4° C. and secondary antibody goat anti-mouse Alexa Fluor 594 (1:1,000) for 45 minutes at 37° C. Prior to imaging, MEFs were incubated with Hoechst 33342 to demarcate cell nuclei. Cells were imaged on a Carl Zeiss Axio Observer Z1 fluorescent microscope. Images were acquired in the red (590ex/617em) and blue (350ex/461em) channels. α-SMA expression was assessed by quantifying fluorescence intensity and the percentage α-SMA positive cells. More than 50 cells per dish were analyzed.

Gel Contraction

Fibroblast contractile activity was assessed by collagen contraction assays in which 112,500 MEFs were seeded into a 2 mg/mL collagen type I gel matrix and cast into a 48 well plate. Once collagen polymerized, the gel was gently released from edges of the well and media was added to the well. Images were taken using a Nikon SMZ1500 stereomicroscope at 0 and 24 after the gel was released from well edges. ImageJ software was used to calculate the surface area, which is presented as percent gel contraction relative to initial size of the gel.

Cell Proliferation Assay

MEFs were seeded at the same density in 96 well plates and quantified using the CyQUANT NF Cell Proliferation Assay Kit.

qPCR mRNA Analysis

RNA was isolated using the RNeasy Mini Kit according to the manufacturer's protocol. RNA (2 μg) was reverse transcribed into cDNA using the High Capacity cDNA Reverse Transcription Kit. Thermocycler conditions were as follows: 25° C. for 10 minutes, 37° C. for 2 hours, 85° C. for 5 minutes. Quantification of cDNA was done using Luminaris HiGreen qPCR Master Mix. Cycling conditions were as follows: 95° C. for 10 minutes followed by 40 cycles of amplification (95° C. denaturation for 15 seconds, 60° C. annealing/extension for 1 minute).

Samples were evaluated for mRNA expression of Collagen type I alpha 1 chain (Col1a1), Collagen type I alpha 2 chain (Col1a2), Collagen type III alpha 1 chain (Col3a1), α-SMA (Acta2), periostin (Postn), lysyl oxidase (Lox), fibronectin (Fn1), and platelet derived growth factor receptor alpha (Pdgfra). Rps13 (Ribosomal Protein S13) was used as a housekeeping gene. All samples were analyzed in duplicate and averaged. Fold change in mRNA expression was measured using the Comparative CT Method (2\hat{ }-ΔΔCT). Primers used are listed in Table 3.

TABLE 3
Primers

| Gene | Forward primer 5′-3′ | Reverse primer 5′-3′ |
|---|---|---|
| Rps13 | Gcaccttgagaggaacagaa (SEQ ID NO: 1) | gagcacccgottagtatatag (SEQ ID NO: 2) |
| Col1a1 | ttcagggaatgcctggtgaa (SEQ ID NO: 3) | acctttgggaccagcatca (SEQ ID NO: 4) |
| Col1a2 | gaaaagggtccctctggagaa (SEQ ID NO: 5) | aataccgggagcaccaagaa (SEQ ID NO: 6) |
| Col3a1 | tgctggaaagaatggggagac (SEQ ID NO: 7) | ggtccagaatctccottgtcac (SEQ ID NO: 8) |
| Acta2 | gtgaagaggaagacagcacag (SEQ ID NO: 9) | gcccattccaaccattactcc (SEQ ID NO: 10) |
| Postn | ccattggaggcaaacaactcc (SEQ ID NO: 11) | ttgcttcctctcaccatgca (SEQ ID NO: 12) |
| Lox | acgtcctgtgactatgggtac (SEQ ID NO: 13) | tctgccgcataggtgtcata (SEQ ID NO: 14) |
| Fn1 | cgtcattgccctgaagaaca (SEQ ID NO: 15) | aagggtaaccagttggggaa (SEQ ID NO: 16) |
| Pdgfra | caaagggaggacgttcaagac (SEQ ID NO: 17) | tgcgtccatctccagattca (SEQ ID NO: 18) |

NFAT Translocation Assay

MEFs were plated on coated 35-mm dishes and infected with Ad-NFATc1-GFP for 24 hours at which time live-cell images were taken followed by treatment with 10 ng m1¹ TGFP or 10 μM AngII for 24 hours. For live-cell imaging, cells were placed in a 37° C. heated chamber on a Carl Zeiss Axio Observer Z1 fluorescent microscope. Prior to imaging, MEFs were incubated with Hoechst 33342 to demarcate cell nuclei. Images were acquired in the green channel (490ex/525em) and blue channel (350ex/460em). NFAT localization was quantified as the nuclear/cytoplasmic ratio of GFP fluorescence. More than 50 cells per dish were analyzed.

Evaluation of ₘCa²⁺ Uptake and Efflux

Before permeabilization, MEFs were washed in extracellular-like Ca²⁺-free buffer (120 mM NaCl, 5 mM KCl, 1 mM KH₂PO₄, 0.2 mM MgCl₂, 0.1 mM EGTA, 20 mM HEPEs-NaOH, pH 7.4). MEFs (1.5 million) were then transferred to intracellular-like medium (ICM) (120 mM KCl, 10 mM NaCl, 1 mM KH₂PO₄, 20 mM HEPES-Tris, protease inhibitors, 5 mM succinate, 2 μM thapsigargin, 40 μg ml⁻¹ digitonin, 10 μM CGP-37157 (NCLX inhibitor), pH 7.2). ICM was cleared with Chelex 100 to remove trace Ca²⁺. MEFs were gently stirred and 1 μM Fura-2 was added to monitor extra-mitochondrial Ca²⁺. At 20 seconds, JC-1 was added to monitor Δψ. Fluorescence signals were monitored in a temperature controlled (37° C.) multi-wavelength-excitation/dual-wavelength-emission spectrofluorometer (Delta RAM, Photon Technology Int.) using 490-nm excitation (ex)/535-nm emission (em) for the JC-1 monomer, 570-nm ex/595-nm em for the J-aggregate of JC-1, and 340- and 380-nm ex/510-nm em for Fura-2. At 350 seconds a Ca²⁺ bolus was added and clearance of extra-mitochondrial Ca²⁺ was representative of ₘCa²⁺ uptake. At completion of the experiment 10 μM of the protonophore FCCP was added to uncouple the Δψ and release matrix free-Ca²⁺.

To quantify actual Ca²⁺ content, a standard curve of Ca²⁺ binding Fura-2 was generated from serial diluted Ca²⁺ standards (0.01-120 μM) in ICM. Fura-2 fluorescence ratio was converted to [Ca²⁺] by the following equation: [Ca²⁺]=Kd*(R−Rₘᵢₙ)/(Rₘₐₓ−R)*Sf2/Sb2. (Rₘᵢₙ (ratio in 0-Ca²⁺)=1.341; Rₘₐₓ (ratio at saturation)=27.915; Sf2 (380/510 reading in 0-Ca²⁺)=15822.14; Sb2 (380/510 reading with Ca²⁺ saturation)=1794.32). The percentage of initial mCa²⁺ uptake (200 s after Ca²⁺ addition) was plotted against the bath Ca²⁺ concentration for each of the different Ca²⁺ boluses to generate a dose response curve.

ECAR and OCR Measurements

A Seahorse Bioscience XF96 extracellular flux analyzer was employed to measure extracellular acidification rates (ECAR) and oxygen consumption rates (OCR). ECAR was measured using the Glycolytic Stress Test Kit and OCR was measured using the Miro Stress Test Kit. To evaluate ECAR, 20,000 MEFs/well were plated in XF media pH 7.4 without supplementation. Non-glycolytic acidification was measured, then 10 mM glucose was injected to measure basal glycolysis, followed by 3 μM oligomycin to inhibit mitochondrial ATP production and reveal maximal glycolytic capacity, and finally 50 mM 2-deoxy glucose was injected to completely inhibit all glycolysis. To evaluate OCR, 20,000 MEFs/well were plated in XF media pH 7.4 supplemented with 10 mM glucose and 1 mM sodium pyruvate. Basal OCR was measured, then 3 μM oligomycin was injected to inhibit ATP-linked respiration, followed by 2 μM FCCP to measure maximal respiration, and finally 1.5 μM rotenone/antimycin A was injected to completely inhibit all mitochondrial respiration. After each experiment, protein concentration was measured and wells were normalized using the Wave software.

Metabolomic Profiling

Cells in a 10 cm dish were washed with 5% (w/w) mannitol (10mL for the first wash, 2 mL for the second wash) and extracted in 800 μL methanol plus 550 μL internal standard solution (Human Metabolome Technologies, HMT). Extracted solution was spun down at 2,300×g at 4° C. for 5 minutes. The supernatant was transferred into centrifugal filter units (HMT) and centrifuged at 9,100×g at 4° C. for ˜3.5 h until no liquid remained in the filter cup. Filtrate was frozen at −80° C. and shipped to HMT for analysis by CE-TOFMS and CE-QqQMS (Boston, Mass.). Filtrate was centrifugally concentrated and resuspended in 50 μl of ultrapure water immediately before the measurement.

Cationic metabolites were analyzed using an Agilent CE-TOFMS system Machine No. 3 and a fused silica capillary (i.d. 50 μm×80 cm) with Cation Buffer Solution as the electrolyte. The sample was injected at a pressure of 50 mbar for 10 seconds. The applied voltage was set at 27 kV. Electrospray ionization-mass spectrometry (ESI-MS) was conducted in the positive ion mode, and the capillary voltage was set at 4,000 V. The spectrometer was scanned from m/z 50 to 1,000.

Anionic metabolites were analyzed using an Agilent Capillary Electrophoresis System equipped with an Agilent 6460 TripleQuad LC/MS Machine No. QqQ3 and a fused silica capillary (i.d. 50 μm×80 cm) with Anion Buffer Solution as the electrolyte. The sample was injected at a pressure of 50 mbar for 25 seconds. The applied voltage was set at 30 kV. ESI-MS was conducted in the positive and negative ion mode, and the capillary voltage was set at 4,000 V for positive and 3, 500 V for negative mode.

Peaks detected in CE-TOFMs analysis were extracted using automatic integration software and those in CE-QqQMS analysis were extracted using automatic integration software in order to obtain peak information including m/z, migration time, and peak area. The peak area was then converted to relative peak area by the following equation: Relative peak area=Metabolite Peak Area/(Internal Standard Peak Area x Normalization Factor). The peaks were annotated based on the migration times in CE and m/z values determined by TOFMS. Putative metabolites were then assigned from HMT metabolite database on the basis of m/z and migration time. All metabolite concentrations were calculated by normalizing the peak area of each metabolite with respect to the area of the internal standard and by using standard curves, which were obtained by three-point calibrations. A heat map was generated using ClustVis. Unit variance was applied to rows. Rows were clustered using Manhattan distance and average linkage.

DNA Methylation

To extract genomic DNA, cells were collected and washed with PBS followed by 2 h incubation at 60° C. in DNA isolation buffer (0.5% SDS, 100 mM NaCl, 50 mM Tris pH 8, 3 mM EDTA, 0.1 mg/mL proteinase K). DNA was extracted using chloroform followed by ethanol precipitation and dissolved in double-distilled water. DNA methylation was quantified using the MethylFlash™ Methylated DNA Quantification Kit. 100 nanograms of input DNA was used per reaction. Absorbance at 450-nm was measured using a Tecan Infinite F50 microplate reader.

ChIP-qPCR

ChIP was performed using the ChIP-IT High Sensitivity. Cells were fixed, lysed and sonicated for 30 minutes (30 seconds on, 30 seconds off) leading to chromatin fragments between 200 and 1200 base pairs. DNA-bound protein was immunoprecipitated using 2 μg anti-H3K27me2 or IgG. Following IP, cross-links were reversed, protein was removed, and DNA was purified. qPCR was performed with equal amounts of H3K27me2-immunoprecipitated sample, IgG-immunoprecipitated sample, and input sample. Values were normalized to input measurements and fold enrichment was calculated. qPCR primers (Table 4) were designed to target gene loci regions flanking or nearby myofibroblast transcription factor predicted binding sites according to Genomatrix-MatInspector Software analysis.

TABLE 4

| Gene | Forward primer 5′-3′ | Reverse primer 5′-3′ |
|---|---|---|
| Postn | CCACAGCCCAGAGCTATATAAAC | CAGCAGCAGCAGAGCATATAA |
| | (SEQ ID NO: 19) | (SEQ ID NO: 20) |
| Pdgfra | AGCAACTACACGGCACTTT | CTGGGCCTCGCTAGAAATATG |
| | (SEQ ID NO: 21) | (SEQ ID NO: 22) |

Echocardiography

Transthoracic echocardiography of the left ventricle was performed and analyzed on a Vevo 2100 imaging system. Mice were anesthetized with 2% isoflurane in 100% oxygen during acquisition. M-mode images were collected in short-axis and analysis was performed using VisualSonics software.

Myocardial Infarction

Ligation of the left coronary artery (LCA) was performed (Gao et al., 2010). Briefly, mice were anesthetized with isoflurane and the heart exposed via a left thoracotomy at the fifth intercostal space. The LCA was permanently ligated to induce a large myocardial infarction.

Chronic Angiotensin II Infusion

Mini-osmotic pumps (Alzet Model 1004) were inserted subcutaneously delivering 1.1 mg/kg/d AngII (Sigma, A9525) for 4 weeks.

Tissue Gravimetrics and Histology

Mice were sacrificed followed by isolation and weighing of the heart and lungs as well as measurement of tibia length. Heart gravimetrics were assessed by heart weight/tibia length ratios. Lungs were weighed at the time of isolation (wet lung weight) and after dehydration at 37° C. for 1 week (dry lung weight). Lung edema was quantified by subtracting wet—dry lung weight. For histological analysis, hearts were collected at the indicated time points and fixed in 10% buffered formalin. Next, hearts were dehydrated and embedded in paraffin followed by collection of serial 7 μm sections. To evaluate fibrosis, sections were stained with Masson's trichrome (Sigma). Sections were examined using a Nikon Eclipse Ni microscope and images were acquired with a high-resolution digital camera (Nikon DS-Ri1). The percentage of fibrosis was quantified using ImageJ software. Blue pixels were expressed as a percentage of the entire image surface area. To quantify myofibroblasts, antigen retrieval was performed and sections were subsequently stained with anti-α-SMA antibody (1:1,000,) and anti-CD31 (1:30). Sections were incubated with antibodies in a humidified chamber overnight at 4° C. followed by 1 hour at room temperature. Sections were washed three times for 5 minutes each in PBS and incubated in secondary antibodies for 1 hour at 37° C. in a humidified chamber. Secondary antibodies used were: Alexa Fluor 488 (1:250) and Alexa Fluor 555 (1:100). After washing three times for 5 minutes each, sections were stained with DAPI. After DAPI staining, sections were washed three times for 5 minutes and then incubated with Sudan black B for 40 minutes at room temperature followed by 6 washes for 10 minutes each. Finally, sections were mounted on slides using Vectashield. Images were taken using a Carl Zeiss Axio Observer Z1 fluorescent microscope. Images were acquired in the green channel (490ex/525em), orange channel (555ex/580em), and blue channel (350ex/460em). Eight images per heart were obtained for quantitative analysis. Myofibroblast percentages were derived by counting the number of single positive α-SMA cells (α-SMA+/CD31−) and dividing by the total number of nuclei.

Statistics and Scientific Rigor

All results are presented as mean±SEM. All experiments were replicated at least 3 times if biological replicates were not appropriate. Statistical powering was initially performed using the nQuery Advisor 3.0 software (Statistical Solutions) along with historical data to estimate sample size. For all experiments, the calculations use α=0.05 and β=0.2 (power=0.80). Statistical analysis was performed using Prism 6.0 (GraphPad Software). Where appropriate, column analyses were performed using an unpaired, 2-tailed t-test (for 2 groups) or one-way ANOVA (for groups of 3 or more). For grouped analyses either multiple unpaired t-tests or where appropriate 2-way ANOVA with a Sidak post-hoc analysis was performed. P values less than 0.05 (95% confidence interval) were considered significant. For all in vivo studies, researchers were blinded from mouse genotypes and a numerical ear tagging system enabled unbiased data collection. Upon completion of the study, mouse ID numbers were cross-referenced with genotype to permit analysis. Mice were excluded from the MI study if they lacked a scar or infarct, as evaluated by histological staining at 4 weeks post-MI.

The results of the experiments are now described.

Ablation of Fibroblast Mcu Inhibits ₘCa²⁺ Uptake

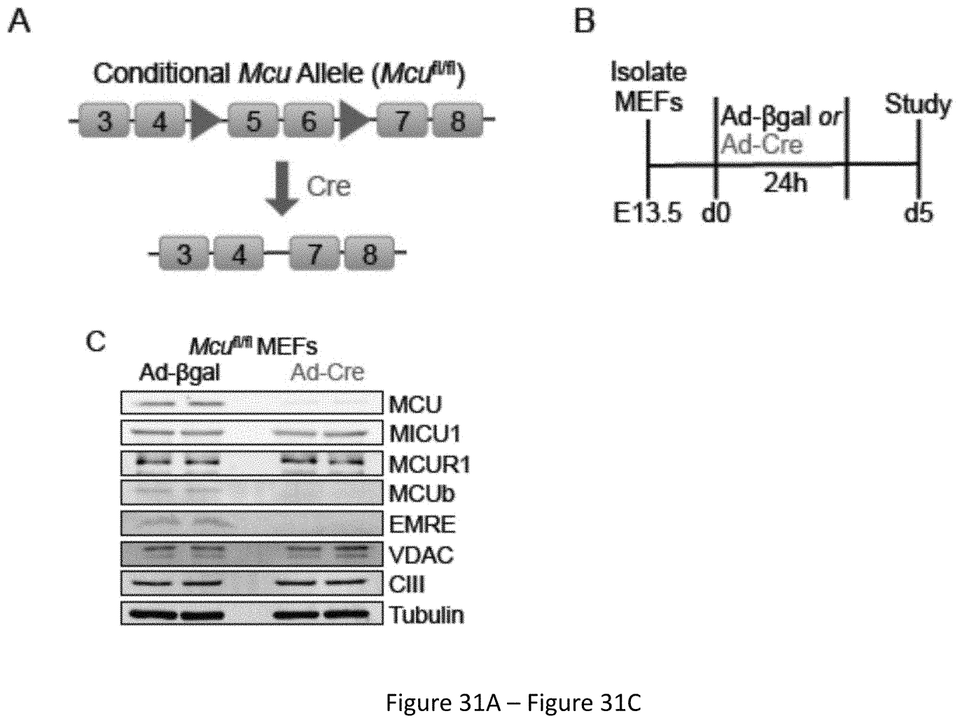

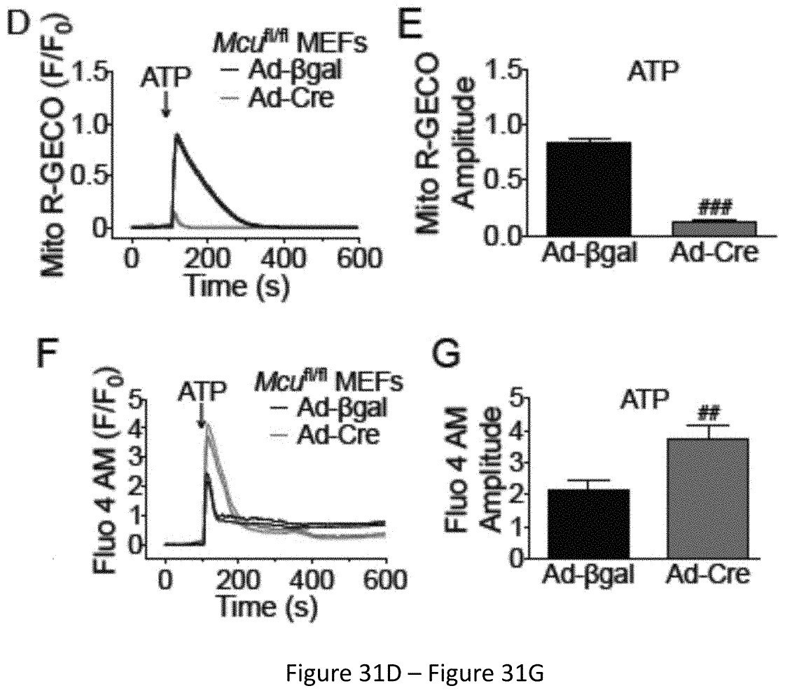

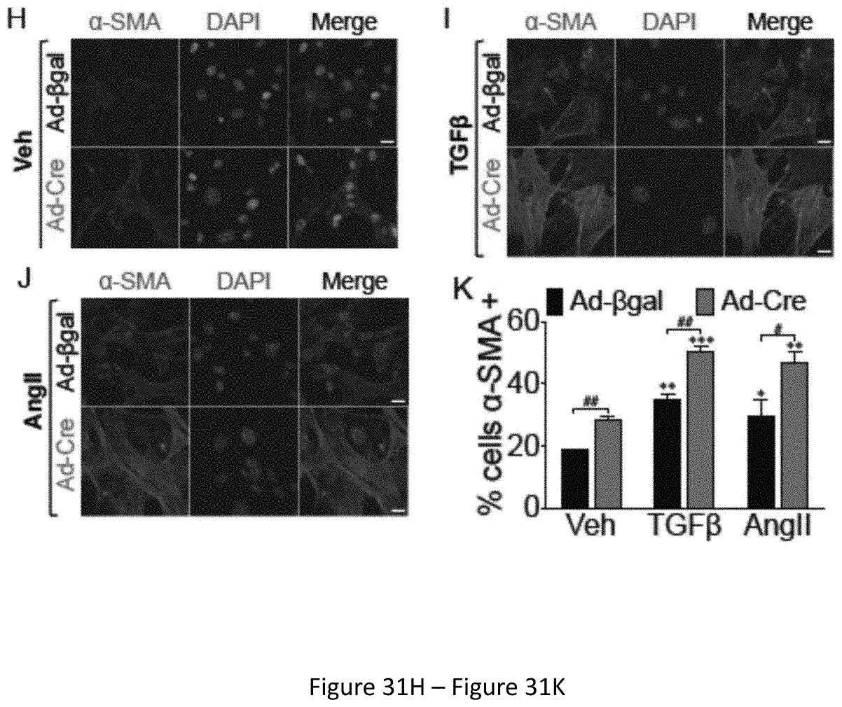

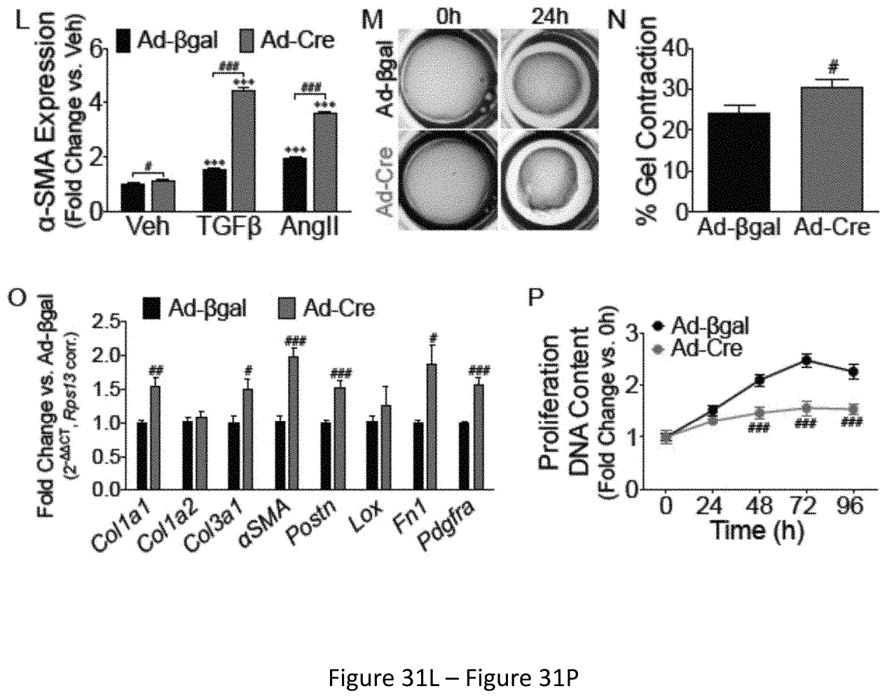

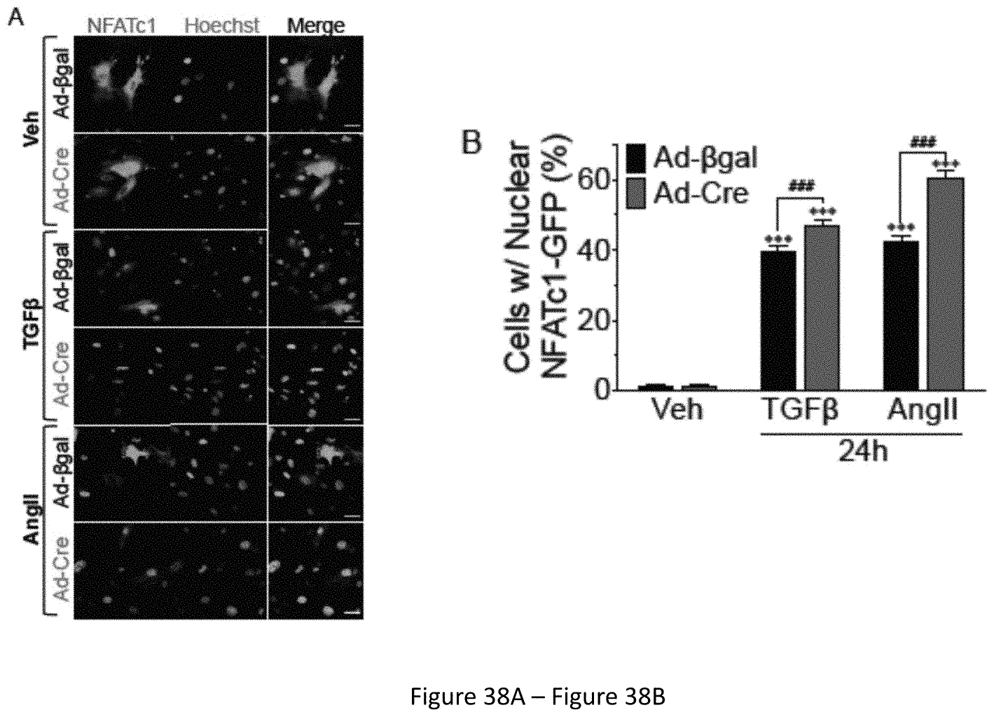

To examine the contribution of ₘCa²⁺ uptake to myofibroblast differentiation, Mcu, the pore-forming subunit of the mitochondrial calcium uniporter (mtCU) that is necessary for ₘCa²⁺ uptake was deleted (FIG. 31A) (Baughman et al., 2011; De Stefani et al., 2011; Luongo et al.; Pan et al., 2013). Mouse embryonic fibroblasts (MEFs) were isolated from E13.5 Mcu^{fl/fl} embryos and infected with adenovirus-encoding cre recombinase (Ad-Cre) or beta-galactosidase (Ad-βgal, control adenoviral infection) for 24 h, and 4 days later cell lysates were analyzed by Western blot. Cre mediated deletion of exons 5-6 caused complete loss of MCU protein (FIG. 31C). A loss of mtCU components MCUb and EMRE (FIG. 31C) was observed, likely attributed to protease mediated degradation of the other structural/channel-forming mtCU components (Tsai et al., 2017). Voltage dependent anion channel (VDAC) and the UQCRC2 (Ubiquinol-cytochrome-c reductase complex core protein 2) subunit of Complex III (CIII) were used as mitochondrial loading controls and tubulin served as a total lysate loading control. Next, Mcu^{fl/fl} MEFs were infected with Ad-Cre or Ad-βgal and 72 hours later transduced with adenovirus encoding a mitochondrial-targeted genetic Ca²⁺ reporter (Miro- R-GECO) for 48 hours. Prior to live-cell imaging, cells were loaded with the calcium sensitive dye Fluo-4 AM to measure cytosolic calcium (_{c}Ca²⁺) transients. After baseline recordings, cells were treated with ATP to initiate purinergic receptor-mediated IP3R Ca²⁺ release. Control MEFs (Ad-βgal) displayed robust ₘCa²⁺ transients, whereas Mcu^{-/-} MEFs (Ad-Cre) displayed complete loss of mCa²⁺ uptake (FIG. 31D-31E). Further, loss of MCU-mediated uptake elicited a significant increase in _{c}Ca²⁺ transients, suggesting that mitochondria buffer _{c}Ca²⁺ signaling in fibroblasts (FIG. 31F-31G). In addition, loss of ₘCa²⁺ uptake enhanced cytosolic signaling. Using an adenovirus-encoding NFATc1-GFP, the nuclear translocation of NFATc1 was measured following fibrotic stimuli. NFATc1 normally resides in the cytoplasm, but upon increased _{c}Ca²⁺ NFATc1 is dephosphorylated and able to translocate into the nucleus to regulate gene transcription (Crabtree and Olson, 2002). Treatment with TGFP or AngII for 24 hours induced nuclear translocation of NFATc1 in control cells (Ad-βgal) and this was potentiated in Mcu^{-/-} fibroblasts (Ad-Cre) (FIG. 38A-38B).

Loss of ₘCa2+ Uptake Promotes Myofibroblast Differentiation

To determine the role of ₘCa²⁺ signaling in myofibroblast differentiation, Mcufl/fl MEFs were infected with Ad-Cre or Ad-βgal and 5 days later treated with pro-fibrotic agonists TGFβ or AngII. MEFs were examined for differentiation into a myofibroblast by quantifying α-smooth muscle actin (α-SMA) stress fiber formation, the prototypical marker of myofibroblasts (Tomasek et al., 2002). Mcu−/− MEFs (Ad-Cre) displayed increased myofibroblast formation at baseline (vehicle) and following 24 hours TGFβ or AngII treatment as evidenced by an increase in the percentage of α-SMA+ cells and a ˜4-fold increase in α-SMA expression versus controls (Ad-βgal) (FIG. 28H-L). Functionally, Mcu^{-/-} MEFs displayed increased contraction of collagen gel matrices, even without TGFβ or AngII treatment, indicative of enhanced acquisition of the myofibroblast phenotype (FIG. 31M-31N). It was observed that loss of mtCU-mediated Ca²⁺-uptake alone was sufficient to increase the expression of key myofibroblast genes including: collagens (Col1a1 and Col3a1), α-SMA (Acta2), periostin (Postn), fibronectin (Fn1) and platelet derived growth factor receptor alpha (P dgfr a) (FIG. 31O). Importantly, the observed enhancement in Mcu^{-/-} α-SMA+ cells and gel contraction was not due to increased proliferation. Mcu^{-/-} MEFs showed significantly reduced proliferation rates, as measured by DNA content, which is also characteristic of a more differentiated cell type (FIG. 31P). Overall, these data show that loss of ₘCa²⁺ uptake promotes myofibroblast differentiation.

Pro-Fibrotic Stimuli Alter mtCU Gating to Reduce ₘCa²⁺ Uptake

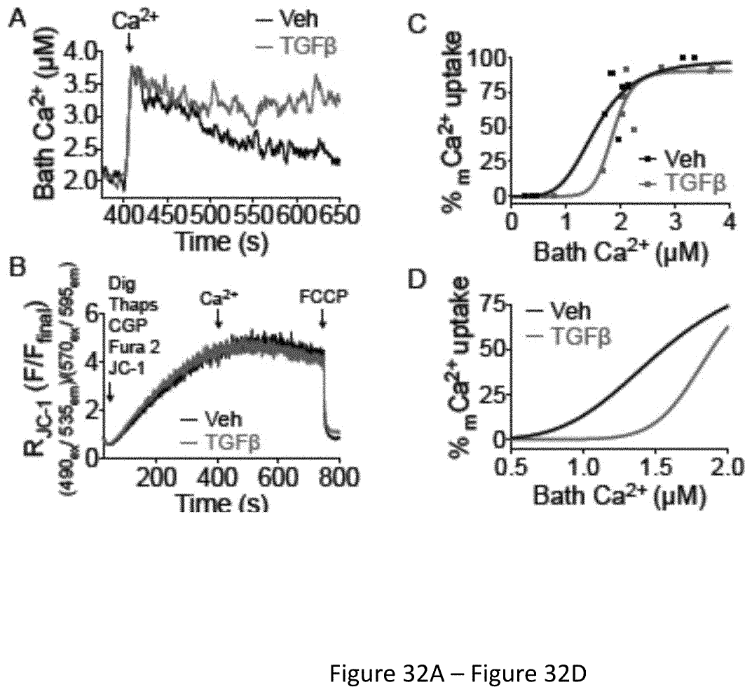

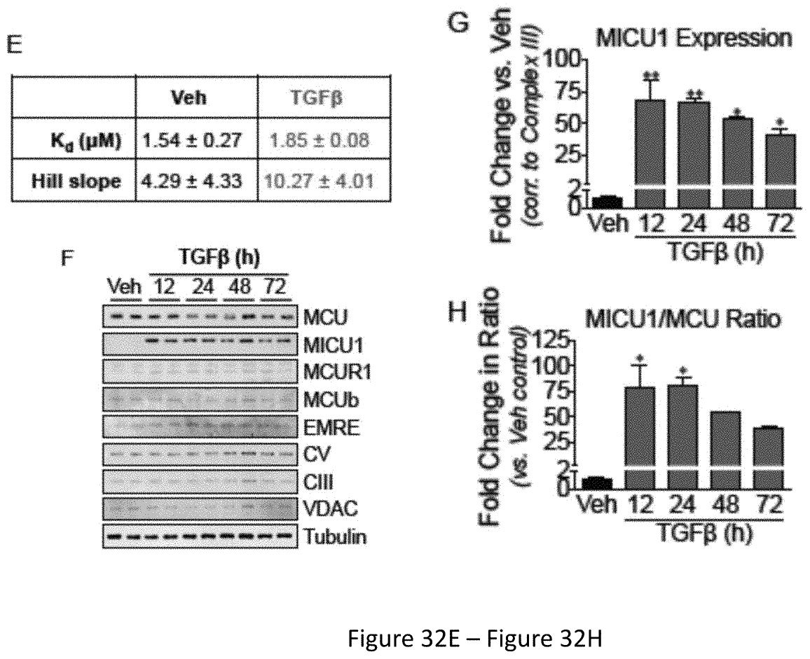

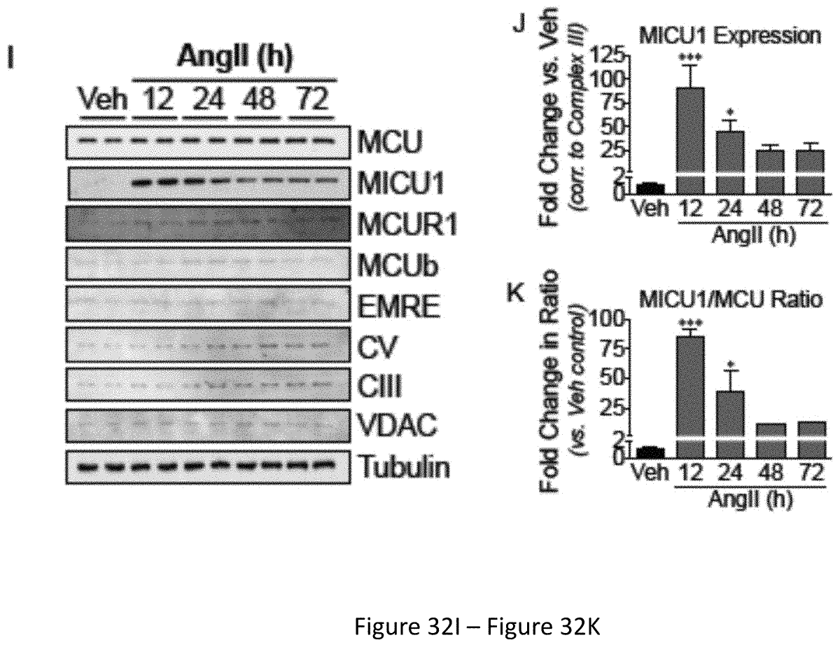

Given the significant impact that loss of ₘCa²⁺ uptake had on myofibroblast formation next it was examined whether acute fibrotic signaling directly altered mtCU function. After treating wildtype (WT) MEFs with TGFβ for 12 hours, fibroblasts were permeabilized with digitonin, in the presence of thapsigargin (SERCA inhibitor to prevent ER Ca²⁺ uptake) and CGP-37157 (NCLX inhibitor to prevent ₘCa²⁺ efflux), and loaded with the Ca²⁺ sensor Fura-2 for ratiometric monitoring using a spectrofluorometer. An increase in Fura-2 signal signifies the increase in bath Ca²⁺ and a decrease in Fura-2 signal after each bolus represents ₘCa²⁺ uptake. This high-fidelity system allows careful monitoring of uptake independent of changes in other calcium transport mechanisms. It was observed that TGFβ-treated fibroblasts displayed a decrease in ₘCa²⁺ uptake following the delivery of ˜0.5-2 μM[Ca²⁺] (representative trace shown in FIG. 32A). Importantly, simultaneous monitoring of mitochondrial membrane potential (Δψ) using the ratiometric reporter, JC-1, showed no difference in the driving force for uptake (FIG. 32B). After calibration of the Fura-2 reporter in the experimental system (FIG. 32A), the percentage of ₘCa²⁺ uptake was quantified over a range of varying bath Ca²⁺ concentrations and data points were fit to the Hill equation using a nonlinear least-squares fit.

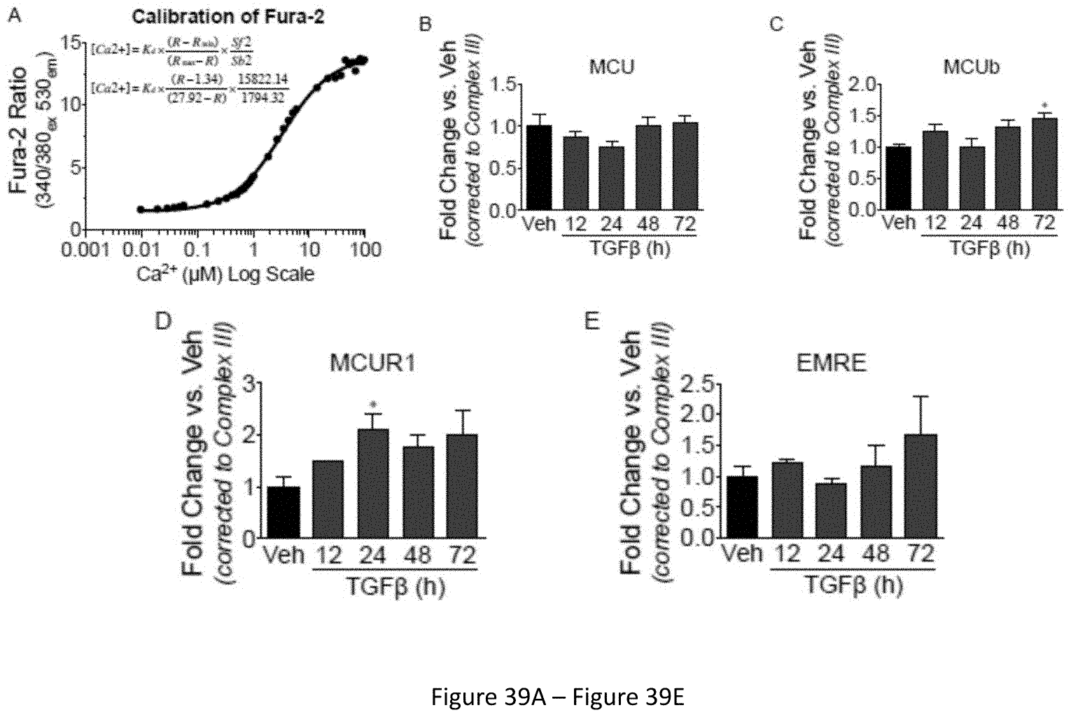

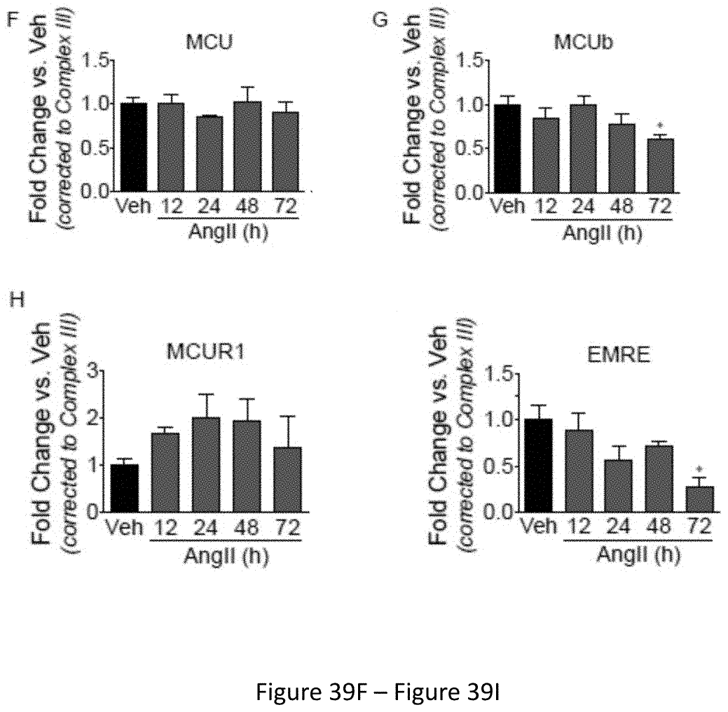

From the dose response curve, it was observed that the nonlinear nature of mtCU-mediated ₘCa²⁺ uptake, consistent with other reports (FIG. 32C) (Antony et al., 2016; Mallilankaraman et al., 2012; Williams et al., 2013). TGFβ treatment for 12 hour shifted the dose-response curve to the right, demonstrating an increase in the [Ca²⁺] threshold for ₘCa²⁺ uptake (FIG. 32C-32D). The calculated Kd value was ˜1.5 μM in control cells and 1.9 μM in TGFβ-treated cells, indicating that following TGFβ a higher [Ca²⁺] was needed to achieve 50% maximal mtCU uptake (FIG. 32E). In addition, the Hill coefficient identified a difference in the slopes of the dose response curves in TGFβ-treated vs. control cells (FIG. 32E), 4.29 in control cells vs. 10.27 in TGFβ-treated cells, demonstrating that TGFβ indeed enhanced mtCU gating, allowing virtually no uptake until a given threshold was reached. To probe the mechanism responsible for TGFβ-induced alterations in ₘCa²⁺ uptake, WT fibroblasts were treated with TGFβ and 12, 24, 48, and 72 hours later extracted protein to examine the expression of mtCU components. Western blot analysis revealed a dramatic increase in MICU1 expression 12 hours after treatment (FIG. 32F-32G). CIII (subunit UQCRC2) and VDAC served as mitochondrial loading controls and tubulin served as a total lysate loading control. Since the MICU1/MCU ratio underlies tissue-specific differences in the mtCU [Ca²⁺] threshold of uptake (Paillard et al., 2017), the relative change in MICU1/MCU ratio was quantified. TGFfβ treatment rapidly increased the MICU1/MCU ratio (FIG. 32H). A similarly large increase in MICU1 expression in MEFs treated with the fibrotic agonist AngII was also observed (FIG. 32I-32K), suggesting this is a conserved mtCU regulatory mechanism during myofibroblast differentiation. The substantial increase in the MICU/MCU ratio is in agreement with the observed change in ₘCa²⁺ uptake following TGFβ treatment and is consistent with other reports ascribing that MICU1 is a gatekeeper restricting mtCU-mediated Ca²⁺ uptake at signaling levels of [_{c}Ca²³⁰ ] (Csordas et al., 2013; Mallilankaraman et al., 2012). Therefore, it is proposed that profibrotic agonists signal to acutely upregulate MICUl expression to inhibit ₘCa²⁺ uptake and initiate the signaling that drives myofibroblast differentiation. The relative expression of additional mtCU components was also quantified (FIG. 39B-39I).

TGFβ/AngII Signaling Elicits Rapid and Dynamic Changes in Fibroblast Metabolism

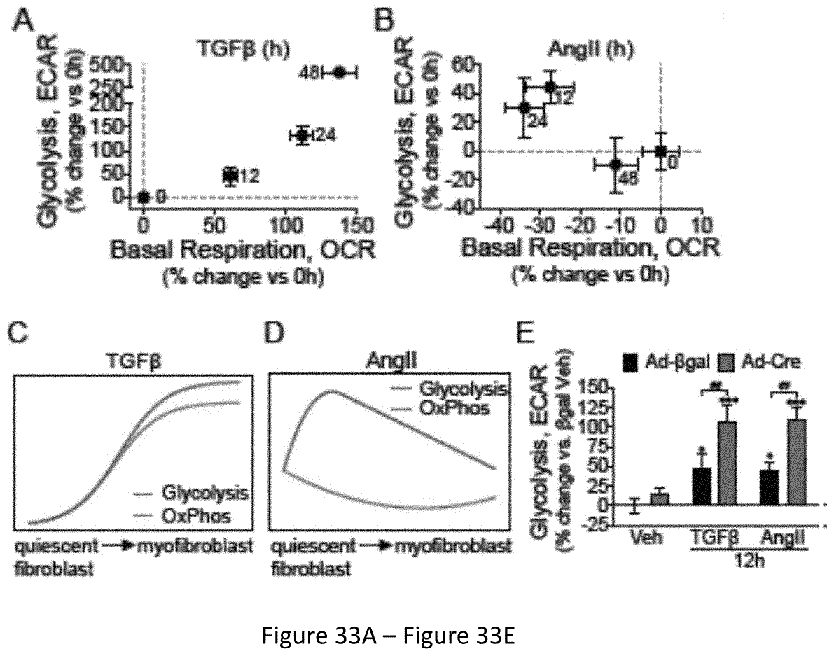

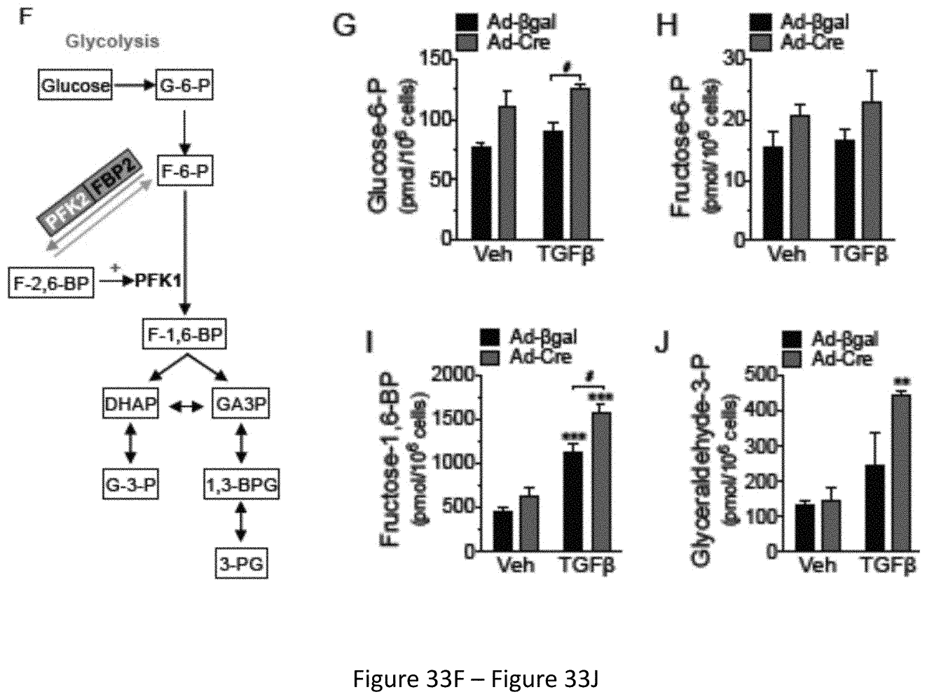

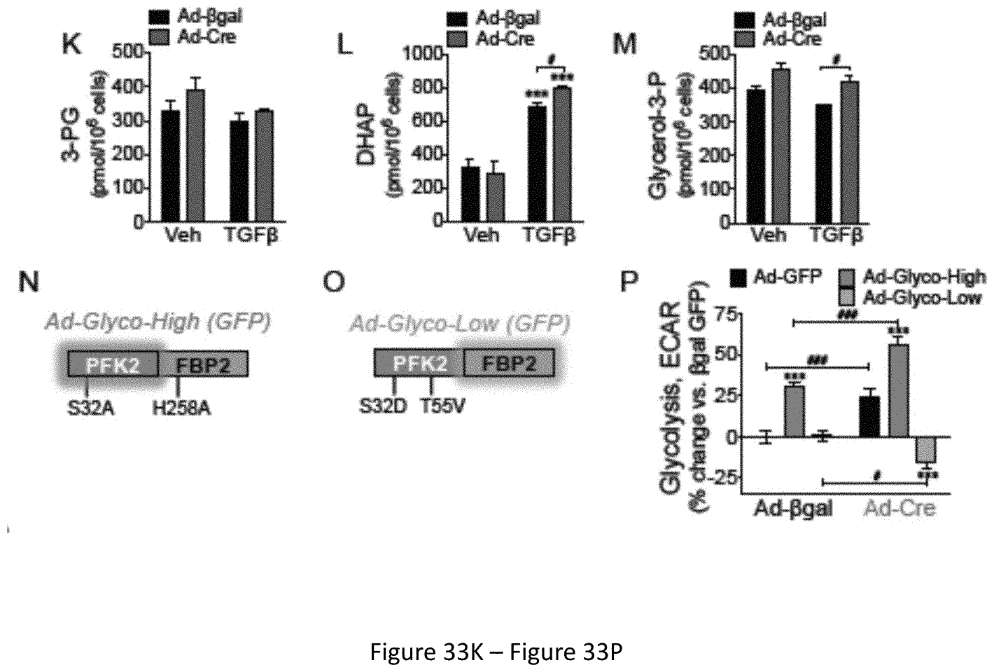

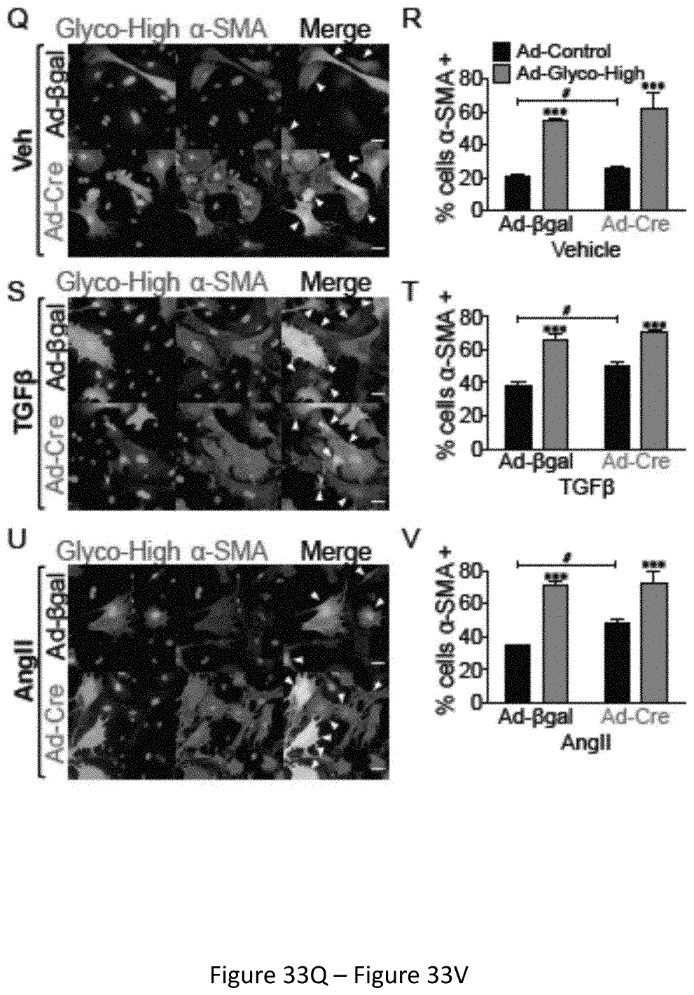

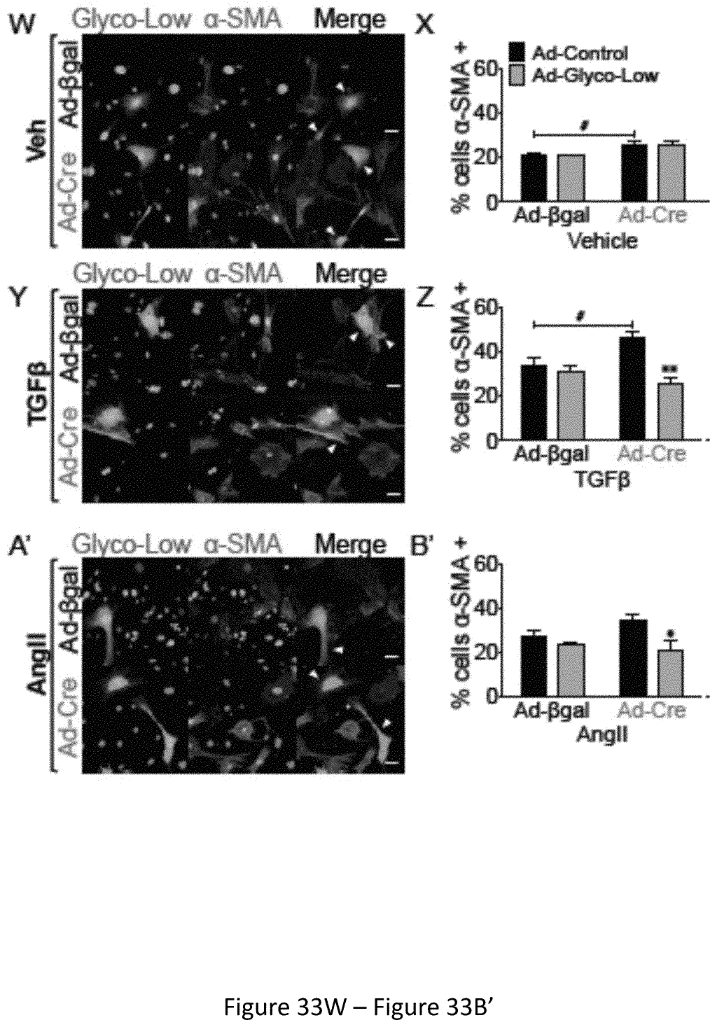

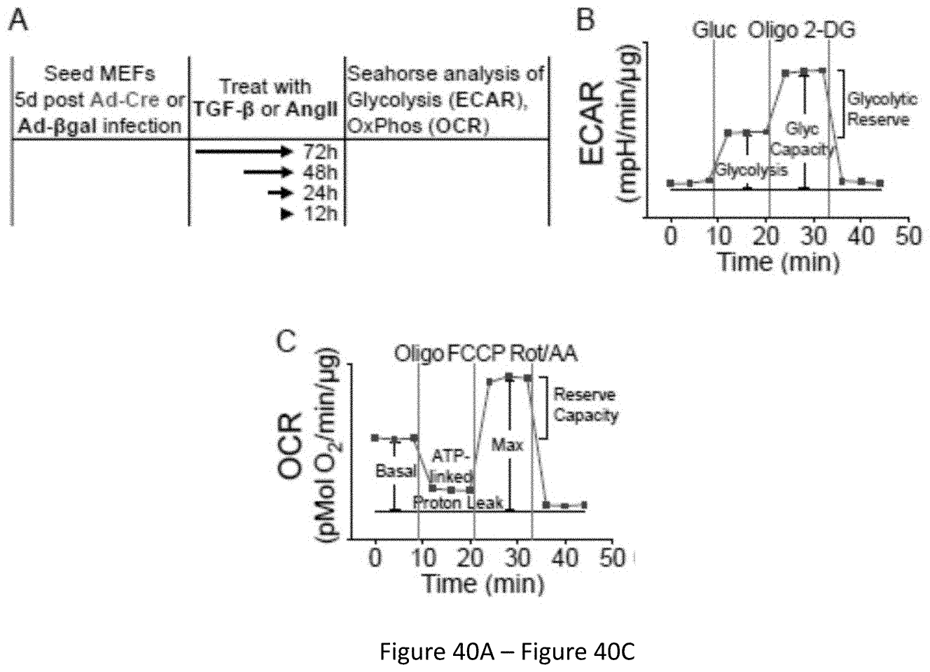

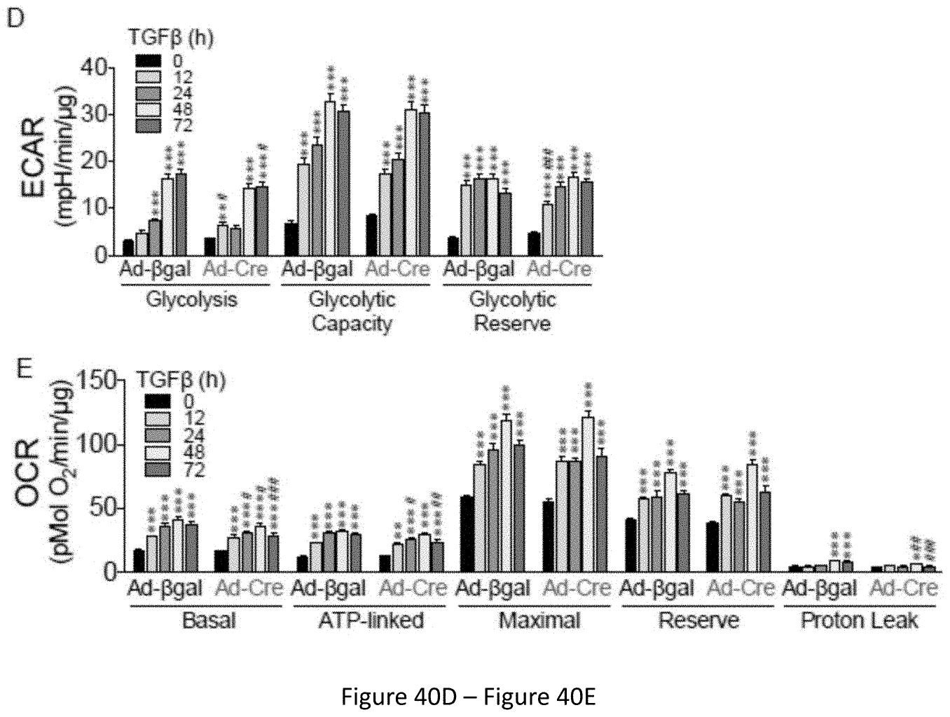

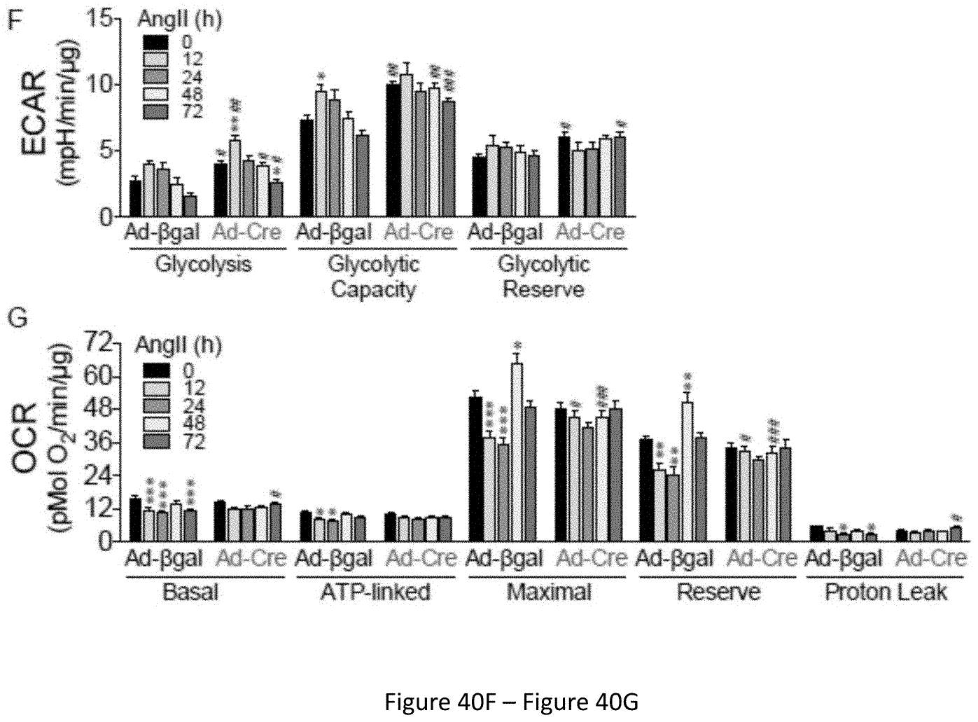

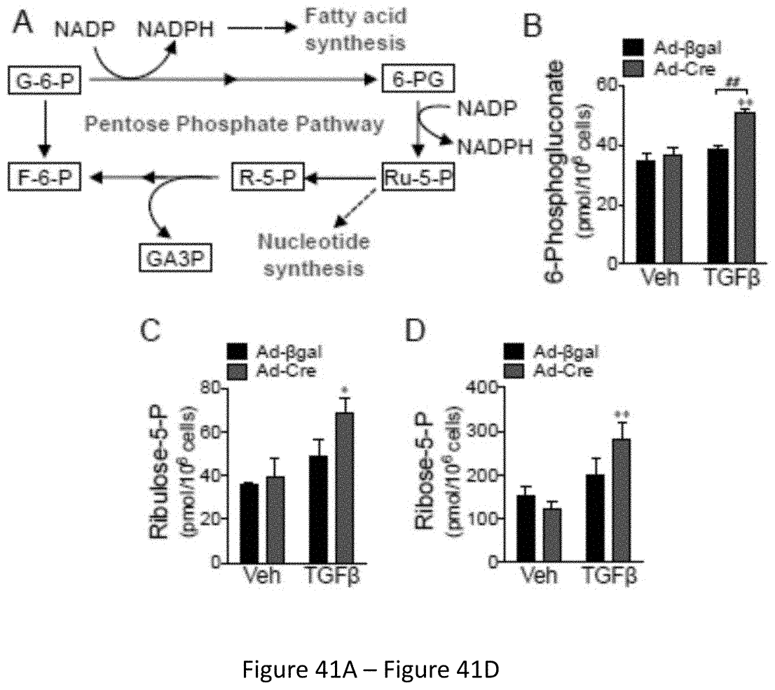

_{c}Ca²⁺ is integrated into the mitochondrial matrix via the mtCU, a mechanism theorized to integrate cellular demand with metabolism and respiration (Balaban, 2009; Hajnoczky et al.; Luongo et al.; Williams et al., 2015). Further, metabolic reprogramming is required for numerous cellular differentiation programs (Moussaieff et al., 2015; Xu et al., 2013; Zhou et al., 2012) and recent studies suggest that enhanced glycolysis promotes fibroblast differentiation (Bernard et al., 2015; Xie et al., 2015). This prompted experiments to examine metabolic changes in glycolysis and oxidative phosphorylation during myofibroblast differentiation. Mcu^{fl/fl} MEFs were transduced with Ad-Cre or Ad-βgal and 5 days later treated with TGFfβ or AngII for 12, 24, 48, or 72 hours, followed by measurement of extracellular acidification rates (ECAR, glycolysis) or oxygen consumption rates (OCR, OxPhos) using a Seahorse XF96 analyzer (FIG. 33A-33B). TGFβ stimulation elicited a significant increase in basal respiration (˜135% increase from baseline) and glycolysis (>400% increase from baseline) peaking 48 hours after treatment (FIG. 33A-33C). AngII likewise caused a rapid increase in glycolysis (45% increase from baseline), peaking ˜12 hours; however, AngII caused a slight decrease in basal respiration (FIG. 33B and 33D). Interestingly, loss of MCU (Ad-Cre) further enhanced the increased glycolysis induced by both TGFβ and AngII>2-fold, as compared to control (Ad-βgal) (FIG. 33E). All other Seahorse measured metabolic parameters under all conditions can be found in FIG. 40D-40G.

Next, using a quantitative metabolomics approach, the concentrations of fibroblast metabolites were quantified by mass spectrometry in Mcu^{-/-} (Ad-Cre) and control (Ad-βgal) MEFs at baseline and 12 hours post-TGFβ. These data confirmed the TGFβ-mediated increase in glycolysis and augmentation by loss of MCU that was observed by Seahorse analysis. Mcu−/− MEFs (Ad-Cre) displayed higher levels of the glycolytic intermediates: glucose-6-phosphate (G-6-P), fructose-6-phosphate (F-6-P), fructose- 1,6-bisphosphate (F-1,6-BP), glyceraldehyde-3-phosphate (GA3P), dihydroxyacetone phosphate (DHAP) and glycerol-3-phosphate (G-3-P) (FIG. 33F-33M). Importantly, F-1,6-BP, the glycolytic intermediate produced in the first committed step of glycolysis, was significantly increased following TGFβ treatment and this increase was potentiated by loss of MCU (Ad-Cre) (FIG. 33I). F-1,6-BP is metabolized into GA3P and DHAP, and concentrations of these metabolites followed a similar trend with an increase post-TGFβ, which was similarly potentiated by loss of MCU (FIG. 33J and 33L). In addition to generating energy, glycolysis contributes metabolic intermediates into ancillary pathways, which are required for the synthesis of cellular components. This is of particular relevance here when considering cellular differentiation from a quiescent fibroblast to a much larger, synthetic, contractile myofibroblast. The pentose phosphate pathway (PPP) is one of the ancillary pathways of glycolysis and generates ribulose-5-phosphate (Ru-5-P) along with NADPH, which are critical for nucleotide and fatty acid/phospholipid synthesis respectively (FIG. 33A) (Eggleston and Krebs, 1974; Patra and Hay, 2014; Stanley et al.). Following TGFβ, Mcu−/− MEFs exhibited increased levels of 6-phosphogluconate (6-PG), Ru-5-P, and ribose-5-phosphate (R-5-P) compared to vehicle treated controls (FIG. 33B-33D).

To determine the necessity of enhanced glycolytic flux on myofibroblast formation, a rate-limiting enzyme of glycolysis, phosphofructokinase 1 (PFK1), was modulated. PFK1 is allosterically activated by fructose-2,6-bisphosphate (F-2,6-BP), the levels of which are regulated by the bi-functional enzyme phosphofructokinase 2 (PFK2)/fructose bisphosphatase 2 (FBP2) (FIG. 33F) (Mor et al., 2011). Employing adenovirus-encoding a phosphatase-deficient PKF2 mutant (S32A, H258A; Ad-Glyco-High) or kinase-deficient PFK2/FBP2 mutant (S32D, T55V; Ad-Glyco-Low) the impact of modulating glycolytic capacity during myofibroblast differentiation was examined (FIG. 33N-330) (Kurland et al., 1992; Salabei et al., 2016). The PFK2/FBP2 mutant adenoviruses also encoded GFP driven by a separate CMV promoter, allowing easily distinguishable transduced cells from uninfected fibroblasts. As expected, Ad-Glyco-High expression increased glycolysis in both control (Ad-βgal) and Mcu^{-/-} (Ad-Cre) MEFs, while Ad-Glyco-Low expression inhibited the increased glycolysis observed in Mcu−/− MEFs (FIG. 33P). Control and Mcu−/− MEFs were infected with either Ad-Glyco-High or Ad-Glyco-Low and 24 h later treated with TGFβ or AngII for 24 h followed by quantification of α-SMA+cells by immunofluorescence. Enhancing glycolysis was sufficient to drive myofibroblast formation (FIG. 33Q-33R) and potentiated cellular differentiation elicited by TGFβ and AngII (FIG. 33S-V). In addition, inhibition of glycolysis (Ad-Glyco-Low) reverted the TGFβ- and AngII-mediated increases in differentiation observed in Mcu−/− MEFs back to control levels (FIG. 33W-33B′).

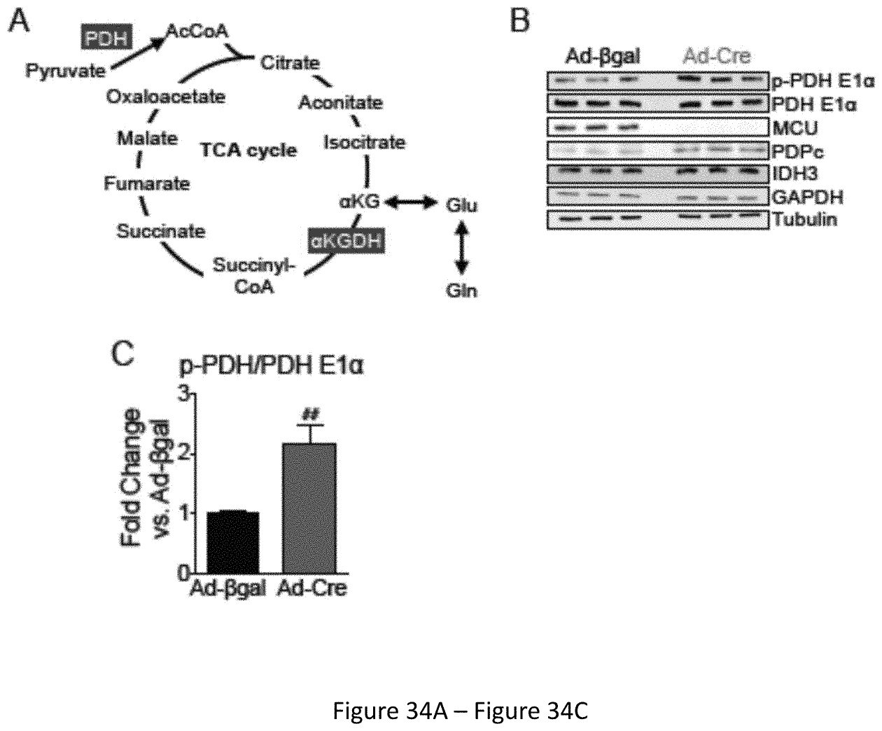

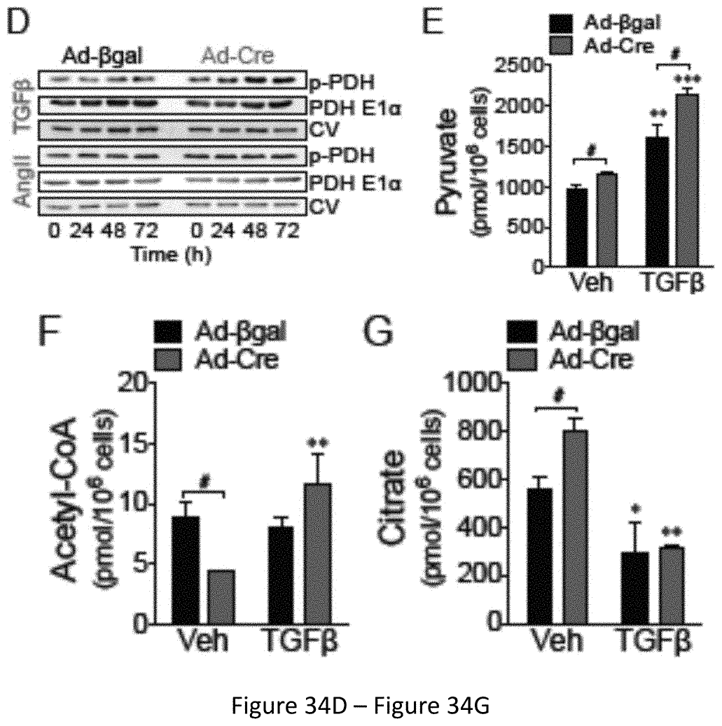

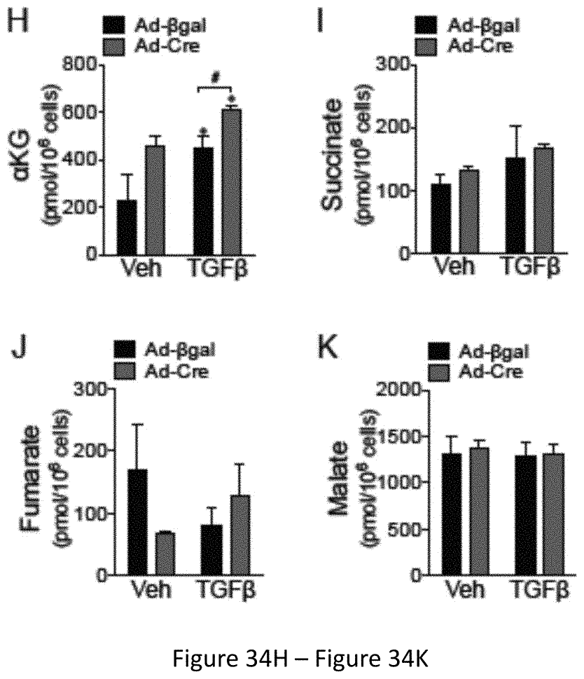

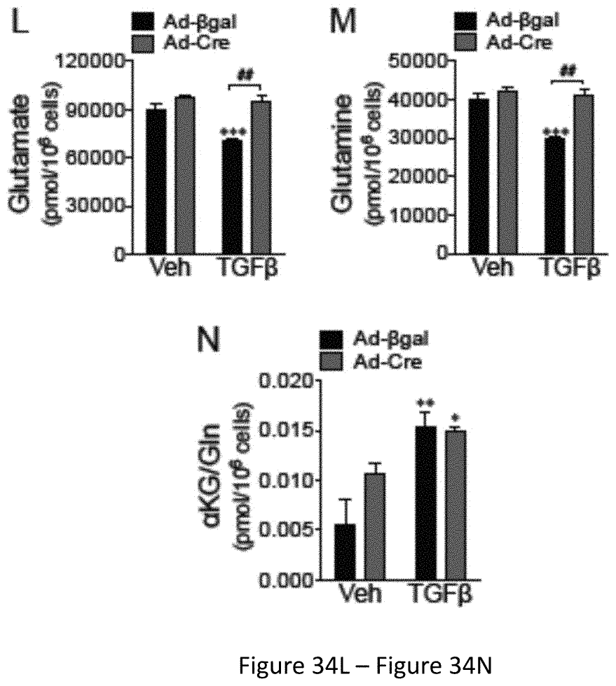

Next, mitochondrial metabolism was evaluated since it is well established that ₘCa²⁺ signaling directly impacts TCA cycle intermediates by the modulation of pyruvate dehydrogenase (PDH) and α-ketoglutarate dehydrogenase (αKGDH) activity (FIG. 34A). mCa²⁺ activates PDH phosphatase (PDP1), which dephosphorylates the PDH E1α subunit and thereby increases PDH activity to convert pyruvate to acetyl-CoA (Denton et al., 1972; Karpova et al., 2003; McCormack and Denton, 1984). Western blot analysis of phosphorylated PDH (p-PDH E1α, inactive) revealed significantly increased p-PDH E1α/PDH in Mcu^{-/-} MEFs (Ad-Cre) at baseline compared to controls (Ad-βgal) (FIG. 34B-34C). Further, both TGFβ and AngII increased the ratio of p-PDH E1α/PDH, which was potentiated in Mcu-null fibroblasts (FIG. 34D). Accordingly, metabolomics analysis revealed that TGFβ increased pyruvate from 1000 to 1600 pmol/million cells, consistent with inhibition of PDH (FIG. 34E). Mcu^{-/-} MEFs had increased pyruvate both at baseline and following TGFβ compared to controls (FIG. 34E). Acetyl-CoA was decreased in Mcu^{-/-} MEFs at baseline, consistent with inactive PDH (FIG. 34F).

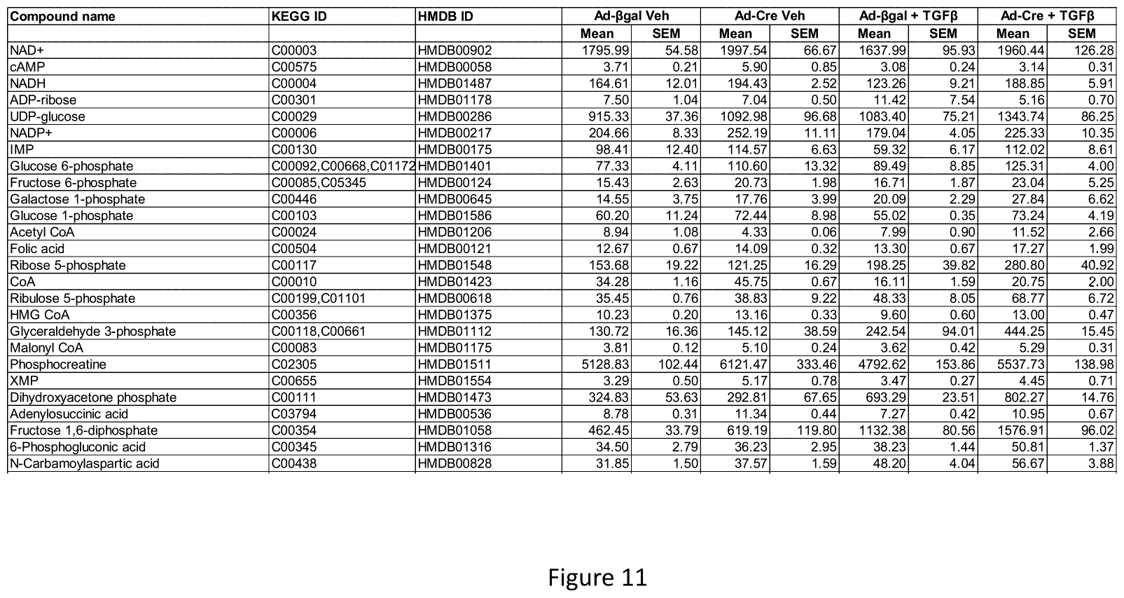

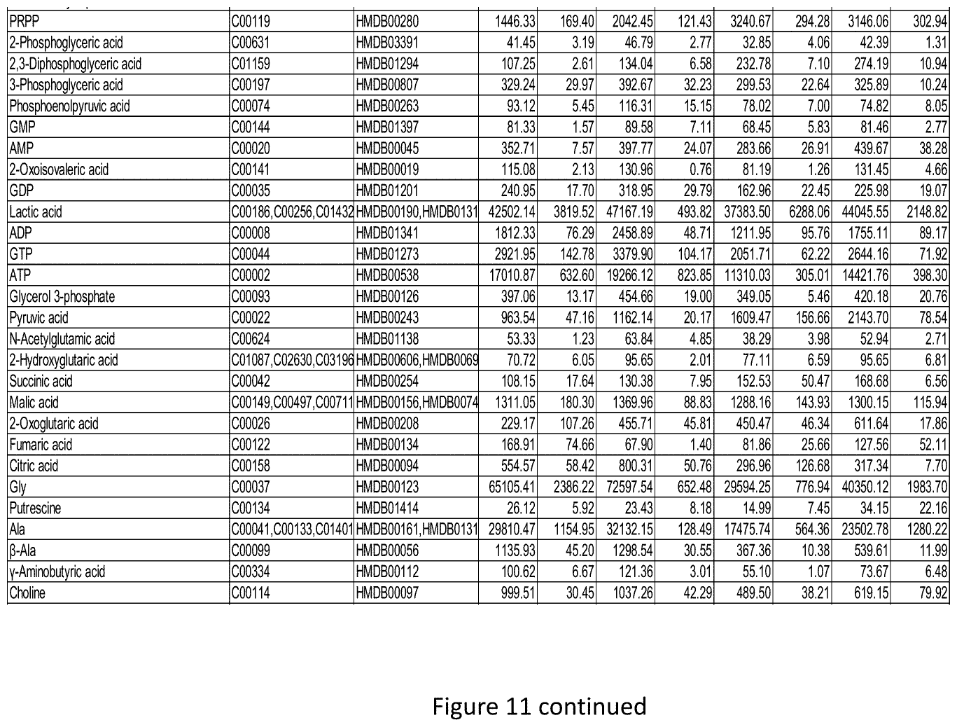

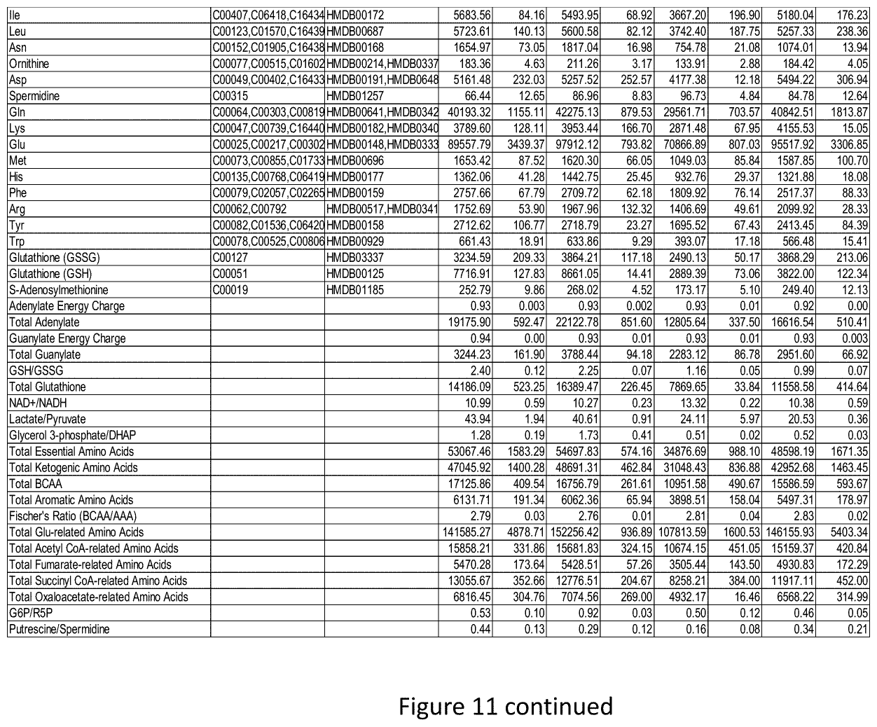

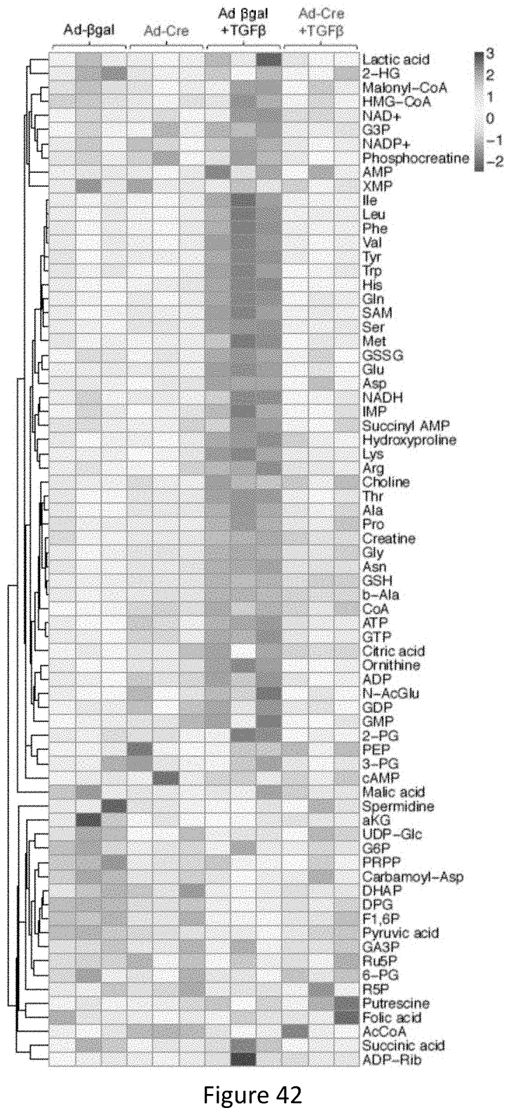

Following TGFβ, acetyl-CoA increased in Mcu^{-/-} MEFs, but did not change in control cells (FIG. 34F). Nonetheless, acetyl-CoA levels were 100 times lower than pyruvate levels, suggesting that pyruvate was not entering the TCA cycle via PDH. Consistent with less overall flux through the TCA cycle, citrate levels were significantly reduced following TGFβ (FIG. 34G). Interestingly, α-ketoglutarate (αKG) was increased 12 h after TGFβ treatment (FIG. 34H). Further, Mcu^{-/-} fibroblasts exhibited increased αKG at baseline and following treatment with TGFβ, as compared to controls (FIG. 31H). Other TCA cycle intermediates succinate, fumarate, and malate were unchanged by TGFβ or loss of MCU (FIG. 31I-K). Reduced glucose-dependent TCA flux has been shown to increase anaplerotic elevations in αKG via glutaminolysis (DeBerardinis et al., 2007; Le et al., 2012; Salabei et al., 2015; Yang et al., 2014). TGFβ decreased glutamine (Gln) and glutamate (Glu) levels in control cells and Ma^{-/-} fibroblasts displayed an increase in the αKG/Gln ratio at baseline and after TGFP treatment (FIG. 34L-34N). These results imply that TGFP may increase the metabolism of Gln to increase cellular levels of aKG. All other metabolite concentrations are reported in FIGS. 11 and 42.

αKG Increases JmjC-KDM-Dependent Histone Demethylation to Activate the Myofibroblast Gene Program

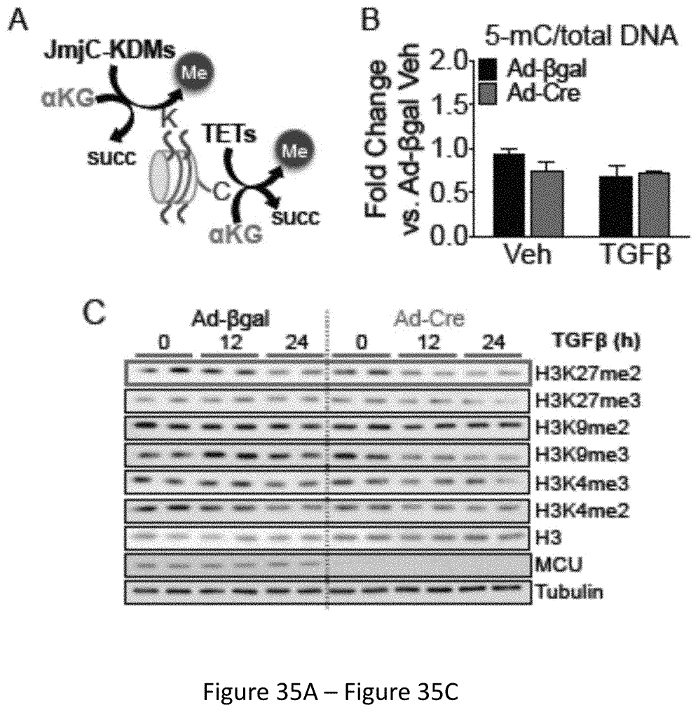

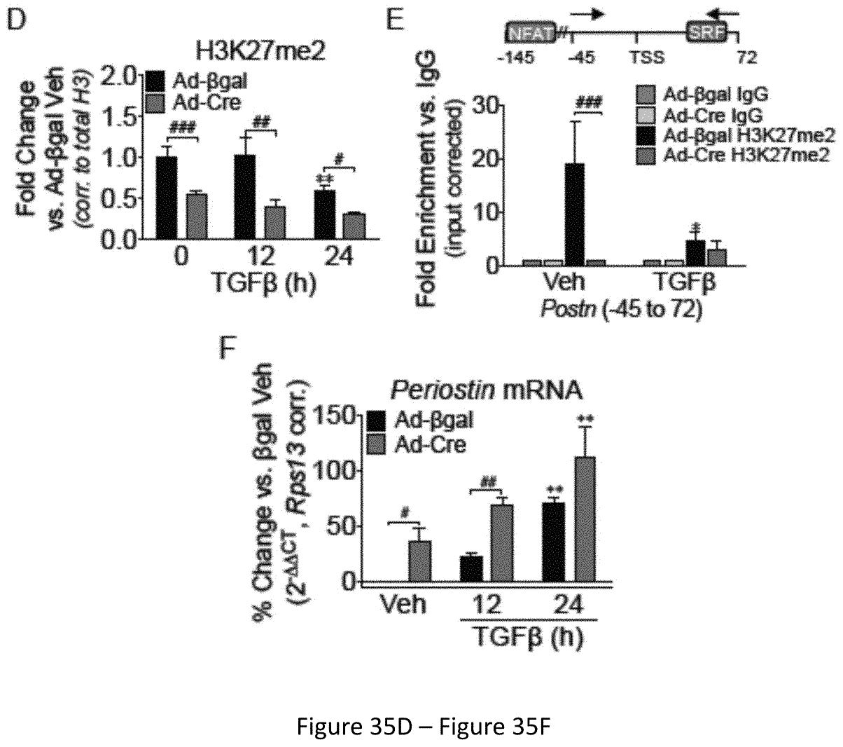

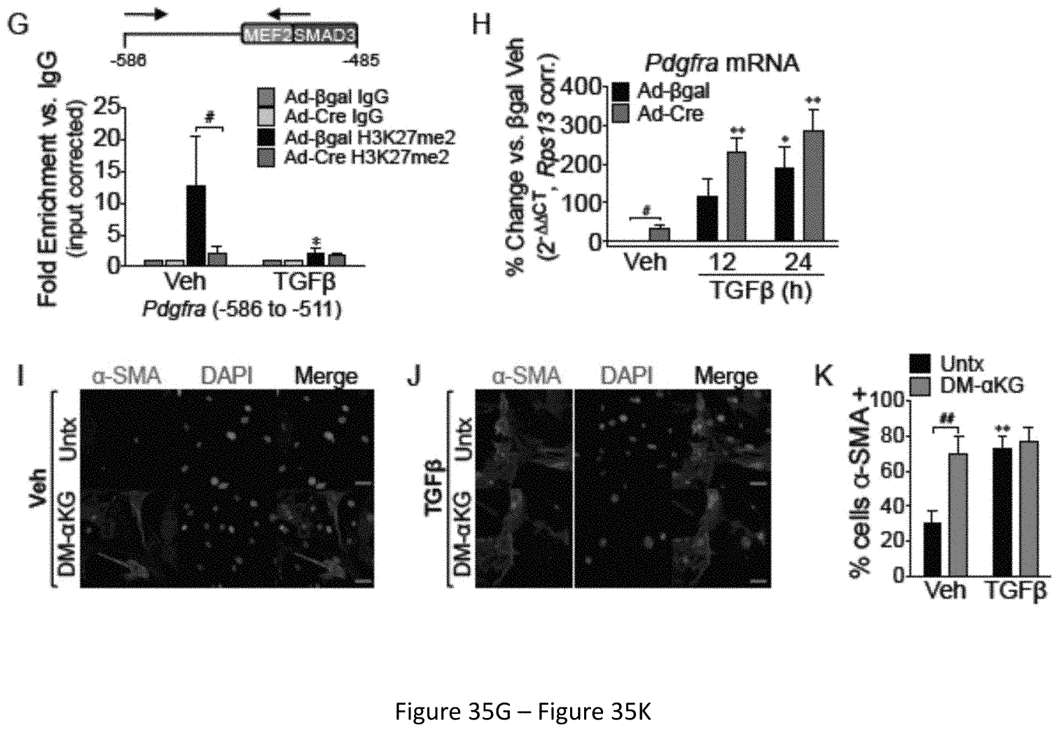

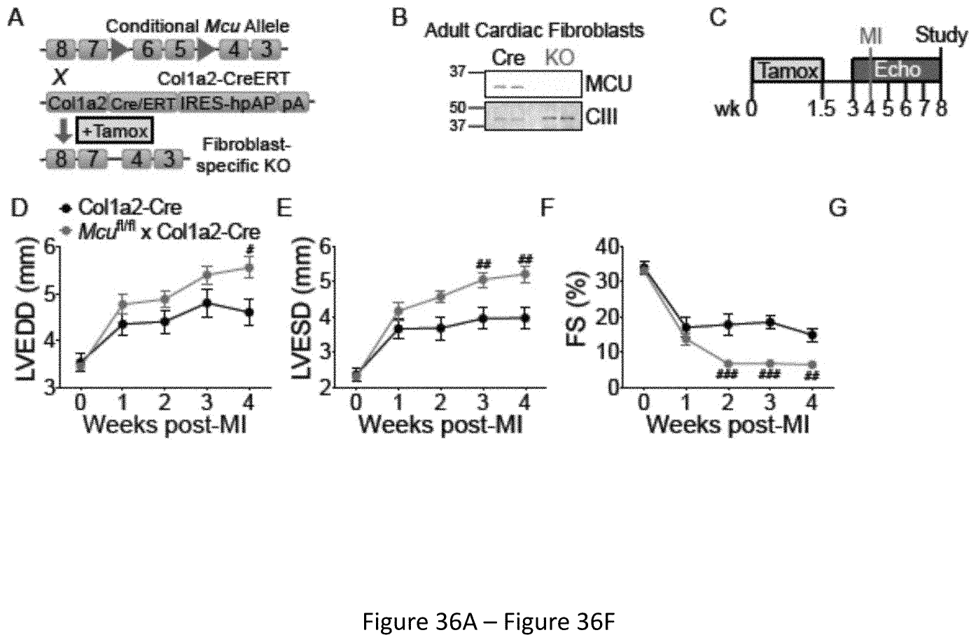

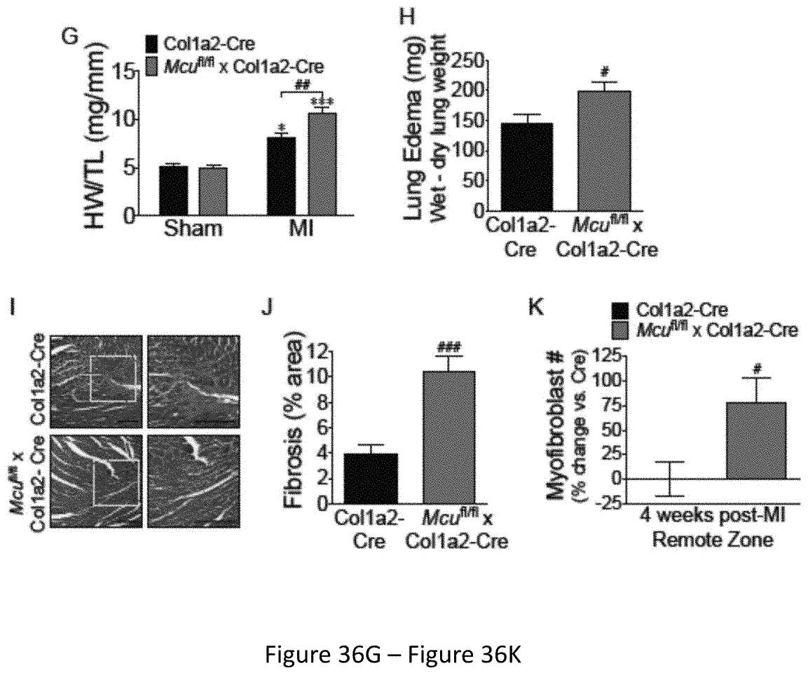

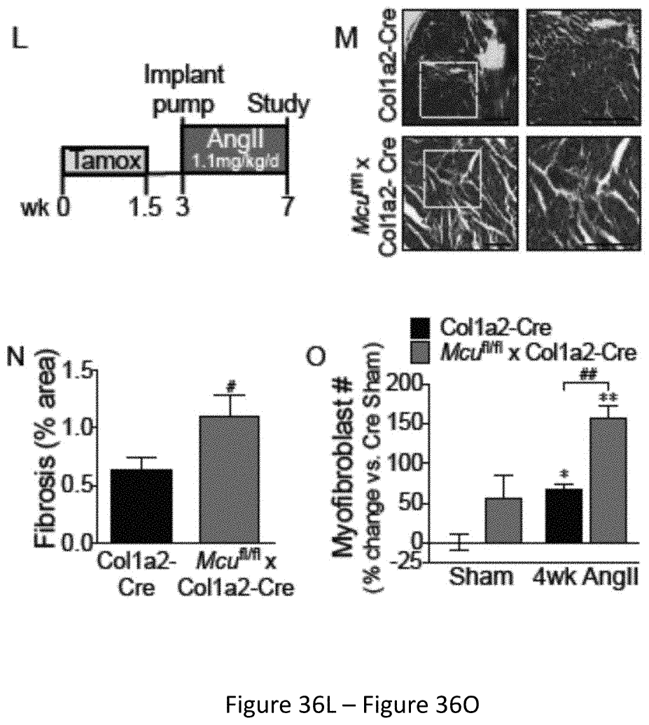

αKG is a cofactor for a family of chromatin-modifying αKG-dependent dioxygenases including ten-eleven translocation (TET) enzymes and Jumonji-C (JmjC)-domain-containing demethylases (JmjCKDMs), which demethylate DNA cytosine residues and histone lysine residues respectively (FIG. 35A) (He et al., 2011; Klose et al., 2006). It is examined whether the observed increase in aKG following TGFβ or loss of MCU altered epigenetic signaling to promote the myofibroblast gene program and differentiation.

Global DNA methylation was first assessed by ELISA in Mcu^{-/-} (Ad-Cre) and control (Ad-βgal) MEFs at baseline and following treatment with TGFβ. Slight, but non-significant, decreases in global DNA methylation was observed with TGFβ and loss of MCU (FIG. 35B). Next, Mcu^{-/-} (Ad-Cre) and control (Ad-βgal) MEFs were treated with TGFβ and cell lysates were examined for histone 3 (H3) lysine (K) methylation at key residues regulated by JmjC-KDMs—H3K27, H3K9 and H3K4 (FIG. 35C). Fibroblasts treated with TGFβ exhibited a progressive decrease in dimethylation of H3K27 (H3K27me2) over time (FIG. 35C-35D). Mcu^{-/-}MEFs exhibited less dimethylation at baseline and post-TGFβ compared to controls (FIG. 35C-35D). H3K27me2 has been implicated in regulating cell fate by preventing inappropriate enhancer activation (Ferrari et al., 2014) and generally is associated with heterochromatin and gene suppression (Barski et al., 2007; Lee et al., 2015). To directly examine the role of H3K27me2 in controlling the myofibroblast gene program, immunoprecipitated chromatin was analyzed using an H3K27me2-specific antibody and ChIP'd DNA by qPCR in key regulatory promoter regions of periostin (Postn) and platelet derived growth factor receptor alpha (Pdgfra), genes which are early and robust indicators fibroblast activation (Kanisicak et al., 2016; Moore-Morris et al., 2014; Tallquist and Molkentin, 2017). In control cells (Ad-βgal), H3K27me2 was enriched at the Postn and Pdgfra loci and these marks were lost after 12 hours of TGFβ with a concordant increase in mRNA expression (FIG. 35E-35H). Furthermore, Mcu^{-/-}MEFs (Ad-Cre) exhibited a lack of H3K27me2 enrichment at the Postn and Pdgfra promoters at baseline, which underlies their enhanced expression of these genes and ultimately increased myofibroblast differentiation (FIG. 35E-35H). Importantly, binding sites for transcription factors known to be prominent drivers of myofibroblast differentiation such as serum response factor (SRF), SMAD family member 3 (SMAD3), nuclear factor for activated T-cells (NFAT), myocyte enhancer factor-2 (MEF2)—were predicted by Matlnspector to be flanked by, or in close approximation, to the regulatory regions probed by the qPCR primer sets (FIG. 35E and 35G). To determine the physiological relevance of aKG dependent histone demethylation on myofibroblast differentiation MEFs was incubated in media containing cell-permeable dimethyl-αKG (DM-αKG) with or without TGFβ for 48 hours and assessed α-SMA formation by immunofluorescence. Strikingly, DM-αKG increased the percentage of α-SMA positive cells to the same extent as 48 hours of TGFβ treatment (FIG. 35I-35K). All together, these data demonstrate that TGFβ-induces metabolic changes that lead to increased αKG levels and subsequent demethylation of repressive H3K27me2 chromatin marks to allow for coordinated genetic reprogramming and myofibroblast differentiation.

Adult Deletion of Fibroblast Mcu Exacerbates Cardiac Dysfunction and Fibrosis Post-MI and Chronic Angiotensin II Administration

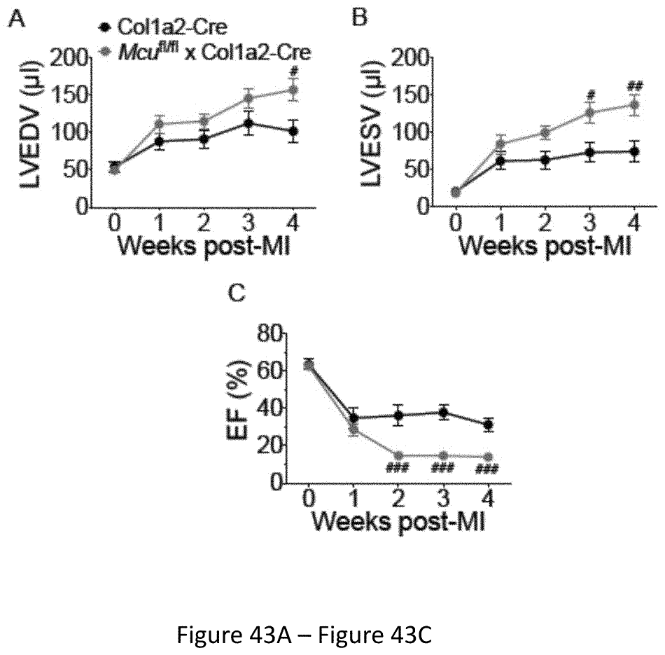

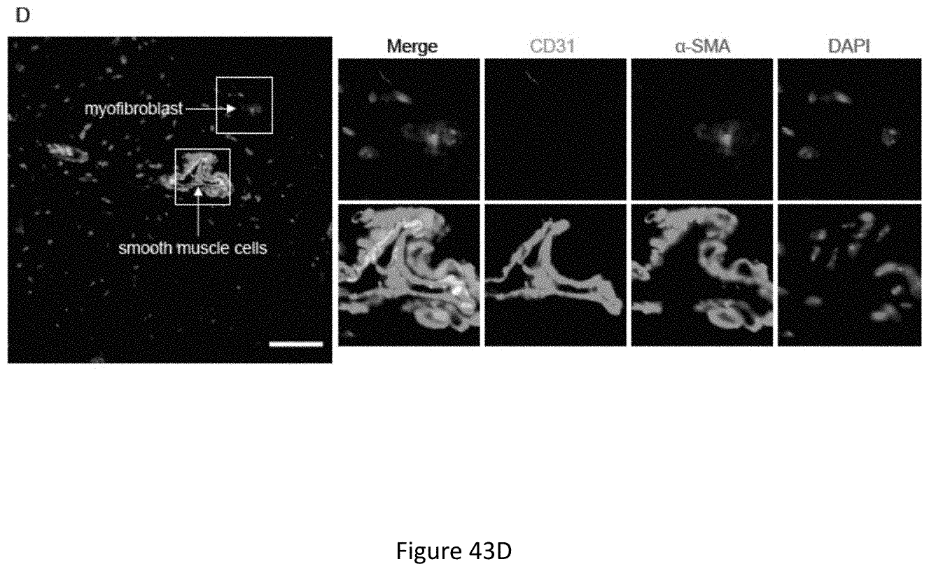

To directly examine myofibroblast differentiation in vivo, Mcu^{-/-} mice were crossbred with a fibroblast specific (Col1a2 cis-acting fibroblast-specific enhancer with minimal promoter), tamoxifen (tamox)-inducible Cre transgenic mouse (Col1a2 CreERT) (FIG. 36A). The Col1a2-CreERT transgenic mouse in genetic fate mapping experiments has been shown to only express Cre in the fibroblast population −99% of labeled cells expressed the cardiac fibroblast markers DDR2 and vimentin and 99% were negative for the endothelial markers VECAD and CD31. Further, no cardiac myocytes were observed to express Cre (Ubil et al., 2014). Following tamoxifen administration, cardiac fibroblasts isolated from Mcu^{fl/fl}×Col1a2-CreERT adult mice showed a near complete loss of MCU (FIG. 36B). CIII (subunit UQCRC2) was used as a mitochondrial loading control. The role of cardiac fibroblast MCU was evaluated using two in vivo models known to promote myofibroblast formation and cardiac fibrosis—myocardial infarction (MI) and chronic infusion of AngII. MI results in significant cell death, initiating myofibroblast differentiation to generate a fibrotic scar to replace lost myocytes and prevent LV wall rupture (Weber et al., 2013). Mice were injected intraperitoneal (i.p.) with tamox (40 mg/kg) for 10 days followed by a 10-day rest period before acquisition of baseline echocardiography. One-week later mice underwent surgical ligation of the left coronary artery (LCA) to induce a large MI and left ventricular (LV) structure and function was tracked weekly by echocardiography (FIG. 36C). In both experimental and control groups, MI induced significant cardiac dysfunction and this was exacerbated in Mcufl/fl×Colla2-CreERT mice (FIG. 33D-F). Loss of fibroblast MCU (Mcufl/fl×Col1a2-CreERT) significantly increased LV dilation, evident by increased LV end-diastolic diameter (LVEDD) and end-systolic diameter (LVESD), as well as reduced fractional shortening (FS) 2-4 weeks post-MI compared to Col1a2-CreERT controls (FIG. 36D-36F). A significant increase in LV end-diastolic volume (LVEDV), LV end-systolic volume (LVESV), and reduced ejection fraction (EF) was observed in Mcu^{fl/fl}×Colla2-CreERT vs. Col1a2-CreERT mice (FIG. 43A-43C). All echocardiographic parameters are reported in Table 5. Loss of fibroblast MCU significantly exacerbated heart weight to tibia length ratios (HW/TL) and lung edema (wet-dry lung weight) 4 weeks post-MI, suggesting an increase in hypertrophy and/or edema and inflammation, both of which are associated with fibrosis (FIG. 36G-36H) (Fujiu and Nagai, 2014; Reed et al., 2010; Wynn and Ramalingam, 2012). Masson's trichrome staining of mid-ventricle cross-sections revealed increased collagen deposition in Mcufl/fl×Colla2-CreERT mice compared to Col1a2-CreERT controls (FIG. 36I).

Quantification of fibrosis in the border and remote zones revealed a more than 2.5-fold increase in Mcu^{fl/fl}×Col1a2-CreERT hearts versus Col1a2-CreERT controls (FIG. 36J). Importantly, the increased fibrosis can be attributed to enhanced myofibroblast formation, which was assessed by immunofluorescence staining for α-SMA and CD31 (PECAM-1, marker of endothelial cells). Using this technique, blood vessels co-stain for both α-SMA and CD31, while myofibroblasts only stain positive for α-SMA (FIG. 43D) (Kanisicak et al., 2016). Mcu^{fl/f}×Col1a2-CreERT hearts displayed increased myofibroblasts compared to Col1a2-CreERT controls in the remote zone 4 weeks post-MI (FIG. 36K).

TABLE 5
Echocardiographic results of left-ventricular (LV) function at baseline (wk 0) and post-MI.

| | Wks | | IVS; d | IVS; s | LVEDD | LVESD | LVEDPW | LVESPW |
|---|---|---|---|---|---|---|---|---|
| | post-MI | n | (mm) | (mm) | (mm) | (mm) | (mm) | (mm) |
| Col1a2-Cre | 0 | 10 | 0.69 ± 0.07 | 1.00 ± 0.09 | 3.53 ± 0.18 | 2.35 ± 0.17 | 0.68 ± 0.03 | 0.96 ± 0.07 |
| | 1 | 10 | 0.70 ± 0.04 | 0.90 ± 0.06 | 4.35 ± 0.22 | 3.66 ± 0.30 | 0.71 ± 0.05 | 0.92 ± 0.10 |
| | | | | | | ** | | |
| | 2 | 10 | 0.69 ± 0.04 | 0.94 ± 0.08 | 4.40 ± 0.24 | 3.67 ± 0.32 | 0.72 ± 0.07 | 0.85 ± 0.09 |
| | | | | | | ** | | |
| | 3 | 10 | 0.67 ± 0.06 | 0.91 ± 0.09 | 4.80 ± 0.29 | 3.94 ± 0.30 | 0.67 ± 0.05 | 0.87 ± 0.08 |
| | | | | | ** | *** | | |
| | 4 | 10 | 0.68 ± 0.04 | 1.00 ± 0.08 | 4.60 ± 0.27 | 3.96 ± 0.32 | 0.78 ± 0.08 | 0.87 ± 0.08 |
| | | | | | * | *** | | |
| Mcu^{fl/fl} × | 0 | 20 | 0.90 ± 0.03 | 1.27 ± 0.04 | 3.44 ± 0.07 | 2.31 ± 0.06 | 0.87 ± 0.06 | 1.18 ± 0.07 |
| Col1a2-Cre | | | ## | | | | | |
| | 1 | 20 | 0.66 ± 0.02 | 0.93 ± 0.06 | 4.77 ± 0.21 | 4.16 ± 0.25 | 0.76 ± 0.05 | 0.90 ± 0.06 |
| | | | *** | *** | *** | *** | | ** |
| | 2 | 20 | 0.54 ± 0.03 | 0.67 ± 0.05 | 4.88 ± 0.16 | 4.56 ± 0.17 | 0.60 ± 0.06 | 0.69 ± 0.06 |
| | | | *** | *** # | *** | *** | ** | *** |
| | 3 | 20 | 0.61 ± 0.04 | 0.78 ± 0.07 | 5.39 ± 0.19 | 5.04 ± 0.21 | 0.64 ± 0.06 | 0.68 ± 0.06 |
| | | | *** | *** | *** | *** ## | ** | *** |
| | 4 | 20 | 0.62 ± 0.05 | 0.72 ± 0.07 | 5.55 ± 0.23 | 5.21 ± 0.24 | 0.61 ± 0.05 | 0.67 ± 0.04 |
| | | | *** | *** # | *** # | *** ## | ** | *** |

| | Wks | EF | FS | LVEDV | LVESV |
|---|---|---|---|---|---|
| | post-MI | (%) | (%) | (μl) | (μl) |
| Col1a2-Cre | 0 | 63.43 ± 2.74 | 33.94 ± 1.92 | 54.11 ± 7.41 | 20.81 ± 4.29 |
| | 1 | 34.83 ± 5.36 | 16.95 ± 2.81 | 88.12 ± 10.45 | 61.86 ± 12.00 |
| | | *** | *** | | |
| | 2 | 36.18 ± 5.56 | 17.79 ± 3.03 | 91.32 ± 11.98 | 63.14 ± 12.63 |
| | | *** | *** | | |
| | 3 | 37.80 ± 3.78 | 18.47 ± 2.01 | 112.43 ± 16.19 | 73.19 ± 13.27 |
| | | *** | *** | * | |
| | 4 | 31.25 ± 3.87 | 14.86 ± 1.98 | 101.94 ± 14.43 | 74.53 ± 14.25 |
| | | *** | *** | | * |
| Mcu^{fl/fl} × | 0 | 62.54 ± 1.24 | 33.06 ± 0.88 | 49.52 ± 2.41 | 18.71 ± 1.20 |
| Col1a2-Cre | | | | | |
| | 1 | 28.72 ± 3.28 | 13.71 ± 1.65 | 111.30 ± 12.27 | 84.58 ± 12.95 |
| | | *** | *** | *** | *** |
| | 2 | 14.68 ± 1.75 | 6.63 ± 0.83 | 115.12 ± 9.72 | 99.42 ± 9.53 |
| | | *** ### | *** ### | *** | *** |
| | 3 | 14.71 ± 2.03 | 6.73 ± 0.97 | 145.79 ± 13.30 | 126.27 ± 13.46 |
| | | *** ### | *** ### | *** | *** # |
| | 4 | 13.96 ± 1.95 | 6.39 ± 0.94 | 157.11 ± 14.40 | 136.95 ± 14.26 |
| | | *** ### | *** ## | *** # | *** ## |

To further define the centrality of ₘCa²⁺ exchange in myofibroblast formation, AngII infusion was employed as a secondary model. AngII is a direct stimulus of myofibroblast formation, and neurohormonal stress resulting from chronic increases in AngII levels is well documented to induce cardiac fibrosis both clinically and experimentally (Crowley et al., 2006; Mehta and Griendling, 2007; Romero et al., 2015). Mice were injected i.p. with tamox (40 mg/kg) for 10 days followed by a 10-day rest period before subcutaneous implantation of Alzet mini-osmotic pumps to deliver AngII (1.1 mg/kg/day) for 4 weeks (FIG. 36L). Mice were sacrificed after 4 weeks and hearts were fixed and stained for fibrosis. Masson's trichrome staining of mid-ventricle cross-sections revealed increased collagen deposition throughout the heart in Mcu^{fl/fl}×Col1a2-CreERT mice compared to Col1a2-CreERT controls (FIG. 33M). Quantification of interstitial fibrosis revealed a significant increase in Mcufl/fl×Colla2-CreERT hearts versus Col1a2-CreERT controls (FIG. 36N). In addition, chronic AngII increased myofibroblast formation in Mcu^{fl/fl}×Col1a2-CreERT hearts versus Col1a2-CreERT as determined by α-SMA⁻/CD31⁻ immunohistochemistry staining (FIG. 36O).

Recently, the ₘCa²⁺ field has been transformed by the discovery of many genes that encode ₘCa²⁺ transporters and channels. The biophysical properties of mtCU-mediated Ca²⁺ influx have been extensively studied in many cell types, and the role of ₘCa²⁺ as a regulator of bioenergetics and cell death is well documented. ₘCa²⁺ is integrated into the mitochondria and directly impacts cellular energetics. In addition, ₘCa²⁺ overload promotes necrotic cell death through opening of the mitochondria permeability transition pore. The data presented herein links changes in ₘCa²⁺ with epigenetic modulation of the gene program to drive cellular differentiation. This study provides evidence that extracellular fibrotic signaling alters mitochondrial function in order to drive transcriptional changes in the nucleus.

Loss of ₘCa²⁺ uptake was sufficient to promote fibroblast to myofibroblast conversion and enhance the myofibroblast phenotype. Fibroblast-specific deletion of Mcu in adult mice augmented myofibroblast formation and fibrosis post-MI and chronic AngII administration. Further, fibrotic agonists signal was found to acutely down-regulate ₘCa²⁺ uptake by rapidly increasing the expression of the mtCU gatekeeper, MICU1. Although attributed to another mechanism, TGFβ-mediated reduction of ₘCa²⁺ uptake was also observed in smooth muscle cells—pretreatment with TGFβ reduced ₘCa²⁺ uptake in the face of increased cCa²⁺ (Pacher et al., 2008). Given the noted role of MICU1 to negatively regulate uptake at signaling levels of _{c}Ca²⁺ [<2 μm], it is hypothesized herein that fibrotic agonists signal to acutely inhibit ₘCa²⁺ uptake to initiate myofibroblast differentiation (Antony et al., 2016; Csordas et al., 2013; Kamer and Mootha, 2014; Mallilankaraman et al., 2012; Patron et al., 2014). The data presented herein suggest that extracellular stimuli are regulating cellular processes by directly altering mitochondrial signaling. The outcome of this is two-fold. In addition to essential changes in mitochondrial metabolism upstream of epigenetic reprogramming, modulation of the ₘCa²⁺ microdomain is a way to enhance canonical cytosolic signaling pathways.

Examination into mechanisms of pluripotency versus differentiation has revealed the importance of metabolism at several levels, prompting evaluation of the relationship between ₘCa²⁺ uptake, metabolism, and myofibroblast differentiation. Fibrotic agonists increased glycolysis and loss of MCU augmented this phenotype. Mechanistically, using mutant PFK2/FBP2 transgenes to constitutively increase or decrease glycolysis, it was shown herein that enhanced glycolysis alone is sufficient to promote differentiation, whereas inhibition of glycolysis reverted the gain-of-function phenotype noted in Mcu^{-/-} fibroblasts. This data is consistent with other studies which have shown glycolytic reprogramming correlates with myofibroblast differentiation and fibrosis (Bernard et al., 2015; Xie et al., 2015). Glycolytic reprogramming is a well-substantiated phenomenon which allows for the diversion of glycolytic intermediates into ancillary metabolic pathways in order to generate building blocks for the biosynthesis of macromolecules (DeBerardinis et al.; Ghesquière et al., 2014; Vander Heiden et al., 2009). These data suggest that increased glycolytic flux is necessary to fulfill cellular anabolic needs, in this case de novo protein translation, required for myofibroblast differentiation. It is hypothesized herein that the loss of ₘCa²⁺ uptake promoted aerobic glycolysis by reducing the activity of key Ca²⁺ dependent enzymes. Indeed the phosphorylation status of PDH in response to fibrotic agonists and Mcu^{-/-} fibroblasts suggested inactivity and thereby pyruvate was hindered from entering the TCA cycle. In correlation with these results, data obtained from ovarian cancer cell lines showed that MICU1 expression promoted the inhibition of PDH and aerobic glycolysis (Chakraborty et al., 2017).

Metabolomic analysis revealed a multitude of changes induced by both TGFP and the loss of MCU. In addition to increased levels of pyruvate, consistent with inactive PDH, metabolite quantification showed TGFP increased αKG ˜2-fold in TGF⊕-treated fibroblasts and this increase was augmented by loss of ₘCa²⁺ uptake. αKG is not restricted to its role as a TCA cycle intermediate but also is a powerful signaling molecule. Of particular interest is the role of αKG in promoting histone and DNA demethylation by modulating αKG-dependent TET enzymes and JmjC-KDMs (Klose et al., 2006; Loenarz and Schofield, 2011). Previous studies have suggested that αKG regulates the balance between pluripotency and lineage-commitment of embryonic stem cells (ESCs). αKG maintained pluripotency of ESCs by promoting JmjC-KDM- and TET-dependent demethylation, permitting gene expression to support pluripotency (Carey et al., 2015). Interestingly, in the same manner, αKG accelerated the differentiation of primed human pluripotent stem cells (TeSlaa et al., 2016). While no major changes were observed in global DNA methylation, TGFβ and loss of MCU induced dynamic changes in histone lysine methylation at residues regulated by JmjC-KDMs. Specifically, TGFP significantly reduced global H3K27me2 marks and Mcu^{-/-} MEFs displayed reduced H3K27me2 compared to controls at baseline and post-TGFβ. Importantly, it is demonstrated herein that TGFβ induces the loss of H3K27me2 at regulatory myofibroblast gene loci (promoter regions associated with gene activation and predicted binding sites for known fibrotic transcription factors). These data suggest that the observed increase in aKG promotes H3K27me2 demethylation at myofibroblast-specific genes in order to promote differentiation. Since PDH-mediated pyruvate entry into the TCA cycle was inhibited, it is suspected that anaplerotic pathways are being activated to replenish TCA cycle intermediates. The data presented herein suggest that the increased level of αKG associated with differentiation is being generated through the pyruvate carboxylase pathway and/or glutaminolysis (DeBerardinis et al.; Owen et al., 2002). Pyruvate carboxylase activity is documented in cancer cells to mediate glucose-derived pyruvate to enter the TCA cycle at the level of oxaloacetate (Cheng et al., 2011). Further, one study showed that cancer cells with inhibited PDH activity have increased anaplerotic contribution through PC (Izquierdo-Garcia et al., 2014). The second major replenishment pathway is through glutaminolysis which is a two-step process that converts glutamine to glutamate to αKG (DeBerardinis et al., 2007; Krebs, 1935; Le et al., 2012; Salabei et al., 2015; Yang et al., 2014). This is a more likely scenario suggested by the increased αKG/Gln ratio post-TGFβ. In addition to providing carbons to the TCA cycle through αKG, glutamine metabolism contributes to many other cellular processes such as nucleotide synthesis, amino acid production, fatty acid synthesis, and control of reactive oxygen species (Altman et al., 2016). While αKG increased post-TGFβ, metabolite levels in the aforementioned pathways were decreased post-TGFβ, including inosine monophosphate (IMP), glutathione (GSH), γ-Aminobutyric acid (GABA), and Asparagine (FIG. 11), suggesting glutamine is mainly being utilized to form αKG. Interestingly, in cancer cells increases in aerobic glycolytic flux is often associated with enhanced glutaminolysis (DeBerardinis et al., 2007; Le et al., 2012). Given the similarities with this model, it's intriguing to conjecture that the mtCU may play a similar role in these cell systems.

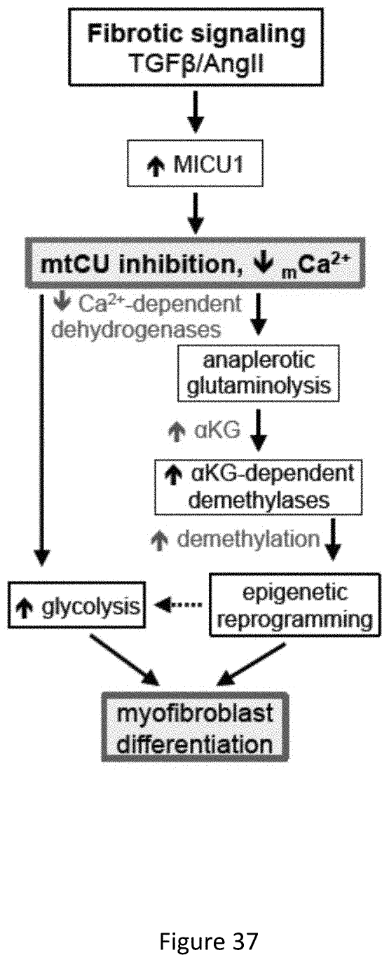

In summary, the data presented herein demonstrates that loss of ₘCa²⁺ uptake promotes myofibroblast differentiation both in vitro and in vivo. Until now, the role of ₘCa²⁺ uptake in cellular differentiation or epigenetic regulation has not been explored, but these studies reveal its importance in the myofibroblast differentiation process through concerted alterations in both metabolism and epigenetics. In addition, these findings support an endogenous role for decreased mtCU-mediated ₘCa²⁺ uptake as an essential element of the differentiation process (FIG. 37).

### Example 5: Mitochondrial Na⁺/Ca²⁺ Exchanger Reverses Neuropathology in Alzheimer's Disease

The data presented herein demonstrates that neuronal deletion of NCLX in 3xTg-AD mouse causes memory impairment followed by increased amyloidosis, tau-pathology and oxidative stress. These studies suggest that ₘCa²⁺ overload is a primary contributor to AD pathology by promoting superoxide generation, metabolic dysfunction and neuronal cell death. Genetic rescue of ₘCa²⁺ efflux via neuronal expression of the NCLX reduced mitochondrial dysfunction and AD pathology. These results provide a potential missing link between the ‘calcium dysregulation’ and ‘mitochondrial cascade’ hypotheses and advocate targeting mitochondrial calcium exchange as a powerful therapeutic to inhibit or reverse AD progression.

The materials and methods are now described.

Generation of Neuronal Specific NCLX Knockout 3xTg-AD Mutant Mouse.

NCLX knockout mouse generated by acquiring targeted ES cells generated by recombinant insertion of a knockout-1st mutant construct containing loxP sites flanking exons 5-7 of the NCLX gene (ch12: 113298759-113359493). ES cell lines (clone EPD0460_4_A08, EUCOMM) were confirmed by PCR and injected into C57BL/6N blastocysts with subsequent transplantation into pseudo- pregnant females. Germline mutant mice were crossed with ROSA26-FLPe knock-in mice for removal of the FRT-flanked splice acceptor site, βgal reporter, and neomycin resistance cassette. Resultant NCLX^{fl/+} mice were interbred to generate homozygous mutant mice with knockout potential (NCLX^{fl/fl}). Homozygous LoxP ‘foxed’ mice (NCLX^{f1/fl}) were crossed with neuron-specific Camk2a-Cre recombinase driver lines (available from Jackson Laboratory, stock no. 005359), resulting in germline neuronal specific deletion of NCLX. The Calcium/calmodulin-dependent protein kinase II alpha (Camk2a) promoter drives Cre recombinase expression in the forebrain, specifically to the CA1 pyramidal cell layer in the hippocampus. These mice were viable and fertile. Resultant neuronal-specific loss-of-function models (NCLX KO-NCLX^{fl/fl}×Camk2a-Cre) were crossed with 3xTg-AD mutant mouse (3xTg-AD; APP_{swe}, PS1_{M146V}, tau_{P301L}), to generate 3xTg-AD×NCLX^{fl/fl}×Camk2a-Cre) mutant mice. 3xTg-AD mice are homozygous for the Psen1 mutation (M146V knock-in), and contain transgenes inserted into the same loci expressing the APP_{swe} mutation (APP KM670/671NL) and tau mutation (MAPT P301L).

Generation of Neuronal Specific NCLX Overexpression 3xTg-AD Mutant Mouse Model.

The human NCLX sequence (NM_024959) (5′ EcoRI, 3′ XmaI) was cloned into a plasmid containing the Ptight Tet-responsive promoter and a SV40 poly(A) sequence and linearized the construct with XhoI digestion followed by gel and Elutip DNA purification. Upon sequence confirmation the purified fragment was injected into the pronucleus of a fertilized ovum and transplanted into pseudo-pregnant females (C57BL/6N). Upon confirmation of germline transmission in founder lines, mutant mice were crossbred with the Camk2a-tTA (neuronal-restricted expression, doxycycline-off) transgenic model. This allowed conditional overexpression upon the withdrawal of chow containing doxycycline (a tetracycline analogue). Resultant neuronal-specific gain-of-function models (NCLX nTg-TRE-NCLX×Camk2a-tTA) were crossed with 3xTg-AD mutant mouse to generate 3xTg-AD×TRE-NCLX×Camk2a-tTA mutant mice. All mice were maintained under pathogen-free conditions on a 12 hour light/12 hour dark cycle with continuous access to food and water.

Human AD Tissue Samples.

Frontal cortex samples were collected post-mortem from non-familial AD patients and age-matched controls with no history of dementia. All tissue samples were rapidly frozen in liquid nitrogen and stored at −80° C. until isolation of protein (n=7 for non-familial AD and n=7 for familial AD).

Cell Cultures and Differentiation

Mouse neuroblastoma N2a cell line as control cells (N2a/con) and N2a cells stably expressing human APP carrying the K670 N, M671 L Swedish mutation (APPswe) were grown in Dulbecco's modified Eagle's medium supplemented with 10% fetal bovine serum, 1% penicillin/streptomycin and in the absence (N2a/con) or presence of 400 μg/mL G418 (APPswe) at 37° C. in the presence of 5% CO2. In differentiation studies, cells were grown in 50% Dulbecco's modified Eagle's medium (DMEM), 50% OPTI-MEM, 1% penicillin/streptomycin (GIBCO) for 72 hours. Only cells with passage number <20 were used. For all imaging studies, cells were plated on glass coverslips pre-coated with poly-D-lysine. For overexpression of NCLX, maturated N2a Con and APPswe were infected cells with adenovirus encoding NCLX (Ad-NCLX) for 48 hours.

qPCR mRNA Analysis.

RNA was extracted using the Qiagen RNeasy Kit. Briefly, 1 μg of total RNA was used to synthesize cDNA in a 20 μL reaction using the High-Capacity cDNA Reverse Transcription Kit (Applied Biosystems). qPCR analysis was conducted following manufacturer instructions (Maxima SYBR, Thermo Scientific). RPS-13 was always used as an internal control gene to normalize for RNA. Each sample was run in duplicate, and analysis of relative gene expression was done by using the 2^{-ΔΔ}Ct method.

Live-Cell Imaging of Ca²⁺ Transients

Maturated neuronal cells were infected with Ad-ₘᵢₜₒR-GECO-1 to measure ₘCa²⁺ dynamics or loaded with the cytosolic Ca²⁺ indicator, 5-μM Fluo4-AM to study cytosolic Ca²⁺ dynamics. Cells were imaged continuously in Tyrode's buffer (150-mM NaCl, 5.4-mM KCl, 5-mM HEPES, 10-mM glucose, 2-mM CaCl2, 2-mM sodium pyruvate at pH 7.4) on a Zeiss 510 confocal microscope. Cell were treated with the depolarizing agent, 100 mM KCl, to activate voltage-gated calcium channels during continuous live-cell imaging.

Evaluation of ₘCa²⁺ Retention Capacity and Content

To evaluate ₘCa²⁺ retention capacity and content, N2a as con, APPswe and APPswe infected with Ad-NCLX for 48 hours were transferred to an intracellular-like medium containing (120-mM KCl, 10-mM NaCl, 1-mM KH2PO₄, 20-mM HEPES-Tris), 3-μM thapsigargin to inhibit SERCA so that the movement of Ca²⁺ was only influenced by mitochondrial uptake, 80-μg/ml digitonin, protease inhibitors (Sigma EGTA-Free Cocktail), supplemented with 10-μM succinate and pH to 7.2. All solutions were cleared with Chelex 100 to remove trace Ca²⁺ (Sigma). For ₘCa²⁺ retention capacity: 2×10⁶ digitonin-permeabilized neuronal cells were loaded with the ratiometric reporters FuraFF at concentration of 1-μM (Ca²⁺). At 20 s JC-1 (Enzo Life Sciences) was added to monitor (Δψm) mitochondrial membrane potential. Fluorescent signals were monitored in a spectrofluorometer at 340- and 380-nm ex/510-nm em. After acquiring baseline recordings, at 400 s, a repetitive series of Ca²⁺ boluses (10 μM) were added at the indicated time points. At completion of the experiment the protonophore, 10-μM FCCP, was added to uncouple the Δψm and release matrix free-Ca²⁺. All experiments (3 replicates) were conducted at 37° C. For ₘCa²⁺ content cells from all the groups were loaded with Fura2 and treated with digitonin and thapsigargin. Upon reaching a steady state recording, the protonophore, FCCP, was used to collapse ΔΨ and initiate the release of all matrix free Ca²⁺.

Western Blot Analysis

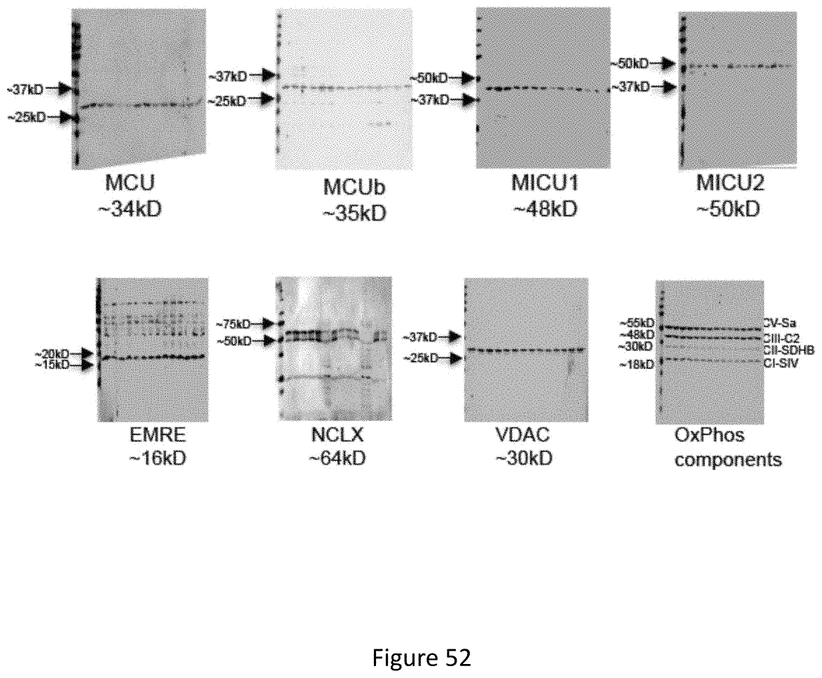

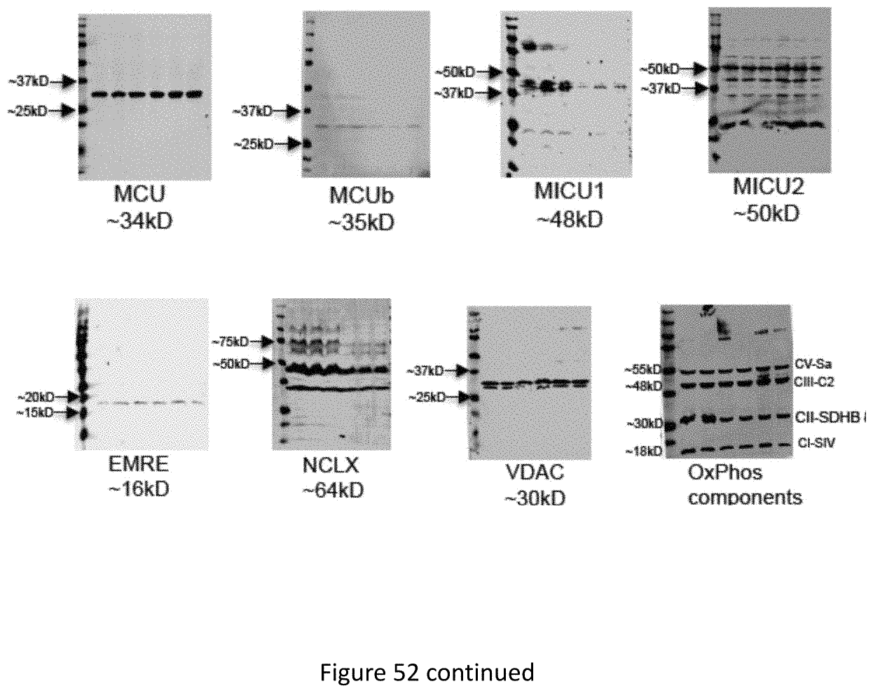

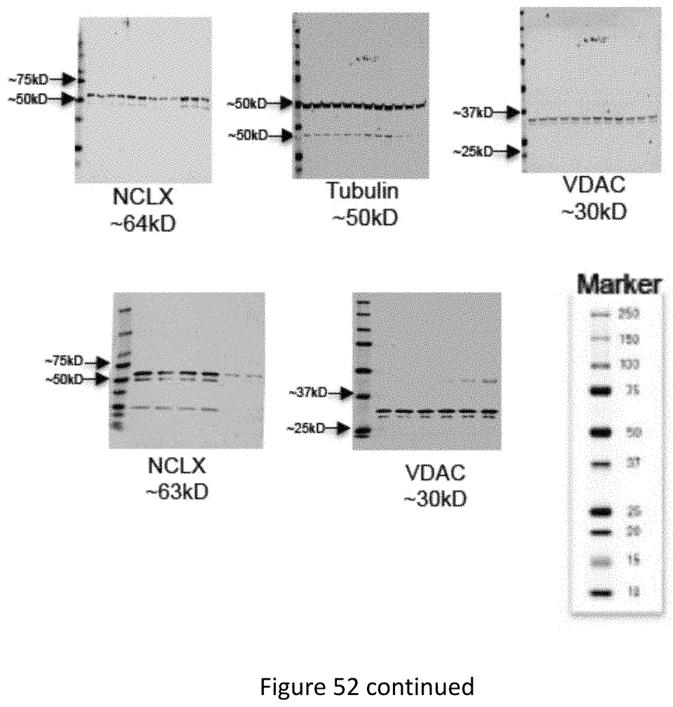

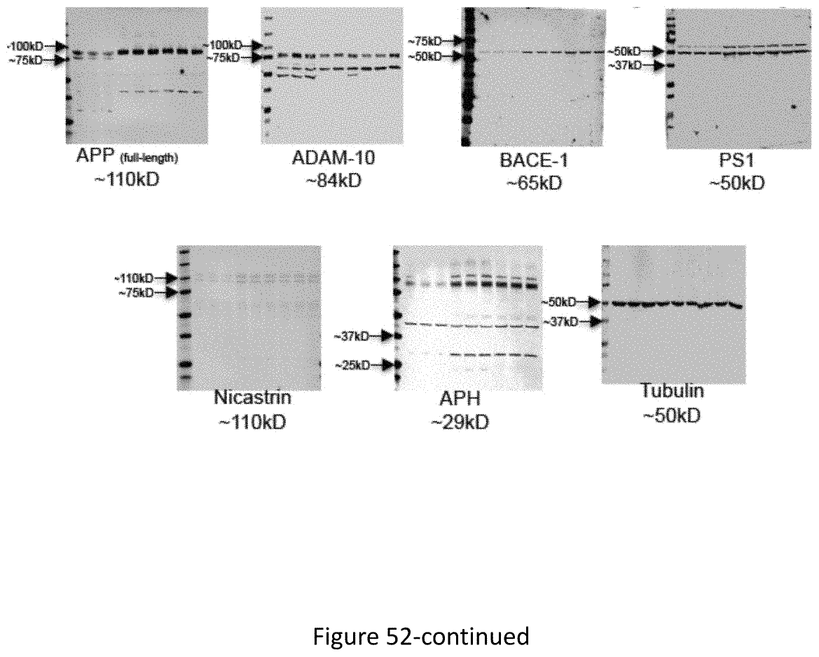

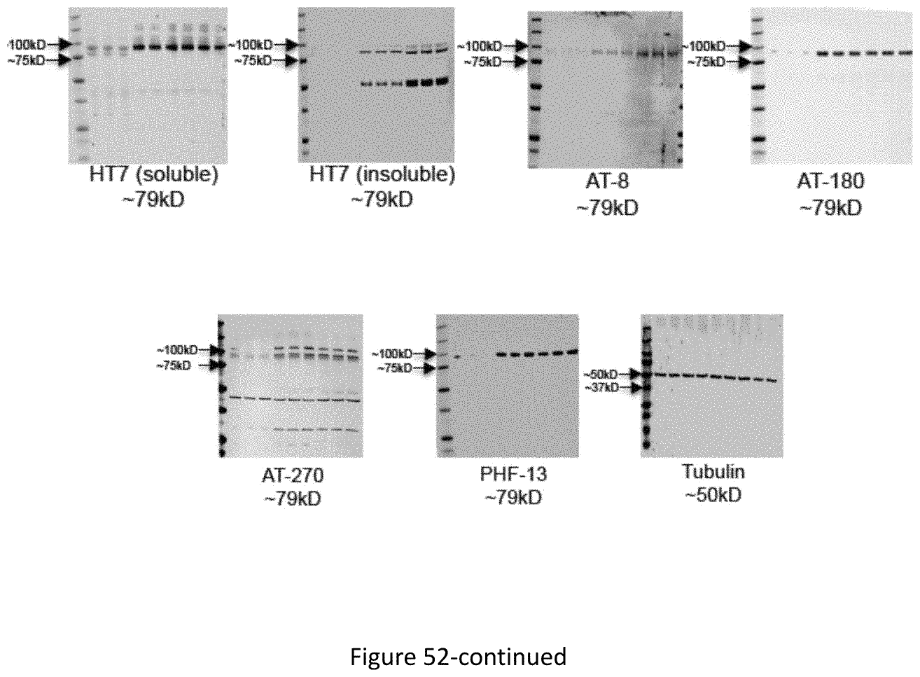

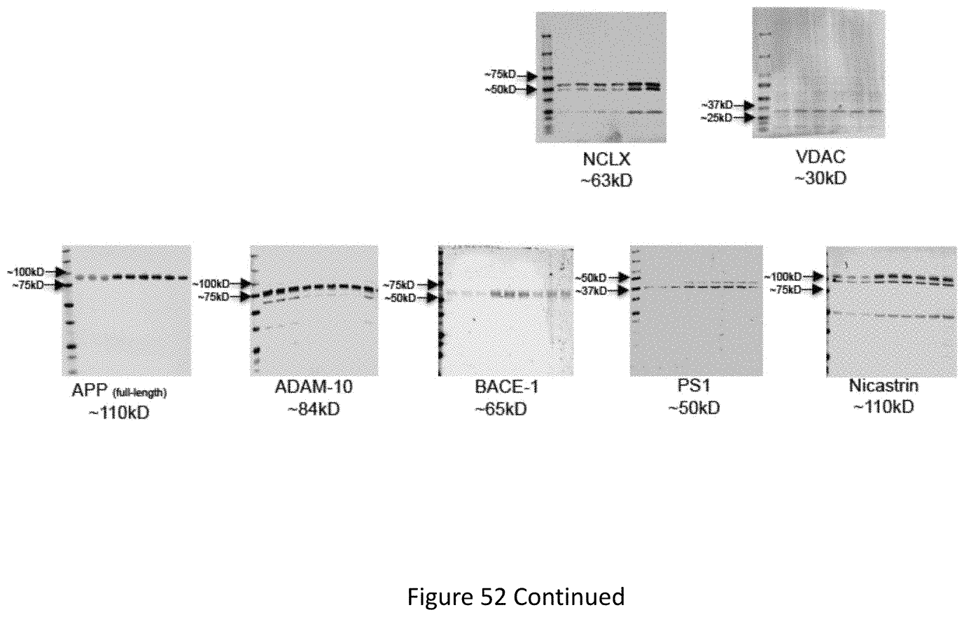

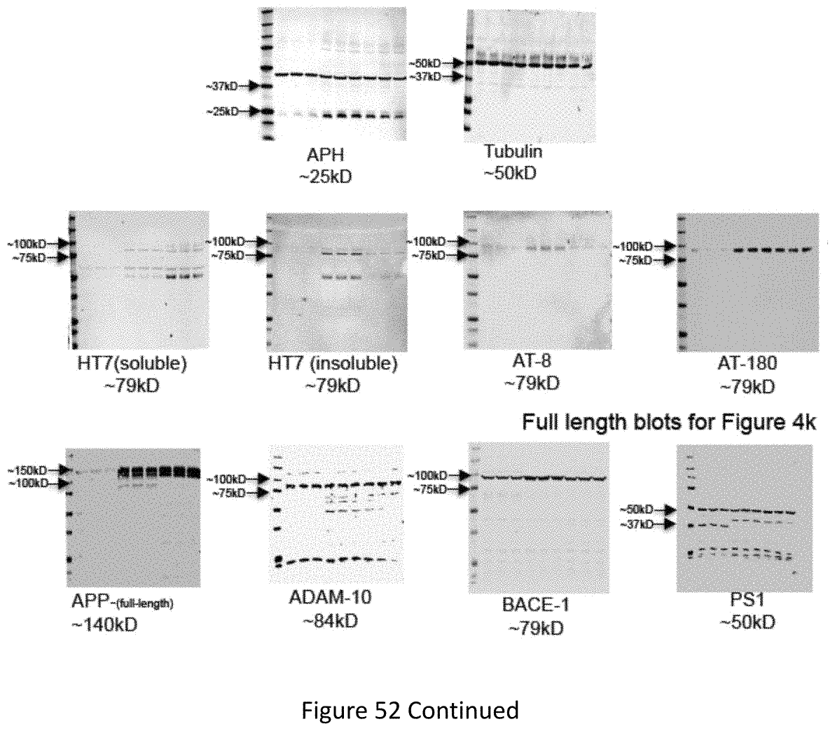

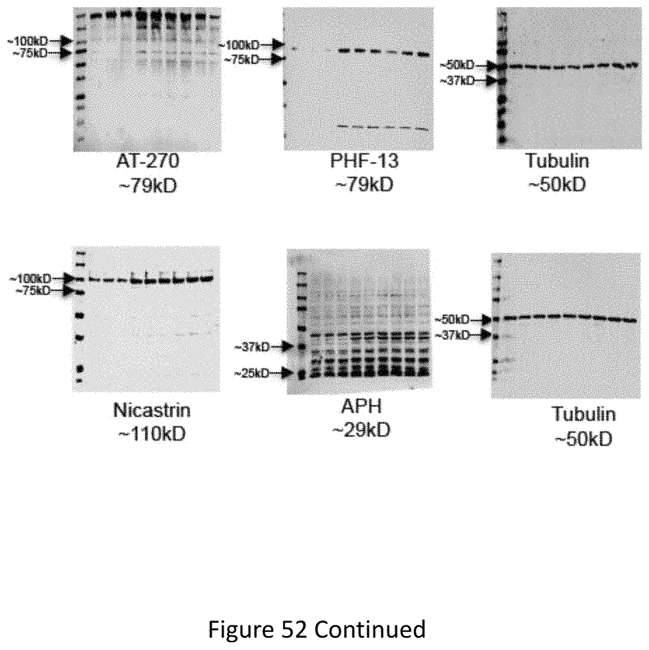

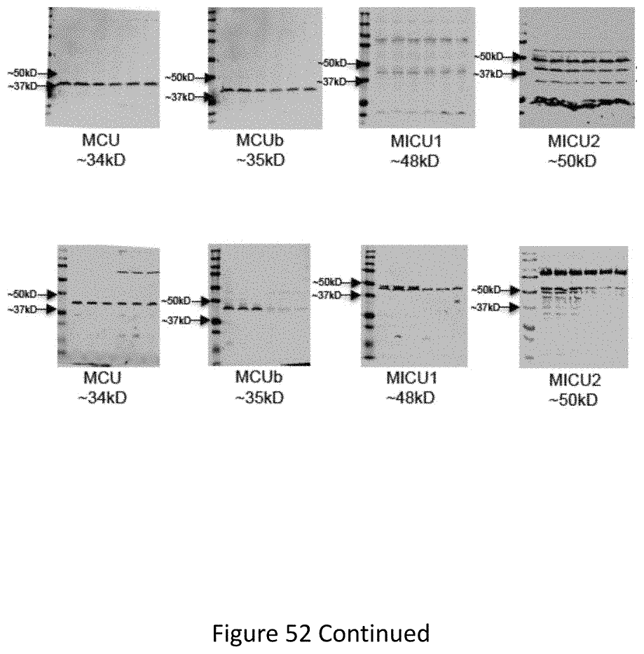

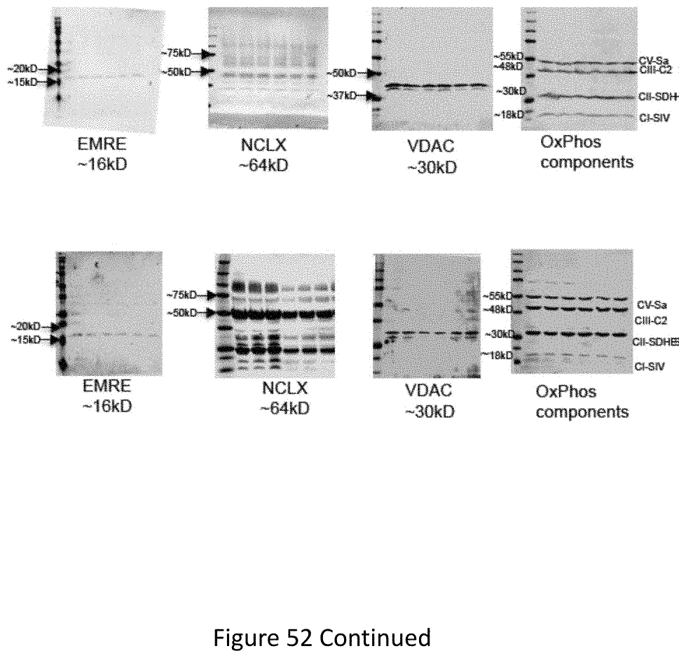

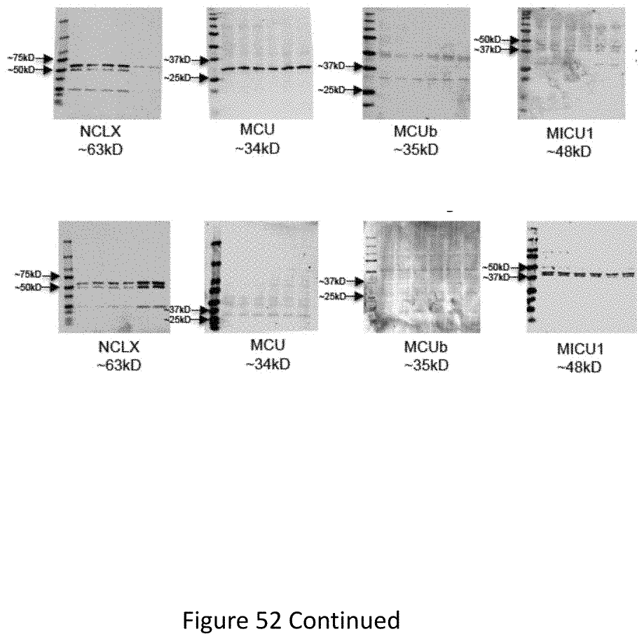

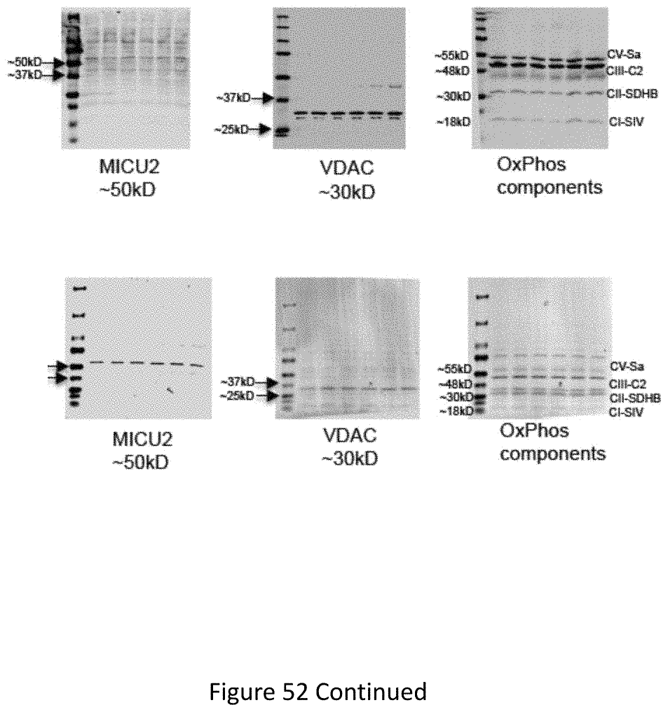

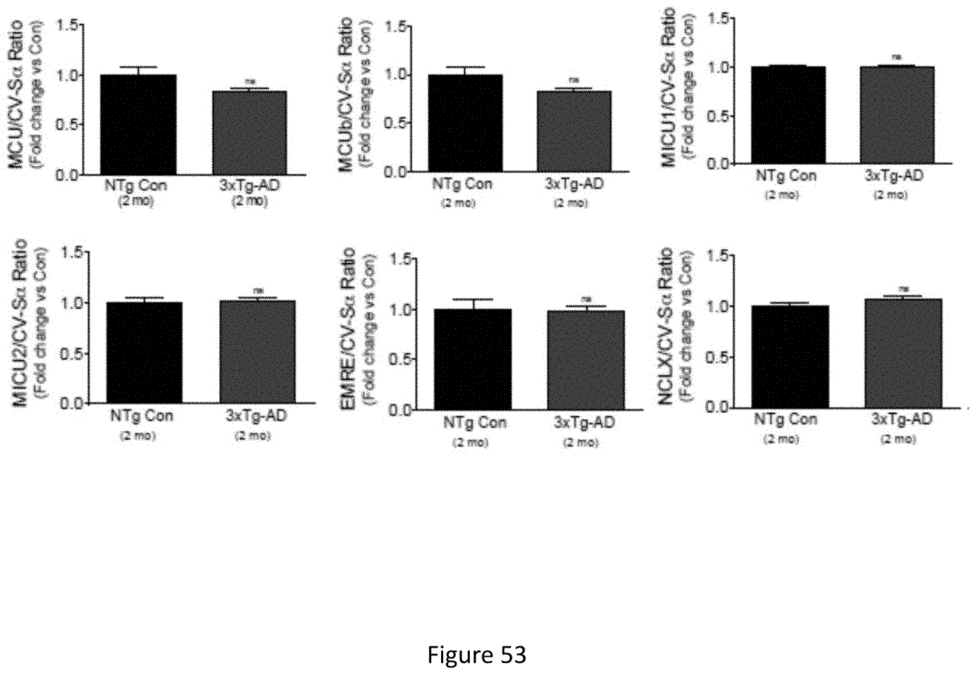

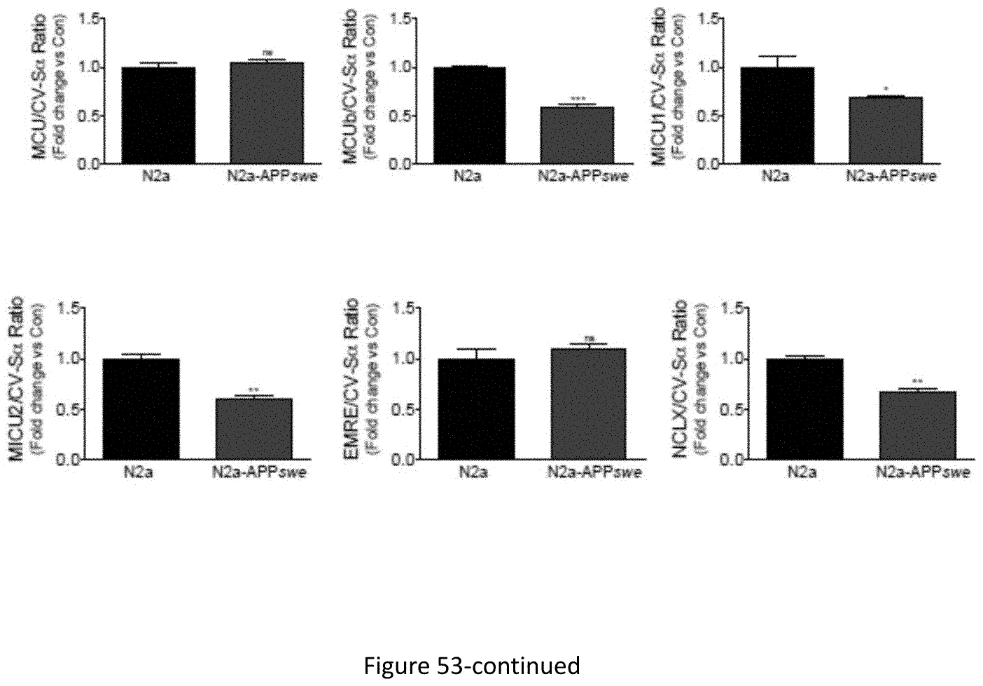

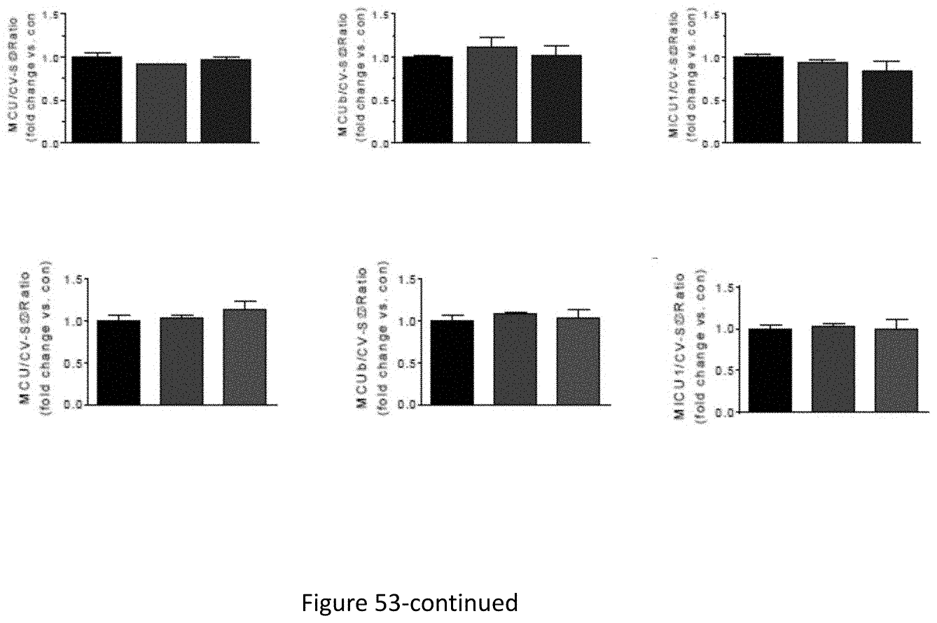

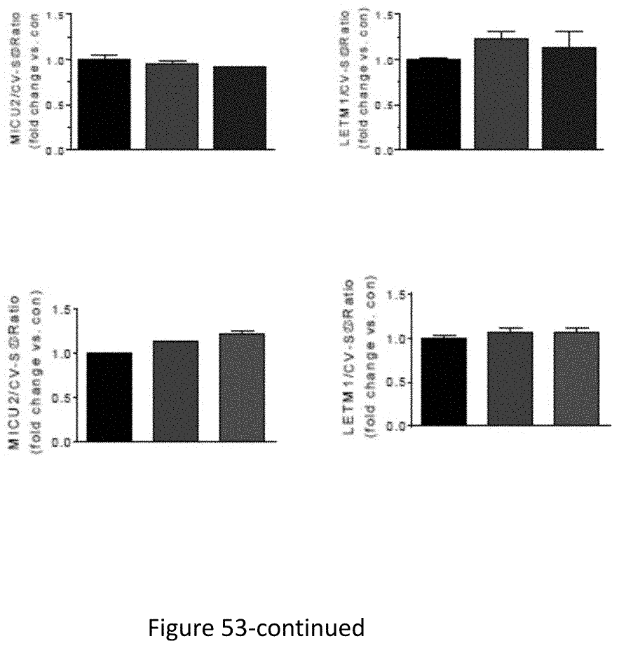

All protein samples from brain or cell lysates were lysed by homogenization in RIPA buffer for the soluble fractions and then in formic acid (FA) for the insoluble fractions and used for western blot analyses. Samples were run by electrophoresis on polyacrylamide Tris-glycine SDS gels. All full length western blots are available in FIG. 52.

Cognition Function Tests

Mice at 6,9 and 12 m of age were assessed for behavioral test in the Y-maze and fear conditioning assay.

Y-Maze

In this test, mice were placed in the center of the Y-maze, and allowed to explore freely through the maze during a 5-min session. This apparatus consisted of three arms 32 cm (long) 610 cm (wide) with 26-cm walls. The sequence and total number of arms entered were recorded. An entry into an arm was considered valid if all four limbs entered the arm. An alternation was defined as three consecutive entries in three different arms (i.e. 1, 2, 3 or 2, 3, 1, etc). The percentage alternation score was calculated using the following formula: Total alternation number/total number of entries-2)*100. Furthermore, total number of arm entries was used as a measure of general activity in the animals. The maze was wiped clean with 70% ethanol between each animal to minimize odor cues.

Fear Conditioning

Briefly, the fear conditioning test was conducted in a chamber equipped with black methacrylate walls, a transparent front door, a speaker, and grid floor. During the training phase, each mouse was placed in the chamber and underwent three cycles of 30 seconds of sound and 10 seconds of electric shock within a 6-minute time interval. The next day, the mouse spent 5 minutes in the chamber without receiving electric shock or hearing the sound (contextual recall). Two hours later, the animal spent 6 minutes in the same chamber but with different flooring, walls, smells, and lighting and heard the cued sound for 30 seconds (cued recall). Freezing activity of the mouse was recorded for each phase.

Immunohistochemistry

Mouse brains were prepared for immunohistochemistry. In brief, serial 6-μm thick sections were deparaffinized, hydrated and blocked in 2% fetal bovine serum before incubation with primary antibody overnight at 4° C. Sections were incubated overnight at 4° C. with primary antibodies Aβ-4G8 (1:150), HT7 (1:150), AT8 (1:50), 4HNE (1:20) then incubated with secondary antibody and developed using the avidinbiotin complex method with 3,30 diaminobenzidine as chromogen.

Biochemical Analysis

Mouse brain homogenates were sequentially extracted first in RIPA for the soluble fractions and then in formic acid (FA) for the insoluble fractions. Briefly, 30 mg of cerebral cortex were sonicated in RIPA buffer added with protease and phosphatase inhibitors cocktail and subsequently ultracentrifuged at 45,000 rpm for 45 minutes. Supernatants were used to measure Aβ and tau soluble fractions by enzyme-linked immunosorbent assay (ELISA) and western blotting, respectively. Pellets were mixed in 70% formic acid, sonicated, neutralized in 6N sodium hydroxide, and used to measure Aβ and tau insoluble fractions by ELISA and Western. Aβ₁₋₄₀ and Aβ₁₋₄₂ levels were assayed by a ELISA kit.

For in vitro analysis of Aβ₁₋₄₀ and Aβ₁₋₄₂ levels, conditioned media from APP_{swe} cells and cells infected Ad-NCLX were collected and analyzed at a 1:100 dilution. Aβ₁₋₄₀ and Aβ₁₋₄₂ in samples were captured with the monoclonal antibody BAN50, which specifically detects the N-terminal of human Aβ₍₁₋₁₆₎. Captured human Aβ is recognized by another antibody, BA27 F(Aβ′)2-HRP, a mAβ specifically detects the C-terminal of Aβ₄₀, or BC05 F(Aβ′)2-HRP, a mAβ specific for the C-terminal of Aβ₄₂, respectively. HRP activity was assayed by color development using TMB. The absorbance was then measured at 450 nm. Values were reported as percentage of Aβ₁₋₄₀ and Aβ₁₋₄₂ secreted relative to control.

Evaluation of Reactive Oxygen Species Production

To measure the total cellular ROS, the fluorogenic probe CellROX Green was used, which is a cell-permeable non-fluorescent or very weakly fluorescent in a reduced state and exhibit strong fluorogenic signal upon oxidation. In this assay, cells were loaded with CellROX green Reagent at a final concentration of 5 μM for 30 min at 37° C. and measured the fluorescence at 485/ex and 520/em using a Tecan Infinite M1000 Pro plate reader. Cells from three groups (n=29 for N2a con; n=30 APPswe; n=31 for APPswe+Ad-NCLX) was stained with 20-μM dihydroethidium for 30 min at 37° C. and imaged on Carl Zeiss 510 confocal microscope at 490/20ex and 632/60em. To measure mitochondrial superoxide production cells were loaded with 10-μM MitoSOX Red for 45 min at 37° C. and imaged at 490/20ex and 585/40em (n=52 for N2a con, n=59 APPswe, and n=59 N2a-APPswe+Ad-NCLX).

Oxygen Consumption Rate

Control (N2a), APPswe and APPswe infected with Ad-NCLX for 48 h were subjected to oxygen consumption rate (OCR) measurement at 37° C. in an XF96 extracellular flux analyzer (Seahorse Bioscience). Cells (3×10⁴) were plated in XF media pH 7.4 supplemented with 25-mM glucose and 1-mM sodium pyruvate and sequentially exposed to oligomycin (1.5 μM), FCCP(1 μM), and rotenone plus antimycin A (0.5 μM).

Membrane Rupture and Cell Viability Assay

Membrane rupture was evaluated using SYTOX Green, a membrane impermeable fluorescent stain, which upon membrane rupture enters the cell, intercalates DNA and increases fluorescence >500-fold and examined general cell viability using Cell Titer Blue (resazurin). This Cell Titer Blue assay uses the indicator dye resazurin to measure the metabolic capacity of cells. Viable cells retain the ability to reduce resazurin into resorufin, which is highly fluorescent. Nonviable cells rapidly lose metabolic capacity, do not reduce the indicator dye, and thus do not generate a fluorescent signal. N2a, APPswe and APPswe infected with Ad-NCLX for 48 h were treated with Iono, (1-5 μM) for 24 h and oxidizing agent tert-Butyl hydroperioxide (TBH) (10-30 μM) for 1 4h and glutamate (NDMAR-agonist, neuroexcitotoxicity agent) (10-50 μM) for 24 h. On the day of the experiment, cells were loaded with 1-μM Sytox green for 15 min at 37° C. and measured the fluorescence at 504/ex and 523/em using a Tecan Infinite M1000 Pro plate reader. To measure number of viable cells, CellTiter-Blue Reagent (10μ1/well in 96 well plate) is added directly to each well, incubated at 37° C. for 2 hrs and the fluorescent signal at (560(20)_{Ex}/590(10)_{Em}) was measured using plate reader.

Fluorometric Detection of β Secretase Activity

β-secretase activity was determined using fluorescent transfer peptides consisting of APP amino acid sequences containing the cleavage sites of BACE secretase. The method is based on the secretase-dependent cleavage of a secretase-specific peptide conjugated to the fluorescent reporter molecules EDANS and DABCYL, which results in the release of a fluorescent signal that was detected using a fluorescent microplate reader with excitation wavelength of 355 nm and emission at 510 nm. The level of secretase enzymatic activity is proportional to the fluorometric reaction, and the data are expressed as fold increase in fluorescence over that of background controls. BACE1 activity was assayed by a fluorescence-based in vitro assay kit.

Detection of Protein Aggregates

For determination of misfolded protein aggregates, cells were fixed with 4% paraformaldehyde at RT for 15 min and, permeabilized in PB ST (0.15% TritonX-100 in PBS) at RT for 15 min. Cells were then stained with proteostat aggresome detection dye at RT for 30 min and Hoechst 33342 nuclear stain, Proteostat, a molecular rotor dye that becomes fluorescent when binding to the β-sheet structure of misfolded proteins. Aggregated protein accumulation was detected using a Carl Zeiss 710 confocal microscope. (standard red laser set for the aggresome signal and DAPI laser set for the nuclear signal imaging). Further quantitative analyses, number of protein aggregates deposits per cell (n=41 for N2a, n=62 APPswe and n=69 APPswe+Ad-NCLX), were counted.

Statistics

All results are presented as mean and +/−SEM. Statistical analysis was performed using Prism 6.0 (Graph Pad Software). All experiments were replicated at least 3 times. Where appropriate column analyses were performed using an unpaired, 2-tailed t-test (for 2 groups) or one-way ANOVA with Bonferroni correction (for groups of 3 or more). For grouped analyses either multiple unpaired t-test with correction for multiple comparisons using the Holm-Sidak method or where appropriate 2-way ANOVA with Tukey post-hoc analysis was performed. P values less than 0.05 (95% confidence interval) were considered significant.

The results are now described.

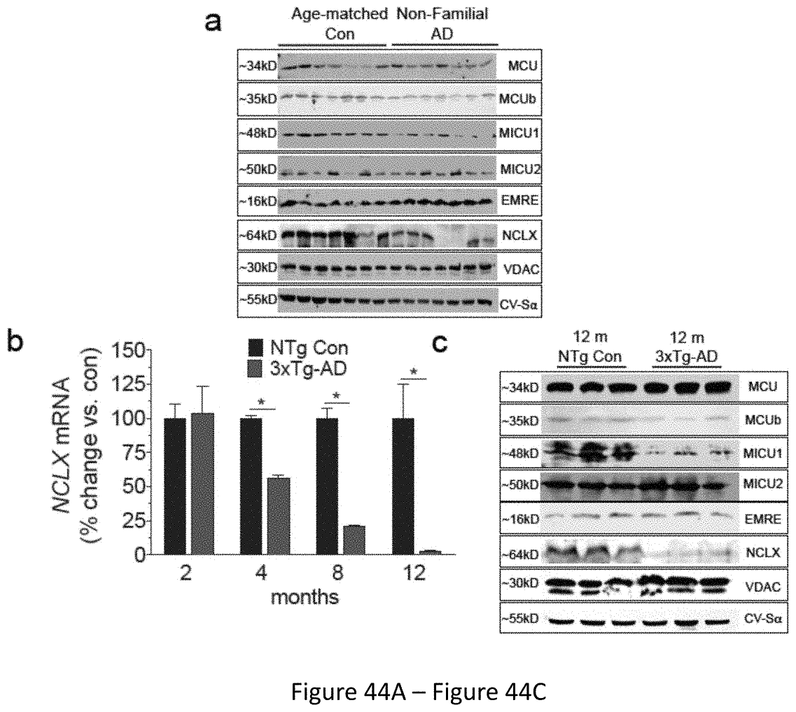

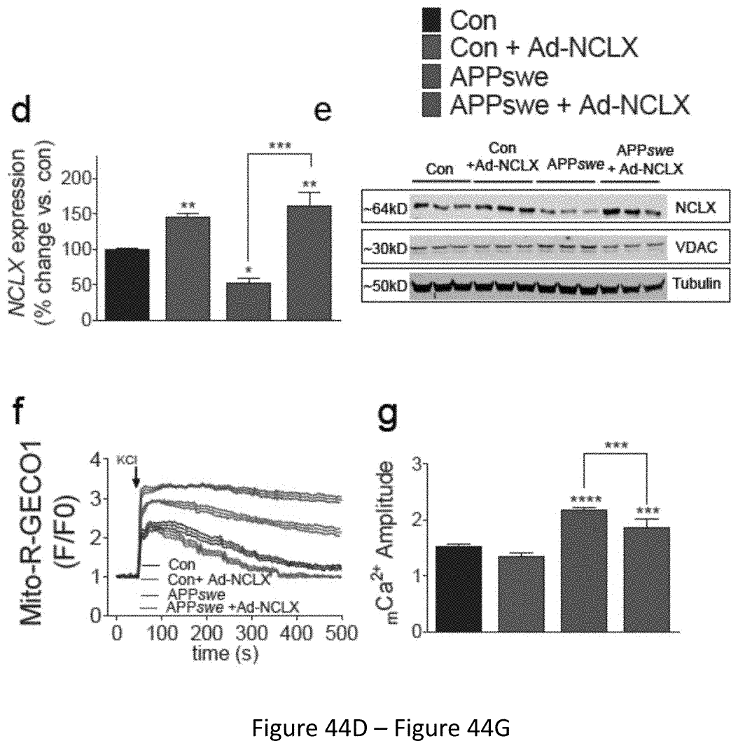

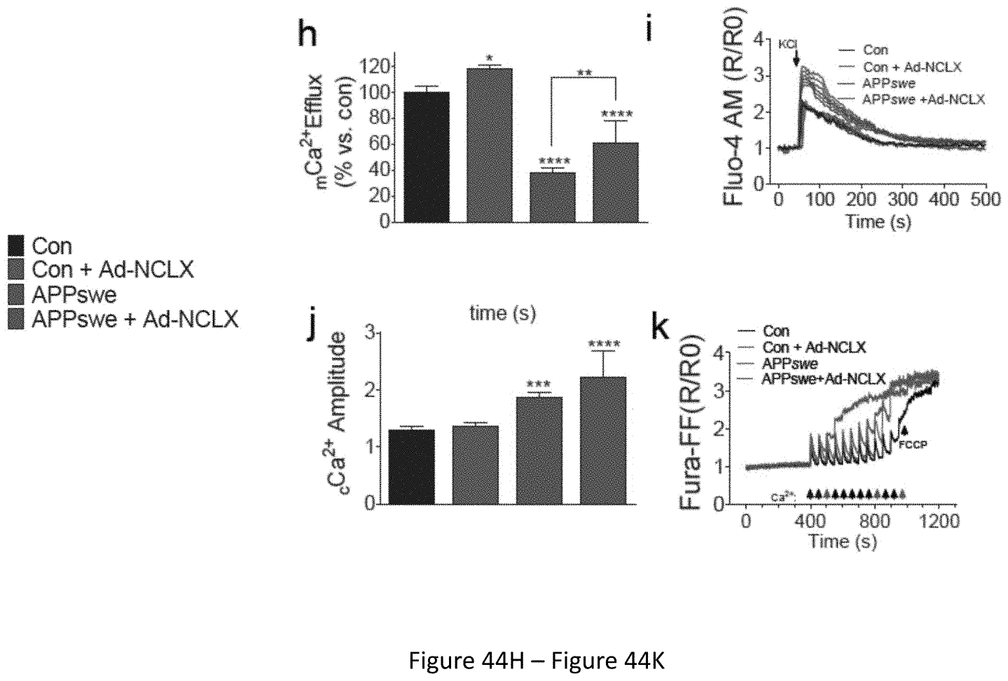

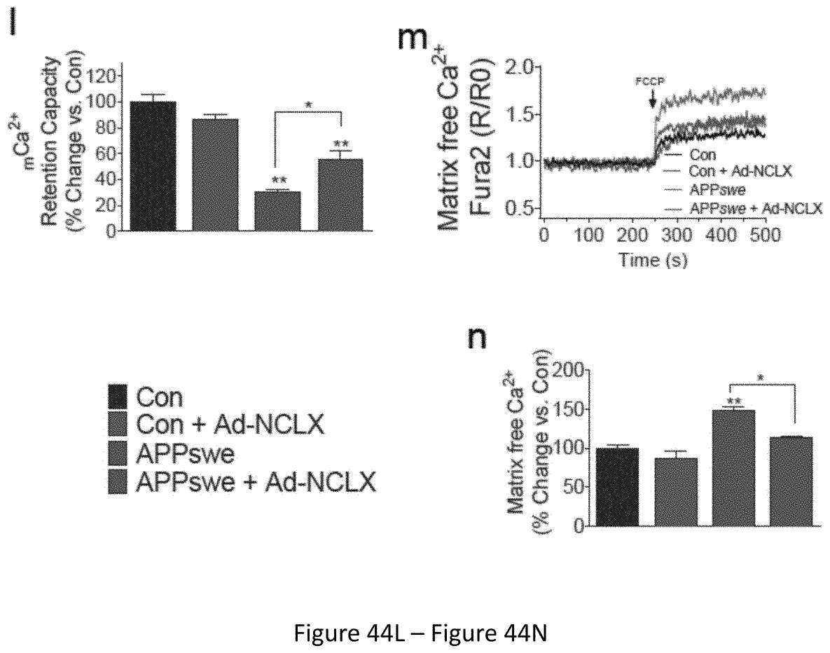

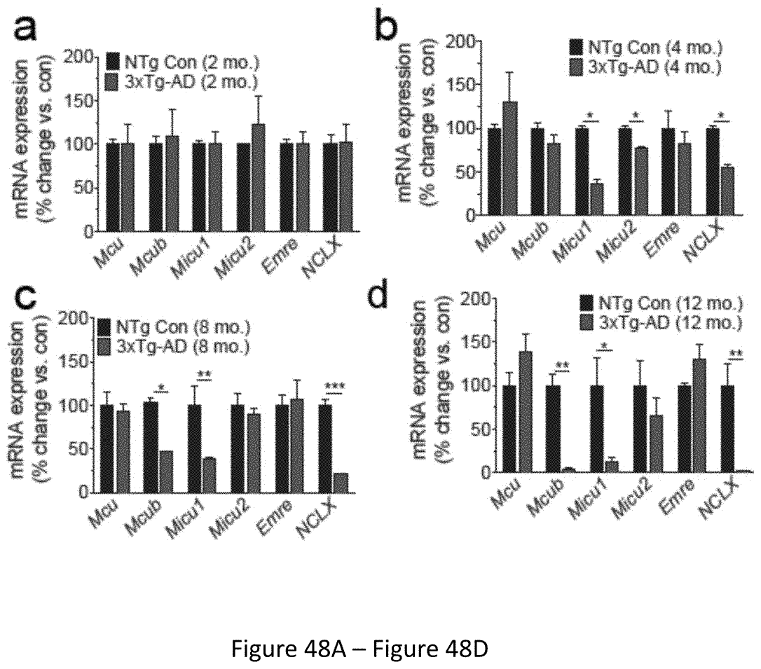

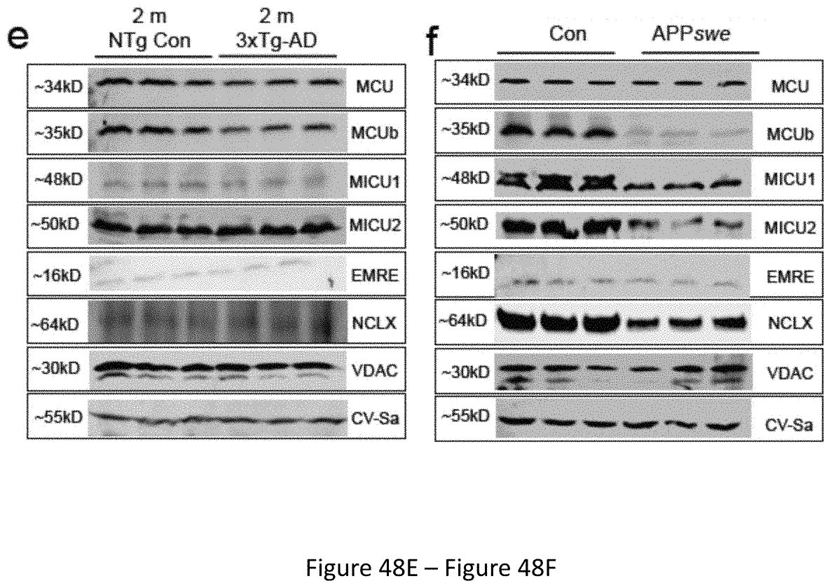

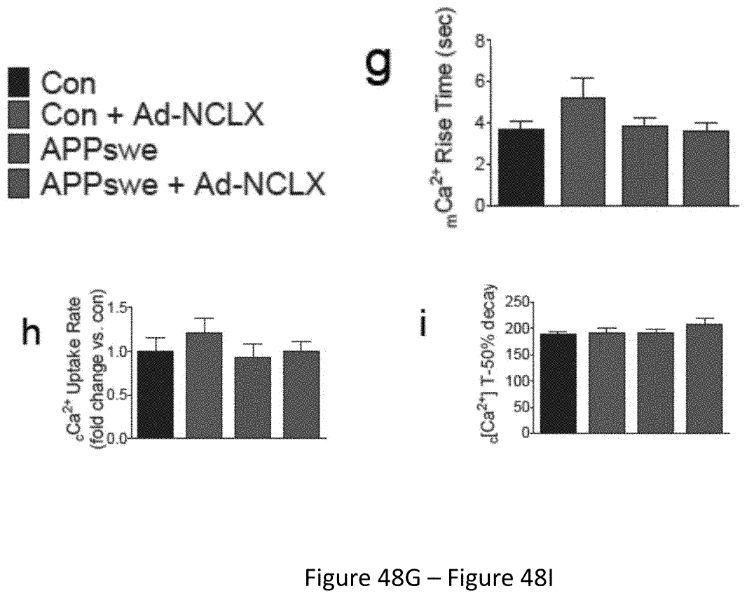

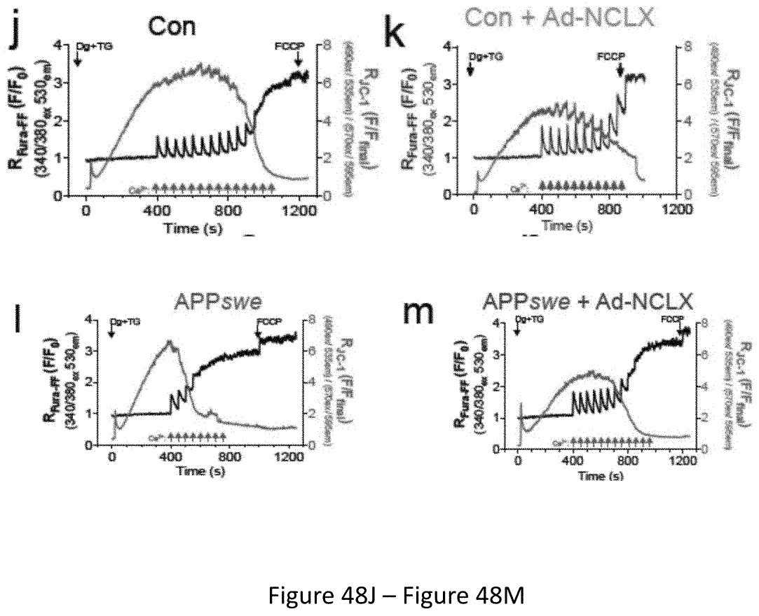

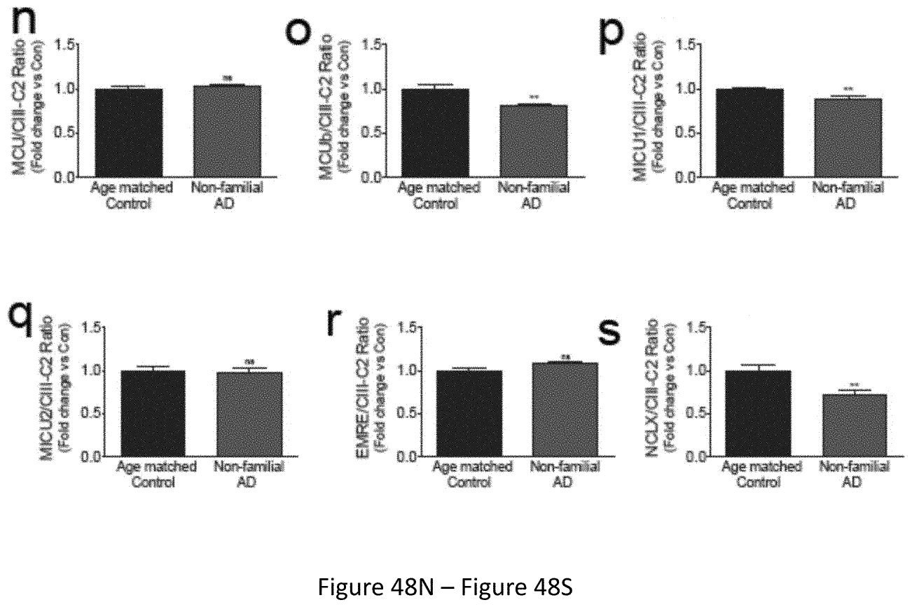

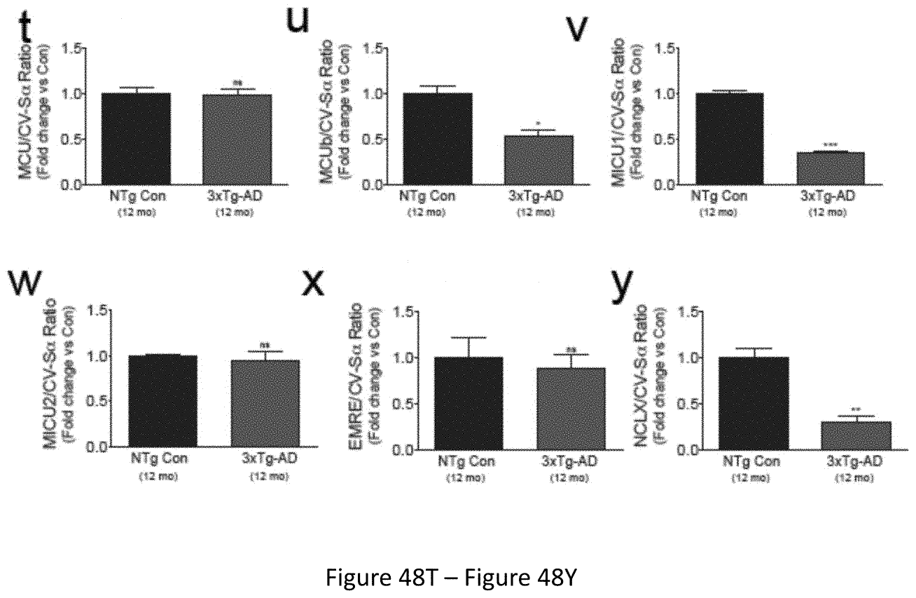

Expression of the mitochondrial Na⁺/Ca²⁺ exchanger (NCLX) is diminished in AD (FIG. 44). Frontal cortex samples were collected post-mortem from non-familial AD patients and age-matched controls with no history of dementia. A substantial reduction in the protein expression of NCLX, the primary mediator of ₘCa²⁺ efflux in excitable cells, was observed in non-familial AD patients (FIG. 44A). In addition, a trend towards a reduction in the MCU negative regulator, MICU1 (inhibitor of uptake at low ᵢCa²⁺), and MCUb (CCDC109B) was also noted. Complex CV-Sα were used as mitochondrial loading controls. These data suggest that alterations in the expression of the ₘCa²⁺ efflux exchange machinery may be a significant contributor to ₘCa²⁺-overload in AD. To examine if the AD patient alterations in ₘCa²⁺ transporter expression is recapitulated in a murine model of AD, mutant mice were acquired which harbored three mutations associated with familial AD (3xTg-AD: Presenilin 1 (Psen1, M146V knock-in), amyloid beta precursor protein (APPswe, KM670/671NL) and microtubule associated protein tau (MAPT, P301L)). These mice develop age-progressive pathology similar to that observed in AD patients including: impaired synaptic transmission, Aβ deposition, plaque/tangle histopathology, and learning/memory deficits beginning around 6 m of age. mRNA and protein were isolated from brain tissue derived from the frontal cortex and hippocampus of 2, 4, 8 and 12 m old 3xTg-AD mutant mice and outbred age-matched non-transgenic controls (NTg) to examine changes in gene expression. 3xTg-AD mice displayed an age-dependent reduction in NCLX expression with a significant decrease noted as early as 4m and near complete loss of mRNA and protein by 12 m of age (FIG. 44B-C; FIG. 48A-48D). MICU1 and MCUb mRNA and proteins levels also displayed a progressive decrease with age (FIG. 44C; FIG. 48A-48D). No significant alteration was found in the expression of ₘCa²⁺ exchanger in samples isolated from the brains of 2 m old 3xTg-AD mice, an age prior to any detectable neuropathology or behavioral alterations (FIG. 44B; FIG. 48A and 48E). This result suggests the changes in gene expression are age-dependent and not merely the result of developmental expression changes associated with this mutant model. In summation, these results suggest a loss of the key mCa²⁺ efflux mediator, NCLX and decrease in the expression of negative regulators of the MCU (MICU1 and MCUb). These changes would promote mitochondrial calcium overload, especially in the high ᵢCa²⁺ environment that is reported to occur in neurons during AD progression. These alterations are in stark contrast to the compensatory alterations in cardiac biopsies isolated from failing hearts at the time of transplant, and suggest that in AD, changes in the expression profile of ₘCa²⁺ exchange genes may be contributing to disease development.

Next, a system more amendable to real-time mechanistic studies was used, employing a neuroblastoma cell line (N2a) stably expressing the APPswe gene (K670N, M671L, APPswe) and subjected to an often-employed maturation protocol. Importantly, maturated APPswe cells displayed a significant reduction in the expression of NCLX, mirroring the results obtained from human AD brains. Importantly, no change in OxPhos component expression was observed, suggesting no change in overall mitochondrial content (FIG. 44D-44E; FIG. 48F). To evaluate if restoring ₘCa²⁺ efflux capacity is sufficient to rescue impairments in ₘCa²⁺ handling APPswe cells were infected with adenovirus encoding NCLX (Ad-NCLX). The expression of NCLX was significantly decreased in APPswe cells to ˜50% of N2a control cells, and this was completely rescued 48 h post-infection with Ad-NCLX (FIG. 44D-44E). Next to evaluate the _{c}Ca²⁺ and ₘCa²⁺ transients, Con (N2a), Con+Ad-NCLX, APPswe and APPswe+Ad-NCLX cells were infected with adenovirus encoding the mitochondrial-targeted ₘCa²⁺ reporter, R-GECO1 (Ad-ₘᵢₜₒR-GECO) (FIG. 44F, solid line=mean, dashed line=SEM), and loaded with the _{c}Ca²⁺ reporter, Fluo4-AM (FIG. 44I solid line=mean, dashed line=SEM) and imaged continuously during stimulation with KCl to depolarize the plasma membrane and activate voltage-gated Ca²⁺ entry. Significant changes in ₘCa²⁺ rise time were not observed in all three groups (FIG. 48H). However, APPswe cells displayed a significant increase (-45%) in ₘCa²⁺ transient peak amplitude as compared to N2a control cells, and this was significantly reduced (˜20%) by Ad-NCLX (FIG. 44G). Quantification of the ₘCa²⁺ efflux rate revealed >60% decrease in APPswe cells as compared to N2a con cells and infection with Ad-NCLX increased the efflux rate in con cells by ˜20% and in APPswe cells by ˜50% (FIG. 44H). Quantification of _{c}Ca²⁺ peak amplitude revealed a significant increase (˜40%) in APPswe vs. N2a (FIG. 44J). While expression of NCLX did not alter the APP-mediated increase in _{c}Ca²⁺ flux, it restored the mitochondrial transient towards that of control N2a cells. In these studies, cells from all the groups didn't show any significant differences on MCU-mediated ₘCa²⁺ uptake rate and _{c}Ca²⁺ time to 50% decay (FIG. 48H-48I). To evaluate if impaired n,Ca efflux may contribute to ₘCa²⁺-overload, a ₘCa²⁺ retention capacity assay was employed using the ratiometric reporters FuraFF (Ca²⁺) and JC1 (mitochondrial membrane potential). APPswe cells underwent permeability transition after the 3^{rd} 10-μM pulse of Ca²⁺ (red arrow, in representative recordings). This was in striking contrast to the control, which sustained 3× the concentration of bath Ca²⁺ before collapse of ΔΨ and loss of ₘCa²⁺. Rescue of NCLX expression greatly increased the mitochondrial calcium retention capacity (˜9 pulses versus ˜3 pulses in APPswe cells, but there is no change in N2a-con cells (FIG. 44K-44L; FIG. 48J-48M). To discover if enhancing NCLX-mediated efflux was sufficient to reduce ₘCa²⁺ overload and restore matrix Ca²⁺ levels, cells from all 4 groups were loaded with Fura2 and treated with digitonin and thapsigargin. Quantification of basal ₘCa²⁺ content found that NCLX expression completely corrected APPswe-mediated Ca²⁺ overload. (FIG. 44M-44N). A recent study demonstrating that loss-of-function mutations in MICU1 (a negative regulator of MCU at low-ᵢCa²⁺; so loss-of-function promotes increased ₘCa²⁺ uptake) led to severe brain and muscle disorders provides direct evidence for ₘCa²⁺ exchange impairment in neuronal dysfunction. The studies herein are in line with previous findings and suggested ₘCa²⁺ overload is a primary contributor to AD pathology.

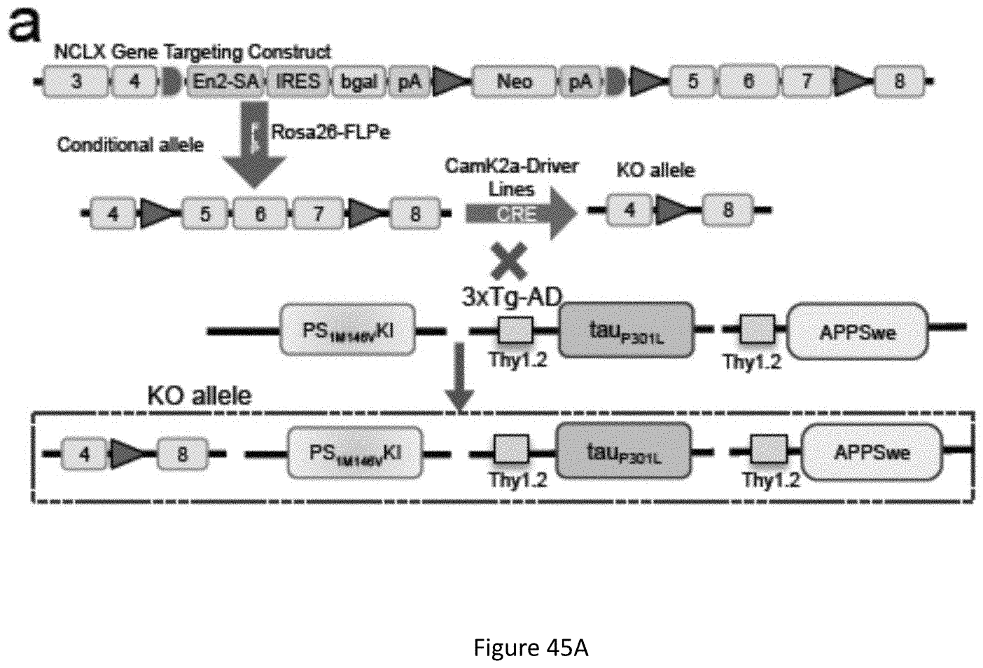

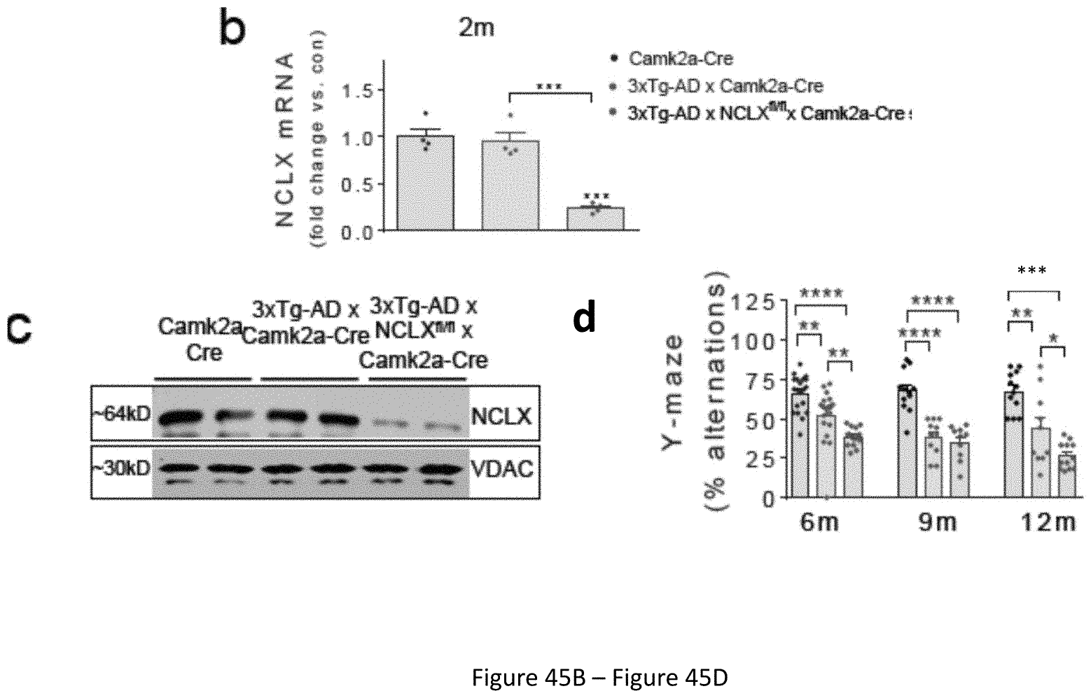

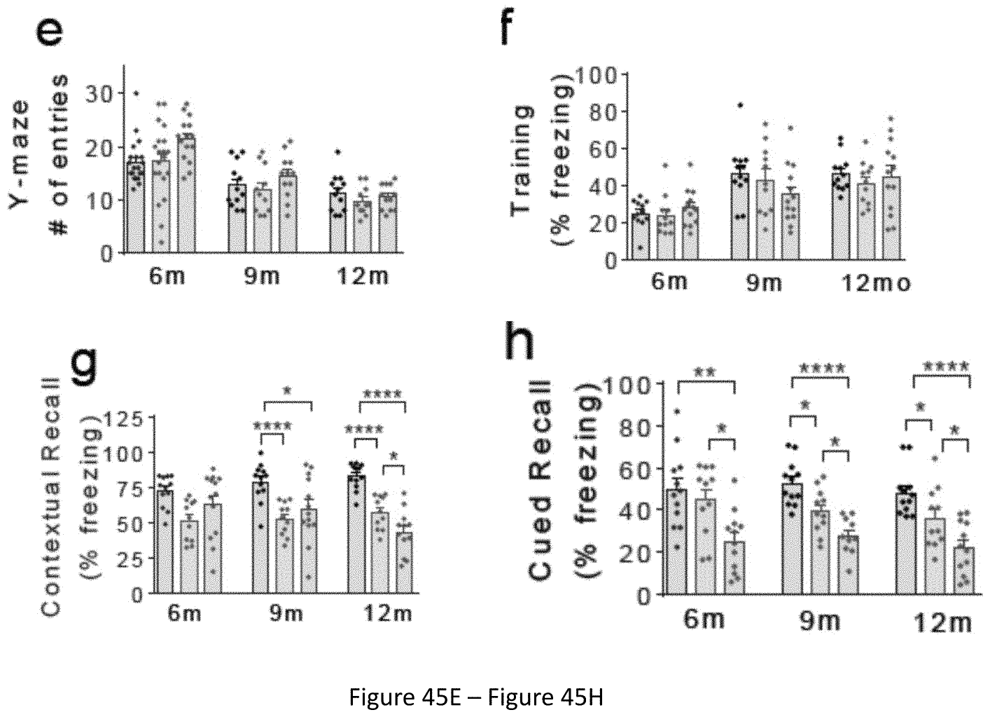

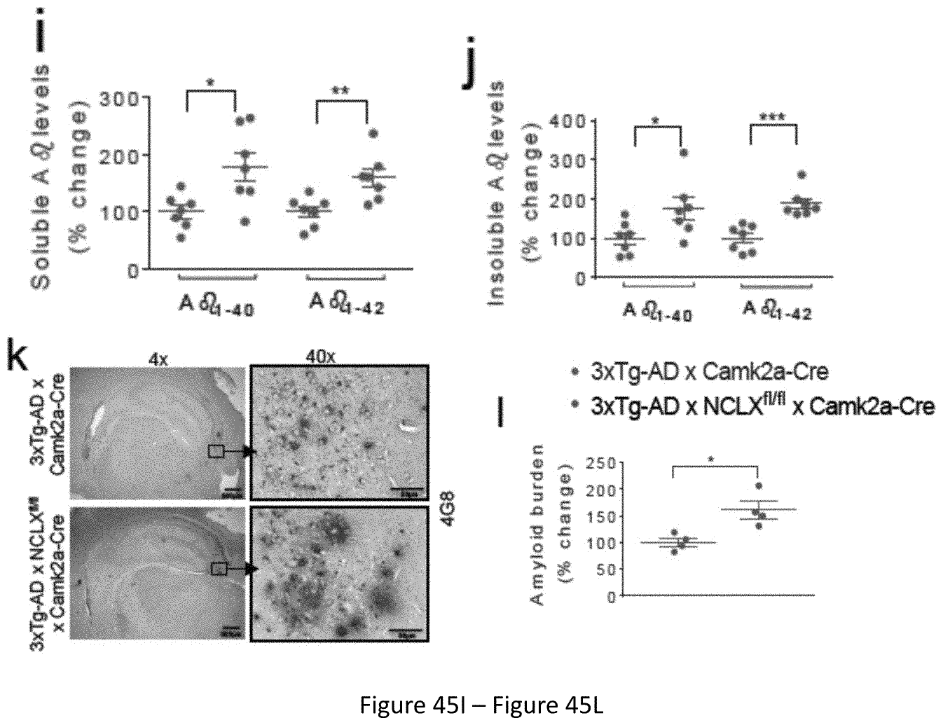

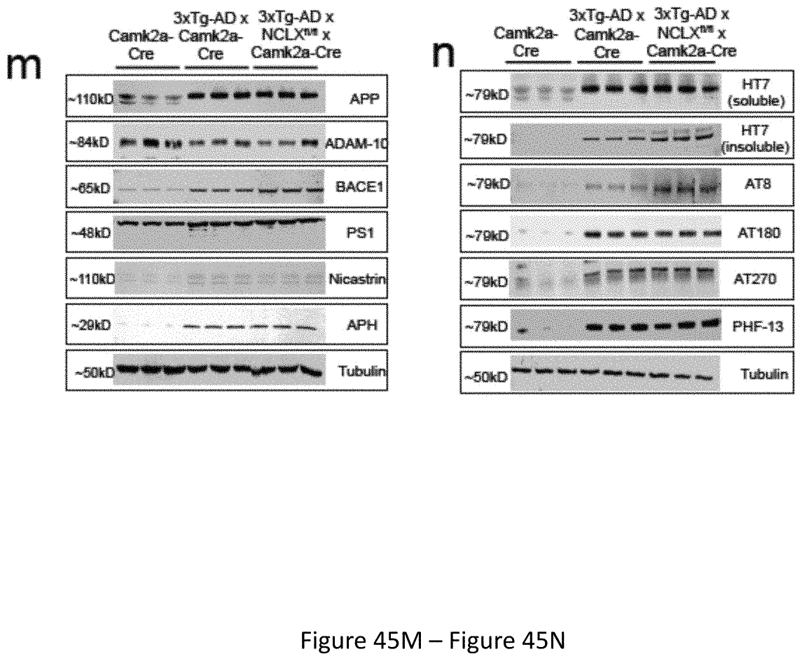

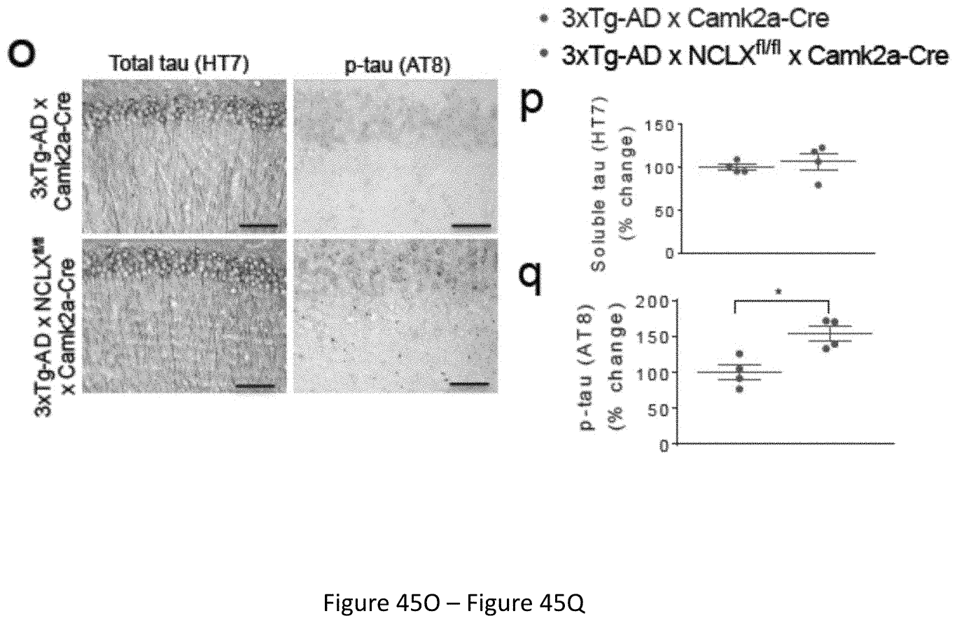

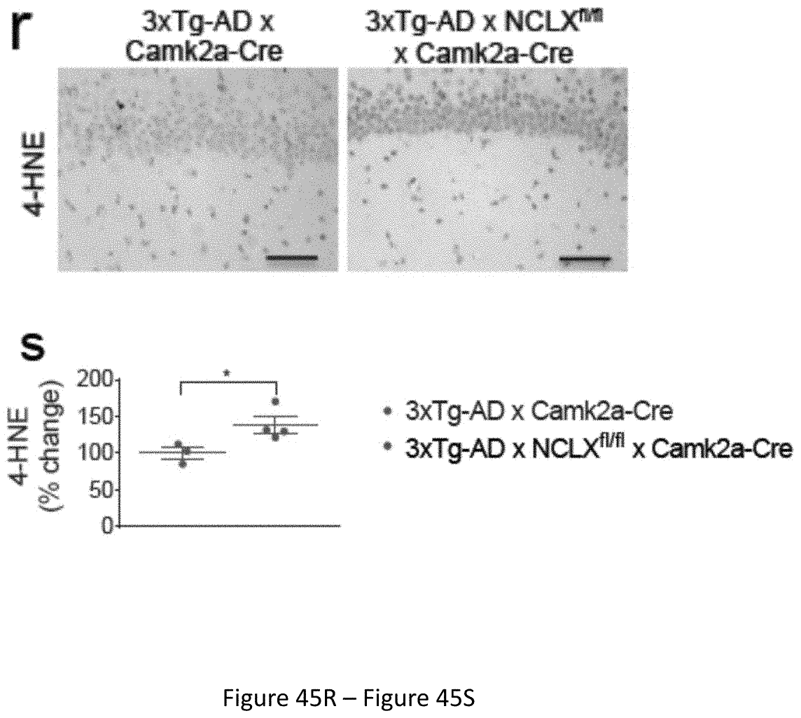

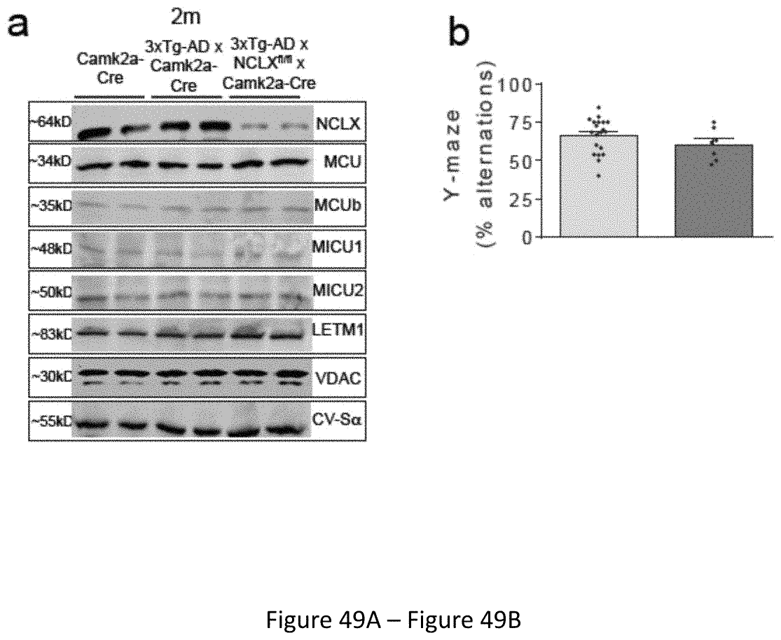

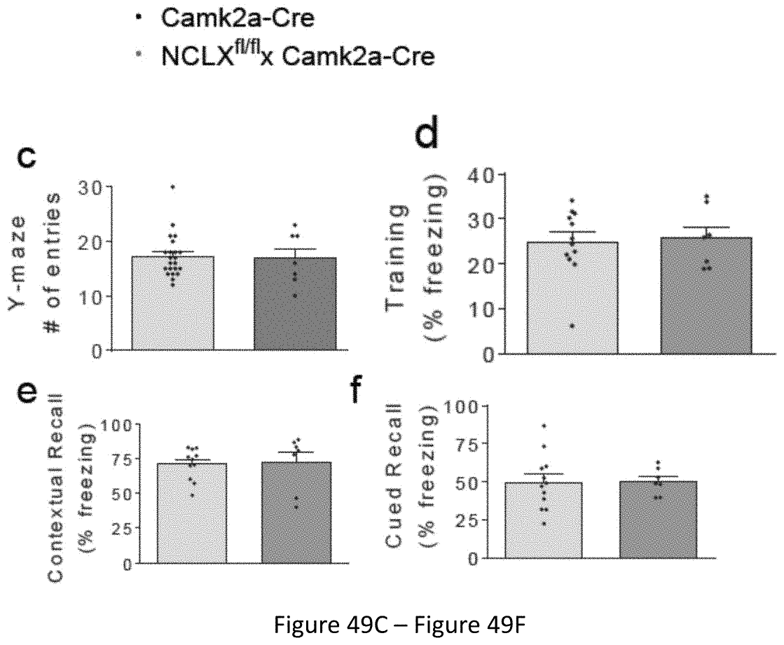

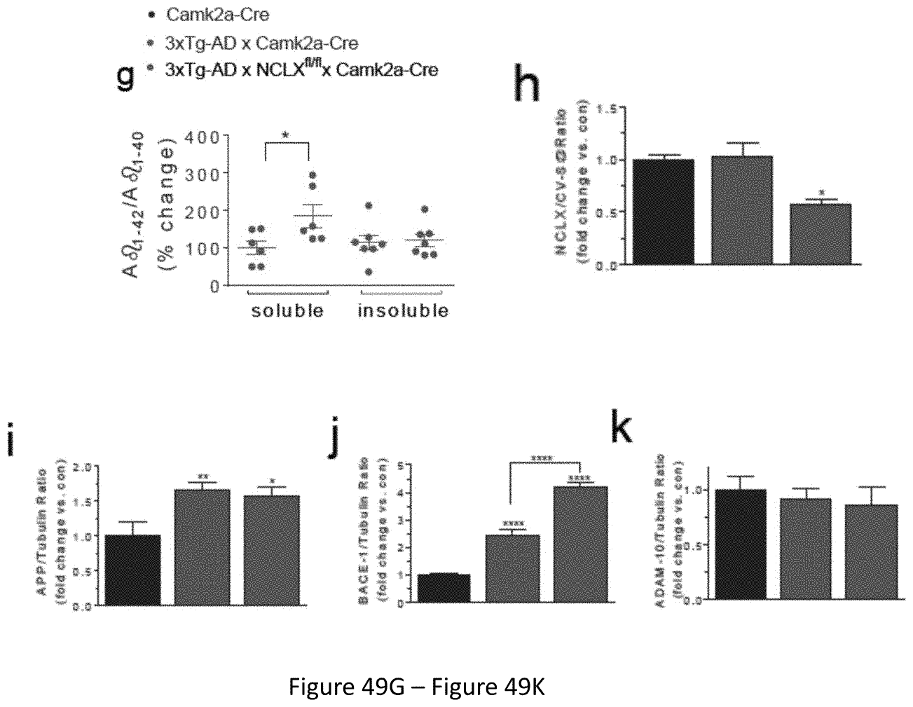

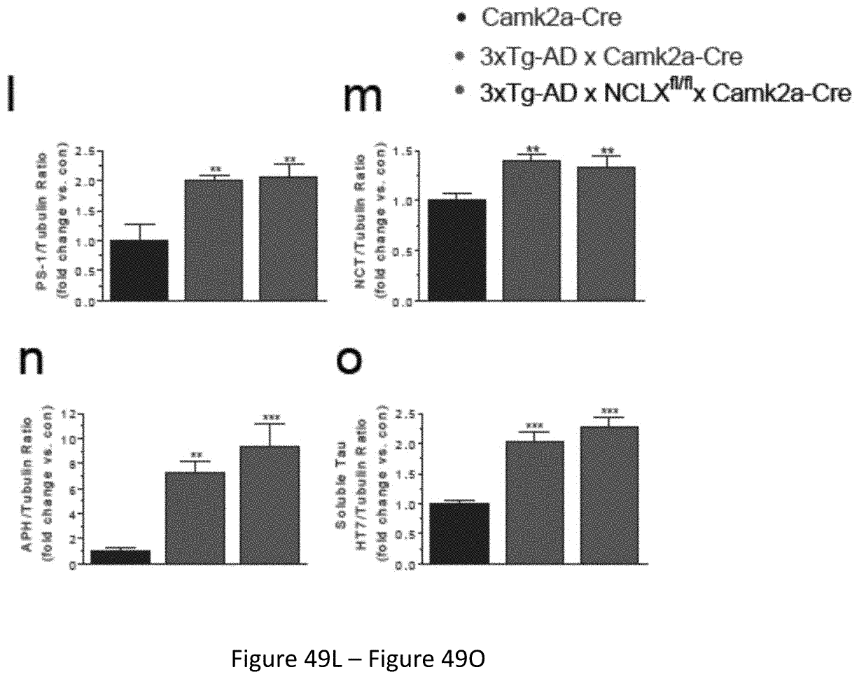

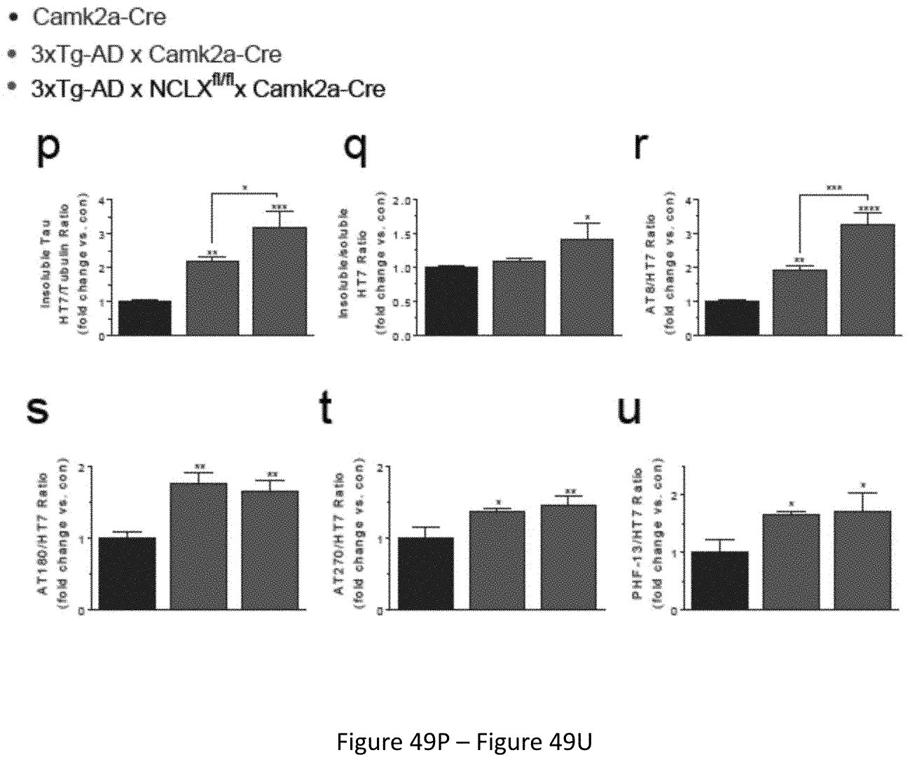

To define if impaired ₘCa²⁺ efflux contribute to the progression of AD, homozygous LoxP ‘foxed’ mice (NCLX^{fl/fl}) were crossed with neuron-specific Camk2a-Cre recombinase driver lines, resulting in germline deletion of NCLX in the forebrain, specifically to the CA1 pyramidal cell layer in the hippocampus. Resultant neuronal-specific loss-of-function models (NCLX^{fl/fl}×Camk2a-Cre) were crossed with 3xTg-AD mutant mouse to generate 3xTg-AD×NCLX^{fl/fl}×Camk2a-Cre mutant mice (FIG. 45A). An approximate 75% loss of NCLX mRNA isolated from the frontal cortex and ˜50% loss of NCLX protein isolated from the hippocampus of 2 m old 3xTg-AD×NCLX^{-/-}×Camk2a-Cre mutant mice was observed compared to age-matched control (FIG. 45B-45C) with no difference in the expression of other proposed ₘCa²⁺ regulators (FIG. 49A). Cognitive function was evaluated in the Y-maze and fear conditioning paradigm of 6, 9 and 12 m-old mice. In the Y-maze, the spatial working memory of mice was examined by measuring the percentage alternations. In this task, 3xTg-AD×Camk2a-Cre mice showed significantly reduced (˜20%) working memory at 6m when compared to control Camk2a-Cre group (FIG. 45D). Spatial memory impairments in the Y-maze have been reported at age of 6m in 3xTg-AD mice than wild type controls. The 3xTg-AD×NCLX^{fl/fl}×Camk2a-Cre mice displayed an age-dependent reduction in working memory as shown by their reduced percentage alternations compared to 3xTg-AD×Camk2a-Cre at the age of 6 (˜25%), and 12mo. (˜40%). However, trends toward slight decreases were seen in 3xTg-AD x NCLX^{fl/fl}×Camk2a-Cre mice at the age of 9m compared to 3xTg-AD×Camk2a-Cre but did not reach statistical significance (FIG. 45D). These results suggest that short term memory was severely impaired in neuronal specific NCLX knockout 3xTg-AD mice. However, no significant differences have been observed among all groups in the total numbers of arm entries, suggesting the normal motor activities (FIG. 45E). No changes were observed in spatial memory at the age of 6m in NCLX^{fl/fl}×Camk2a-Cre mice as compared to Camk2a-Cre mice (FIG. 49B-49C) suggesting that NCLX knockout in control mice has no effect. Next, fear conditioning test were performed for the contextual and cued recall. The formation of context fear is associated with hippocampus and recall of associations to cues is linked with amygdala, therefore, hippocampus and amygdala dependent associated memory can be assessed using this test. In this assay, if the mouse remembers and connects the environment with the stimulus, it will freeze, and freezing response is measured as a read-out. In this study, significant differences were not observed in the training session among all groups, showing normal motor function (FIG. 45F). 3xTg-AD×Camk2a-Cre mice showed significantly impaired contextual recall as shown by decreased (˜30% of decrease) freezing response at the age of 9 and 12m when compared to age-matched Camk2a-Cre control (FIG. 45G). However, 3xTg-AD×NCLX^{fl/fl}×Camk2a-Cre mice showed significantly impaired contextual recall at age 12 m (˜25% of decrease) as compared to 3xTg-AD×Camk2a-Cre. But, reduced freezing response was observed in cued recall at the all age including 6 m (-40% of decrease), 9 m (-30% of decrease) and 12 m (-40% of decrease) in 3xTg-AD×NCLX^{fl/fl}×Camk2a-Cre compared to age-matched 3xTg-AD×Camk2a-Cre group. NCLX knockout AD mice did not remember the cued recall even at age of 6 m suggesting the amygdala is affected at early stage of disease. No changes were observed at the age of 6 m between NCLX^{fl/fl}×Camk2a-Cre and Camk2a-Cre groups in this test (FIG. 49D-49F). These assays suggest that loss of neuronal ₘCa²⁺ efflux exacerbates cognition decline in an animal model of AD.

An intense research effort has been placed on identifying the link between Ca²⁺ dysregulation and the Aβ amyloidogenic pathway. Studies have suggested that AP increases ᵢCa²⁺ levels by numerous mechanisms and vice versa, increased ᵢCa²⁺ augments Aβ production and tau hyper-phosphorylation, two hallmarks of AD. Here, the effect of neuronal NCLX knockout on brain amyloidosis was determined by measuring Aβ peptide levels, APP processing, immunohistochemistry. To examine the effect of genetic absence of NCLX on Aβ formation in vivo, the concentrations of soluble and insoluble Aβ₁₋₄₀ and Aβ₁₋₄₂ peptides was determined in homogenates of frontal cortex of 12 m old 3xTg-AD×Camk2a-Cre and 3xTg-AD×NCLX^{fl/fl}×Camk2a-Cre mice by sandwich ELISA. Compared with 3xTg-AD×Camk2a-Cre mice, RIPA-soluble Aβ₁₋₄₀ (-80% of increase) and Aβ₁₋₄₂ (˜60% of increase) and formic acid extractable (FA) Aβ₁₋₄₀ (˜75% of increase) and Aβ₁₋₄₂ (˜85% of increase) levels were significantly increased in the cortex of 12 m old 3xTg-AD×NCLX^{fl/fl}×Camk2a-Cre (FIG. 45I-45J). NCLX knockout in AD mice led to ˜80% increase of the soluble Aβ₄₂/Aβ₄₀ ratio with no changes in the insoluble Aβ₄₂/Aβ₄₀ ratio in 12 m old mice (FIG. 49G). Immunohistochemistry was performed to study the effect of the NCLX knockout on Aβ deposition using 4G8 staining. This antibody detects amino acid residues 17-24 of β amyloid and used to examine the abnormally processed isoforms, as well as precursor forms of amyloid beta. Amyloid deposits were widely present in the cerebral cortex and hippocampus of 3xTg-AD mice at 12 m of age. In these study, the area occupied by 4G8-immunopositive reactions was significantly higher (˜60% increase) in 3xTg-AD×NCLX^{fl/fl}×Cam2a-Cre mice suggesting increased amyloid plagues as compared to 3xTg-AD×Camk2a-Cre mice (FIG. 45K-45L). The expression of Aβ precursor protein (APP), and different proteases involved in its metabolism were examined to investigate the mechanism of APP processing in these conditions. No changes were observed in the expression of total APP, α-secretase (ADAM10), the components of γ-secretase (i.e., PS1, APH1 and nicastrin) between 3xTg-AD×Camk2a-Cre and 3xTg-AD×NCLX^{fl/fl}×Camk2a-Cre mice. A significant increase was observed in β-secretase (BACE1) expression in 3xTg-AD×NCLX^{fl/fl}×Camk2a-Cre compared to 3xTg-AD×Camk2a-Cre (FIG. 45M; FIG. 49I-49N). Beta-secretase (BACE1), is the key rate-limiting enzyme to produce the beta-amyloid (abeta) peptide. Increased levels and activity of BACE1 protein in the brain of sporadic and familial AD patients and under a variety of experimental conditions such as oxidative stress, cellular and mitochondrial stress have been observed. This study concludes that NCLX deletion increases amyloidogenesis and modulates APP processing via β-secretase pathway. Next, to study tau pathology, the expression of total tau (soluble vs insoluble) and phosphorylation of tau at several epitopes was analyzed in soluble homogenate samples using western blot and immunohistochemistry. The intracellular neurofibrillary tangles are made mainly by the hyperphosphorylated microtubule-associated protein tau. In western blot, there was no change in steady-state levels of total soluble tau levels as recognized by the antibody HT7, and tau phosphorylation at Thr181 (AT270), Thr23/ser235 (AT180) and, Ser396 (PHF-13) tau between the two groups (3xTg-AD×Camk2a-Cre vs 3xTg-AD×NCLX^{fl/fl}×Cre (FIG. 45N; FIG. 49O-49U). A significant increase was observed in total insoluble tau (˜45%) as recognized by the antibody HT7 (FIG. 45N; FIG. 49P), and tau phosphorylated at residues Ser202/Thr205 (˜65%), as recognized by AT8 antibody (FIG. 45N; FIG. 49R), in 3xTg-AD×NCLX^{fl/fl}×Camk2a-Cre compared to 3xTg-AD×Camk2a-Cre. Consistent with the immunoblot results, immunohistochemical staining show no changes in somatodendritic accumulations of total soluble tau in CA1 pyramidal neurons of the 12 m-old 3xTg-AD×NCLX^{fl/fl}×Camk2a-Cre mice compared with 3xTg-AD×Camk2a-Cre (FIG. 45O-45P). Moreover, a significant increase in tau phosphorylation (50%) at Ser202/Thr205 (as detected by phospho-specific anti-tau antibody AT8) was found in the hippocampus of 3xTg-AD×NCLX^{fl/fl}×Camk2a-Cre mice compared with 3xTg-AD×Camk2a-Cre (FIG. 45O-45Q). Insoluble tau forms aggregate to develop NFTs and abnormal hyper phosphorylation of tau has also been proposed to initiate the aggregation of fibrillar and paired-helical fragments in AD. These data suggest that NCLX deletion exacerbates tau pathology in vivo. Numerous studies have demonstrated increased lipid peroxidation as an important mechanism for AD pathology. Therefore, immunohistochemistry was performed to study the effect of the NCLX knockout AD mice on ROS levels using 4-HNE staining (4-Hydroxy-2-Nonenal), as a marker for lipid peroxidation. A ˜1.4-fold increases was observed in 4-HNE staining in 3xTg-AD×NCLX^{fl/fl}×Cam2a-Cre mice suggesting increased lipid peroxidation as compared to 3xTg-AD×Camk2a-Cre mice (FIG. 45R-45S).

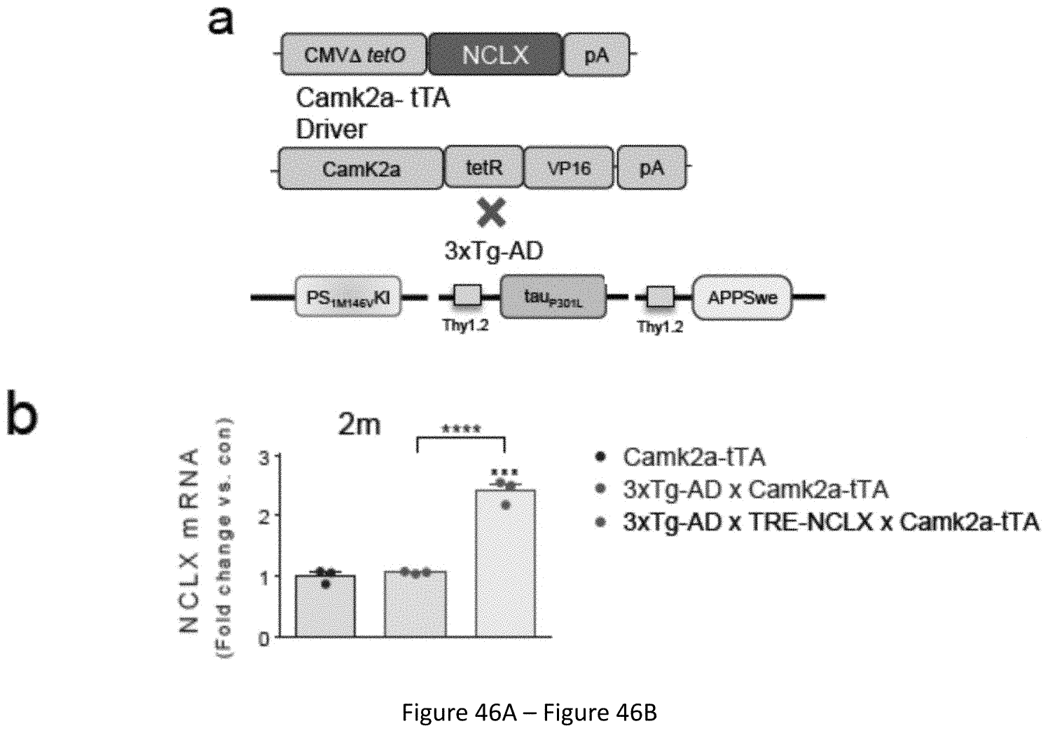

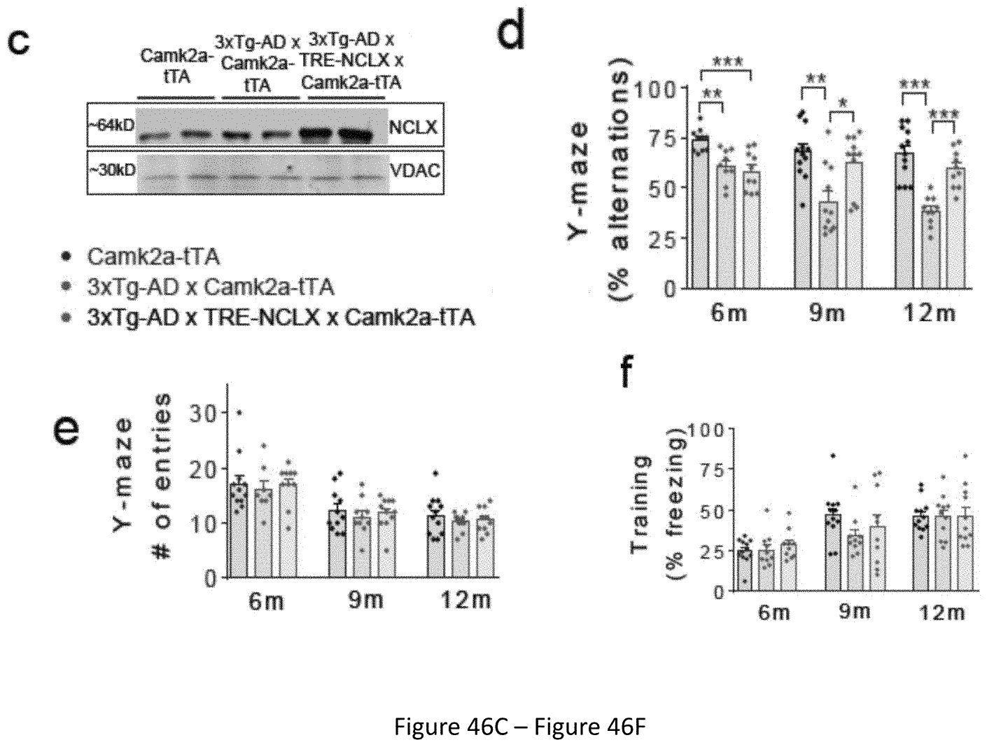

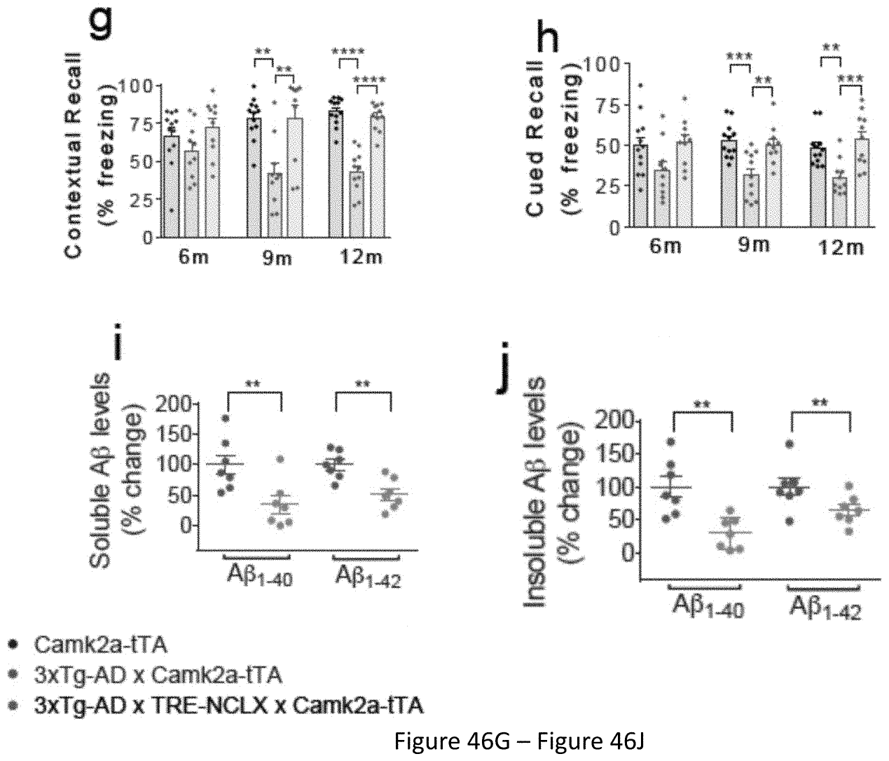

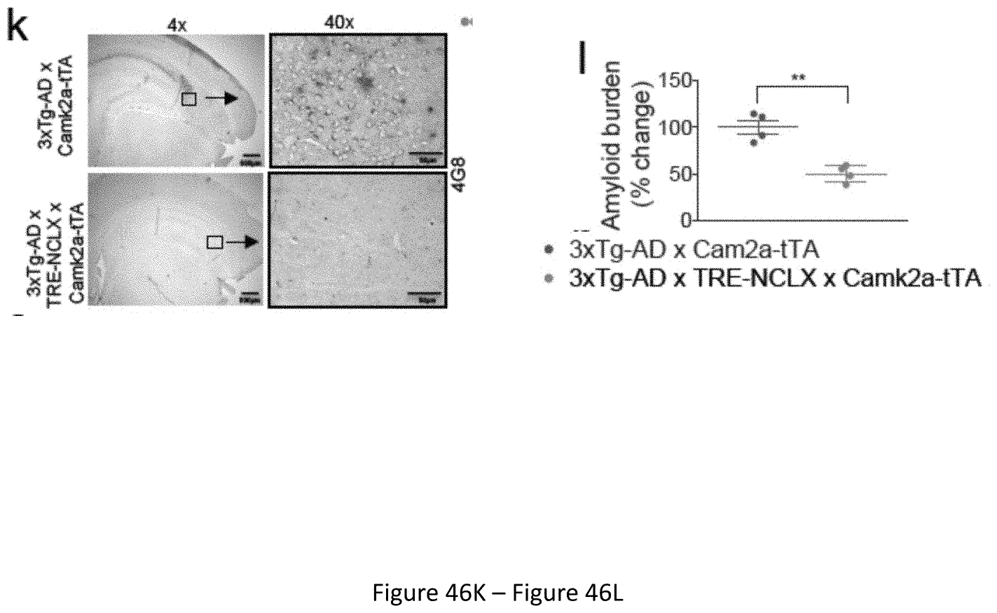

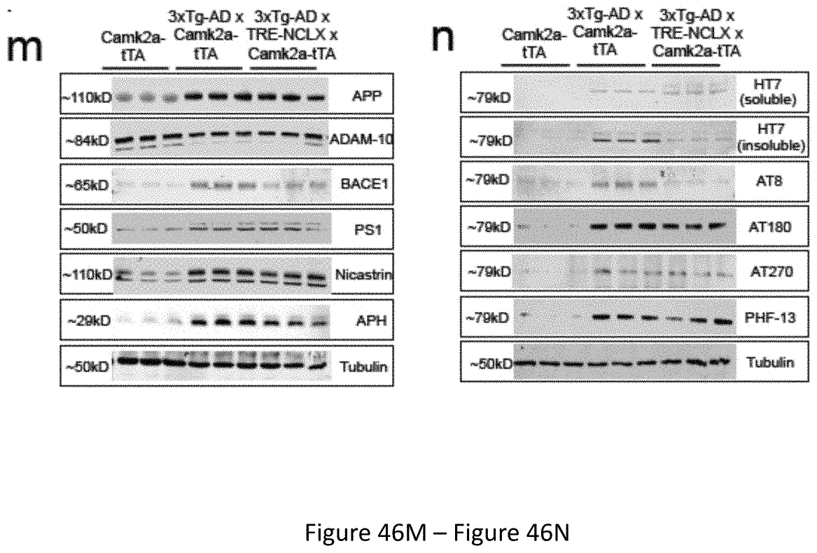

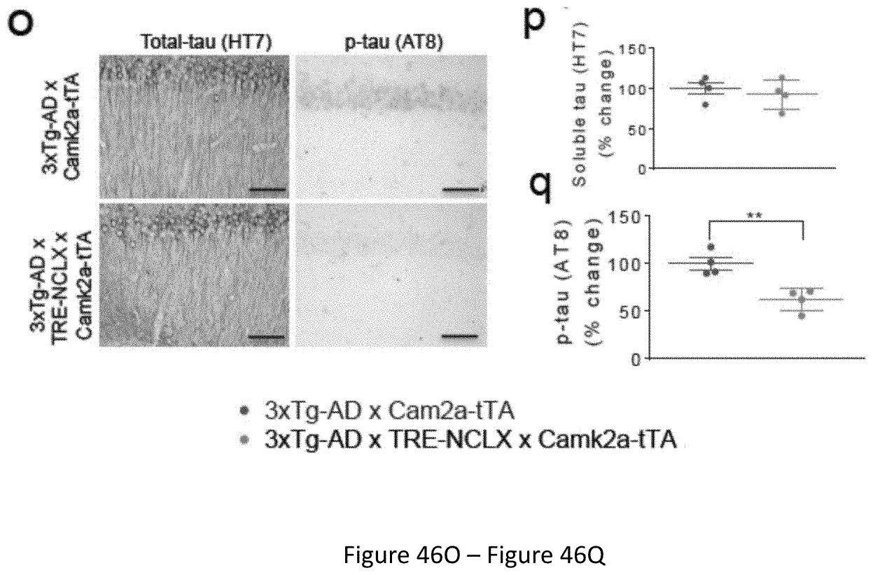

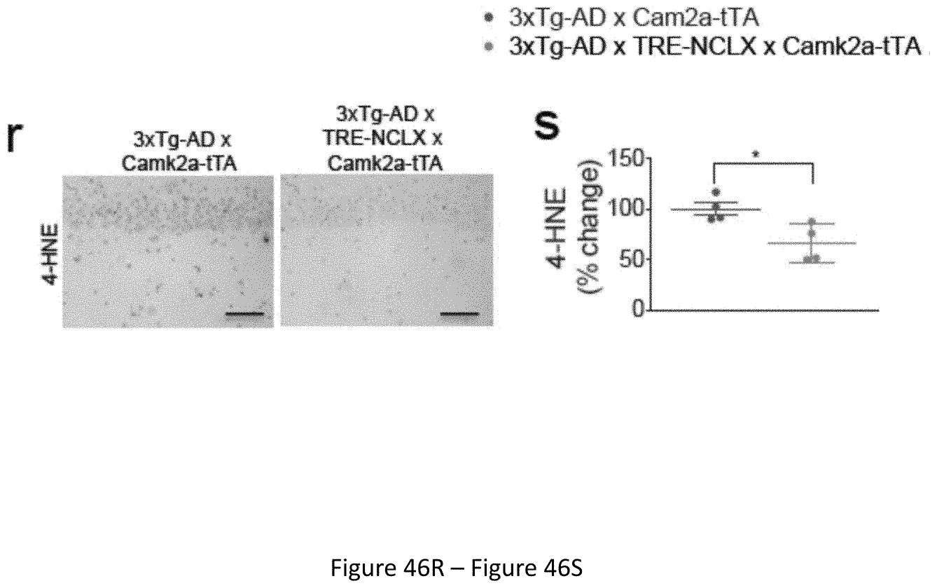

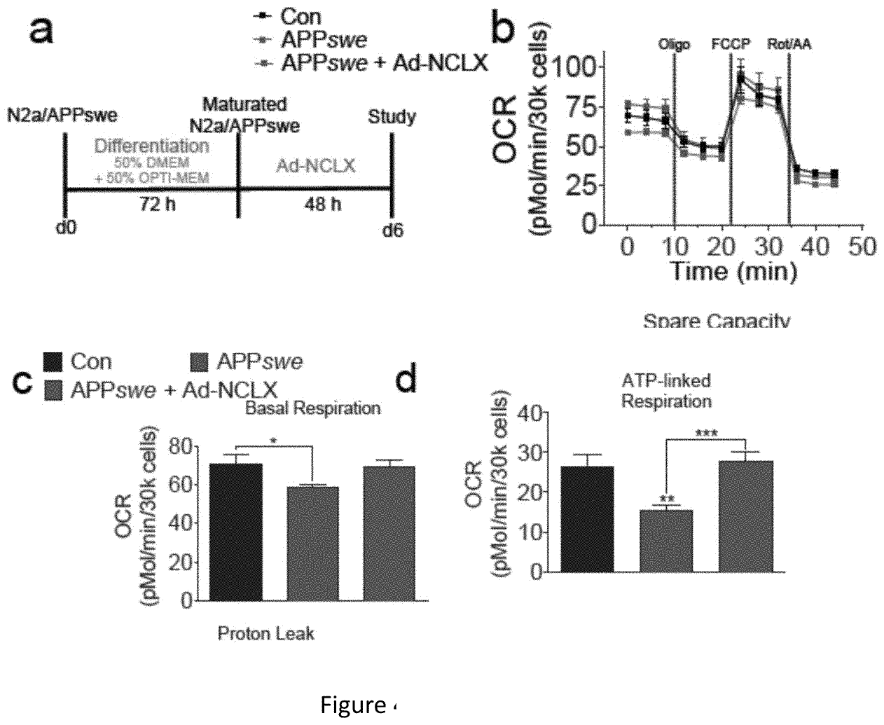

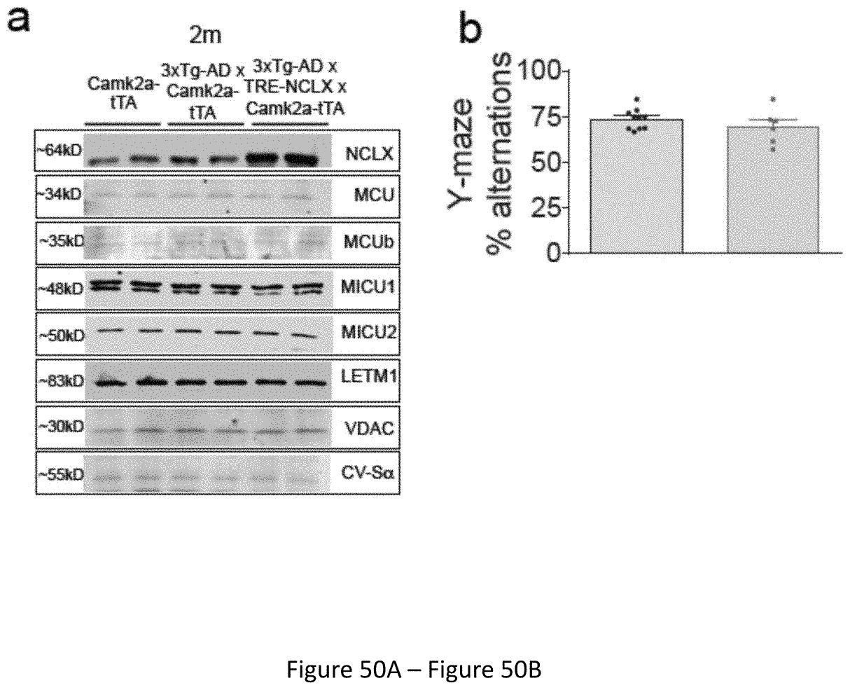

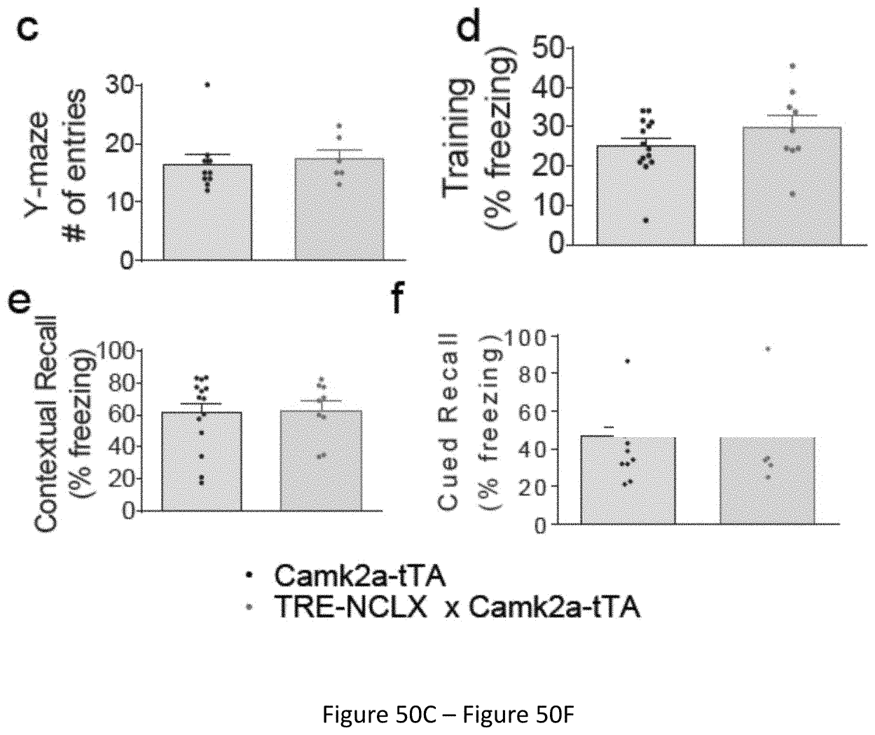

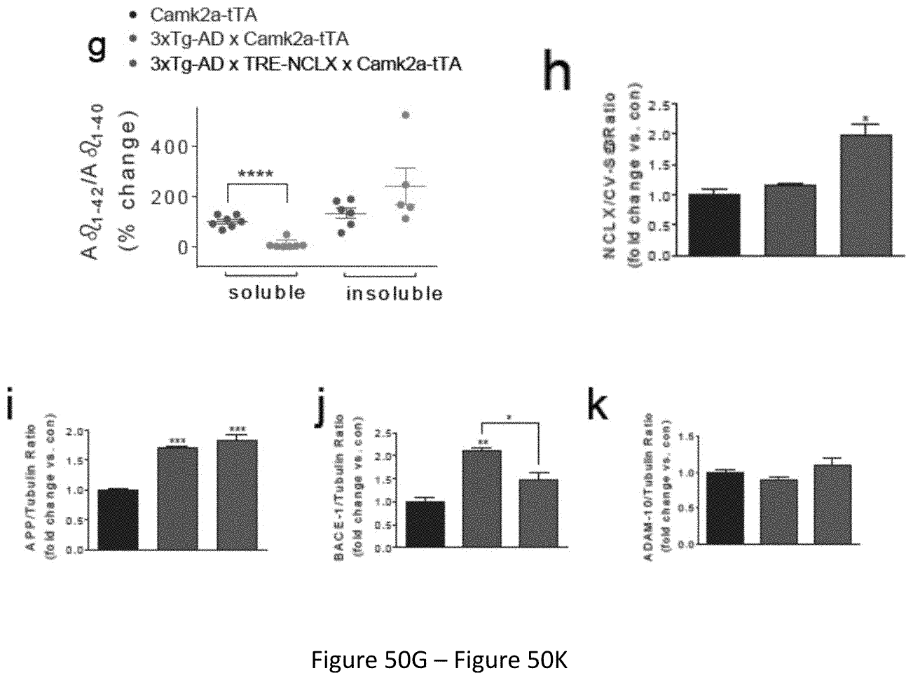

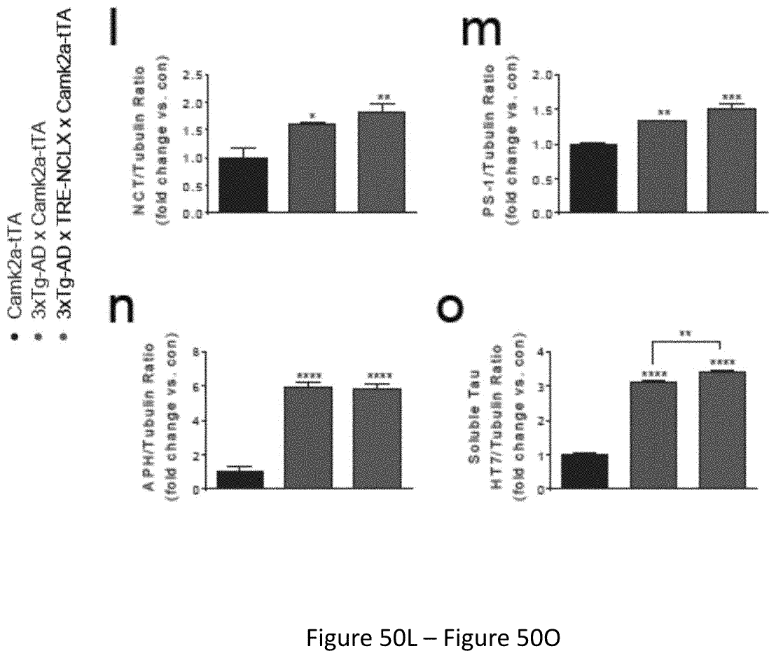

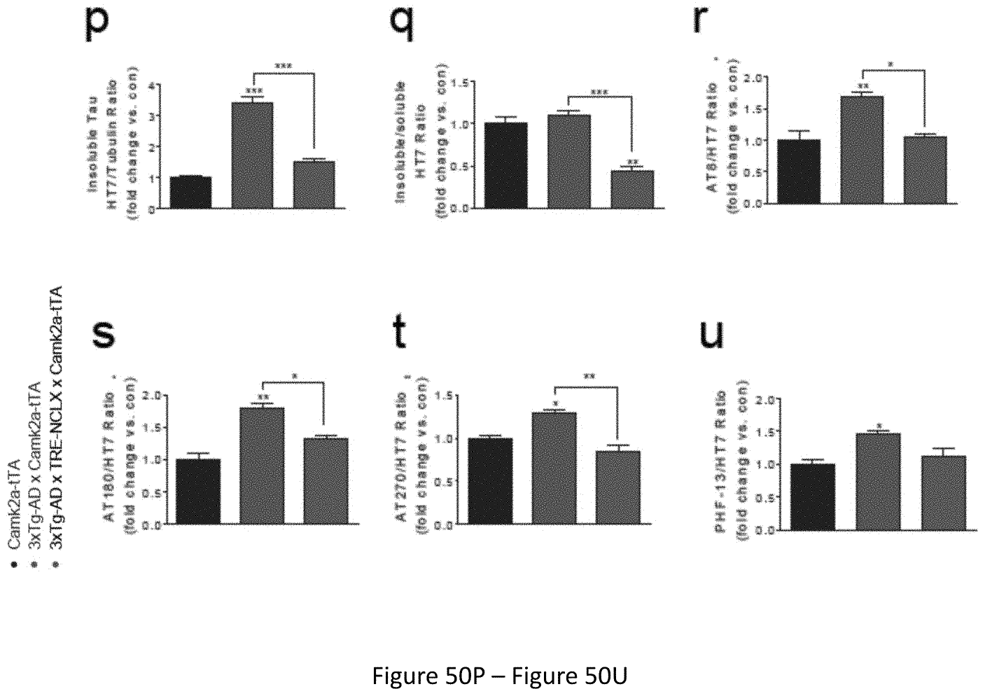

To further assess whether NCLX overexpression could rescue the AD pathology in 3xTg-AD mice, a neuron-specific, doxycycline-controlled, mouse model was generated that overexpresses NCLX. Resultant neuronal-specific NCLX gain-of-function models (TRE-NCLX×Camk2a-tTA) were crossed with 3xTg-AD mutant mouse to generate 3xTg-AD×TRE-NCLX×Camk2a-tTA mice (FIG. 46A). A ˜2.4-fold increase was observed of NCLX mRNA isolated from the frontal cortex and ˜2-fold increase of NCLX protein isolated from the hippocampus of 2 m old 3xTg-AD×TRE-NCLX×Camk2a-tTA mutant mice compared to age-matched control (FIG. 46B-46C), with no changes in the expression of other ₘCa²⁺ regulators (FIG. 50A). These mice were further tested to examine the spontaneous alternative behavior and freezing response. Neuronal specific overexpression of NCLX in 3xTg-AD mice completely rescued the cognitive decline as shown by their significantly increased percentage alternations at the age of 9 m (˜40% increase) and 12 m (˜50% increase) compared to the age-matched 3xTg-AD×Camk2a-tTA group in the Y-maze (FIG. 46D). No significant changes were observed for this parameter at 6 m (3xTg-AD×Camk2a-tTA vs 3xTg-AD×TRE-NCLX×Camk2a-tTA. No significant differences have been observed among all groups in the total numbers of arm entries (FIG. 46E). TRE-NCLX×Camk2a-tTA mice showed no changes in spatial memory as compared to Camk2a-tTA mice at 6m (FIG. 50B-50C). These results suggest that NCLX overexpression improved the spatial working memory performance in 3xTg-AD mice. In fear conditioning test, neuronal specific overexpression of NCLX in 3xTg-AD mice showed significantly increase in contextual (˜80% increase) and cued recall freezing response (˜61% increase) at the age of 9 m compared to 3xTg-AD×Camk2a-tTA (FIG. 46G-46H). Similarly, at the age of 12 m, 3xTg-AD×TRE-NCLX×Camk2a-tTA mice showed significantly increased contextual (˜80% increase) and cued recall freezing response (˜75% increase) compared to age-matched 3xTg-AD×Camk2a-tTA group (FIG. 46G-46H). This study suggests that neuronal specific overexpression of NCLX completely rescued the cognitive decline associated with the disease progression at the late or advanced stage of AD. No significant differences were observed among all groups in the training suggesting the normal motor activities (FIG. 46G). There were no changes between TRE-NCLX×Camk2a-tTA mice and Camk2a-tTA mice at 6 m in fear conditioned test (FIG. 50D-50F). Next, the neuronal NCLX overexpression effect on AD neuropathology was determined. RIPA-soluble Aβ₁₋₄₀ (65%) and Aβ₁₋₄₂ (45%) levels and formic acid extractable Aβ₁₋₄₀ (70%) and Aβ₁₋₄₂ (35%) levels were significantly decreased in the 12-m old 3xTg-AD×TRE-NCLX×Camk2a-tTa as compared to 3xTg-AD×Camk2a-tTA mice (FIG. 46I-46J). Neuronal NCLX overexpression in AD mice led to a ˜90% reduction of the soluble Aβ₄₂/Aβ₄₀ ratio without any significant reduction of the insoluble Aβ₄₂/Aβ₄₀ ratio in 12 m old mice (FIG. 50G). Similarly, 4G8-immunopositive reactions was significantly reduced (˜50%) in 3xTg-AD×TRE-NCLX×Cam2a-tTA mice suggesting reduced amyloid burden as compared to 3xTg-AD×Camk2a-tTA mice (FIG. 46K-46L). To determine the mechanism responsible for this effect on Aβ, the metabolism of its precursor, the Aβ precursor protein (APP) was examined. 3xTg-AD×TRE-NCLX×Camk2a-tTA mice have reduced β-secretase (BACE1) expression compared to 3xTg-AD×Camk2a-tTa (FIG. 46M; FIG. 50J). This conclude that enhancing ₘCa²⁺ efflux decreases the amyloidogenic Aβ pathway via BACE1 dependent mechanism. One of the therapeutic approach for AD, is to reduce Aβ production by either inhibiting β-secretase or γ-secretase activity. In these studies, no change in full-length APP expression, α and γ-secretase expression was observed (FIG. 46M; FIG. 50I-50N), suggesting NCLX an important therapeutic target. It has been reported that inhibition of γ-secretase has multiple off-target effects and showed severe developmental abnormalities. On the other side, mice deficient in BACE1, develop normally without any detectable physiological defects with a significant reduction in Aβ formation. Next, the NCLX overexpression effect was determined on onset of the tau pathology. In this study, steady-state levels of total soluble tau and phosphorylation of tau at Ser396 (PHF-13) residues were unaffected and showed similar expression in western blot between the 3xTg-AD×Camk2a-tTA vs 3xTg-AD×TRE-NCLX×Camk2a-tTA mice. However, there was marked reduction in the insoluble tau (˜50% of reduction-detected by HT7) and phosphorylated tau immunoreactivity at Ser202/Thr205 residue (˜40% of reduction—detected by AT8), Thr181 (˜35% of reduction—detected by AT270) and Thr231/ser235 (˜25% of reduction—detected by AT180) (FIG. 46N; FIG. 50O-50U). Brain immunohistochemistry analyses further supported biochemical results, showing reduced levels (˜50%) of phosphorylated tau at Ser202/Thr205 (as detected by AT8 antibody) in 3xTg-AD×TRE-NCLX×Camk2a-tTA mice compared to 3xTg-AD×Camk2a-tTA without any significant effect on somatodendritic labelling of total soluble tau in CA1 pyramidal neurons (FIG. 46O-46Q). This study suggests that NCLX overexpression significantly reduced the insoluble tau and its abnormal phosphorylation, which have been implicated in the development of neurofibrillary tangles in AD. In these studies, levels of 4-hydroxy-2-nonenol, an indicator of lipid peroxidation, were significantly decreased by ˜1.5-fold in 3xTg-AD×TRE-NCLX×Camk2atTA mice relative to 3xTg-AD×Camk2a-tTA mice (FIG. 46R-46S). These observations indicate that NCLX overexpression could rescue the lipid peroxidation in AD mice.

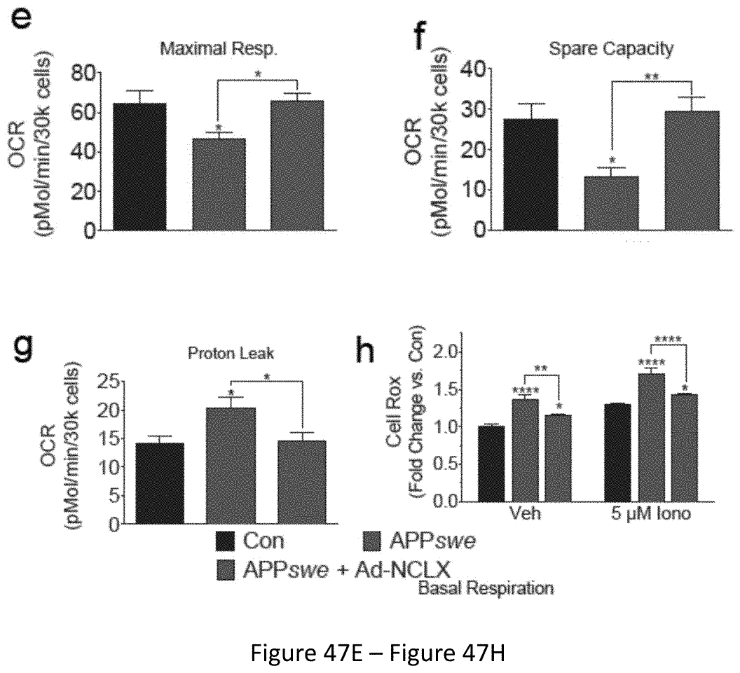

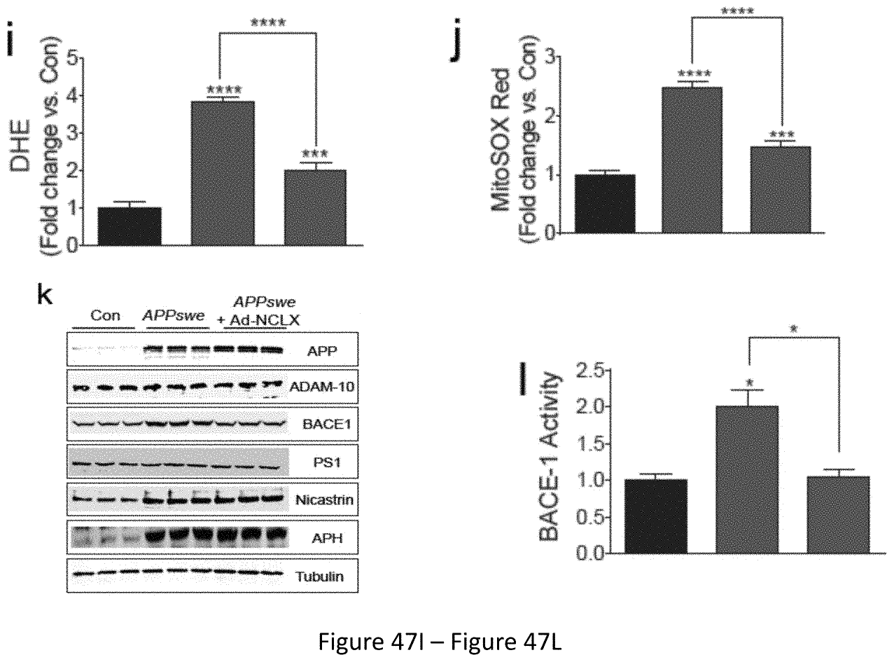

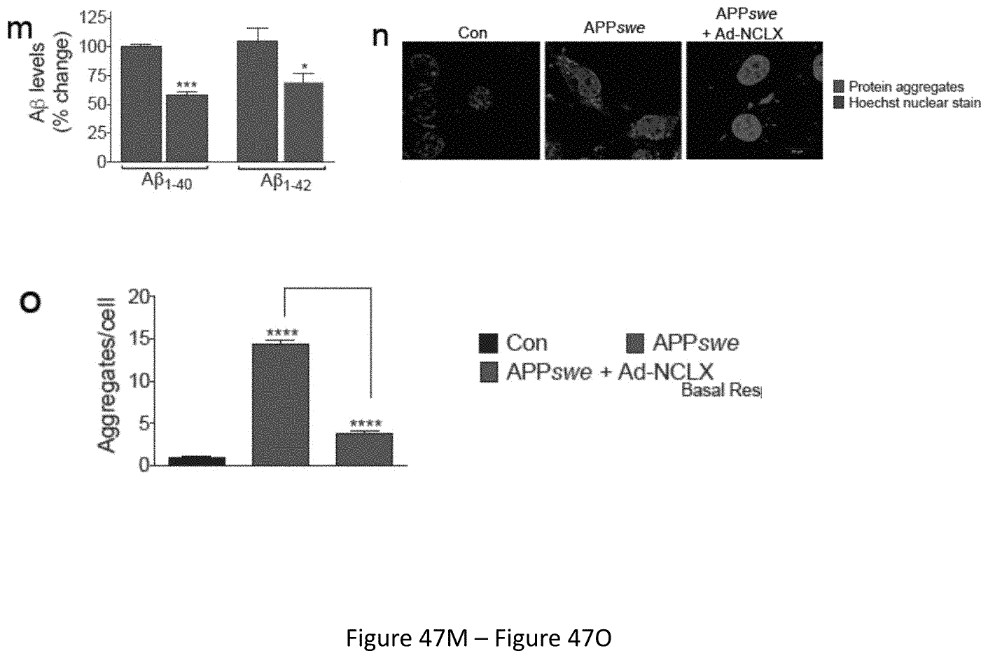

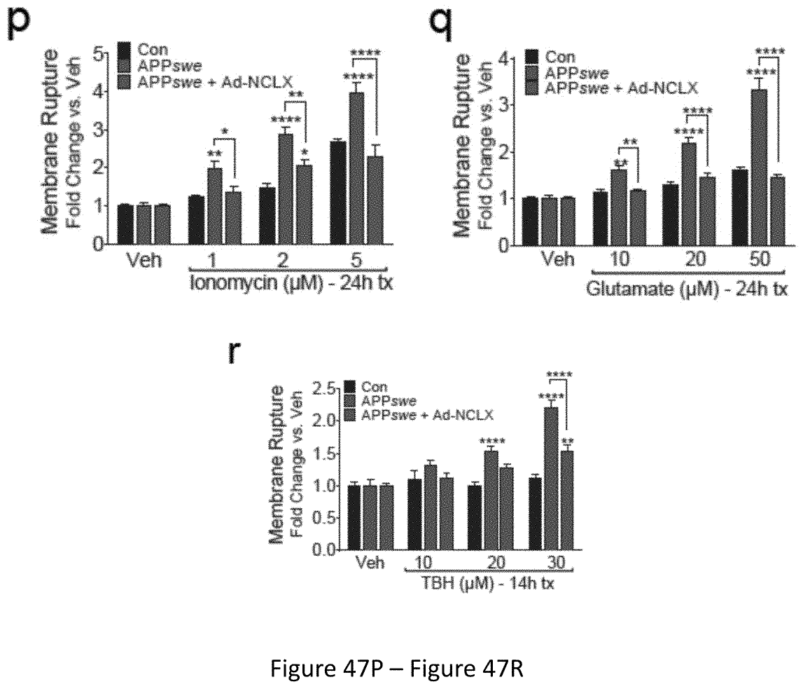

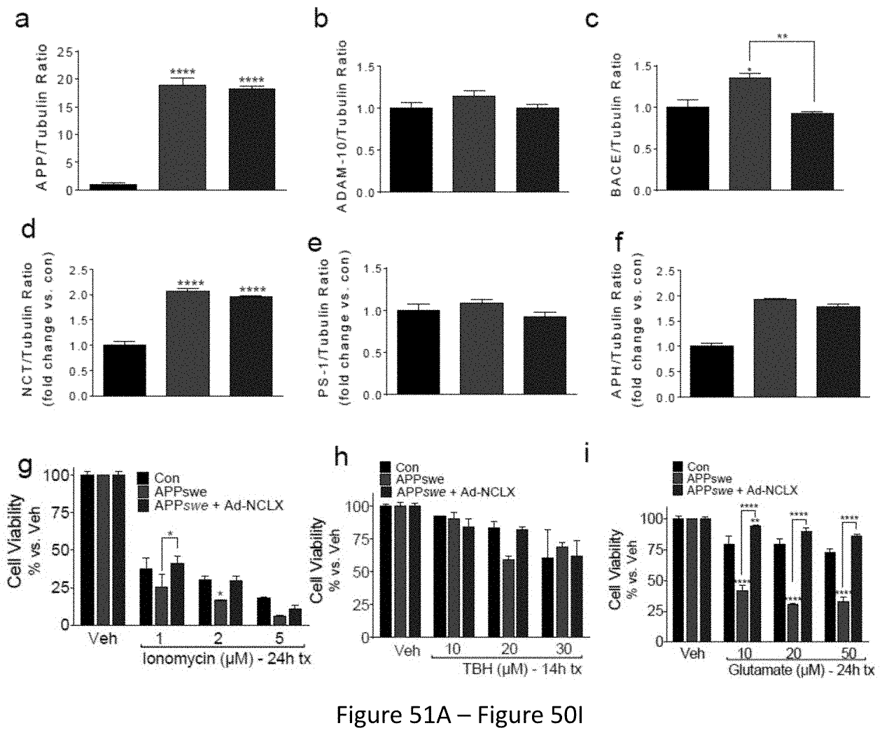

AD is characterized by neuronal metabolic dysfunction, with studies suggesting that mitochondrial defects in energy production may underlie neurodegeneration and cognitive decline. Therefore, the maturated APPswe cells were examined for changes in OxPhos using a Seahorse XF96 extracellular flux analyzer to monitor oxygen consumption rates (OCR) (FIG. 47B-47G). APPswe mutant cells displayed a significant decrease in all respiratory parameters examined. Specifically, ˜1.5 fold lower basal respiration, 2-fold lower ATP-linked respiration, 1.5 fold lower max respiratory capacity and 1.5 fold lower spare respiratory capacity in APPswe vs. N2a controls. Amazingly, rescue of ₘCa²⁺ efflux with Ad-NCLX infection for 48 h corrected all OCR measurements back to N2a control levels (FIG. 47B-47G). These results show that mCa²⁺ overload is a significant contributor to AD-mediated impairments in OxPhos and that NCLX is sufficient to restore bioenergetics. Similarly, ₘCa²⁺-overload is known to elicit increased ROS generation and suppression of ROS scavenging pathways via numerous molecular mechanisms. Here maturated cells (Con, APPswe, and APPswe+Ad-NCLX) were examined for changes in redox status utilizing 3 different ROS sensors. 30 m following treatment with vehicle (Veh) or the Ca²⁺ ionophore, ionomycin (Iono), cells were loaded with the total cellular ROS indicator, CellROX Green. APPswe displayed an increase in total ROS that was significantly reduced in APPswe cells expressing NCLX (48h post-adeno) (FIG. 47H). Next, the O₂^{⋅-} specific probe dihydroethidium (DHE) was used. APPswe had a ˜4-fold increase in O^{⋅-} production that was reduced by ˜50% with NCLX expression (Ad-NCLX) (FIG. 47I). To further define the subcellular site of ROS generation the mito-targeted O₂^{⋅-} indicator, MitoSOX Red was used. Quantification of MitoSOX fluorescent intensity showed ˜3-fold increase in O₂^{⋅-} production in APPswe vs. con that was reduced by ˜50% with NCLX expression (Ad-NCLX) (FIG. 47J). These results support the notion that expression of NCLX, in the context of AD-like stress, reduces mitochondrial O₂^{⋅-} production. It was next investigated how altering ₘCa²⁺ levels impacts Aβ production, toxicity and clearance. APP processing was examined by Western blot. Enhancing ₘCa²⁺ efflux (NCLX expression for 48 h) reduced β-secretase (BACE1) expression in APPswe cells without any significant change in the levels of other components (FIG. 47K; FIG. 51A-51F). In addition, a fluorescence enzymatic assay was performed using a synthetic peptide, which has previously been shown to be highly specific. BACE1 activity was significantly increased in APPswe cells by ˜2-fold vs con. The AD associated swedish mutant APP is associated with increased β-secretase activity as was observed in APP swe cells. A significant ˜50% decrease was observed in BACE1 activity in APPswe infected with Ad-NCLX vs. APPswe (FIG. 47L). These results suggest a direct involvement of the BACE-1 protease in the observed biological effect. To further evaluate the effect of NCLX expression on Aβ generation, an ELISA was used for quantification of extracellular Aβ₁₋₄₀ and Aβ₁₋₄₂ levels. Compared with APPswe controls a significant decrease was observed in Aβ₁₋₄₀ ˜( 40% of decrease) and Aβ₁₋₄₂ formation (˜40% of decrease) in APPswe infected with Ad-NCLX (FIG. 47M). Moreover, it is the Aβ aggregate formation that plays a central role in the pathogenesis of AD. To determine whether the NCLX have any effect on Aβ oligomerization, a fluorescence-based assay using Proteostat dye, was used to detect aggregated protein. This dye is essentially non-fluorescent unless it binds to a β-sheet structure of misfolded proteins in which case it fluoresces as a punctate pattern of cytoplasmic staining. APPswe cells showed increased accumulation of cytoplasmic inclusion bodies/aggregates vs con. Rescue of NCLX expression in APPswe significantly decreased the protein aggregation ˜70% as compared to APPswe cells (FIG. 47N-47O). These results are intriguing and suggest that elevated ₘCa²⁺ signaling may contribute to the amyloid cascade. In total this data demonstrates that NCLX modulates Aβ formation by regulating BACE1 activity and protein levels. ₘCa²⁺-overload has been shown to augment neuronal cell death both through primary (MPTP and ROS) and secondary signaling mechanisms (metabolic derangement, etc.). Given that NCLX expression reduced O₂^{⋅-} production and MPTP activation and enhanced OxPhos capacity it was tested if these protective mechanisms coalesced to reduce neuronal demise. Con, APPswe and APPswe infected with Ad-NCLX for 48 h were treated with Iono, (1-5 μM) for 24 h and examined for plasma membrane rupture (hallmark of cell death) using the cell membrane impermeable dye, Sytox Green. Iono significantly increased membrane rupture in APPswe expressing cells over the N2a control at all doses and this was attenuated with NCLX expression (FIG. 47P). General cell viability was also examined using Cell Titer Blue and found that rescue of NCLX expression in APPswe profoundly increased cell viability at all doses as compared to APPswe cells (FIG. 51G). Similarly, all groups were treated with the oxidizing agent and free-radical generator, tert-Butyl hydroperioxide (TBH), which is preferred over H₂O₂ due to its increased stability in solution. Treatment with 20 and 30 μM TBH for 14 h significantly increased membrane rupture in APPswe expressing cells over the control, which was reduced with increased NCLX expression (FIG. 47Q). Cell Titer Blue was used to monitor cell viability and found that NCLX expression partially increased cell viability in APPswe in response to oxidative stress (FIG. 51H). Likewise, treatment with glutamate (NDMAR-agonist, neuroexcitotoxicity agent) significantly increased cell death in APPswe expressing cells across all doses and this was completely ablated by NCLX expression (FIG. 47R). Similarly, cell viability in APPswe with increased NCLX expression was significantly enhanced at all doses of glutamate as compared to APPswe cells (FIG. 51C). These results strongly support that rescue of NCLX expression in the context of AD may be a powerful therapeutic to impede cell loss and AD progression.

Taken together, these studies demonstrate the loss of mitochondrial Na⁺/Ca²⁺ exchanger (NCLX) and the severe ₘCa²⁺ signaling abnormalities in AD. This study confirms that reduced ₘCa²⁺ efflux capacity can cause neuronal dysfunction and AD progression in 3xTg-AD mice. Genetic rescue of NCLX expression in 3xTg-AD mice restores cognitive function, and significantly reduces AD-pathology. In addition, restoring ₘCa²⁺ efflux capacity using NCLX reduces pathogenic ₘCa²⁺ overload, OxPhoS defects and oxidative stress in AD. Previous evidences provide a link between AD and mitochondrial dysfunction together with a perturbed cellular calcium homeostasis, deregulation of energy metabolism and oxidative stress. Earlier, postmortem AD brain patients have shown increased oxidative and metabolic compromise which makes neurons vulnerable to excitotoxicity and cell death. ₘCa²⁺ has been shown to significantly alter metabolism and cell death both of which have been shown to contribute to neurodegeneration. This suggests NCLX is a good target to rescue ₘCa²⁺ load in these neurons. Previously, it has been shown that Aβ depletes Ca²⁺ amounts in the ER, resulting in increased cytosolic Ca²⁺ levels that lead to depolarization of mitochondrial membrane potential, induction of mitochondrial apoptotic events and ROS formation. Evidences also suggest that Aβ either interact directly with mitochondria or indirectly by elevated _{c}Ca²⁺ levels. Oxidative stress impairs mitochondrial metabolism via inhibiting the activity of key enzymes of energy metabolism such as pyruvate dehydrogenase, α-ketoglutarate dehydrogenase and cytochrome oxidase. In number of studies, oxidative stress has been shown to precede Aβ accumulation and tau phosphorylation even at the early stage of AD. It can alter both APP and tau processing possibly via activation of various signaling pathway. Oxidative stress has been shown to increase the BACE-1 expression through the c-Jun N-terminal kinases and p38(MAPK) signaling and abnormal phosphorlytion of tau by activation of glycogen synthase kinase and p38 (AT8). It has been shown that PHF-tau (AT8) interact with p38 in AD in presence of oxidative stress. In these studies, NCLX knockout mice showed increased PHF-tau (AT8, an early marker for phosphorylated tau) suggesting oxidative stress may be important mediator for AD pathology in these conditions. Recently, oxidation induced downregulation of Pin1, the prolyl isomerase, has also been shown to increase amyloidogenic APP processing and tau hyper phosphorylation in AD suggesting the different possible pathways connecting oxidative stress and AD pathology. Besides oxidative stress, increased levels BACE1 protein and tau phosphorylation has also been reported under an energy depletion, cellular and mitochondrial stress condition. These experiments provide the first biological evidence that the enhancing the clearance of pathogenic ₘCa²⁺ via rescuing NCLX expression preserved mitochondria function, biogenetics and reduced oxidative stress. These preservative functions ultimately decreased tau hyper phosphorylation and BACE1 expression and in turn regulates APP processing to generate Aβ. Furthermore, these results suggest that rescuing NCLX expression may provide significant rationale towards the future development of therapeutics aimed at increasing ₘCa²⁺ efflux in neurodegenerative AD diseases.

### Example 6: Loss of the Mitochondrial Sodium/Calcium Exchanger in the Adult Heart Causes Sudden Death and Overexpression Protects Against Heart Failure

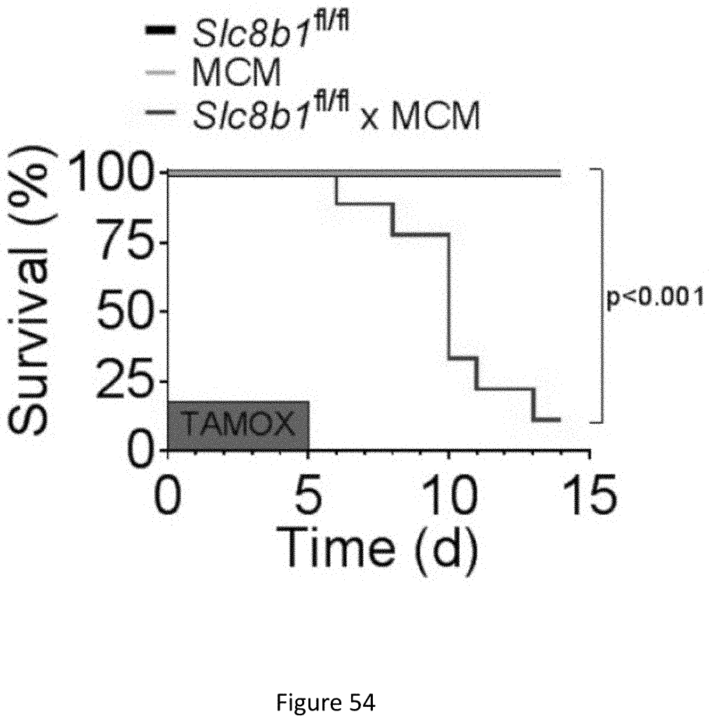

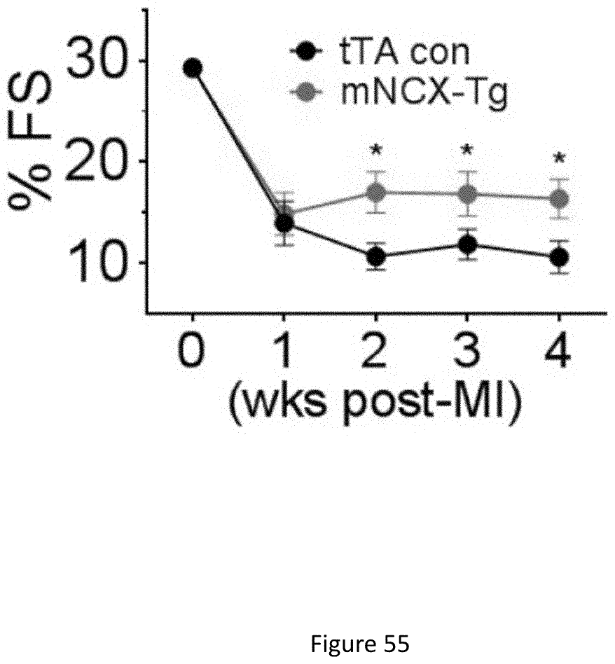

Mitochondrial calcium (ₘCa²⁺) signaling is critical for both energy production and the activation of cell death pathways. Further, metabolic derangement and gradual cell dropout are mechanistically implicated as significant contributors to the development and progression of heart failure (HF). The mitochondrial sodium/calcium exchanger (mNCX) is hypothesized to be the primary mechanism of ₘCa²⁺ efflux, but to date no study has confirmed its identity or function in an in vivo system. To investigate the role of mNCX in HF, mutant mice were generated with loxP sites flanking exons 5-7 of the candidate gene, Slc8b1 (also known as NCLX), and crossed them with a tamoxifen (tamox)-inducible cardiomyocyte-specific Cre mouse to delete mNCX in the adult heart (mNCX-cKO). Biophysical study of cardiomyocytes isolated from mNCX-cKO mice revealed a significant reduction in ₘCa²⁺ efflux rate and ₘCa²⁺ uptake capacity. Tamoxifen-induced ablation of mNCX resulted in sudden death with most mice dying the first week after cre-mediated deletion (FIG. 54). Echocardiographic evaluation of mNCX-cKO hearts 3d post-tamox revealed significant left ventricular (LV) remodeling characterized by significant dilation and a substantial decrease in function. Implantation of radiotelemeters revealed severe cardiac arrhythmias in mNCX-cKO mice prior to sinus arrest. In addition, mNCX-cKO hearts exhibited increased reactive oxygen species generation when assessed by DHE imaging of live tissue and mitoSOX Red imaging in isolated adult cardiomyocytes. Using an Evan's blue dye exclusion technique, we found that mNCX-cKO hearts displayed significant sarcolemmal rupture, indicative of cellular necrosis. Next, a conditional, cardiac-specific mNCX overexpression mouse model was generated (mNCX-Tg) to evaluate if increasing ₘCa²⁺ efflux would alter the progression of HF. mNCX-Tg and controls were subjected to in vivo myocardial infarction (LCA ligation) and pressure-overload induced HF (transverse aortic constriction). mNCX-Tg mice displayed preserved LV function, structure and a reduction in HF indices in both models (MI % FS, FIG. 55). For the first time, the data presented herein show that mNCX is essential for maintenance of the ₘCa²⁺ microdomain in cardiomyocytes and that mNCX represents a novel therapeutic target in HF.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for treating or preventing neurodegeneration or a neurodegeneration-related disease or disorder the method comprising administering a composition comprising an activator of mitochondrial Na⁺/Ca²⁺ exchanger (mNCX) to a subject in need thereof.

2. The method of claim 1, wherein the activator is selected from the group consisting of a chemical compound, a protein, a peptide, a peptidomemetic, an antibody, a ribozyme, a small molecule chemical compound, a nucleic acid, a vector, and an antisense nucleic acid molecule.

3. The method of claim 1, wherein the neurodegeneration-related disease or disorder is selected from the group consisting of Alzheimer's Disease, amyotrophic lateral sclerosis, Parkinson's, Alzheimer's, Huntington's, Batten disease, prion disease, motor neuron diseases, traumatic brain injury, blast injury, dementia, Tay-Sachs, Niemann-Pick, PDH deficiency, aggregation disorders, encephalopathies, ataxia disorders, and neurodegeneration associated with aging

4. The method of claim 1, wherein the activator increases one or more of transcription, translation, and activity of mNCX.

5. A method for treating or preventing fibrosis or a fibrosis-related disease or disorder the method comprising administering a composition comprising an modulator of a target to a subject in need thereof, wherein the target is selected from the group consisting of mitochondrial Na⁺/Ca²⁺ exchanger (mNCX), a PDH kinase, a PDH phosphatase, an alpha-ketoglutarate dependent demethylase, phosphofructokinase-2 (PFK-2), calcium sensitive alpha-ketoglutarate dehydrogenase, and the ratio of alpha-ketoglutarate to succinate.

6. The method of claim 5, wherein the alpha-ketoglutarate dependent demethylase is selected from the group consisting of a Ten-eleven translocation (TET) enzyme and a Jmj C-domain containing histone demethylase (JHDM).

7. The method of claim 5, wherein the modulator is an activator.

8. The method of claim 5, wherein the modulator is an inhibitor.

9. The method of claim 8, wherein the inhibitor prevents one or more of transcription, translation, and activity of mNCX.

10. The method of claim 6, wherein the modulator is selected from the group consisting of a chemical compound, a protein, a peptide, a peptidomemetic, an antibody, a ribozyme, a small molecule chemical compound, a nucleic acid, a vector, and an antisense nucleic acid molecule.

11. The method of claim 6, wherein the fibrosis-related disease or disorder is selected from the group consisting of cardiac fibrosis, interstitial lung diseases, liver cirrhosis, wound healing, systemic scleroderma, and Sjogren syndrome.

12. A method for treating or preventing neurodegeneration or a cardiovascular disease or disorder the method comprising administering a composition comprising a modulator of mitochondrial Na⁺/Ca²⁺ exchanger (mNCX) to a subject in need thereof

13. The method of claim 12, wherein the modulator decreases one or more of transcription, translation, and activity of mNCX.

14. The method of claim 12, wherein the modulator increases one or more of transcription, translation, and activity of mNCX.

15. The method of claim 12, wherein the wherein the modulator is selected from the group consisting of a small interfering RNA (siRNA), a microRNA, an antisense nucleic acid, a ribozyme, an expression vector encoding a transdominant negative mutant, an antibody, a peptide, a nucleic acid, a protein, a peptide, a peptidomemetic, a chemical compound and a small molecule.

16. The method of claim 12, wherein the cardiovascular disease or disorder is selected from the group consisting of carotid artery disease, arteritis, myocarditis, cardiovascular inflammation, myocardial infarction, and ischemia.


SEQUENCE LISTING (22 sequences)

SEQ ID NO: 1 - DNA, 20 bp, Artificial Sequence
  Chemically Synthesized
gcaccttgagaggaacagaa

SEQ ID NO: 2 - DNA, 22 bp, Artificial Sequence
  Chemically Synthesized
gagcacccgcttagtcttatag

SEQ ID NO: 3 - DNA, 20 bp, Artificial Sequence
  Chemically Synthesized
ttcagggaatgcctggtgaa

SEQ ID NO: 4 - DNA, 19 bp, Artificial Sequence
  Chemically Synthesized
acctttgggaccagcatca

SEQ ID NO: 5 - DNA, 21 bp, Artificial Sequence
  Chemically Synthesized
gaaaagggtccctctggagaa

SEQ ID NO: 6 - DNA, 20 bp, Artificial Sequence
  Chemically Synthesized
aataccgggagcaccaagaa

SEQ ID NO: 7 - DNA, 21 bp, Artificial Sequence
  Chemically Synthesized
tgctggaaagaatggggagac

SEQ ID NO: 8 - DNA, 22 bp, Artificial Sequence
  Chemically Synthesized
ggtccagaatctcccttgtcac

SEQ ID NO: 9 - DNA, 21 bp, Artificial Sequence
  Chemically Synthesized
gtgaagaggaagacagcacag

SEQ ID NO: 10 - DNA, 21 bp, Artificial Sequence
  Chemically Synthesized
gcccattccaaccattactcc

SEQ ID NO: 11 - DNA, 21 bp, Artificial Sequence
  Chemically Synthesized
ccattggaggcaaacaactcc

SEQ ID NO: 12 - DNA, 20 bp, Artificial Sequence
  Chemically Synthesized
ttgcttcctctcaccatgca

SEQ ID NO: 13 - DNA, 21 bp, Artificial Sequence
  Chemically Synthesized
acgtcctgtgactatgggtac

SEQ ID NO: 14 - DNA, 20 bp, Artificial Sequence
  Chemically Synthesized
tctgccgcataggtgtcata

SEQ ID NO: 15 - DNA, 20 bp, Artificial Sequence
  Chemically Synthesized
cgtcattgccctgaagaaca

SEQ ID NO: 16 - DNA, 20 bp, Artificial Sequence
  Chemically Synthesized
aagggtaaccagttggggaa

SEQ ID NO: 17 - DNA, 21 bp, Artificial Sequence
  Chemically Synthesized
caaagggaggacgttcaagac

SEQ ID NO: 18 - DNA, 20 bp, Artificial Sequence
  Chemically Synthesized
tgcgtccatctccagattca

SEQ ID NO: 19 - DNA, 23 bp, Artificial Sequence
  Chemically Synthesized
ccacagcccagagctatataaac

SEQ ID NO: 20 - DNA, 21 bp, Artificial Sequence
  Chemically Synthesized
cagcagcagcagagcatataa

SEQ ID NO: 21 - DNA, 19 bp, Artificial Sequence
  Chemically Synthesized
agcaactacacggcacttt

SEQ ID NO: 22 - DNA, 21 bp, Artificial Sequence
  Chemically Synthesized
ctgggcctcgctagaaatatg